(19) United States
(12) Patent Application Publication
(10) Pub. No.: US 2024/0335475 A1
(43) Pub. Date: Oct. 10, 2024

(54) METHODS OF USING CHIMERIC ANTIGEN RECEPTORS TARGETING B-CELL MATURATION ANTIGEN AND USES THEREOF

(71) Applicants: MEMORIAL SLOAN KETTERING CANCER CENTER, New York, NY (US); EUREKA THERAPEUTICS, INC., Emeryville, CA (US)

(72) Inventors: Renier J. Brentjens, New York, NY (US); Eric L. Smith, Brookline, MA (US); Cheng Liu, Emeryville, CA (US)

(73) Assignees: MEMORIAL SLOAN KETTERING CANCER CENTER, New York, NY (US); EUREKA THERAPEUTICS, INC., Emeryville, CA (US)

(21) Appl. No.: 18/749,513

(22) Filed: Jun. 20, 2024

Related U.S. Application Data
(63) Continuation of application No. 17/228,213, filed on Apr. 12, 2021.
Application No. 17/228,213 is a division of application No. 16/844,610, filed on Apr. 9, 2020, now Pat. No. 11,000,549.
Application No. 16/844,610 is a division of application No. 15/613,638, filed on Jun. 5, 2017, now Pat. No. 10,918,665.
Application No. 15/613,638 is a continuation of application No. PCT/US2015/064112, filed on Dec. 4, 2015.
(60) Provisional application No. 62/088,309, filed on Dec. 5, 2014.

(51) Int. Cl. A61K 35/17; A61P 35/00; C07K 14/705; C07K 14/725; C07K 16/28

(52) U.S. Cl. CPC A61K 35/17; A61P 35/00; C07K 14/7051; C07K 14/70521; C07K 16/2878; C07K 2317/34; C07K 2317/622; C07K 2317/73; C07K 2317/92; C07K 2319/02; C07K 2319/03; C07K 2319/33; C12N 2740/10043; C12N 2840/203

(57) ABSTRACT

The presently disclosed subject matter provides for methods and compositions for treating multiple myeloma. It relates to chimeric antigen receptors (CARs) that specifically target B cell maturation antigen (BCMA), and immunoresponsive cells comprising such CARs. The presently disclosed BCMA-specific CARs have enhanced immune-activating properties, including anti-tumor activity.

### CROSS-REFERENCE TO RELATED APPLICATIONS

This application is a Continuation Application of U.S. patent application Ser. No. 17/228,213, filed Apr. 12, 2021, which is a Divisional of U.S. Pat. No. 11,000,549, issued May 11, 2021, which is a Divisional of U.S. Pat. No. 10,918,665, issued Feb. 16, 2021, which is a Continuation of International Application Serial No. PCT/US2015/064112, filed Dec. 4, 2015, which claims priority to U.S. Provisional Patent Application Ser. No. 62/088,309, filed Dec. 5, 2014, the contents of each of which are incorporated by reference in their entirety, and to each of which priority is claimed.

### SEQUENCE LISTING

The specification further incorporates by reference the Sequence Listing electronically submitted herewith. The Sequence Listing XML file, identified as 0893330524.xml, is 409,600 bytes and was created on Jun. 20, 2024. The Sequence Listing electronically filed herewith, does not extend beyond the scope of the specification and thus does not contain new matter.

### INTRODUCTION

The presently disclosed subject matter provides for methods and compositions for treating cancer. It relates to chimeric antigen receptors (CARs) that specifically target B-cell maturation antigen (BCMA), immunoresponsive cells comprising such CARs, and methods of using such cells for treating cancer (e.g., multiple myeloma).

### BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Multiple myeloma (MM) is the second most common hematologic malignancy.⁸ Approximately 25% of patients have high-risk cytogenetics, which portends a median survival of less then 2 years.^{9,10} While recent strides have been made, regardless of cytogenetics, the disease is still considered incurable outside the immuno-therapeutic graft versus myeloma (GvM) effect of an allogeneic transplant. However, allogeneic transplants are limited by ineligibility and high rates of transplant-associated morbidity and mortality.¹¹ Similar to the GvM effect, a potentially curative T cell effect may be achieved with minimal toxicity through autologous adoptive T cell therapy.

Myeloma may be an ideal disease to test adoptive T cell therapy. First, as indicated above, allogeneic transplants demonstrate that the T cell can be a curative treatment, even with minimal or no concomitant chemotherapy such as after non-myeloablative transplants or post-transplantation donor lymphocyte infusions. Second, conditioning chemotherapy, possibly through the mechanism of depleting regulatory T cells (Tregs), enhances the efficacy of adoptive T cell therapy,^{4,12} as such, the immediate post-autologous transplant period could be an optimal time to administer T cells, and myeloma is one of the few diseases where autologous stem cell transplantation is the standard of care. Third, the immunomodulatory drug lenalidomide may improve CAR based therapy, as has been shown in mice,¹³ and lenalidomide is commonly used to treat MM. Fourth, adoptive T cell therapy works best in bone marrow predominant disease such as ALL,^{6,7} when compared to solid tumors or extra-medullary CLL,⁴ and similar to ALL, myeloma is a disease of the bone marrow.

While there are various reasons to expect that adoptive T cell therapy may work well in MM, expanding adoptive T cell therapy to myeloma also poses unique challenges. Unlike other B-cell malignancies, CD19 expression is seen in only 2% of myeloma patients.¹⁴ Furthermore, unlike CD19, the common extracellular immunophenotypic markers in myeloma (CD138, CD38, and CD56) are all co-expressed on other essential cell types, and we predict CARs to any of these targets would lead to unacceptable “off tumor, on target” toxicity⁶ which can be fatal even in targets where antibodies are well tolerated, as was the case with a HER2 targeted CAR.¹⁵ Accordingly, there are needs for novel therapeutic strategies to design CARs targeting antigens that are highly expressed in MM cells and limited expression in normal tissues for treating multiple myeloma, which strategies capable of inducing potent tumor eradication with minimal toxicity and immunogenicity.

### SUMMARY OF THE INVENTION

The presently disclosed subject matter generally provides chimeric antigen receptors (CARs) that specifically target B-cell maturation antigen (BCMA), immunoresponsive cells comprising such CARs, and uses of these CARs and immunoresponsive cells for treating multiple myeloma.

The presently disclosed subject matter provides CARs. In one non-limiting example, the CAR comprises an extracellular antigen-binding domain, a transmembrane domain and an intracellular domain, where the extracellular antigen-binding domain is a human single-chain variable fragment (scFv) that specifically binds to B cell maturation antigen (BCMA). In certain embodiments, the human scFv specifically binds to BCMA with a binding affinity (K_{D}) of from about 1×10⁻⁹ M to about 3×10⁻⁶M. In certain embodiments, the human scFv specifically binds to BCMA with a binding affinity (K_{D}) of from about 1×10⁻⁹ M to about 1×10⁻⁸ M.

In certain embodiments, the human scFv comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence selected from the group consisting of SEQ ID NOS: 1, 5, 9, 13, 17, 21, 25, 29, 33, 37, 41, 45, 49, 53, 57, 61, and 65.

In certain embodiments, the human scFv comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence selected from the group consisting of SEQ ID NOS: 2, 6, 10, 14, 18, 22, 26, 30, 34, 38, 42, 46, 50, 54, 58, 62, and 66.

In certain embodiments, the human scFv comprises (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence selected from the group consisting of SEQ ID NO: 1, 5, 9, 13, 17, 21, 25, 29, 33, 37, 41, 45, 49, 53, 57, 61, and 65; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence selected from the group consisting of SEQ ID NOS: 2, 6, 10, 14, 18, 22, 26, 30, 34, 38, 42, 46, 50, 54, 58, 62, and 66.

In certain embodiments, the human scFv comprises a heavy chain variable region comprising an amino acid sequence selected from the group consisting of SEQ ID NOS: 1, 5, 9, 13, 17, 21, 25, 29, 33, 37, 41, 45, 49, 53, 57, 61, and 65, and conservative modifications thereof.

In certain embodiments, the human scFv comprises a light chain variable region comprising an amino acid sequence selected from the group consisting of SEQ ID NOS: 2, 6, 10, 14, 18, 22, 26, 30, 34, 38, 42, 46, 50, 54, 58, 62, and 66, and conservative modifications thereof.

In certain embodiments, the human scFv comprises (a) a heavy chain variable region comprising an amino acid sequence selected from the group consisting of SEQ ID NOS: 1, 5, 9, 13, 17, 21, 25, 29, 33, 37, 41, 45, 49, 53, 57, 61, and 65, and conservative modifications thereof, and (b) a light chain variable region comprising an amino acid sequence selected from the group consisting of SEQ ID NOS: 2, 6, 10, 14, 18, 22, 26, 30, 34, 38, 42, 46, 50, 54, 58, 62, and 66, and conservative modifications thereof.

In certain embodiments, the human scFv comprises a heavy chain variable region comprising amino acids having a sequence selected from the group consisting of: SEQ ID NOS: 1, 5, 9, 13, 17, 21, 25, 29, 33, 37, 41, 45, 49, 53, 57, 61, and 65. In certain embodiments, the human scFv comprises a light chain variable region comprising amino acids having a sequence selected from the group consisting of: SEQ ID NOS: 2, 6, 10, 14, 18, 22, 26, 30, 34, 38, 42, 46, 50, 54, 58, 62, and 66. In certain embodiments, the human scFv comprises a heavy chain variable region comprising amino acids having the sequence set forth in SEQ ID NO: 21. In certain embodiments, the human scFv comprises a heavy chain variable region comprising amino acids having the sequence set forth in SEQ ID NO: 53. In certain embodiments, the human scFv comprises a heavy chain variable region comprising amino acids having the sequence set forth in SEQ ID NO: 57. In certain embodiments, the human scFv comprises a heavy chain variable region comprising amino acids having the sequence set forth in SEQ ID NO: 61. In certain embodiments, the human scFv comprises a heavy chain variable region comprising amino acids having the sequence set forth in SEQ ID NO: 65. In certain embodiments, the human scFv comprises a light chain variable region comprising amino acids having the sequence set forth in SEQ ID NO: 22. In certain embodiments, the human scFv comprises a light chain variable region comprising amino acids having the sequence set forth in SEQ ID NO: 54. In certain embodiments, the human scFv comprises a light chain variable region comprising amino acids having the sequence set forth in SEQ ID NO: 58. In certain embodiments, the human scFv comprises a light chain variable region comprising amino acids having the sequence set forth in SEQ ID NO: 62. In certain embodiments, the human scFv comprises a light chain variable region comprising amino acids having the sequence set forth in SEQ ID NO: 66. In certain embodiments, the extracellular antigen-binding domain comprises (a) a heavy chain variable region comprising amino acids having a sequence set forth in SEQ ID NO: 1, and a light chain variable region comprising amino acids having a sequence set forth in SEQ ID NO: 2; (b) a heavy chain variable region comprising amino acids having a sequence set forth in SEQ ID NO: 5, and a light chain variable region comprising amino acids having a sequence set forth in SEQ ID NO: 6; (c) a heavy chain variable region comprising amino acids having a sequence set forth in SEQ ID NO: 9, and a light chain variable region comprising amino acids having a sequence set forth in SEQ ID NO: 10; (d) a heavy chain variable region comprising amino acids having a sequence set forth in SEQ ID NO: 13, and a light chain variable region comprising amino acids having a sequence set forth in SEQ ID NO: 14; (e) a heavy chain variable region comprising amino acids having a sequence set forth in SEQ ID NO: 17, and a light chain variable region comprising amino acids having a sequence set forth in SEQ ID NO: 18; (f) a heavy chain variable region comprising amino acids having a sequence set forth in SEQ ID NO: 21, and a light chain variable region comprising amino acids having a sequence set forth in SEQ ID NO: 22; (g) a heavy chain variable region comprising amino acids having a sequence set forth in SEQ ID NO: 25, and a light chain variable region comprising amino acids having a sequence set forth in SEQ ID NO: 26; (h) a heavy chain variable region comprising amino acids having a sequence set forth in SEQ ID NO: 29, and a light chain variable region comprising amino acids having a sequence set forth in SEQ ID NO: 30; (i) a heavy chain variable region comprising amino acids having a sequence set forth in SEQ ID NO: 33, and a light chain variable region comprising amino acids having a sequence set forth in SEQ ID NO: 34; (j) a heavy chain variable region comprising amino acids having a sequence set forth in SEQ ID NO: 37, and a light chain variable region comprising amino acids having a sequence set forth in SEQ ID NO: 38; (k) a heavy chain variable region comprising amino acids having a sequence set forth in SEQ ID NO: 41, and a light chain variable region comprising amino acids having a sequence set forth in SEQ ID NO: 42; (1) a heavy chain variable region comprising amino acids having a sequence set forth in SEQ ID NO: 45, and a light chain variable region comprising amino acids having a sequence set forth in SEQ ID NO: 46; (m) a heavy chain variable region comprising amino acids having a sequence set forth in SEQ ID NO: 49, and a light chain variable region comprising amino acids having a sequence set forth in SEQ ID NO: 50; (n) a heavy chain variable region comprising amino acids having a sequence set forth in SEQ ID NO: 53, and a light chain variable region comprising amino acids having a sequence set forth in SEQ ID NO: 54; (o) a heavy chain variable region comprising amino acids having a sequence set forth in SEQ ID NO: 57, and a light chain variable region comprising amino acids having a sequence set forth in SEQ ID NO: 58; (p) a heavy chain variable region comprising amino acids having a sequence set forth in SEQ ID NO: 61, and a light chain variable region comprising amino acids having a sequence set forth in SEQ ID NO: 62; or (q) a heavy chain variable region comprising amino acids having a sequence set forth in SEQ ID NO: 65, and a light chain variable region comprising amino acids having a sequence set forth in SEQ ID NO: 66. In certain embodiments, the human scFv comprises a heavy chain variable region comprising amino acids having the sequence set forth in SEQ ID NO: 21; and a light chain variable region comprising amino acids having the sequence set forth in SEQ ID NO: 22. In certain embodiments, the human scFv comprises a heavy chain variable region comprising amino acids having the sequence set forth in SEQ ID NO: 53; and a light chain variable region comprising amino acids having the sequence set forth in SEQ ID NO: 54. In certain embodiments, the human scFv comprises a heavy chain variable region comprising amino acids having the sequence set forth in SEQ ID NO: 57; and a light chain variable region comprising amino acids having the sequence set forth in SEQ ID NO: 58. In certain embodiments, the human scFv comprises a heavy chain variable region comprising amino acids having the sequence set forth in SEQ ID NO: 61; and a light chain variable region comprising amino acids having the sequence set forth in SEQ ID NO: 62. In certain embodiments, the human scFv comprises a heavy chain variable region comprising amino acids having the sequence set forth in SEQ ID NO: 65; and a light chain variable region comprising amino acids having the sequence set forth in SEQ ID NO: 66.

In certain non-limiting embodiments, the human scFv comprises both of said heavy and light chains, optionally with a linker sequence, for example a linker peptide, between the heavy chain variable region and the light chain variable region. For example, in certain non-limiting embodiments, the human scFv comprises (i) a heavy chain variable region comprising amino acids having the sequence set forth in SEQ ID NO: 53 and (ii) a light chain variable region comprising amino acids having the sequence set forth in SEQ ID NO: 54, optionally with (iii) a linker sequence, for example a linker peptide, between the heavy chain variable region and the light chain variable region. In certain embodiments, the human scFv comprises (i) a heavy chain variable region comprising amino acids having the sequence set forth in SEQ ID NO: 21 and (ii) a light chain variable region comprising amino acids having the sequence set forth in SEQ ID NO: 22, optionally with (iii) a linker sequence, for example a linker peptide, between the heavy chain variable region and the light chain variable region. In certain embodiments, the human scFv comprises (i) a heavy chain variable region comprising amino acids having the sequence set forth in SEQ ID NO: 57 and (ii) a light chain variable region comprising amino acids having the sequence set forth in SEQ ID NO: 58, optionally with (iii) a linker sequence, for example a linker peptide, between the heavy chain variable region and the light chain variable region. In certain embodiments, the human scFv comprises (i) a heavy chain variable region comprising amino acids having the sequence set forth in SEQ ID NO: 61 and (ii) a light chain variable region comprising amino acids having the sequence set forth in SEQ ID NO: 62, optionally with (iii) a linker sequence, for example a linker peptide, between the heavy chain variable region and the light chain variable region. In certain embodiments, the human scFv comprises (i) a heavy chain variable region comprising amino acids having the sequence set forth in SEQ ID NO: 65 and (ii) a light chain variable region comprising amino acids having the sequence set forth in SEQ ID NO: 66, optionally with (iii) a linker sequence, for example a linker peptide, between the heavy chain variable region and the light chain variable region.

In certain embodiments, the human scFv comprises (a) a heavy chain variable region CDR3 comprising an amino acid sequence selected from the group consisting of SEQ ID NOS: 91, 97, 103, 109, 115, 121, 127, 133, 139, 145, 151, 157, 163, 169, 175, 181, and 187; and (b) a light chain variable region CDR3 comprising an amino acid sequence selected from the group consisting of SEQ ID NOS: 94, 100, 106, 112, 118, 124, 130, 136, 142, 148, 154, 160, 166, 172, 178, 184, and 190. In certain embodiments, the human scFv comprises (a) a heavy chain variable region CDR2 comprising an amino acid sequence selected from the group consisting of SEQ ID NOs: 90, 96, 102, 108, 114, 120, 126, 132, 138, 144, 150, 156, 162, 168, 174, 180, and 186, and conservative modifications thereof; and (b) a light chain variable region CDR2 comprising an amino acid sequence selected from the group consisting of SEQ ID NOS: 93, 99, 105, 111, 117, 123, 129, 135, 141, 147, 153, 159, 165, 171, 177, 183, and 189, and conservative modifications thereof.

In certain embodiments, the human scFv comprises (a) a heavy chain variable region CDR1 comprising an amino acid sequence selected from the group consisting of SEQ ID NOs: 89, 95, 101, 107, 113, 119, 125, 131, 137, 143, 149, 155, 161, 167, 173, 179, and 185, and conservative modifications thereof; and (b) a light chain variable region CDR1 comprising an amino acid sequence selected from the group consisting of SEQ ID NOS: 92, 98, 104, 110, 116, 122, 128, 134, 140, 146, 152, 158, 164, 170, 176, 182, and 188, and conservative modifications thereof. In certain embodiments, the human scFv comprises: (a) a heavy chain variable region CDR1 comprising an amino acid sequence selected from the group consisting of SEQ ID NOS: 89, 95, 101, 107, 113, 119, 125, 131, 137, 143, 149, 155, 161, 167, 173, 179, and 185; (b) a heavy chain variable region CDR2 comprising an amino acid sequence selected from the group consisting of SEQ ID NOs: 90, 96, 102, 108, 114, 120, 126, 132, 138, 144, 150, 156, 162, 168, 174, 180, and 186; (c) a heavy chain variable region CDR3 comprising an amino acid sequence selected from the group consisting of SEQ ID NOS: 91, 97, 103, 109, 115, 121, 127, 133, 139, 145, 151, 157, 163, 169, 175, 181, and 187; (d) a light chain variable region CDR1 comprising an amino acid sequence selected from the group consisting of SEQ ID NOs: 92, 98, 104, 110, 116, 122, 128, 134, 140, 146, 152, 158, 164, 170, 176, 182, and 188; (e) a light chain variable region CDR2 comprising an amino acid sequence selected from the group consisting of SEQ ID NOs: 93, 99, 105, 111, 117, 123, 129, 135, 141, 147, 153, 159, 165, 171, 177, 183, and 189; and (f) a light chain variable region CDR3 comprising an amino acid sequence selected from the group consisting of SEQ ID NOs: 94, 100, 106, 112, 118, 124, 130, 136, 142, 148, 154, 160, 166, 172, 178, 184, and 190. In certain embodiments, the human scFv comprises (a) a heavy chain variable region CDR1 comprising amino acids having the sequence set forth in SEQ ID NO: 89 or conservative modifications thereof; a heavy chain variable region CDR2 comprising amino acids having the sequence set forth in SEQ ID NO: 90 or conservative modifications thereof; and a heavy chain variable region CDR3 comprising amino acids having the sequence set forth in SEQ ID NO: 91 or conservative modifications thereof; (b) a heavy chain variable region CDR1 comprising amino acids having the sequence set forth in SEQ ID NO: 95 or conservative modifications thereof; a heavy chain variable region CDR2 comprising amino acids having the sequence set forth in SEQ ID NO: 96 or conservative modifications thereof; and a heavy chain variable region CDR3 comprising amino acids having the sequence set forth in SEQ ID NO: 97 or conservative modifications thereof; (c) a heavy chain variable region CDR1 comprising amino acids having the sequence set forth in SEQ ID NO: 101 or conservative modifications thereof; a heavy chain variable region CDR2 comprising amino acids having the sequence set forth in SEQ ID NO: 102 or conservative modifications thereof; and a heavy chain variable region CDR3 comprising amino acids having the sequence set forth in SEQ ID NO: 103 or conservative modifications thereof; (d) a heavy chain variable region CDR1 comprising amino acids having the sequence set forth in SEQ ID NO: 107 or conservative modifications thereof; a heavy chain variable region CDR2 comprising amino acids having the sequence set forth in SEQ ID NO: 108 or conservative modifications thereof; and a heavy chain variable region CDR3 comprising amino acids having the sequence set forth in SEQ ID NO: 109 or conservative modifications thereof; (e) a heavy chain variable region CDR1 comprising amino acids having the sequence set forth in SEQ ID NO: 113 or conservative modifications thereof; a heavy chain variable region CDR2 comprising amino acids having the sequence set forth in SEQ ID NO: 114 or conservative modifications thereof; and a heavy chain variable region CDR3 comprising amino acids having the sequence set forth in SEQ ID NO: 115 or conservative modifications thereof; (f) a heavy chain variable region CDR1 comprising amino acids having the sequence set forth in SEQ ID NO: 119 or conservative modifications thereof; a heavy chain variable region CDR2 comprising amino acids having the sequence set forth in SEQ ID NO: 120 or conservative modifications thereof; and a heavy chain variable region CDR3 comprising amino acids having the sequence set forth in SEQ ID NO: 121 or conservative modifications thereof; (g) a heavy chain variable region CDR1 comprising amino acids having the sequence set forth in SEQ ID NO: 125 or conservative modifications thereof; a heavy chain variable region CDR2 comprising amino acids having the sequence set forth in SEQ ID NO: 126 or conservative modifications thereof; and a heavy chain variable region CDR3 comprising amino acids having the sequence set forth in SEQ ID NO: 127 or conservative modifications thereof; (h) a heavy chain variable region CDR1 comprising amino acids having the sequence set forth in SEQ ID NO: 131 or conservative modifications thereof; a heavy chain variable region CDR2 comprising amino acids having the sequence set forth in SEQ ID NO: 132 or conservative modifications thereof; and a heavy chain variable region CDR3 comprising amino acids having the sequence set forth in SEQ ID NO: 133 or conservative modifications thereof; (i) a heavy chain variable region CDR1 comprising amino acids having the sequence set forth in SEQ ID NO: 137 or conservative modifications thereof; a heavy chain variable region CDR2 comprising amino acids having the sequence set forth in SEQ ID NO: 138 or conservative modifications thereof; and a heavy chain variable region CDR3 comprising amino acids having the sequence set forth in SEQ ID NO: 139 or conservative modifications thereof; (j) a heavy chain variable region CDR1 comprising amino acids having the sequence set forth in SEQ ID NO: 143 or conservative modifications thereof; a heavy chain variable region CDR2 comprising amino acids having the sequence set forth in SEQ ID NO: 144 or conservative modifications thereof; and a heavy chain variable region CDR3 comprising amino acids having the sequence set forth in SEQ ID NO: 145 or conservative modifications thereof; (k) a heavy chain variable region CDR1 comprising amino acids having the sequence set forth in SEQ ID NO: 149 or conservative modifications thereof; a heavy chain variable region CDR2 comprising amino acids having the sequence set forth in SEQ ID NO: 150 or conservative modifications thereof; and a heavy chain variable region CDR3 comprising amino acids having the sequence set forth in SEQ ID NO: 151 or conservative modifications thereof; (1) a heavy chain variable region CDR1 comprising amino acids having the sequence set forth in SEQ ID NO: 155 or conservative modifications thereof; a heavy chain variable region CDR2 comprising amino acids having the sequence set forth in SEQ ID NO: 156 or conservative modifications thereof; and a heavy chain variable region CDR3 comprising amino acids having the sequence set forth in SEQ ID NO: 157 or conservative modifications thereof; (m) a heavy chain variable region CDR1 comprising amino acids having the sequence set forth in SEQ ID NO: 161 or conservative modifications thereof; a heavy chain variable region CDR2 comprising amino acids having the sequence set forth in SEQ ID NO: 162 or conservative modifications thereof; and a heavy chain variable region CDR3 comprising amino acids having the sequence set forth in SEQ ID NO: 163 or conservative modifications thereof; (n) a heavy chain variable region CDR1 comprising amino acids having the sequence set forth in SEQ ID NO: 167 or conservative modifications thereof; a heavy chain variable region CDR2 comprising amino acids having the sequence set forth in SEQ ID NO: 168 or conservative modifications thereof; and a heavy chain variable region CDR3 comprising amino acids having the sequence set forth in SEQ ID NO: 169 or conservative modifications thereof; (o) a heavy chain variable region CDR1 comprising amino acids having the sequence set forth in SEQ ID NO: 173 or conservative modifications thereof; a heavy chain variable region CDR2 comprising amino acids having the sequence set forth in SEQ ID NO: 174 or conservative modifications thereof; and a heavy chain variable region CDR3 comprising amino acids having the sequence set forth in SEQ ID NO: 175 or conservative modifications thereof; (p) a heavy chain variable region CDR1 comprising amino acids having the sequence set forth in SEQ ID NO: 179 or conservative modifications thereof; a heavy chain variable region CDR2 comprising amino acids having the sequence set forth in SEQ ID NO: 180 or conservative modifications thereof; and a heavy chain variable region CDR3 comprising amino acids having the sequence set forth in SEQ ID NO: 181 or conservative modifications thereof; or (q) a heavy chain variable region CDR1 comprising amino acids having the sequence set forth in SEQ ID NO: 185 or conservative modifications thereof; a heavy chain variable region CDR2 comprising amino acids having the sequence set forth in SEQ ID NO: 186 or conservative modifications thereof; and a heavy chain variable region CDR3 comprising amino acids having the sequence set forth in SEQ ID NO: 187 or conservative modifications thereof. In certain embodiments, the human scFv comprises: a heavy chain variable region CDR1 comprising amino acids having the sequence set forth in SEQ ID NO: 167; a heavy chain variable region CDR2 comprising amino acids having the sequence set forth in SEQ ID NO: 168; and a heavy chain variable region CDR3 comprising amino acids having the sequence set forth in SEQ ID NO: 169. In certain embodiments, the human scFv comprises: a heavy chain variable region CDR1 comprising amino acids having the sequence set forth in SEQ ID NO: 119; a heavy chain variable region CDR2 comprising amino acids having the sequence set forth in SEQ ID NO: 120; and a heavy chain variable region CDR3 comprising amino acids having the sequence set forth in SEQ ID NO: 121. In certain embodiments, the human scFv comprises: a heavy chain variable region CDR1 comprising amino acids having the sequence set forth in SEQ ID NO: 173; a heavy chain variable region CDR2 comprising amino acids having the sequence set forth in SEQ ID NO: 174; and a heavy chain variable region CDR3 comprising amino acids having the sequence set forth in SEQ ID NO: 175. In certain embodiments, the human scFv comprises: a heavy chain variable region CDR1 comprising amino acids having the sequence set forth in SEQ ID NO: 179; a heavy chain variable region CDR2 comprising amino acids having the sequence set forth in SEQ ID NO: 180; and a heavy chain variable region CDR3 comprising amino acids having the sequence set forth in SEQ ID NO: 181. In certain embodiments, the human scFv comprises: a heavy chain variable region CDR1 comprising amino acids having the sequence set forth in SEQ ID NO: 185; a heavy chain variable region CDR2 comprising amino acids having the sequence set forth in SEQ ID NO: 186; and a heavy chain variable region CDR3 comprising amino acids having the sequence set forth in SEQ ID NO: 187.

In certain embodiments, the human scFv comprises (a) a light chain variable region CDR1 comprising amino acids having the sequence set forth in SEQ ID NO: 92 or conservative modifications thereof; a light chain variable region CDR2 comprising amino acids having the sequence set forth in SEQ ID NO: 93 or conservative modifications thereof; and a light chain variable region CDR3 comprising amino acids having the sequence set forth in SEQ ID NO: 94 or conservative modifications thereof; (b) a light chain variable region CDR1 comprising amino acids having the sequence set forth in SEQ ID NO: 98 or conservative modifications thereof; a light chain variable region CDR2 comprising amino acids having the sequence set forth in SEQ ID NO: 99 or conservative modifications thereof; and a light chain variable region CDR3 comprising amino acids having the sequence set forth in SEQ ID NO: 100 or conservative modifications thereof; (c) a light chain variable region CDR1 comprising amino acids having the sequence set forth in SEQ ID NO: 104 or conservative modifications thereof; a light chain variable region CDR2 comprising amino acids having the sequence set forth in SEQ ID NO: 105 or conservative modifications thereof; and a light chain variable region CDR3 comprising amino acids having the sequence set forth in SEQ ID NO: 106 or conservative modifications thereof; (d) a light chain variable region CDR1 comprising amino acids having the sequence set forth in SEQ ID NO: 110 or conservative modifications thereof; a light chain variable region CDR2 comprising amino acids having the sequence set forth in SEQ ID NO: 111 or conservative modifications thereof; and a light chain variable region CDR3 comprising amino acids having the sequence set forth in SEQ ID NO: 112 or conservative modifications thereof; (e) a light chain variable region CDR1 comprising amino acids having the sequence set forth in SEQ ID NO: 116 or conservative modifications thereof; a light chain variable region CDR2 comprising amino acids having the sequence set forth in SEQ ID NO: 117 or conservative modifications thereof; and a light chain variable region CDR3 comprising amino acids having the sequence set forth in SEQ ID NO: 118 or conservative modifications thereof; (f) a light chain variable region CDR1 comprising amino acids having the sequence set forth in SEQ ID NO: 122 or conservative modifications thereof; a light chain variable region CDR2 comprising amino acids having the sequence set forth in SEQ ID NO: 123 or conservative modifications thereof; and a light chain variable region CDR3 comprising amino acids having the sequence set forth in SEQ ID NO: 124 or conservative modifications thereof; (g) a light chain variable region CDR1 comprising amino acids having the sequence set forth in SEQ ID NO: 128 or conservative modifications thereof; a light chain variable region CDR2 comprising amino acids having the sequence set forth in SEQ ID NO: 129 or conservative modifications thereof; and a light chain variable region CDR3 comprising amino acids having the sequence set forth in SEQ ID NO: 130 or conservative modifications thereof; (h) a light chain variable region CDR1 comprising amino acids having the sequence set forth in SEQ ID NO: 134 or conservative modifications thereof; a light chain variable region CDR2 comprising amino acids having the sequence set forth in SEQ ID NO: 135 or conservative modifications thereof; and a light chain variable region CDR3 comprising amino acids having the sequence set forth in SEQ ID NO: 136 or conservative modifications thereof; (i) a light chain variable region CDR1 comprising amino acids having the sequence set forth in SEQ ID NO: 140 or conservative modifications thereof; a light chain variable region CDR2 comprising amino acids having the sequence set forth in SEQ ID NO: 141 or conservative modifications thereof; and a light chain variable region CDR3 comprising amino acids having the sequence set forth in SEQ ID NO: 142 or conservative modifications thereof; (j) a light chain variable region CDR1 comprising amino acids having the sequence set forth in SEQ ID NO: 146 or conservative modifications thereof; a light chain variable region CDR2 comprising amino acids having the sequence set forth in SEQ ID NO: 147 or conservative modifications thereof; and a light chain variable region CDR3 comprising amino acids having the sequence set forth in SEQ ID NO: 148 or conservative modifications thereof; (k) a light chain variable region CDR1 comprising amino acids having the sequence set forth in SEQ ID NO: 152 or conservative modifications thereof; a light chain variable region CDR2 comprising amino acids having the sequence set forth in SEQ ID NO: 153 or conservative modifications thereof; and a light chain variable region CDR3 comprising amino acids having the sequence set forth in SEQ ID NO: 154 or conservative modifications thereof; (1) a light chain variable region CDR1 comprising amino acids having the sequence set forth in SEQ ID NO: 158 or conservative modifications thereof; a light chain variable region CDR2 comprising amino acids having the sequence set forth in SEQ ID NO: 159 or conservative modifications thereof; and a light chain variable region CDR3 comprising amino acids having the sequence set forth in SEQ ID NO: 160 or conservative modifications thereof; (m) a light chain variable region CDR1 comprising amino acids having the sequence set forth in SEQ ID NO: 164 or conservative modifications thereof; a light chain variable region CDR2 comprising amino acids having the sequence set forth in SEQ ID NO: 165 or conservative modifications thereof; and a light chain variable region CDR3 comprising amino acids having the sequence set forth in SEQ ID NO: 166 or conservative modifications thereof; (n) a light chain variable region CDR1 comprising amino acids having the sequence set forth in SEQ ID NO: 170 or conservative modifications thereof; a light chain variable region CDR2 comprising amino acids having the sequence set forth in SEQ ID NO: 171 or conservative modifications thereof; and a light chain variable region CDR3 comprising amino acids having the sequence set forth in SEQ ID NO: 172 or conservative modifications thereof; (o) a light chain variable region CDR1 comprising amino acids having the sequence set forth in SEQ ID NO: 176 or conservative modifications thereof; a light chain variable region CDR2 comprising amino acids having the sequence set forth in SEQ ID NO: 177 or conservative modifications thereof; and a light chain variable region CDR3 comprising amino acids having the sequence set forth in SEQ ID NO: 178 or conservative modifications thereof; (p) a light chain variable region CDR1 comprising amino acids having the sequence set forth in SEQ ID NO: 182 or conservative modifications thereof; a light chain variable region CDR2 comprising amino acids having the sequence set forth in SEQ ID NO: 183 or conservative modifications thereof; and a light chain variable region CDR3 comprising amino acids having the sequence set forth in SEQ ID NO: 184 or conservative modifications thereof; or (q) a light chain variable region CDR1 comprising amino acids having the sequence set forth in SEQ ID NO: 188 or conservative modifications thereof; a light chain variable region CDR2 comprising amino acids having the sequence set forth in SEQ ID NO: 189 or conservative modifications thereof; and a light chain variable region CDR3 comprising amino acids having the sequence set forth in SEQ ID NO: 190 or conservative modifications thereof. In certain embodiments, the human scFv comprises: a light chain variable region CDR1 comprising amino acids having the sequence set forth in SEQ ID NO: 122; a light chain variable region CDR2 comprising amino acids having the sequence set forth in SEQ ID NO: 123; and a light chain variable region CDR3 comprising amino acids having the sequence set forth in SEQ ID NO: 124. In certain embodiments, the human scFv comprises: a light chain variable region CDR1 comprising amino acids having the sequence set forth in SEQ ID NO: 170; a light chain variable region CDR2 comprising amino acids having the sequence set forth in SEQ ID NO: 171; and a light chain variable region CDR3 comprising amino acids having the sequence set forth in SEQ ID NO: 172. In certain embodiments, the human scFv comprises: a light chain variable region CDR1 comprising amino acids having the sequence set forth in SEQ ID NO: 176; a light chain variable region CDR2 comprising amino acids having the sequence set forth in SEQ ID NO: 177; and a light chain variable region CDR3 comprising amino acids having the sequence set forth in SEQ ID NO: 178. In certain embodiments, the human scFv comprises: a light chain variable region CDR1 comprising amino acids having the sequence set forth in SEQ ID NO: 182; a light chain variable region CDR2 comprising amino acids having the sequence set forth in SEQ ID NO: 183; and a light chain variable region CDR3 comprising amino acids having the sequence set forth in SEQ ID NO: 184. In certain embodiments, the human scFv comprises: a light chain variable region CDR1 comprising amino acids having the sequence set forth in SEQ ID NO: 188; a light chain variable region CDR2 comprising amino acids having the sequence set forth in SEQ ID NO: 189; and a light chain variable region CDR3 comprising amino acids having the sequence set forth in SEQ ID NO: 190.

In certain embodiments, the human scFv comprises: (a) a heavy chain variable region CDR1 comprising amino acids having the sequence set forth in SEQ ID NO: 89; a heavy chain variable region CDR2 comprising amino acids having the sequence set forth in SEQ ID NO: 90; a heavy chain variable region CDR3 comprising amino acids having the sequence set forth in SEQ ID NO: 91; a light chain variable region CDR1 comprising amino acids having the sequence set forth in SEQ ID NO: 92; a light chain variable region CDR2 comprising amino acids having the sequence set forth in SEQ ID NO: 93; and a light chain variable region CDR3 comprising amino acids having the sequence set forth in SEQ ID NO: 94; (b) a heavy chain variable region CDR1 comprising amino acids having the sequence set forth in SEQ ID NO: 95; a heavy chain variable region CDR2 comprising amino acids having the sequence set forth in SEQ ID NO: 96; a heavy chain variable region CDR3 comprising amino acids having the sequence set forth in SEQ ID NO: 97; a light chain variable region CDR1 comprising amino acids having the sequence set forth in SEQ ID NO: 98; a light chain variable region CDR2 comprising amino acids having the sequence set forth in SEQ ID NO: 99; and a light chain variable region CDR3 comprising amino acids having the sequence set forth in SEQ ID NO: 100; (c) a heavy chain variable region CDR1 comprising amino acids having the sequence set forth in SEQ ID NO: 101; a heavy chain variable region CDR2 comprising amino acids having the sequence set forth in SEQ ID NO: 102; a heavy chain variable region CDR3 comprising amino acids having the sequence set forth in SEQ ID NO: 103; a light chain variable region CDR1 comprising amino acids having the sequence set forth in SEQ ID NO: 104; a light chain variable region CDR2 comprising amino acids having the sequence set forth in SEQ ID NO: 105; and a light chain variable region CDR3 comprising amino acids having the sequence set forth in SEQ ID NO: 106; (d) a heavy chain variable region CDR1 comprising amino acids having the sequence set forth in SEQ ID NO: 107; a heavy chain variable region CDR2 comprising amino acids having the sequence set forth in SEQ ID NO: 108; a heavy chain variable region CDR3 comprising amino acids having the sequence set forth in SEQ ID NO: 109; a light chain variable region CDR1 comprising amino acids having the sequence set forth in SEQ ID NO: 110; a light chain variable region CDR2 comprising amino acids having the sequence set forth in SEQ ID NO: 111; and a light chain variable region CDR3 comprising amino acids having the sequence set forth in SEQ ID NO: 112; (e) a heavy chain variable region CDR1 comprising amino acids having the sequence set forth in SEQ ID NO: 113; a heavy chain variable region CDR2 comprising amino acids having the sequence set forth in SEQ ID NO: 114; a heavy chain variable region CDR3 comprising amino acids having the sequence set forth in SEQ ID NO: 115; a light chain variable region CDR1 comprising amino acids having the sequence set forth in SEQ ID NO: 116; a light chain variable region CDR2 comprising amino acids having the sequence set forth in SEQ ID NO: 117; and a light chain variable region CDR3 comprising amino acids having the sequence set forth in SEQ ID NO: 118; (f) a heavy chain variable region CDR1 comprising amino acids having the sequence set forth in SEQ ID NO: 119; a heavy chain variable region CDR2 comprising amino acids having the sequence set forth in SEQ ID NO: 120; a heavy chain variable region CDR3 comprising amino acids having the sequence set forth in SEQ ID NO: 121; a light chain variable region CDR1 comprising amino acids having the sequence set forth in SEQ ID NO: 122; a light chain variable region CDR2 comprising amino acids having the sequence set forth in SEQ ID NO: 123; and a light chain variable region CDR3 comprising amino acids having the sequence set forth in SEQ ID NO: 124; (g) a heavy chain variable region CDR1 comprising amino acids having the sequence set forth in SEQ ID NO: 125; a heavy chain variable region CDR2 comprising amino acids having the sequence set forth in SEQ ID NO: 126; a heavy chain variable region CDR3 comprising amino acids having the sequence set forth in SEQ ID NO: 127; a light chain variable region CDR1 comprising amino acids having the sequence set forth in SEQ ID NO: 128; a light chain variable region CDR2 comprising amino acids having the sequence set forth in SEQ ID NO: 129; and a light chain variable region CDR3 comprising amino acids having the sequence set forth in SEQ ID NO: 130; (h) a heavy chain variable region CDR1 comprising amino acids having the sequence set forth in SEQ ID NO: 131; a heavy chain variable region CDR2 comprising amino acids having the sequence set forth in SEQ ID NO: 132; a heavy chain variable region CDR3 comprising amino acids having the sequence set forth in SEQ ID NO: 133; a light chain variable region CDR1 comprising amino acids having the sequence set forth in SEQ ID NO: 134; a light chain variable region CDR2 comprising amino acids having the sequence set forth in SEQ ID NO: 135; and a light chain variable region CDR3 comprising amino acids having the sequence set forth in SEQ ID NO: 136; (i) a heavy chain variable region CDR1 comprising amino acids having the sequence set forth in SEQ ID NO: 137; a heavy chain variable region CDR2 comprising amino acids having the sequence set forth in SEQ ID NO: 138; a heavy chain variable region CDR3 comprising amino acids having the sequence set forth in SEQ ID NO: 139; a light chain variable region CDR1 comprising amino acids having the sequence set forth in SEQ ID NO: 140; a light chain variable region CDR2 comprising amino acids having the sequence set forth in SEQ ID NO: 141; and a light chain variable region CDR3 comprising amino acids having the sequence set forth in SEQ ID NO: 142; (j) a heavy chain variable region CDR1 comprising amino acids having the sequence set forth in SEQ ID NO: 143; a heavy chain variable region CDR2 comprising amino acids having the sequence set forth in SEQ ID NO: 144; a heavy chain variable region CDR3 comprising amino acids having the sequence set forth in SEQ ID NO: 145; a light chain variable region CDR1 comprising amino acids having the sequence set forth in SEQ ID NO: 146; a light chain variable region CDR2 comprising amino acids having the sequence set forth in SE ID NO: 147; and a light chain variable region CDR3 comprising amino acids having the sequence set forth in SEQ ID NO: 148; (k) a heavy chain variable region CDR1 comprising amino acids having the sequence set forth in SEQ ID NO: 149; a heavy chain variable region CDR2 comprising amino acids having the sequence set forth in SEQ ID NO: 150; a heavy chain variable region CDR3 comprising amino acids having the sequence set forth in SEQ ID NO: 151; a light chain variable region CDR1 comprising amino acids having the sequence set forth in SEQ ID NO: 152; a light chain variable region CDR2 comprising amino acids having the sequence set forth in SEQ ID NO: 153; and a light chain variable region CDR3 comprising amino acids having the sequence set forth in SEQ ID NO: 154; (1) a heavy chain variable region CDR1 comprising amino acids having the sequence set forth in SEQ ID NO: 155; a heavy chain variable region CDR2 comprising amino acids having the sequence set forth in SEQ ID NO: 156; a heavy chain variable region CDR3 comprising amino acids having the sequence set forth in SEQ ID NO: 157; a light chain variable region CDR1 comprising amino acids having the sequence set forth in SEQ ID NO: 158; a light chain variable region CDR2 comprising amino acids having the sequence set forth in SEQ ID NO: 159; and a light chain variable region CDR3 comprising amino acids having the sequence set forth in SEQ ID NO: 160; (m) a heavy chain variable region CDR1 comprising amino acids having the sequence set forth in SEQ ID NO: 161; a heavy chain variable region CDR2 comprising amino acids having the sequence set forth in SEQ ID NO: 162; a heavy chain variable region CDR3 comprising amino acids having the sequence set forth in SEQ ID NO: 163; a light chain variable region CDR1 comprising amino acids having the sequence set forth in SEQ ID NO: 164; a light chain variable region CDR2 comprising amino acids having the sequence set forth in SEQ ID NO: 165; and a light chain variable region CDR3 comprising amino acids having the sequence set forth in SEQ ID NO: 166; (n) a heavy chain variable region CDR1 comprising amino acids having the sequence set forth in SEQ ID NO: 167; a heavy chain variable region CDR2 comprising amino acids having the sequence set forth in SEQ ID NO: 168; a heavy chain variable region CDR3 comprising amino acids having the sequence set forth in SEQ ID NO: 169; a light chain variable region CDR1 comprising amino acids having the sequence set forth in SEQ ID NO: 170; a light chain variable region CDR2 comprising amino acids having the sequence set forth in SEQ ID NO: 171; and a light chain variable region CDR3 comprising amino acids having the sequence set forth in SEQ ID NO: 172; (o) a heavy chain variable region CDR1 comprising amino acids having the sequence set forth in SEQ ID NO: 173; a heavy chain variable region CDR2 comprising amino acids having the sequence set forth in SEQ ID NO: 174; a heavy chain variable region CDR3 comprising amino acids having the sequence set forth in SEQ ID NO: 175; a light chain variable region CDR1 comprising amino acids having the sequence set forth in SEQ ID NO: 176; a light chain variable region CDR2 comprising amino acids having the sequence set forth in SEQ ID NO: 177; and a light chain variable region CDR3 comprising amino acids having the sequence set forth in SEQ ID NO: 178; (p) a heavy chain variable region CDR1 comprising amino acids having the sequence set forth in SEQ ID NO: 179; a heavy chain variable region CDR2 comprising amino acids having the sequence set forth in SEQ ID NO: 180; a heavy chain variable region CDR3 comprising amino acids having the sequence set forth in SEQ ID NO: 181; a light chain variable region CDR1 comprising amino acids having the sequence set forth in SEQ ID NO: 182; a light chain variable region CDR2 comprising amino acids having the sequence set forth in SEQ ID NO: 183; and a light chain variable region CDR3 comprising amino acids having the sequence set forth in SEQ ID NO: 184; or (q) a heavy chain variable region CDR1 comprising amino acids having the sequence set forth in SEQ ID NO: 185; a heavy chain variable region CDR2 comprising amino acids having the sequence set forth in SEQ ID NO: 186; a heavy chain variable region CDR3 comprising amino acids having the sequence set forth in SEQ ID NO: 187; a light chain variable region CDR1 comprising amino acids having the sequence set forth in SEQ ID NO: 188; a light chain variable region CDR2 comprising amino acids having the sequence set forth in SEQ ID NO: 189; and a light chain variable region CDR3 comprising amino acids having the sequence set forth in SEQ ID NO: 190. In certain embodiments, the human scFv comprises: a heavy chain variable region CDR1 comprising amino acids having the sequence set forth in SEQ ID NO: 119; a heavy chain variable region CDR2 comprising amino acids having the sequence set forth in SEQ ID NO: 120; a heavy chain variable region CDR3 comprising amino acids having the sequence set forth in SEQ ID NO: 121; a light chain variable region CDR1 comprising amino acids having the sequence set forth in SEQ ID NO: 122; a light chain variable region CDR2 comprising amino acids having the sequence set forth in SEQ ID NO: 123; and a light chain variable region CDR3 comprising amino acids having the sequence set forth in SEQ ID NO: 124. In certain embodiments, the human scFv comprises: a heavy chain variable region CDR1 comprising amino acids having the sequence set forth in SEQ ID NO: 167; a heavy chain variable region CDR2 comprising amino acids having the sequence set forth in SEQ ID NO: 168; a heavy chain variable region CDR3 comprising amino acids having the sequence set forth in SEQ ID NO: 169; a light chain variable region CDR1 comprising amino acids having the sequence set forth in SEQ ID NO: 170; a light chain variable region CDR2 comprising amino acids having the sequence set forth in SEQ ID NO: 171; and a light chain variable region CDR3 comprising amino acids having the sequence set forth in SEQ ID NO: 172. In certain embodiments, the human scFv comprises: a heavy chain variable region CDR1 comprising amino acids having the sequence set forth in SEQ ID NO: 173; a heavy chain variable region CDR2 comprising amino acids having the sequence set forth in SEQ ID NO: 174; a heavy chain variable region CDR3 comprising amino acids having the sequence set forth in SEQ ID NO: 175; a light chain variable region CDR1 comprising amino acids having the sequence set forth in SEQ ID NO: 176; a light chain variable region CDR2 comprising amino acids having the sequence set forth in SEQ ID NO: 177; and a light chain variable region CDR3 comprising amino acids having the sequence set forth in SEQ ID NO: 178. In certain embodiments, the human scFv comprises: a heavy chain variable region CDR1 comprising amino acids having the sequence set forth in SEQ ID NO: 179; a heavy chain variable region CDR2 comprising amino acids having the sequence set forth in SEQ ID NO: 180; a heavy chain variable region CDR3 comprising amino acids having the sequence set forth in SEQ ID NO: 181; a light chain variable region CDR1 comprising amino acids having the sequence set forth in SEQ ID NO: 182; a light chain variable region CDR2 comprising amino acids having the sequence set forth in SEQ ID NO: 183; and a light chain variable region CDR3 comprising amino acids having the sequence set forth in SEQ ID NO: 184. In certain embodiments, the human scFv comprises: a heavy chain variable region CDR1 comprising amino acids having the sequence set forth in SEQ ID NO: 185; a heavy chain variable region CDR2 comprising amino acids having the sequence set forth in SEQ ID NO: 186; a heavy chain variable region CDR3 comprising amino acids having the sequence set forth in SEQ ID NO: 187; a light chain variable region CDR1 comprising amino acids having the sequence set forth in SEQ ID NO: 188; a light chain variable region CDR2 comprising amino acids having the sequence set forth in SEQ ID NO: 189; and a light chain variable region CDR3 comprising amino acids having the sequence set forth in SEQ ID NO: 190.

In certain embodiments, the BCMA comprises the amino acid sequence set forth in SEQ ID NO: 71. In certain embodiments, the human scFv binds to an epitope region comprising amino acids 14-22 of SEQ ID NO: 71. In certain embodiments, the human scFv that binds to an epitope region comprising amino acids 14-22 of SEQ ID NO: 71 comprises a V_{H} Comprising amino acids having the sequence set forth in SEQ ID NO: 21 and a V_{L} comprising amino acids having the sequence set forth in SEQ ID NO: 22, In certain embodiments, the human scFv that binds to an epitope region comprising amino acids 14-22 of SEQ ID NO: 71 comprises a V_{H} CDR1 comprising amino acids having the sequence set forth in SEQ ID NO: 119, a V_{H} CDR2 comprising amino acids having the sequence set forth in SEQ ID NO: 120, a V_{H} CDR3 comprising amino acids having the sequence set forth in SEQ ID NO: 121, a V_{L} CDR1 comprising amino acids having the sequence set forth in SEQ ID NO: 122, a V_{L} CDR2 comprising amino acids having the sequence set forth in SEQ ID NO: 123, and a V_{L} CDR3 comprising amino acids having the sequence set forth in SEQ ID NO: 124.

In accordance with the presently disclosed subject matter, the extracellular antigen-binding domain is covalently joined to a transmembrane domain. The extracellular antigen-binding domain can comprise a signal peptide that is covalently joined to the 5′ terminus of the extracellular antigen-binding domain. In certain embodiments, the transmembrane domain of the CAR comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof. In certain embodiments, the transmembrane domain comprises a CD8 polypeptide. In certain embodiments, the transmembrane domain comprises a CD28 polypeptide.

In accordance with the presently disclosed subject matter, the intracellular domain comprises a CD3ζ polypeptide. In certain embodiments, the intracellular domain further comprises at least one signaling region. In certain embodiments, the at least one signaling region comprises a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof. In certain embodiments, the signaling region is a co-stimulatory signaling region. In certain embodiments, the co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain embodiments, the at least one signaling region comprises a 4-1BB polypeptide. In one specific non-limiting embodiment, the transmembrane domain comprises a CD28 polypeptide, the intracellular domain comprises a CD3ζ polypeptide, and the at least one signaling domain comprises a CD28 polypeptide.

In certain embodiments, the CAR is recombinantly expressed. The CAR can be expressed from a vector. In certain embodiments, the vector is a γ-retroviral rector.

The presently disclosed subject matter also provides isolated immunoresponsive cells comprising the above-described CARs. In certain embodiments, the isolated immunoresponsive cell is transduced with the CAR, for example, the CAR is constitutively expressed on the surface of the immunoresponsive cell. In certain embodiments, the isolated immunoresponsive cell is further transduced with at least one co-stimulatory ligand such that the immunoresponsive cell expresses the at least one co-stimulatory ligand. In certain embodiments, the at least one co-stimulatory ligand is selected from the group consisting of 4-1BBL, CD80, CD86, CD70, OX40L, CD48, TNFRSF14, and combinations thereof. In certain embodiments, the isolated immunoresponsive cell is further transduced with at least one cytokine such that the immunoresponsive cell secrets the at least one cytokine. In certain embodiments, the at least cytokine is selected from the group consisting of IL-2, IL-3, IL-6, IL-7, IL-11, IL-12, IL-15, IL-17, IL-21, and combinations thereof. In certain embodiments, the isolated immunoresponsive cell is selected from the group consisting of a T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated. In certain embodiments, the immunoresponsive cell is a T cell.

The presently disclosed subject matter further provides nucleic acid molecules encoding the presently disclosed CARs, vectors comprising the nucleic acid molecules, and host cells expressing such nucleic acid molecules. In certain embodiments, the nucleic acid molecule comprises nucleic acids having the sequence set forth in SEQ ID NO: 207 In certain embodiments, the nucleic acid molecule comprises nucleic acids having the sequence set forth in SEQ ID NO: 208. In certain embodiments, the nucleic acid molecule comprises nucleic acids having the sequence set forth in SEQ ID NO: 209. In certain embodiments, the nucleic acid molecule comprises nucleic acids having the sequence set forth in SEQ ID NO: 229. In certain embodiments, the nucleic acid molecule comprises nucleic acids having the sequence set forth in SEQ ID NO: 230. In certain embodiments, the nucleic acid molecule comprises nucleic acids having the sequence set forth in SEQ ID NO: 231. In certain embodiments, the nucleic acid molecule comprises nucleic acids having the sequence set forth in SEQ ID NO: 232. In certain embodiments, the nucleic acid molecule comprises nucleic acids having the sequence set forth in SEQ ID NO: 233. In certain embodiments, the nucleic acid molecule comprises nucleic acids having the sequence set forth in SEQ ID NO: 234. In certain embodiments, the nucleic acid molecule comprises nucleic acids having the sequence set forth in SEQ ID NO: 235. In certain embodiments, the vector is a γ-retroviral vector. In certain embodiments, the host cell is a T cell.

Furthermore, the presently disclosed subject matter provides methods of using the above-described immunoresponsive cell for reducing tumor burden in a subject. For example, the presently disclosed subject matter provides methods of reducing tumor burden in a subject, where the method comprises administering an effective amount of the presently disclosed immunoresponsive cell to the subject, thereby inducing tumor cell death in the subject. In certain embodiments, the method reduces the number of tumor cells. In another embodiment, the method reduces the tumor size. In yet another embodiment, the method eradicates the tumor in the subject. In certain embodiments, the tumor is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the tumor is multiple myeloma. In certain embodiments, the subject is a human. In certain embodiments, the immunoresponsive cell is a T cell.

Furthermore, the presently disclosed subject matter provides methods of using the above-described immunoresponsive cell for increasing or lengthening survival of a subject having neoplasia. For example, the presently disclosed subject matter provides methods of increasing or lengthening survival of a subject having neoplasia, where the method comprises administering an effective amount of the presently disclosed immunoresponsive cell to the subject, thereby increasing or lengthening survival of the subject. In certain embodiments, the neoplasia is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the neoplasia is multiple myeloma. In certain embodiments, the method reduces or eradicates tumor burden in the subject.

The presently disclosed subject matter also provides methods for producing an immunoresponsive cell that binds to BCMA. In one non-limiting example, the method comprises introducing into the immunoresponsive cell a nucleic acid sequence that encodes a chimeric antigen receptor (CAR), which comprises an extracellular antigen-binding domain, a transmembrane domain and an intracellular domain, wherein the extracellular antigen-binding domain comprises a human scFv that specifically binds to BCMA.

The presently disclosed subject matter further provides pharmaceutical compositions comprising an effective amount of the presently disclosed immunoresponsive cells and a pharmaceutically acceptable excipient. In certain embodiments, the pharmaceutical compositions are for treating a neoplasia. In certain embodiments, the neoplasia is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the neoplasia is multiple myeloma.

The presently disclosed subject matter further provides kits for treating a neoplasia, comprising the presently disclosed immunoresponsive cells. In certain embodiments, the kit further include written instructions for using the immunoresponsive cell for treating a neoplasia. In certain embodiments, the neoplasia is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the neoplasia is multiple myeloma.

### BRIEF DESCRIPTION OF THE FIGURES

The following Detailed Description, given by way of example, but not intended to limit the invention to specific embodiments described, may be understood in conjunction with the accompanying drawings.

FIG. 1 shows a chimeric antigen receptor targeting BCMA in accordance with one non-limiting embodiment of the presently disclosed subject matter.

FIGS. 2A-2D depict the human BCMA expression in normal tissues and human cancer cell lines.

FIG. 3 depicts expression of the presently disclosed BCMA CAR on human T cells.

FIG. 4 depicts the cross-reacting activity of human scFv's targeting human BCMA with mouse BCMA.

FIG. 5 depicts the killing activity of the presently disclosed BCMA for 3T3 cells overexpressing BCMA.

FIG. 6 depicts the killing activity of the presently disclosed BCMA for a human multiple myeloma cell line.

FIG. 7 shows a chimeric antigen receptor targeting BCMA in accordance with one non-limiting embodiment of the presently disclosed subject matter.

FIG. 8 depicts a nucleic acid molecule that encodes a BCMA-targeted CAR in accordance with one non-limiting embodiment of the presently disclosed subject matter.

FIG. 9 depicts a nucleic acid molecule that encodes a BCMA-targeted CAR in accordance with one non-limiting embodiment of the presently disclosed subject matter.

FIG. 10 depicts a nucleic acid molecule that encodes a BCMA-targeted CAR in accordance with one non-limiting embodiment of the presently disclosed subject matter.

FIG. 11 depicts a nucleic acid molecule that encodes a BCMA-targeted CAR in accordance with one non-limiting embodiment of the presently disclosed subject matter.

FIG. 12 depicts a nucleic acid molecule that encodes a BCMA-targeted CAR in accordance with one non-limiting embodiment of the presently disclosed subject matter.

FIG. 13 depicts the cytotoxicity of BCMA targeted CAR T cells for human multiple myeloma cell lines.

FIG. 14 depicts induction of cytokine secretion of BCMA targeted CAR T cells.

FIG. 15 depicts anti-tumor activity of BCMA targeted CAR T cells.

FIGS. 16A and 16B depict the killing activity of BCMA targeted CAR T cells. (A) Shows the percent of GFP⁺ tumor line at time 0. (B) Shows the killing the percent of GFP⁺ tumor line at time 36 hours.

FIG. 17 depicts epitope mapping of ET140-3.

FIG. 18 depicts epitope mapping of ET140-24.

FIG. 19 depicts epitope mapping of ET140-54.

FIG. 20 depicts epitope mapping of ET140-3, ET140-24, and ET140-54.

FIG. 21 depicts ELISA screening data of ET140-3, ET140-24, ET140-37, ET140-40, and ET140-54.

FIGS. 22A-22D depict FCAS screening data of ET140-3 (FIG. 22B), ET140-24 (FIG. 22C), ET140-37 (FIG. 22C), ET140-40 (FIG. 22D), and ET140-54 (FIG. 22D).

### DETAILED DESCRIPTION OF THE INVENTION

The presently disclosed subject matter generally provides chimeric antigen receptors (CARs) targeting BCMA. In one non-limiting example, the CAR comprises an extracellular antigen-binding domain, a transmembrane domain and an intracellular domain, where the extracellular antigen-binding domain specifically binds to BCMA. The presently disclosed subject matter also provides immunoresponsive cells (e.g., T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated) expressing the BCMA-targeted CARs, and methods of using such immunoresponsive cells for treating a tumor, e.g., multiple myeloma.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In a non-limiting example, the linker comprises amino acids having the sequence set forth in SEQ ID NO: 210 as provided below.

[SEQ ID NO: 210]
GGGGSGGGGSGGGGS.

In certain embodiments, the nucleic acid sequence encoding the amino acid sequence of SEQ ID NO: 210 is set forth in SEQ ID NO: 211, which is provided below:

[SEQ ID NO: 211]
GGTGGAGGTGGATCAGGTGGAGGTGGATCTGGTGGAGGTGGATCT.

In one non-limiting example, the linker comprises amino acids having the sequence set forth in SEQ ID NO: 69 as provided below.

[SEQ ID NO: 69]
SRGGGGSGGGGSGGGGSLEMA

In certain embodiments, the nucleic acid sequence encoding the amino acid sequence of SEQ ID NO: 69 is set forth in SEQ ID NO: 70, which is provided below:

[SEQ ID NO: 70]
tctagaggtggtggtggtagcggcggcggcggctctggtggtggtggat
ccctcgagatggcc

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 August 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, i.e. recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

As used herein, the term “analog” refers to a structurally related polypeptide or nucleic acid molecule having the function of a reference polypeptide or nucleic acid molecule.

As used herein, the term “ligand” refers to a molecule that binds to a receptor. In particular, the ligand binds a receptor on another cell, allowing for cell-to-cell recognition and/or interaction.

As used herein, the term “disease” refers to any condition or disorder that damages or interferes with the normal function of a cell, tissue, or organ. Examples of diseases include neoplasia or pathogen infection of cell.

As used herein, the term “effective amount” refers to an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasia.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which naturally includes a polypeptide of the invention.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### II. B Cell Maturation Antigen (BCMA)

BCMA is an ideal target for Adoptive T cell therapy (e.g., CAR therapy) as BCMA is involved in B cell differentiation and signaling and is known to be expressed on non-malignant differentiated B cells and plasma cells. While there might be risk of inducing a B cell aplasia, B cell aplasias induced by the CD19 CAR have been remarkably well tolerated. Several groups have confirmed BCMA multiple myeloma (MM) surface expression, with one group finding it as an alternative to CD138 as a FACS marker for malignant plasma cells from fresh or frozen patient bone marrow samples with mean relative mean fluorescence intensity (MFI) between 9-16 (n=35).^{1,2}

In certain non-limiting embodiments, BCMA is human BCMA having the amino acid sequence set forth in SEQ ID NO: 71, or fragments thereof.

SEQ ID NO: 71 is provided below:

[SEQ ID NO: 71]
MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

### III. Chimeric Antigen Receptor (CAR).

Chimeric antigen receptors (CARs) are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen binding domain (e.g., a single-chain variable fragments (scFv)) fused to a transmembrane domain, fused to cytoplasmic/intracellular domain of the T cell receptor chain. “First generation” CARs typically have the intracellular domain from the CD3ζ-chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3ζ).

In accordance with the presently disclosed subject matter, the CARs comprise an extracellular antigen-binding domain, a transmembrane domain and an intracellular domain, where the extracellular antigen-binding domain binds to BCMA. In certain embodiments, the extracellular antigen-binding domain is a scFv. In certain embodiments, the extracellular antigen-binding domain is a Fab, which is optionally crosslinked. In a certain embodiments, the extracellular binding domain is a F(ab)₂. In certain embodiments, any of the foregoing molecules may be comprised in a fusion protein with a heterologous sequence to form the extracellular antigen-binding domain. In one specific non-limiting embodiment, the extracellular antigen-binding domain comprises a human scFv that binds specifically to human BCMA.

In certain non-limiting embodiments, the extracellular antigen-binding domain of a CAR has a high binding specificity as well as high binding affinity to the BCMA. For example, in such embodiments, the extracellular antigen-binding domain of the CAR (embodied, for example, in a human scFv or an analog thereof) binds to BCMA with a dissociation constant (K_{D}) of about 3×10⁻⁶ M or less. In certain embodiments, the K_{D} is about 1×10⁻⁶ M or less, about 1×10⁻⁷ M or less, about 1×10⁻⁸ M or less, or about 1×10⁻⁹ M or less, about 1×10⁻¹⁰ M or less, or about 1×10⁻¹¹ M or less. In certain embodiments, the K_{D} is about 1×10⁻⁸ M or less. In certain embodiments, the K_{D} is from about 1×10⁻¹¹ M to about 3×10⁻⁶ M, such as from about 1×10⁻¹¹ M to about 1×10⁻¹⁰ M, from about 1×10⁻¹⁰ M to about 1×10⁻⁹ M, from about 1×10⁻⁹ M to about 1×10⁻⁸ M, from about 1×10⁻⁸ M to about 1×10⁻⁷ M, or from about 1×10⁻⁷ M to about 1×10⁻⁶ M, or from about 1×10⁻⁶ M to about 3×10⁻⁶ M. In certain embodiments, the K_{D} is from about 1×10⁻⁹ M to about 1×10⁻⁸ M. In certain embodiments, the K_{D} is from about 1×10⁻⁹ M to about 1.5×10⁻⁹ M. In certain embodiments, the K_{D} is about 1.2×10⁻⁹ M. In certain embodiments, the K_{D} is from about 4×10⁻⁹ M to about 5×10⁻⁹ M. In certain embodiments, the K_{D} is about 5×10⁻⁹ M. In certain embodiments, the K_{D} is about 4.8×10⁻⁹ M. In certain embodiments, the K_{D} is from about 8×10⁻⁹ M to about 9×10⁻⁹ M. In certain embodiments, the K_{D} is about 8×10⁻⁹ M. In certain embodiments, the K_{D} is about 8.1×10⁻⁹ M.

Binding of the extracellular antigen-binding domain (embodiment, for example, in a human scFv or an analog thereof) of a presently disclosed CAR to BCMA can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the BCMA-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the BCMA-targeted human scFv is labeled with GFP.

In certain embodiments, the extracellular antigen-binding domain of a presently disclosed CAR comprises a single-chain variable fragment (scFv). In one specific non-limiting embodiment, the extracellular antigen-binding domain of a presently disclosed CAR comprises a human scFv that specifically binds to human BCMA. In certain embodiments, the scFv are identified by screening scFv phage library with BCMA-Fc fusion protein.

### Extracellular Antigen-Binding Domain of A CAR

In certain embodiments, the extracellular antigen-binding domain (e.g., human scFv) comprises a heavy chain variable region comprising amino acids having a sequence selected from the group consisting of: SEQ ID NOS: 1, 5, 9, 13, 17, 21, 25, 29, 33, 37, 41, 45, 49, 53, 57, 61, and 65. The nucleic acid sequences encoding the amino acid sequence of SEQ ID NOS: 1, 5, 9, 13, 17, 21, 25, 29, 33, 37, 41, 45, 49, 53, 57, 61, and 65 are 3, 7, 11, 15, 19, 23, 27, 31, 35, 39, 43, 47, 51, 55, 59, 63, and 67, respectively. In certain embodiments, the extracellular antigen-binding domain (e.g., human scFv) comprises a light chain variable region comprising amino acids having a sequence selected from the group consisting of SEQ ID NOS: 2, 6, 10, 14, 18, 22, 26, 30, 34, 38, 42, 46, 50, 54, 58, 62, and 66. The nucleic acid sequences encoding the amino acid sequence of SEQ ID NOS: 2, 6, 10, 14, 18, 22, 26, 30, 34, 38, 42, 46, 50, 54, 58, 62, and 66 are 4, 8, 12, 16, 20, 24, 28, 32, 36, 40, 44, 48, 52, 56, 60, 64, and 68, respectively. The sequences of SEQ ID NOS: 1-68 are described in the following Tables 1-17.

In certain embodiments, the extracellular antigen-binding domain (e.g., scFv) comprises heavy and light chain variable regions comprising amino acid sequences that are homologous to the amino acid sequences described herein and as disclosed in Tables 1-17. For example, and not by way of limitation, the extracellular antigen-binding domain (e.g., scFv) comprises a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 1, 5, 9, 13, 17, 21, 25, 29, 33, 37, 41, 45, 49, 53, 57, 61, and 65.

In certain embodiments, the extracellular antigen-binding domain (e.g., scFv) comprises a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 2, 6, 10, 14, 18, 22, 26, 30, 34, 38, 42, 46, 50, 54, 58, 62, and 66 are 4, 8, 12, 16, 20, 24, 28, 32, 36, 40, 44, 48, 52, 56, 60, 64, and 68.

In certain embodiments, the extracellular antigen-binding domain (e.g., scFv) comprises (a) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 1, 5, 9, 13, 17, 21, 25, 29, 33, 37, 41, 45, 49, 53, 57, 61, and 65; and (b) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 2, 6, 10, 14, 18, 22, 26, 30, 34, 38, 42, 46, 50, 54, 58, 62, and 66 are 4, 8, 12, 16, 20, 24, 28, 32, 36, 40, 44, 48, 52, 56, 60, 64, and 68.

The presently disclosed subject matter further provides extracellular antigen-binding domains (e.g., scFv) that comprise heavy chain variable region and light chain variable region CDRs, e.g., CDR1s, CDR2s and CDR3s, as disclosed herein in Tables 1-17. The CDR regions are delineated using the Kabat system (Kabat, E. A., et al. (1991) Sequences of Proteins of Immunological Interest, Fifth Edition, U.S. Department of Health and Human Services, NIH Publication No. 91-3242). The presently disclosed subject matter further provides extracellular antigen-binding domains (e.g., scFv) that comprise conservative modifications of the antibody sequences disclosed herein. For example, and not by way of limitation, an extracellular antigen-binding domains (e.g., scFv) of the presently disclosed subject matter comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences disclosed herein, or conservative modifications thereof, and wherein the extracellular antigen-binding domains retain the desired functional properties.

In certain embodiments, the presently disclosed subject matter provides an extracellular antigen-binding domain (e.g., scFv) comprising a heavy chain variable region, wherein the heavy chain variable region comprises: (a) a CDR1 comprising an amino acid sequence selected from the group consisting of SEQ ID NOS: 89, 95, 101, 107, 113, 119, 125, 131, 137, 143, 149, 155, 161, 167, 173, 179, and 185, and conservative modifications thereof; (b) a CDR2 comprising an amino acid sequence selected from the group consisting of SEQ ID NOS: 90, 96, 102, 108, 114, 120, 126, 132, 138, 144, 150, 156, 162, 168, 174, 180, and 186, and conservative modifications thereof; and (c) a CDR3 comprising an amino acid sequence selected from the group consisting of SEQ ID NOS: 91, 97, 103, 109, 115, 121, 127, 133, 139, 145, 151, 157, 163, 169, 175, 181, and 187, and conservative modifications thereof.

In certain embodiments, the extracellular antigen-binding domain (e.g., scFv) comprises a light chain variable region, wherein the light chain variable region comprises: (a) a CDR1 comprising an amino acid sequence selected from the group consisting of SEQ ID NOS: 92, 98, 104, 110, 116, 122, 128, 134, 140, 146, 152, 158, 164, 170, 176, 182, and 188, and conservative modifications thereof; (b) a CDR2 comprising an amino acid sequence selected from the group consisting of SEQ ID NOS: 93, 99, 105, 111, 117, 123, 129, 135, 141, 147, 153, 159, 165, 171, 177, 183, and 189, and conservative modifications thereof; and (c) a CDR3 comprising an amino acid sequence selected from the group consisting of SEQ ID NOS: 94, 100, 106, 112, 118, 124, 130, 136, 142, 148, 154, 160, 166, 172, 178, 184, and 190, and conservative modifications thereof.

The presently disclosed subject matter provides an extracellular antigen-binding domain (e.g., scFv) comprising a heavy chain variable region comprising CDR1, CDR2, and CDR3 sequences and a light chain variable region comprising CDR1, CDR2, and CDR3 sequences, wherein: (a) the heavy chain variable region CDR3 comprises an amino acid sequence selected from the group consisting of SEQ ID NOs: 91, 97, 103, 109, 115, 121, 127, 133, 139, 145, 151, 157, 163, 169, 175, 181, and 187, and conservative modifications thereof; and (b) the light chain variable region CDR3 comprises an amino acid sequence selected from the group consisting of SEQ ID NOS: 94, 100, 106, 112, 118, 124, 130, 136, 142, 148, 154, 160, 166, 172, 178, 184, and 190, and conservative modifications thereof; wherein the extracellular antigen-binding domain specifically binds to a BCMA polypeptide (e.g., a human BCMA polypeptide). In certain embodiments, the heavy chain variable region CDR2 comprises an amino acid sequence selected from the group consisting of SEQ ID NOs: 125, 131, 137, 143, 149, 155, 161, 167, 173, 179, 185, 191, 197, 203, 209, 215, 221, 227, 233, 239, 245, 251, 257, 263, 305, 317, 329, 341, 353, 365, 377, and 389, and conservative modifications thereof; and (b) the light chain variable region CDR2 comprises an amino acid sequence selected from the group consisting of SEQ ID NOS: 93, 99, 105, 111, 117, 123, 129, 135, 141, 147, 153, 159, 165, 171, 177, 183, and 189, and conservative modifications thereof; wherein the extracellular antigen-binding domain specifically binds to a BCMA polypeptide (e.g., a human BCMA polypeptide). In certain embodiments, the heavy chain variable region CDR1 comprises an amino acid sequence selected from the group consisting of SEQ ID NOs: 89, 95, 101, 107, 113, 119, 125, 131, 137, 143, 149, 155, 161, 167, 173, 179, and 185, and conservative modifications thereof; and (b) the light chain variable region CDR1 comprises an amino acid sequence selected from the group consisting of SEQ ID NOS: 92, 98, 104, 110, 116, 122, 128, 134, 140, 146, 152, 158, 164, 170, 176, 182, and 188, and conservative modifications thereof; wherein the extracellular antigen-binding domain specifically binds to a BCMA polypeptide (e.g., a human BCMA polypeptide).

In certain embodiments, the extracellular antigen-binding domain is a human scFv, which comprises the amino acid sequence of SEQ ID NO: 72 and specifically binds to a BCMA polypeptide (e.g., a BCMA polypeptide having the amino acid sequence SEQ ID NO: 71, or fragments thereof), which is designated as ET140-192 scFv (also referred to as “ET140-42 scFv”).

In certain embodiments, the extracellular antigen-binding domain is a human scFv, which comprises a heavy chain variable region comprising amino acids having the sequence set forth in SEQ ID NO: 1 and a light chain variable region comprising amino acids having the sequence set forth in SEQ ID NO: 2, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 1. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 1, as shown in Table 1. In certain embodiments, the extracellular antigen-binding domain comprises a V_{H} comprising amino acids having the sequence set forth in SEQ ID NO: 1, as shown in Table 1. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 2, as shown in Table 1. In certain embodiments, the extracellular antigen-binding domain comprises a V_{L} comprising amino acids having the sequence set forth in SEQ ID NO: 2, as shown in Table 1. In certain embodiments, the extracellular antigen-binding domain comprises a V_{H} comprising amino acids having the sequence set forth in SEQ ID NO: 1 and a V_{L} comprising amino acids having the sequence set forth in SEQ ID NO: 2, as shown in Table 1. In certain embodiments, the extracellular antigen-binding domain comprises a V_{H} CDR1 comprising amino acids having the sequence set forth in SEQ ID NO: 89 or conservative modifications thereof, a V_{H} CDR2 comprising amino acids having the sequence set forth in SEQ ID NO: 90 or conservative modifications thereof, and a V_{H} CDR3 comprising amino acids having the sequence set forth in SEQ ID NO: 91 or conservative modifications thereof, as shown in Table 1. In certain embodiments, the extracellular antigen-binding domain comprises a V_{L} CDR1 comprising amino acids having the sequence set forth in SEQ ID NO: 92 or conservative modifications thereof, a V_{L} CDR2 comprising amino acids having the sequence set forth in SEQ ID NO: 93 or conservative modifications thereof, and a V_{L} CDR3 comprising amino acids having the sequence set forth in SEQ ID NO: 94 or conservative modifications thereof, as shown in Table 1. In certain embodiments, the extracellular antigen-binding domain comprises a V_{H} CDR1 comprising amino acids having the sequence set forth in SEQ ID NO: 89 or conservative modifications thereof, a V_{H} CDR2 comprising amino acids having the sequence set forth in SEQ ID NO: 90 or conservative modifications thereof, a V_{H} CDR3 comprising amino acids having the sequence set forth in SEQ ID NO: 91 or conservative modifications thereof, a V_{L} CDR1 comprising amino acids having the sequence set forth in SEQ ID NO: 92 or conservative modifications thereof, a V_{L} CDR2 comprising amino acids having the sequence set forth in SEQ ID NO: 93 or conservative modifications thereof, and a V_{L} CDR3 comprising amino acids having the sequence set forth in SEQ ID NO: 94 or conservative modifications thereof, as shown in Table 1. In certain embodiments, the extracellular antigen-binding domain comprises a V_{H} CDR1 comprising amino acids having the sequence set forth in SEQ ID NO: 89, a V_{H} CDR2 comprising amino acids having the sequence set forth in SEQ ID NO: 90, a V_{H} CDR3 comprising amino acids having the sequence set forth in SEQ ID NO: 91, a V_{L} CDR1 comprising amino acids having the sequence set forth in SEQ ID NO: 92, a V_{L} CDR2 comprising amino acids having the sequence set forth in SEQ ID NO: 93, and a V_{L} CDR3 comprising amino acids having the sequence set forth in SEQ ID NO: 94.

TABLE 1

| | A BCMA polypeptide having the amino acid | | |
|---|---|---|---|
| | sequence of SEQ ID NO: 71 | | |
| | CDRs | | |
| Antigen | 1 | 2 | 3 |
| V_{H} | VSSNSAAWN | YRSKWYN [SEQ | ARQGYSYYGYSDV |
| | [SEQ ID NO: | ID NO: 90] | [SEQ ID NO: |
| | 89] | | 91] |
| V_{L} | SSNIGHND | FDD [SEQ | AAWDGSLNAFV |
| | [SEQ ID NO: | ID NO: 93 | [SEQ ID NO: |
| | 92] | | 94] |

| | |
|---|---|
| Full V_{H} | QVQLQQSGPGLVKPSQTLSLTCAISGDSVSSNSAAWNWIR |
| | QSPSRGLEWLGRTYYRSKWYNDYAVSVKSRITINPDTSKN |
| | QFSLQLNSVTPEDTAVYYCARQGYSYYGYSDVWGQGTLVT |
| | VSS [SEQ ID NO: 1] |
| DNA | Caggtacagctgcagcagtcaggtccaggactggtgaagc |
| | cctcgcagaccctctcactcacctgtgccatctccgggga |
| | cagtgtctctagcaacagtgctgcttggaactggatcagg |
| | cagtccccatcgagaggccttgagtggctgggaaggacat |
| | actacaggtccaagtggtataatgattatgcagtatctgt |
| | gaaaagtcgaataaccatcaacccagacacatccaagaac |
| | cagttctccctgcagctgaactctgtgactcccgaggaca |
| | cggctgtgtattactgtgcgcgccagggttactcttacta |
| | cggttactctcgatgtttggggtcaaggtactctggtgac |
| | cgtctcctca [SEQ ID NO: 3] |
| Full V_{L} | QSVLTQPPSVSVAPRQRVTISCSGSSSNIGHNDVSWYQHL |
| | PGKAPRLLIYFDDLLPSGVSDRFSASKSGTSASLAISGLQ |
| | SEDEADYYCAAWDGSLNAFVFGTGTKVTVLG [SEQ ID |
| | NO: 2] |
| DNA | Cagtctgtgctgactcagccaccctcggtgtctgtagccc |
| | ccaggcagagggtcaccatctcgtgttctggaagcagctc |
| | caacatcggacataatgatgtaagctggtaccagcatctc |
| | ccagggaaggctcccagactcctcatctattttgatgacc |
| | tgctgccgtcaggggtctctgaccgattctctgcctccaa |
| | gtctggcacctcagcctccctggccatcagtgggctccag |
| | tctgaggatgaggctgattattactgtgcagcatgggatg |
| | gcagcctgaatgctttgtcttcggaactgggaccaaggtc |
| | accgtcctaggt [SEQ ID NO: 4] |
| scFv | QSVLTQPPSVSVAPRQRVTISCSGSSSNIGHNDVSWYQHL |
| | PGKAPRLLIYFDDLLPSGVSDRFSASKSGTSASLAISGLQ |
| | SEDEADYYCAAWDGSLNAFVFGTGTKVTVLGSRGGGGSGG |
| | GGSGGGGSLEMAQVQLQQSGPGLVKPSQTLSLTCAISGDS |
| | VSSNSAAWNWIRQSPSRGLEWLGRTYYRSKWYNDYAVSVK |
| | SRITINPDTSKNQFSLQLNSVTPEDTAVYYCARQGYSYYG |
| | YSDVWGQGTLVTVSS [SEQ ID NO: 72] |

In certain embodiments, the extracellular antigen-binding domain is a human scFv, which comprises the amino acid sequence of SEQ ID NO: 73 and specifically binds to a BCMA polypeptide (e.g., a BCMA polypeptide having the amino acid sequence SEQ ID NO: 71, or fragments thereof), which is designated as ET140-197 scFv (also referred to as “ET140-47 scFv”).

In certain embodiments, the extracellular antigen-binding domain is a human scFv, which comprises a heavy chain variable region comprising amino acids having the sequence set forth in SEQ ID NO: 5 and a light chain variable region comprising amino acids having the sequence set forth in SEQ ID NO: 6, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 2. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 5, as shown in Table 2. In certain embodiments, the extracellular antigen-binding domain comprises a V_{H} comprising amino acids having the sequence set forth in SEQ ID NO: 5, as shown in Table 2. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 6, as shown in Table 2. In certain embodiments, the extracellular antigen-binding domain comprises a V_{L} comprising amino acids having the sequence set forth in SEQ ID NO: 6, as shown in Table 2. In certain embodiments, the extracellular antigen-binding domain comprises a V_{H} comprising amino acids having the sequence set forth in SEQ ID NO: 5 and a V_{L} comprising amino acids having the sequence set forth in SEQ ID NO: 6, as shown in Table 2. In certain embodiments, the extracellular antigen-binding domain comprises a V_{H} CDR1 comprising amino acids having the sequence set forth in SEQ ID NO: 95 or conservative modifications thereof, a V_{H} CDR2 comprising amino acids having the sequence set forth in SEQ ID NO: 96 or conservative modifications thereof, and a V_{H} CDR3 comprising amino acids having the sequence set forth in SEQ ID NO: 97 or conservative modifications thereof, as shown in Table 2. In certain embodiments, the extracellular antigen-binding domain comprises a V_{L} CDR1 comprising amino acids having the sequence set forth in SEQ ID NO: 98 or conservative modifications thereof, a V_{L} CDR2 comprising amino acids having the sequence set forth in SEQ ID NO: 99 or conservative modifications thereof, and a V_{L} CDR3 comprising amino acids having the sequence set forth in SEQ ID NO: 100 or conservative modifications thereof, as shown in Table 2. In certain embodiments, the extracellular antigen-binding domain comprises a V_{H} CDR1 comprising amino acids having the sequence set forth in SEQ ID NO: 95 or conservative modifications thereof, a V_{H} CDR2 comprising amino acids having the sequence set forth in SEQ ID NO: 96 or conservative modifications thereof, a V_{H} CDR3 comprising amino acids having the sequence set forth in SEQ ID NO: 97 or conservative modifications thereof, a V_{L} CDR1 comprising amino acids having the sequence set forth in SEQ ID NO: 98 or conservative modifications thereof, a V_{L} CDR2 comprising amino acids having the sequence set forth in SEQ ID NO: 99 or conservative modifications thereof, and a V_{L} CDR3 comprising amino acids having the sequence set forth in SEQ ID NO: 100 or conservative modifications thereof, as shown in Table 2. In certain embodiments, the extracellular antigen-binding domain comprises a V_{H} CDR1 comprising amino acids having the sequence set forth in SEQ ID NO: 95, a VA CDR2 comprising amino acids having the sequence set forth in SEQ ID NO: 96, a V N CDR3 comprising amino acids having the sequence set forth in SEQ ID NO: 97, a V CDR1 comprising amino acids having the sequence set forth in SEQ ID NO: 98, a V_{L} CDR2 comprising amino acids having the sequence set forth in SEQ ID NO: 99, and a V_{L} CDR3 comprising amino acids having the sequence set forth in SEQ ID NO: 100.

TABLE 2

| | A BCMA polypeptide having the amino | | |
|---|---|---|---|
| | acid sequence of SEQ ID NO: 71 | | |
| | CDRs | | |
| Antigen | 1 | 2 | 3 |
| V_{H} | VSSNSAAWN | YRSKWYN | ARYGFSGSRFYDT |
| | [SEQ ID NO: | [SEQ ID NO: | [SEQ ID NO: |
| | 95] | 96] | 97] |
| V_{L} | SSNIGNNA | FDD [SEQ ID | AAWDDSLNGYV |
| | [SEQ ID NO: | NO: 99] | [SEQ ID NO: |
| | 98] | | 100] |

| | |
|---|---|
| Full V_{H} | QVQLQQSGPGLVKPSQTLSLTCAISGDSVSSNSAAWNWIR |
| | QSPSRGLEWLGRTYYRSKWYNDYAVSVKSRITINPDTSKN |
| | QFSLQLNSVTPEDTAVYYCARYGFSGSRFYDTWGQGTLVT |
| | VSS [SEQ ID NO: 5] |
| DNA | Caggtacagctgcagcagtcaggtccaggactggtgaagc |
| | cctcgcagaccctctcactcacctgtgccatctccgggga |
| | cagtgtctctagcaacagtgctgcttggaactggatcagg |
| | cagtccccatcgagaggccttgagtggctgggaaggacat |
| | actacaggtccaagtggtataatgattatgcagtatctgt |
| | gaaaagtcgaataaccatcaacccagacacatccaagaac |
| | cagttctccctgcagctgaactctgtgactcccgaggaca |
| | cggctgtgtattactgtgcgcgctacggtttctctggttc |
| | tcgtttctacgatacttggggtcaaggtactctggtgacc |
| | gtctcctca [SEQ ID NO: 7] |
| Full V_{L} | QPVLTQPPSVSEAPRQRVTISCSGSSSNIGNNAVNWYQQL |
| | PGKAPKLLIYFDDLLSSGVSDRFSGSKSGTSASLAISGLQ |
| | SEDEADYYCAAWDDSLNGYVFGTGTKVTVLG [SEQ |
| | ID NO: 6] |
| DNA | Cagcctgtgctgactcagccaccctcggtgtctgaagccc |
| | ccaggcagagggtcaccatctcctgttctggaagcagctc |
| | caacatcggaaataatgctgtaaactggtaccagcagctc |
| | ccaggaaaggctcccaaactcctcatctattttgatgatc |
| | tgctgtcctcaggggtctctgaccgattctctggctccaa |
| | gtctggcacctcagcctccctggccatcagtgggctccag |
| | tctgaagatgaggctgattattactgtgcagcatgggatg |
| | acagcctgaatggttatgtcttcggaactgggaccaaggt |
| | caccgtcctaggt [SEQ ID NO: 8] |
| scFv | QPVLTQPPSVSEAPRQRVTISCSGSSSNIGNNAVNWYQQL |
| | PGKAPKLLIYFDDLLSSGVSDRFSGSKSGTSASLAISGLQ |
| | SEDEADYYCAAWDDSLNGYVFGTGTKVTVLGSRGGGGSGG |
| | GGSGGGGSLEMAQVQLQQSGPGLVKPSQTLSLTCAISGDS |
| | VSSNSAAWNWIRQSPSRGLEWLGRTYYRSKWYNDYAVSVK |
| | SRITINPDTSKNQFSLQLNSVTPEDTAVYYCARYGFSGSR |
| | FYDTWGQGTLVTVSS [SEQ ID NO: 73] |

In certain embodiments, the extracellular antigen-binding domain is a human scFv, which comprises the amino acid sequence of SEQ ID NO: 74 and specifically binds to a BCMA polypeptide (e.g., a BCMA polypeptide having the amino acid sequence SEQ ID NO: 71, or fragments thereof), which is designated as ET140-180 scFv (also referred to as “ET140-30 scFv”).

In certain embodiments, the extracellular antigen-binding domain is a human scFv, which comprises a heavy chain variable region comprising amino acids having the sequence set forth in SEQ ID NO: 9 and a light chain variable region comprising amino acids having the sequence set forth in SEQ ID NO: 10, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 3. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 9, as shown in Table 3. In certain embodiments, the extracellular antigen-binding domain comprises a V_{H} comprising amino acids having the sequence set forth in SEQ ID NO: 9, as shown in Table 3. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 10, as shown in Table 3. In certain embodiments, the extracellular antigen-binding domain comprises a V_{L} comprising amino acids having the sequence set forth in SEQ ID NO: 10, as shown in Table 3. In certain embodiments, the extracellular antigen-binding domain comprises a V_{H} comprising amino acids having the sequence set forth in SEQ ID NO: 9 and a V_{L} comprising amino acids having the sequence set forth in SEQ ID NO: 10, as shown in Table 3. In certain embodiments, the extracellular antigen-binding domain comprises a V_{H} CDR1 comprising amino acids having the sequence set forth in SEQ ID NO: 101 or conservative modifications thereof, a V_{H} CDR2 comprising amino acids having the sequence set forth in SEQ ID NO: 102 or conservative modifications thereof, and a V_{H} CDR3 comprising amino acids having the sequence set forth in SEQ ID NO: 103 or conservative modifications thereof, as shown in Table 3. In certain embodiments, the extracellular antigen-binding domain comprises a V_{L} CDR1 comprising amino acids having the sequence set forth in SEQ ID NO: 104 or conservative modifications thereof, a V_{L} CDR2 comprising amino acids having the sequence set forth in SEQ ID NO: 105 or conservative modifications thereof, and a V_{L} CDR3 comprising amino acids having the sequence set forth in SEQ ID NO: 106 or conservative modifications thereof, as shown in Table 3. In certain embodiments, the extracellular antigen-binding domain comprises a V_{H} CDR1 comprising amino acids having the sequence set forth in SEQ ID NO: 101 or conservative modifications thereof, a V_{H} CDR2 comprising amino acids having the sequence set forth in SEQ ID NO: 102 or conservative modifications thereof, a V_{H} CDR3 comprising amino acids having the sequence set forth in SEQ ID NO: 103 or conservative modifications thereof, a V_{L} CDR1 comprising amino acids having the sequence set forth in SEQ ID NO: 104 or conservative modifications thereof, a V_{L} CDR2 comprising amino acids having the sequence set forth in SEQ ID NO: 105 or conservative modifications thereof, and a V_{L} CDR3 comprising amino acids having the sequence set forth in SEQ ID NO: 106 or conservative modifications thereof, as shown in Table 3. In certain embodiments, the extracellular antigen-binding domain comprises a V_{H} CDR1 comprising amino acids having the sequence set forth in SEQ ID NO: 101, a V_{H} CDR2 comprising amino acids having the sequence set forth in SEQ ID NO: 9102, a V_{H} CDR3 comprising amino acids having the sequence set forth in SEQ ID NO: 103, a V_{L} CDR1 comprising amino acids having the sequence set forth in SEQ ID NO: 104, a V_{L} CDR2 comprising amino acids having the sequence set forth in SEQ ID NO: 105, and a V_{L} CDR3 comprising amino acids having the sequence set forth in SEQ ID NO: 106.

TABLE 3

| | A BCMA polypeptide having the amino acid | | |
|---|---|---|---|
| | sequence of SEQ ID NO: 71 | | |
| | CDRs | | |
| Antigen | 1 | 2 | 3 |
| V_{H} | GGTFSSYA | IIPILGIA | ARSGYSKSIVS |
| | [SEQ ID NO: | [SEQ ID NO: | YMDY [SEQ ID |
| | 101] | 102] | NO: 103] |
| V_{L} | SSNIGSNV | RNN [SEQ ID | AAWDDSLSGYV |
| | [SEQ ID NO: | NO: 105] | [SEQ ID NO: |
| | 104] | | 106] |

| | |
|---|---|
| Full V_{H} | EVQLVQSGAEVKKPGSSVKVSCKASGGTFSSYAISWVRQA |
| | PGQGLEWMGRIIPILGIANYAQKFQGRVTMTEDTSTDTAY |
| | MELSSLRSEDTAVYYCARSGYSKSIVSYMDYWGQGTLVTV |
| | SS [SEQ ID NO: 9] |
| DNA | Gaggtccagctggtgcagtctggagctgaggtgaagaagc |
| | ctgggtcctcggtgaaggtctcctgcaaggcttctggagg |
| | caccttcagcagctatgctatcagctgggtgcgacaggcc |
| | cctggacaagggcttgagtggatgggaaggatcatcccta |
| | tccttggtatagcaaactacgcacagaagttccagggcag |
| | agtcaccatgaccgaggacacatctacagacacagcctac |
| | atggagctgagcagcctgagatctgaggacacggccgtgt |
| | attactgtgcgcgctctggttactctaaatctatcgtttc |
| | ttacatggattactggggtcaaggtactctggtgaccgtc |
| | tcctca [SEQ ID NO: 11] |
| Full V_{L} | LPVLTQPPSTSGTPGQRVTVSCSGSSSNIGSNVVFWYQQL |
| | PGTAPKLVIYRNNQRPSGVPDRFSVSKSGTSASLAISGLR |
| | SEDEADYYCAAWDDSLSGYVFGTGTKVTVLG [SEQ |
| | ID NO: 10] |
| DNA | Ctgcctgtgctgactcagcccccctccacgtctgggaccc |
| | ccgggcagagggtcaccgtctcttgttctggaagcagctc |
| | caacatcggaagtaatgttgtattctggtaccagcagctc |
| | ccaggcacggcccccaaacttgtcatctataggaataatc |
| | aacggccctcaggggtccctgaccgattctctgtctccaa |
| | gtctggcacctcagcctccctggccatcagtgggctccgg |
| | tccgaggacgaggctgattattattgtgcagcttgggatg |
| | acagcctgagtggttatgtcttcggaactgggaccaaggt |
| | caccgtcctaggt [SEQ ID NO: 12] |
| scFv | LPVLTQPPSTSGTPGQRVTVSCSGSSSNIGSNVVFWYQQL |
| | PGTAPKLVIYRNNQRPSGVPDRFSVSKSGTSASLAISGLR |
| | SEDEADYYCAAWDDSLSGYVFGTGTKVTVLGSRGGGGSGG |
| | GGSGGGGSLEMAEVQLVQSGAEVKKPGSSVKVSCKASGGT |
| | FSSYAISWVRQAPGQGLEWMGRIIPILGIANYAQKFQGRV |
| | TMTEDTSTDTAYMELSSLRSEDTAVYYCARSGYSKSIVSY |
| | MDYWGQGTLVTVSS [SEQ ID NO: 74] |

In certain embodiments, the extracellular antigen-binding domain is a human scFv, which comprises the amino acid sequence of SEQ ID NO: 75 and specifically binds to a BCMA polypeptide (e.g., a BCMA polypeptide having the amino acid sequence SEQ ID NO: 71, or fragments thereof), which is designated as ET140-172 scFv (also referred to as “ET140-22 scFv”).

In certain embodiments, the extracellular antigen-binding domain is a human scFv, which comprises a heavy chain variable region comprising amino acids having the sequence set forth in SEQ ID NO: 13 and a light chain variable region comprising amino acids having the sequence set forth in SEQ ID NO: 14, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 4. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 13, as shown in Table 4. In certain embodiments, the extracellular antigen-binding domain comprises a V_{H} comprising amino acids having the sequence set forth in SEQ ID NO: 13, as shown in Table 4. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 14, as shown in Table 4. In certain embodiments, the extracellular antigen-binding domain comprises a V_{L} comprising amino acids having the sequence set forth in SEQ ID NO: 14, as shown in Table 4. In certain embodiments, the extracellular antigen-binding domain comprises a V_{H} comprising amino acids having the sequence set forth in SEQ ID NO: 13 and a V_{L} comprising amino acids having the sequence set forth in SEQ ID NO: 14, as shown in Table 4. In certain embodiments, the extracellular antigen-binding domain comprises a V_{H} CDR1 comprising amino acids having the sequence set forth in SEQ ID NO: 107 or conservative modifications thereof, a V_{H} CDR2 comprising amino acids having the sequence set forth in SEQ ID NO: 108 or conservative modifications thereof, and a V_{H} CDR3 comprising amino acids having the sequence set forth in SEQ ID NO: 109 or conservative modifications thereof, as shown in Table 4. In certain embodiments, the extracellular antigen-binding domain comprises a V_{L} CDR1 comprising amino acids having the sequence set forth in SEQ ID NO: 110 or conservative modifications thereof, a V_{L} CDR2 comprising amino acids having the sequence set forth in SEQ ID NO: 111 or conservative modifications thereof, and a V_{L} CDR3 comprising amino acids having the sequence set forth in SEQ ID NO: 112 or conservative modifications thereof, as shown in Table 4. In certain embodiments, the extracellular antigen-binding domain comprises a V_{H} CDR1 comprising amino acids having the sequence set forth in SEQ ID NO: 107 or conservative modifications thereof, a V_{H} CDR2 comprising amino acids having the sequence set forth in SEQ ID NO: 108 or conservative modifications thereof, a V_{H} CDR3 comprising amino acids having the sequence set forth in SEQ ID NO: 109 or conservative modifications thereof, a V_{L} CDR1 comprising amino acids having the sequence set forth in SEQ ID NO: 110 or conservative modifications thereof, a V_{L} CDR2 comprising amino acids having the sequence set forth in SEQ ID NO: 111 or conservative modifications thereof, and a V_{L} CDR3 comprising amino acids having the sequence set forth in SEQ ID NO: 112 or conservative modifications thereof, as shown in Table 4. In certain embodiments, the extracellular antigen-binding domain comprises a V_{H} CDR1 comprising amino acids having the sequence set forth in SEQ ID NO: 107, a V_{H} CDR2 comprising amino acids having the sequence set forth in SEQ ID NO: 108, a V_{H} CDR3 comprising amino acids having the sequence set forth in SEQ ID NO: 109, a V_{L} CDR1 comprising amino acids having the sequence set forth in SEQ ID NO: 110, a V_{L} CDR2 comprising amino acids having the sequence set forth in SEQ ID NO: 111, and a V_{L} CDR3 comprising amino acids having the sequence set forth in SEQ ID NO: 112.

TABLE 4

| | A BCMA polypeptide having the amino acid sequence of SEQ ID NO: 71 | | |
|---|---|---|---|
| Antigen | | | |
| CDRs | 1 | 2 | 3 |
| V_{H} | GYTFTSYY [SEQ | INPSGGST [SEQ | ARSQWGGVLDY |
| | ID NO: 107] | ID NO: 108] | [SEQ ID NO: 109] |
| V_{L} | SSNIGARYD [SEQ | GNN [SEQ ID | QSYDSSLSASV |
| | ID NO: 110] | NO: 111] | [SEQ ID NO: 112] |

| | |
|---|---|
| Full V_{H} | EVQLVQSGAEVKKPGASVKVSCKASGYTFTSYYMHWVRQAPGQ |
| | GLEWMGIINPSGGSTSYAQKFQGRVTMTRDTSTSTVYMELSSLRS |
| | EDTAVYYCARSQWGGVLDYWGQGTLVTVSS [SEQ ID NO: 13] |
| DNA | Gaggtccagctggtacagtctggggctgaggtgaagaagcctggggcctcagtgaaggtttcctgcaa |
| | ggcatctggatacaccttcaccagctactatatgcactgggtgcgacaggcccctggacaagggcttgag |
| | tggatgggaataatcaaccctagtggtggtagcacaagctacgcacagaagttccagggcagagtcacc |
| | atgaccagggacacgtccacgagcacagtctacatggagctgagcagcctgagatctgaggacacggc |
| | cgtgtattactgtgcgcgctctcagtggggtggtgttctggattactggggtcaaggtactctggtgaccgt |
| | ctcctca [SEQ ID NO: 15] |
| Full V_{L} | QSVVTQPPSVSGAPGQRVTISCSGSSSNIGARYDVQWYQQLPGTAP |
| | KLLIFGNNNRPSGVPDRFSGSKSGTSASLAITGLQAEDEADYYCQS |
| | YDSSLSASVFGGGTKLTVLG [SEQ ID NO: 14] |
| DNA | Cagtctgtcgtgacgcagccgccctcagtgtctggggccccagggcagagggtcaccatctcctgcag |
| | tgggagcagctccaacatcggggcacgttatgatgttcagtggtaccagcagcttccaggaacagcccc |
| | caaactcctcatctttggtaacaacaatcggccctcaggggtccctgaccgattctctggctccaagtctgg |
| | cacgtcagcctccctggccatcactgggctccaggctgaggatgaggctgattattactgccagtcctatg |
| | acagcagcctgagtgcttcggtgttcggcggagggaccaagctgaccgtcctaggt [SEQ ID NO: 16] |
| scFv | QSVVTQPPSVSGAPGQRVTISCSGSSSNIGARYDVQWYQQLPGTAP |
| | KLLIFGNNNRPSGVPDRFSGSKSGTSASLAITGLQAEDEADYYCQS |
| | YDSSLSASVFGGGTKLTVLGSRGGGGSGGGGSGGGGSLEMAEVQ |
| | LVQSGAEVKKPGASVKVSCKASGYTFTSYYMHWVRQAPGQGLE |
| | WMGIINPSGGSTSYAQKFQGRVTMTRDTSTSTVYMELSSLRSEDT |
| | AVYYCARSQWGGVLDYWGQGTLVTVSS [SEQ ID NO: 75] |

In certain embodiments, the extracellular antigen-binding domain is a human scFv, which comprises the amino acid sequence of SEQ ID NO: 76 and specifically binds to a BCMA polypeptide (e.g., a BCMA polypeptide having the amino acid sequence SEQ ID NO: 71, or fragments thereof), which is designated as ET140-157 scFv (also referred to as “ET140-7 scFv”).

In certain embodiments, the extracellular antigen-binding domain is a human scFv, which comprises a heavy chain variable region comprising amino acids having the sequence set forth in SEQ ID NO: 17 and a light chain variable region comprising amino acids having the sequence set forth in SEQ ID NO: 18, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 5. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 17, as shown in Table 5. In certain embodiments, the extracellular antigen-binding domain comprises a V_{H} comprising amino acids having the sequence set forth in SEQ ID NO: 17, as shown in Table 5. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 18, as shown in Table 5. In certain embodiments, the extracellular antigen-binding domain comprises a V_{L} comprising amino acids having the sequence set forth in SEQ ID NO: 18, as shown in Table 5. In certain embodiments, the extracellular antigen-binding domain comprises a V_{H} comprising amino acids having the sequence set forth in SEQ ID NO: 17 and a V_{L} comprising amino acids having the sequence set forth in SEQ ID NO: 18, as shown in Table 5. In certain embodiments, the extracellular antigen-binding domain comprises a V_{H} CDR1 comprising amino acids having the sequence set forth in SEQ ID NO: 113 or conservative modifications thereof, a V_{H} CDR2 comprising amino acids having the sequence set forth in SEQ ID NO: 114 or conservative modifications thereof, and a V_{H} CDR3 comprising amino acids having the sequence set forth in SEQ ID NO: 115 or conservative modifications thereof, as shown in Table 5. In certain embodiments, the extracellular antigen-binding domain comprises a V_{L} CDR1 comprising amino acids having the sequence set forth in SEQ ID NO: 116 or conservative modifications thereof, a V_{L} CDR2 comprising amino acids having the sequence set forth in SEQ ID NO: 117 or conservative modifications thereof, and a V_{L} CDR3 comprising amino acids having the sequence set forth in SEQ ID NO: 118 or conservative modifications thereof, as shown in Table 5. In certain embodiments, the extracellular antigen-binding domain comprises a V_{H} CDR1 comprising amino acids having the sequence set forth in SEQ ID NO: 113 or conservative modifications thereof, a V_{H} CDR2 comprising amino acids having the sequence set forth in SEQ ID NO: 114 or conservative modifications thereof, a V_{H} CDR3 comprising amino acids having the sequence set forth in SEQ ID NO: 115 or conservative modifications thereof, a V_{L} CDR1 comprising amino acids having the sequence set forth in SEQ ID NO: 116 or conservative modifications thereof, a V_{L} CDR2 comprising amino acids having the sequence set forth in SEQ ID NO: 117 or conservative modifications thereof, and a V_{L} CDR3 comprising amino acids having the sequence set forth in SEQ ID NO: 118 or conservative modifications thereof, as shown in Table 5. In certain embodiments, the extracellular antigen-binding domain comprises a V_{H} CDR1 comprising amino acids having the sequence set forth in SEQ ID NO: 113, a V_{H} CDR2 comprising amino acids having the sequence set forth in SEQ ID NO: 114, a V_{H} CDR3 comprising amino acids having the sequence set forth in SEQ ID NO: 115, a V_{L} CDR1 comprising amino acids having the sequence set forth in SEQ ID NO: 116, a V_{L} CDR2 comprising amino acids having the sequence set forth in SEQ ID NO: 117, and a V_{L} CDR3 comprising amino acids having the sequence set forth in SEQ ID NO: 118.

TABLE 5

| | A BCMA polypeptide having the amino acid sequence of SEQ ID NO: 71 | | |
|---|---|---|---|
| Antigen | | | |
| CDRs | 1 | 2 | 3 |
| V_{H} | GGTFSSYA [SEQ | IIPILGIA [SEQ ID | ARTGYESWGSYEVIDR |
| | ID NO: 113] | NO: 114] | [SEQ ID NO: 115] |
| V_{L} | SSNIGSNT [SEQ | SNN [SEQ ID NO: 117] | AAWDDSLNGVV |
| | ID NO: 116] | | [SEQ ID NO: 118] |

| | |
|---|---|
| Full V_{H} | QVQLVESGAEVKKPGSSVKVSCKASGGTFSSYAISWVRQAPGQGL |
| | EWMGRIIPILGIANYAQKFQGRVTITADESTSTAYMELSSLRSEDTA |
| | VYYCARTGYESWGSYEVIDRWGQGTLVTVSS [SEQ ID NO: 17] |
| DNA | Caggtgcagctggtggagtctggggctgaggtgaagaagcctgggtcctcggtgaaggtctcctgcaa |
| | ggcttctggaggcaccttcagcagctatgctatcagctgggtgcgacaggcccctggacaagggcttga |
| | gtggatgggaaggatcatccctatccttggtatagcaaactacgcacagaagttccagggcagagtcacg |
| | attaccgcggacgaatccacgagcacagcctacatggagctgagcagcctgagatctgaggacacggc |
| | cgtatattactgtgcgcgcactggttacgaatcttggggttcttacgaagttatcgatcgttggggtcaaggt |
| | actctggtgaccgtctcctca [SEQ ID NO: 19] |
| Full V_{L} | QAVLTQPPSASGTPGQRVTISCSGSSSNIGSNTVNWYRQLPGTAPK |
| | LLIYSNNQRPSGVPDRFSGSKSGTSASLAISGLQSEDEADYYCAAW |
| | DDSLNGVVFGGGTKLTVLG [SEQ ID NO: 18] |
| DNA | Caggctgtgctgactcagccaccctcagcgtctgggacccccgggcagagggtcaccatctcttgttct |
| | ggaagcagctccaacatcggaagtaatactgtaaactggtaccggcagctcccaggaacggcccccaa |
| | actcctcatctatagtaataatcagcggccctcaggggtccctgaccgattctctggctccaagtctggcac |
| | ctcagcctccctggccatcagtgggctccagtctgaggatgaggctgattattactgtgcagcatgggatg |
| | acagcctgaatggtgtggtattcggcggagggaccaagctgaccgtcctaggt [SEQ ID NO: 20] |
| scFv | QAVLTQPPSASGTPGQRVTISCSGSSSNIGSNTVNWYRQLPGTAPK |
| | LLIYSNNQRPSGVPDRFSGSKSGTSASLAISGLQSEDEADYYCAAW |
| | DDSLNGVVFGGGTKLTVLGSRGGGGSGGGGSGGGGSLEMAQVQ |
| | LVESGAEVKKPGSSVKVSCKASGGTFSSYAISWVRQAPGQGLEW |
| | MGRIIPILGIANYAQKFQGRVTITADESTSTAYMELSSLRSEDTAVY |
| | YCARTGYESWGSYEVIDRWGQGTLVTVSS [SEQ ID NO: 76] |

In certain embodiments, the extracellular antigen-binding domain is a human scFv, which comprises the amino acid sequence of SEQ ID NO: 77 and specifically binds to a BCMA polypeptide (e.g., a BCMA polypeptide having the amino acid sequence SEQ ID NO: 71, or fragments thereof), which is designated as ET140-153 scFv (also referred to as “ET140-3 scFv”).

In certain embodiments, the extracellular antigen-binding domain is a human scFv, which comprises a heavy chain variable region comprising amino acids having the sequence set forth in SEQ ID NO: 21 and a light chain variable region comprising amino acids having the sequence set forth in SEQ ID NO: 22, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 6. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 21, as shown in Table 6. In certain embodiments, the extracellular antigen-binding domain comprises a V_{H} comprising amino acids having the sequence set forth in SEQ ID NO: 21, as shown in Table 6. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 22, as shown in Table 6. In certain embodiments, the extracellular antigen-binding domain comprises a V_{L} comprising amino acids having the sequence set forth in SEQ ID NO: 22, as shown in Table 6. In certain embodiments, the extracellular antigen-binding domain comprises a V_{H} comprising amino acids having the sequence set forth in SEQ ID NO: 21 and a V_{L} comprising amino acids having the sequence set forth in SEQ ID NO: 22, as shown in Table 6. In certain embodiments, the extracellular antigen-binding domain comprises a V_{H} CDR1 comprising amino acids having the sequence set forth in SEQ ID NO: 119 or conservative modifications thereof, a V_{H} CDR2 comprising amino acids having the sequence set forth in SEQ ID NO: 120 or conservative modifications thereof, and a V_{H} CDR3 comprising amino acids having the sequence set forth in SEQ ID NO: 121 or conservative modifications thereof, as shown in Table 6. In certain embodiments, the extracellular antigen-binding domain comprises a V_{L} CDR1 comprising amino acids having the sequence set forth in SEQ ID NO: 122 or conservative modifications thereof, a V_{L} CDR2 comprising amino acids having the sequence set forth in SEQ ID NO: 123 or conservative modifications thereof, and a V_{L} CDR3 comprising amino acids having the sequence set forth in SEQ ID NO: 124 or conservative modifications thereof, as shown in Table 6. In certain embodiments, the extracellular antigen-binding domain comprises a V_{H} CDR1 comprising amino acids having the sequence set forth in SEQ ID NO: 119 or conservative modifications thereof, a V_{H} CDR2 comprising amino acids having the sequence set forth in SEQ ID NO: 120 or conservative modifications thereof, a V_{H} CDR3 comprising amino acids having the sequence set forth in SEQ ID NO: 121 or conservative modifications thereof, a V_{L} CDR1 comprising amino acids having the sequence set forth in SEQ ID NO: 122 or conservative modifications thereof, a V_{L} CDR2 comprising amino acids having the sequence set forth in SEQ ID NO: 123 or conservative modifications thereof, and a V_{L} CDR3 comprising amino acids having the sequence set forth in SEQ ID NO: 124 or conservative modifications thereof, as shown in Table 6. In certain embodiments, the extracellular antigen-binding domain comprises a V_{H} CDR1 comprising amino acids having the sequence set forth in SEQ ID NO: 119, a V_{H} CDR2 comprising amino acids having the sequence set forth in SEQ ID NO: 120, a V_{H} CDR3 comprising amino acids having the sequence set forth in SEQ ID NO: 121, a V_{L} CDR1 comprising amino acids having the sequence set forth in SEQ ID NO: 122, a V_{L} CDR2 comprising amino acids having the sequence set forth in SEQ ID NO: 123, and a V_{L} CDR3 comprising amino acids having the sequence set forth in SEQ ID NO: 124.

TABLE 6

| | A BCMA polypeptide having the amino acid sequence of SEQ ID NO: 71 | | |
|---|---|---|---|
| Antigen | | | |
| CDRs | 1 | 2 | 3 |
| V_{H} | GGTFSSYA [SEQ | IIPILGIA [SEQ ID | ARGGYYSHDMWSED |
| | ID NO: 119] | NO: 120] | [SEQ ID |
| | | | NO: 121] |
| V_{L} | SSNIGSNS [SEQ | SNN [SEQ ID NO: | ATWDDNLNVHYV |
| | ID NO: 122] | 123] | [SEQ ID NO: 124] |

| | |
|---|---|
| Full V_{H} | QVQLVQSGAEVKKPGSSVKVSCKASGGTFSSYAISWVRQAPGQGL |
| | EWMGRIIPILGIANYAQKFQGRVTITADKSTSTAYMELSSLRSEDTA |
| | VYYCARGGYYSHDMWSEDWGQGTLVTVSS [SEQ ID NO: 21] |
| DNA | Caggtgcagctggtgcagtctggggctgaggtgaagaagcctgggtcctcggtgaaggtctcctgcaag |
| | gcttctggaggcaccttcagcagctatgctatcagctgggtgcgacaggcccctggacaagggcttgagt |
| | ggatgggaaggatcatccctatccttggtatagcaaactacgcacagaagttccagggcagagtcacgatt |
| | accgcggacaaatccacgagcacagcctacatggagctgagcagcctgagatctgaggacacggccgt |
| | gtattactgtgcgcgcggtggttactactctcatgacatgtggtctgaagattggggtcaaggtactctggtg |
| | accgtctcctca [SEQ ID NO: 23] |
| Full V_{L} | LPVLTQPPSASGTPGQRVTISCSGRSSNIGSNSVNWYRQLPGAAPKL |
| | LIYSNNQRPPGVPVRFSGSKSGTSASLAISGLQSEDEATYYCATWD |
| | DNLNVHYVFGTGTKVTVLG [SEQ ID NO: 22] |
| DNA | Ctgcctgtgctgactcagccaccctcagcgtctgggacccccgggcagagggtcaccatctcttgttctg |
| | gacgcagttccaacatcgggagtaattctgttaactggtatcgacaactcccaggagcggcccccaaactc |
| | ctcatctatagtaataatcagcggcccccaggggtccctgtgcgattctctggctccaagtctggcacctca |
| | gcctccctggccatcagtgggctccagtctgaagatgaggccacttattactgtgcaacatgggatgacaa |
| | tctgaatgttcactatgtcttcggaactgggaccaaggtcaccgtcctaggt [SEQ ID NO: 24] |
| scFv | LPVLTQPPSASGTPGQRVTISCSGRSSNIGSNSVNWYRQLPGAAPKL |
| | LIYSNNQRPPGVPVRFSGSKSGTSASLAISGLQSEDEATYYCATWD |
| | DNLNVHYVFGTGTKVTVLGSRGGGGSGGGGSGGGGSLEMAQVQ |
| | LVQSGAEVKKPGSSVKVSCKASGGTFSSYAISWVRQAPGQGLEW |
| | MGRIIPILGIANYAQKFQGRVTITADKSTSTAYMELSSLRSEDTAVY |
| | YCARGGYYSHDMWSEDWGQGTLVTVSS [SEQ ID NO: 77] |

In certain embodiments, the extracellular antigen-binding domain is a human scFv, which comprises the amino acid sequence of SEQ ID NO: 78 and specifically binds to a BCMA polypeptide (e.g., a BCMA polypeptide having the amino acid sequence SEQ ID NO: 71, or fragments thereof), which is designated as ET140-201 scFv (also referred to as “ET140-51 scFv”).

In certain embodiments, the extracellular antigen-binding domain is a human scFv, which comprises a heavy chain variable region comprising amino acids having the sequence set forth in SEQ ID NO: 25 and a light chain variable region comprising amino acids having the sequence set forth in SEQ ID NO: 26, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 7. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 25, as shown in Table 7. In certain embodiments, the extracellular antigen-binding domain comprises a V_{H} comprising amino acids having the sequence set forth in SEQ ID NO: 25, as shown in Table 7. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 26, as shown in Table7. In certain embodiments, the extracellular antigen-binding domain comprises a V_{L} comprising amino acids having the sequence set forth in SEQ ID NO: 26, as shown in Table 7. In certain embodiments, the extracellular antigen-binding domain comprises a V_{H} comprising amino acids having the sequence set forth in SEQ ID NO: 25 and a V_{L} comprising amino acids having the sequence set forth in SEQ ID NO: 26, as shown in Table 7. In certain embodiments, the extracellular antigen-binding domain comprises a V_{H} CDR1 comprising amino acids having the sequence set forth in SEQ ID NO: 125 or conservative modifications thereof, a V_{H} CDR2 comprising amino acids having the sequence set forth in SEQ ID NO: 126 or conservative modifications thereof, and a V_{H} CDR3 comprising amino acids having the sequence set forth in SEQ ID NO: 127 or conservative modifications thereof, as shown in Table 7. In certain embodiments, the extracellular antigen-binding domain comprises a V_{L} CDR1 comprising amino acids having the sequence set forth in SEQ ID NO: 128 or conservative modifications thereof, a V_{L} CDR2 comprising amino acids having the sequence set forth in SEQ ID NO: 129 or conservative modifications thereof, and a V_{L} CDR3 comprising amino acids having the sequence set forth in SEQ ID NO: 130 or conservative modifications thereof, as shown in Table 7. In certain embodiments, the extracellular antigen-binding domain comprises a V_{H} CDR1 comprising amino acids having the sequence set forth in SEQ ID NO: 125 or conservative modifications thereof, a V_{H} CDR2 comprising amino acids having the sequence set forth in SEQ ID NO: 126 or conservative modifications thereof, a V_{H} CDR3 comprising amino acids having the sequence set forth in SEQ ID NO: 127 or conservative modifications thereof, a V_{L} CDR1 comprising amino acids having the sequence set forth in SEQ ID NO: 128 or conservative modifications thereof, a V_{L} CDR2 comprising amino acids having the sequence set forth in SEQ ID NO: 129 or conservative modifications thereof, and a V_{L} CDR3 comprising amino acids having the sequence set forth in SEQ ID NO: 130 or conservative modifications thereof, as shown in Table 7. In certain embodiments, the extracellular antigen-binding domain comprises a V_{H} CDR1 comprising amino acids having the sequence set forth in SEQ ID NO: 125, a V_{H} CDR2 comprising amino acids having the sequence set forth in SEQ ID NO: 126, a V_{H} CDR3 comprising amino acids having the sequence set forth in SEQ ID NO: 127, a V_{L} CDR1 comprising amino acids having the sequence set forth in SEQ ID NO: 128, a V_{L} CDR2 comprising amino acids having the sequence set forth in SEQ ID NO: 129, and a V_{L} CDR3 comprising amino acids having the sequence set forth in SEQ ID NO: 130.

TABLE 7

| | A BCMA polypeptide having the amino acid sequence of SEQ ID NO: 71 | | |
|---|---|---|---|
| Antigen | | | |
| CDRs | 1 | 2 | 3 |
| V_{H} | GGSISNSNW [SEQ | IYHSGST [SEQ | ARRDNWKTPTTKID |
| | ID NO: 125] | ID NO: 126] | GFDI [SEQ ID |
| | | | NO: 127] |
| V_{L} | SGYSNYK [SEQ | VGTGGIVG [SEQ | GADHGSGSNFVYV |
| | ID NO: 128] | ID NO: 129] | SEQ ID NO: 130] |

| | |
|---|---|
| Full V_{H} | QVQLQESGPGLVKPSGTLSLTCGVSGGSISNSNWWSWVRQPPGKG |
| | LEWIGEIYHSGSTKYNPSLRSRVTISVDKSKNQFSLKLSSVTAADT |
| | AVYYCARRDNWKTPTTKIDGFDIWGQGTMVTVSS [SEQ ID NO: 25] |
| DNA | Caggtgcagctgcaggagtcgggcccaggactggtgaagccttcggggaccctgtccctcacctgcg |
| | gtgtctctggtggctccatcagcaatagtaactggtggagttgggtccgccagccccccgggaaggggc |
| | tggagtggattggggaaatctatcatagtgggagcaccaagtacaacccgtccctcaggagtcgagtcac |
| | catatcagtagacaagtccaagaaccagttctccctaaaattgagctctgtgaccgccgcggacacggcc |
| | gtatattactgtgcgagacgagataactggaagacccccactaccaaaattgatggttttgatatctggggc |
| | caagggacaatggtcaccgtctcttca [SEQ ID NO: 27] |
| Full V_{L} | |
| | QPVLTQPPSASASLGASVTLTCTLSSGYSNYKVDWYQQRPGKGPR |
| | FVMRVGTGGIVGSKGDGIPDRFSVLGSGLNRYLTIKNIQEEDEGDY |
| | HCGADHGSGSNFVYVFGTGTKVTVLG [SEQ ID NO: 26] |
| DNA | Cagcctgtgctgactcagccaccttctgcatcagcctccctgggagcctcggtcacactcacctgcaccc |
| | tgagcagcggctacagtaattataaagtggactggtaccagcagagaccagggaagggcccccggfttg |
| | tgatgcgagtgggcactggtgggattgtgggatccaagggggatggcatccctgatcgcttctcagtcttg |
| | ggctcaggcctgaatcggtacctgaccatcaagaacatccaggaagaagatgagggtgactatcactgt |
| | ggggcagaccatggcagtgggagcaacttcgtgtatgtcttcggaactgggaccaaggtcaccgtccta |
| | ggt [SEQ ID NO: 28] |
| scFv | QPVLTQPPSASASLGASVTLTCTLSSGYSNYKVDWYQQRPGKGPR |
| | FVMRVGTGGIVGSKGDGIPDRFSVLGSGLNRYLTIKNIQEEDEGDY |
| | HCGADHGSGSNFVYVFGTGTKVTVLGSRGGGGSGGGGSGGGGSL |
| | EMAQVQLQESGPGLVKPSGTLSLTCGVSGGSISNSNWWSWVRQP |
| | PGKGLEWIGEIYHSGSTKYNPSLRSRVTISVDKSKNQFSLKLSSVTA |
| | ADTAVYYCARRDNWKTPTTKIDGFDIWGQGTMVTVSS [SEQ ID NO: 78] |

In certain embodiments, the extracellular antigen-binding domain is a human scFv, which comprises the amino acid sequence of SEQ ID NO: 79 and specifically binds to a BCMA polypeptide (e.g., a BCMA polypeptide having the amino acid sequence SEQ ID NO: 71, or fragments thereof), which is designated as ET140-167 scFv (also referred to as “ET140-17 scFv”).

In certain embodiments, the extracellular antigen-binding domain is a human scFv, which comprises a heavy chain variable region comprising amino acids having the sequence set forth in SEQ ID NO: 29 and a light chain variable region comprising amino acids having the sequence set forth in SEQ ID NO: 30, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 8. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 29, as shown in Table 8. In certain embodiments, the extracellular antigen-binding domain comprises a V_{H} comprising amino acids having the sequence set forth in SEQ ID NO: 29, as shown in Table 8. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 30, as shown in Table 8. In certain embodiments, the extracellular antigen-binding domain comprises a V_{L} comprising amino acids having the sequence set forth in SEQ ID NO: 30, as shown in Table 8. In certain embodiments, the extracellular antigen-binding domain comprises a V_{H} comprising amino acids having the sequence set forth in SEQ ID NO: 29 and a V_{L} comprising amino acids having the sequence set forth in SEQ ID NO: 30, as shown in Table 8. In certain embodiments, the anti-BCMA comprises a V_{H} CDR1 comprising amino acids having the sequence set forth in SEQ ID NO: 131 or conservative modifications thereof, a V_{H} CDR2 comprising amino acids having the sequence set forth in SEQ ID NO: 132 or conservative modifications thereof, and a V_{H} CDR3 comprising amino acids having the sequence set forth in SEQ ID NO: 133 or conservative modifications thereof, as shown in Table 8. In certain embodiments, the extracellular antigen-binding domain comprises a V_{L} CDR1 comprising amino acids having the sequence set forth in SEQ ID NO: 134 or conservative modifications thereof, a V_{L} CDR2 comprising amino acids having the sequence set forth in SEQ ID NO: 135 or conservative modifications thereof, and a V_{L} CDR3 comprising amino acids having the sequence set forth in SEQ ID NO: 136 or conservative modifications thereof, as shown in Table 8. In certain embodiments, the extracellular antigen-binding domain comprises a V_{H} CDR1 comprising amino acids having the sequence set forth in SEQ ID NO: 131 or conservative modifications thereof, a V_{H} CDR2 comprising amino acids having the sequence set forth in SEQ ID NO: 132 or conservative modifications thereof, a V_{H} CDR3 comprising amino acids having the sequence set forth in SEQ ID NO: 133 or conservative modifications thereof, a V_{L} CDR1 comprising amino acids having the sequence set forth in SEQ ID NO: 134 or conservative modifications thereof, a V_{L} CDR2 comprising amino acids having the sequence set forth in SEQ ID NO: 135 or conservative modifications thereof, and a V_{L} CDR3 comprising amino acids having the sequence set forth in SEQ ID NO: 136 or conservative modifications thereof, as shown in Table 8. In certain embodiments, the extracellular antigen-binding domain comprises a V_{H} CDR1 comprising amino acids having the sequence set forth in SEQ ID NO: 131, a V_{H} CDR2 comprising amino acids having the sequence set forth in SEQ ID NO: 132, a V_{H} CDR3 comprising amino acids having the sequence set forth in SEQ ID NO: 133, a V_{L} CDR1 comprising amino acids having the sequence set forth in SEQ ID NO: 134, a V_{L} CDR2 comprising amino acids having the sequence set forth in SEQ ID NO: 135, and a V_{L} CDR3 comprising amino acids having the sequence set forth in SEQ ID NO: 136.

TABLE 8

| | A BCMA polypeptide having the amino acid sequence of SEQ ID NO: 71 | | |
|---|---|---|---|
| Antigen | | | |
| CDRs | 1 | 2 | 3 |
| V_{H} | GYTFTGYY [SEQ ID | INPNSGGT [SEQ | ARSQWGSSWDY |
| | NO: 131] | ID NO: 132] | [SEQ ID NO: 133] |
| V_{L} | QSISSY [SEQ ID | AAS [SEQ ID NO: | QQSYSTPPT [SEQ |
| | NO: 134] | 135] | ID NO: 136] |

| | |
|---|---|
| Full V_{H} | QVQLVQSGAEVKKPGASVKVSCKASGYTFTGYYMHWVRQAPGQ |
| | GLEWMGWINPNSGGTNYAQKFQGRVTMTRDTSISTAYMELSRLR |
| | SDDTAVYYCARSQWGSSWDYWGQGTLVTVSS [SEQ ID NO: 29] |
| DNA | Caggtccagctggtacagtctggggctgaggtgaagaagcctggggcctcagtgaaggtctcctgcaa |
| | ggcttctggatacaccttcaccggctactatatgcactgggtgcgacaggcccctggacaagggcttgag |
| | tggatgggatggatcaaccctaacagtggtggcacaaactatgcacagaagtttcagggcagggtcacc |
| | atgaccagggacacgtccatcagcacagcctacatggagctgagcaggctgagatctgacgacacggc |
| | cgtgtattactgtgcgcgctctcagtggggttcttcttgggattactggggtcaaggtactctggtgaccgtc |
| | tcctca [SEQ ID NO: 31] |
| Full V_{L} | DIQLTQSPSSLSASVGDRVTITCRASQSISSYLNWYQQKPGKAPKL |
| | LIYAASSLQSGVPSRFSGSGSGTDFTLTISSLQPEDFATYYCQQSYS |
| | TPPTFGQGTKVEIKR [SEQ ID NO: 30] |
| DNA | Gacatccagttgacccagtctccatcctccctgtctgcatctgtaggagacagagtcaccatcacttgccg |
| | ggcaagtcagagcattagcagctatttaaattggtatcagcagaaaccagggaaagcccctaagctcctg |
| | atctatgctgcatccagtttgcaaagtggggtcccatcaaggttcagtggcagtggatctgggacagatttc |
| | actctcaccatcagcagtctgcaacctgaagattttgcaacttactactgtcaacagagttacagtacccctc |
| | cgacgttcggccaagggaccaaggtggagatcaaacgt [SEQ ID NO: 32] |
| scFv | DIQLTQSPSSLSASVGDRVTITCRASQSISSYLNWYQQKPGKAPKL |
| | LIYAASSLQSGVPSRFSGSGSGTDFTLTISSLQPEDFATYYCQQSYS |
| | TPPTFGQGTKVEIKRSRGGGGSGGGGSGGGGSLEMAQVQLVQSG |
| | AEVKKPGASVKVSCKASGYTFTGYYMHWVRQAPGQGLEWMGW |
| | INPNSGGTNYAQKFQGRVTMTRDTSISTAYMELSRLRSDDTAVYY |
| | CARSQWGSSWDYWGQGTLVTVSS [SEQ ID NO: 79] |

In certain embodiments, the extracellular antigen-binding domain is a human scFv, which comprises the amino acid sequence of SEQ ID NO: 80 and specifically binds to a BCMA polypeptide (e.g., a BCMA polypeptide having the amino acid sequence SEQ ID NO: 71, or fragments thereof), which is designated as ET140-163 scFv (also referred to as “ET140-13 scFv”).

In certain embodiments, the extracellular antigen-binding domain is a human scFv, which comprises a heavy chain variable region comprising amino acids having the sequence set forth in SEQ ID NO: 33 and a light chain variable region comprising amino acids having the sequence set forth in SEQ ID NO: 34, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 9. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 33, as shown in Table 9. In certain embodiments, the extracellular antigen-binding domain comprises a V_{H} comprising amino acids having the sequence set forth in SEQ ID NO: 33, as shown in Table 9. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 34, as shown in Table 9. In certain embodiments, the extracellular antigen-binding domain comprises a V_{L} comprising amino acids having the sequence set forth in SEQ ID NO: 34, as shown in Table 9. In certain embodiments, the extracellular antigen-binding domain comprises a V_{H} comprising amino acids having the sequence set forth in SEQ ID NO: 33 and a V_{L} comprising amino acids having the sequence set forth in SEQ ID NO: 34, as shown in Table 9. In certain embodiments, the extracellular antigen-binding domain comprises a V_{H} CDR1 comprising amino acids having the sequence set forth in SEQ ID NO: 137 or conservative modifications thereof, a V_{H} CDR2 comprising amino acids having the sequence set forth in SEQ ID NO: 138 or conservative modifications thereof, and a V_{H} CDR3 comprising amino acids having the sequence set forth in SEQ ID NO: 139 or conservative modifications thereof, as shown in Table 9. In certain embodiments, the extracellular antigen-binding domain comprises a V_{L} CDR1 comprising amino acids having the sequence set forth in SEQ ID NO: 140 or conservative modifications thereof, a V_{L} CDR2 comprising amino acids having the sequence set forth in SEQ ID NO: 141 or conservative modifications thereof, and a V_{L} CDR3 comprising amino acids having the sequence set forth in SEQ ID NO: 142 or conservative modifications thereof, as shown in Table 9. In certain embodiments, the extracellular antigen-binding domain comprises a V_{H} CDR1 comprising amino acids having the sequence set forth in SEQ ID NO: 137 or conservative modifications thereof, a V_{H} CDR2 comprising amino acids having the sequence set forth in SEQ ID NO: 138 or conservative modifications thereof, a V_{H} CDR3 comprising amino acids having the sequence set forth in SEQ ID NO: 139 or conservative modifications thereof, a V_{L} CDR1 comprising amino acids having the sequence set forth in SEQ ID NO: 140 or conservative modifications thereof, a V_{L} CDR2 comprising amino acids having the sequence set forth in SEQ ID NO: 141 or conservative modifications thereof, and a V_{L} CDR3 comprising amino acids having the sequence set forth in SEQ ID NO: 142 or conservative modifications thereof, as shown in Table 9. In certain embodiments, the extracellular antigen-binding domain comprises a V_{H} CDR1 comprising amino acids having the sequence set forth in SEQ ID NO: 137, a V_{H} CDR2 comprising amino acids having the sequence set forth in SEQ ID NO: 138, a V_{H} CDR3 comprising amino acids having the sequence set forth in SEQ ID NO: 139, a V_{L} CDR1 comprising amino acids having the sequence set forth in SEQ ID NO: 140, a V_{L} CDR2 comprising amino acids having the sequence set forth in SEQ ID NO: 141, and a V_{L} CDR3 comprising amino acids having the sequence set forth in SEQ ID NO: 142.

TABLE 9

| | A BCMA polypeptide having the amino acid sequence of SEQ ID NO: 71 | | |
|---|---|---|---|
| Antigen | | | |
| CDRs | 1 | 2 | 3 |
| V_{H} | GYTFTGYY [SEQ ID | INPNSGGT [SEQ ID | ARSSYHLYGYDS |
| | NO: 137] | NO: 138] | [SEQ ID NO: 139] |
| V_{L} | NDYTNYK [SEQ ID | VGPGGIVG [SEQ | GADHGTGSNFVYV |
| | NO: 140] | ID NO: 141] | [SEQ ID NO: 142] |

| | |
|---|---|
| Full V_{H} | EVQLVESGAEVKKPGASVKVSCKASGYTFTGYYMHWVRQAPGQ |
| | GLEWMGWINPNSGGTNYAQKFQGRVTMTRDTSISTAYMELSRLR |
| | SDDTAVYYCARSSYHLYGYDSWGQGTLVTVSS [SEQ ID NO: 33] |
| DNA | Gaggtgcagctggtggagtccggggctgaggtgaagaagcctggggcctcagtgaaggtctcctgca |
| | aggcttctggatacaccttcaccggctactatatgcactgggtgcgacaggcccctggacaagggcttga |
| | gtggatgggatggatcaaccctaacagtggtggcacaaactatgcacagaagtttcagggcagggtcac |
| | catgaccagggacacgtccatcagcacagcctacatggagctgagcaggctgagatctgacgacacgg |
| | ccgtatattactgtgcgcgctatataccatctgtacggttacgattcttggggtcaaggtactctggtga |
| | ccgtctcctca [SEQ ID NO: 35] |
| Full V_{L} | QPVLTQPPSASASLGASVTLTCTLSNDYTNYKVDWYQQRPGKGPR |
| | FVMRVGPGGIVGSKGDGIPDRFSVLGSGLNRYLTIKNIQEEDESDY |
| | HCGADHGTGSNFVYVFGGGTKLTVLG [SEQ ID NO: 34] |
| DNA | Cagcctgtgctgactcagccaccttctgcatcagcctccctgggagcctcggtcactctcacctgcaccct |
| | gagcaacgactacactaattataaagtggactggtaccagcagagaccagggaagggcccccggtttgt |
| | gatgcgagtgggccctggtgggattgtgggatccaagggggatggcatccctgatcgcttctcagtcttg |
| | ggctcaggcctgaatcgatacctgaccatcaagaacatccaggaggaggatgagagtgactaccactgt |
| | ggggcggaccatggcaccgggagcaacttcgtgtacgtgttcggcggagggaccaagctgaccgtcct |
| | aggt [SEQ ID NO: 36] |
| scFv | QPVLTQPPSASASLGASVTLTCTLSNDYTNYKVDWYQQRPGKGPR |
| | FVMRVGPGGIVGSKGDGIPDRFSVLGSGLNRYLTIKNIQEEDESDY |
| | HCGADHGTGSNFVYVFGGGTKLTVLGSRGGGGSGGGGSGGGGSL |
| | EMAEVQLVESGAEVKKPGASVKVSCKASGYTFTGYYMHWVRQA |
| | PGQGLEWMGWINPNSGGTNYAQKFQGRVTMTRDTSISTAYMELS |
| | RLRSDDTAVYYCARSSYHLYGYDSWGQGTLVTVSS [SEQ ID NO: 80] |

In certain embodiments, the extracellular antigen-binding domain is a human scFv, which comprises the amino acid sequence of SEQ ID NO: 81 and specifically binds to a BCMA polypeptide (e.g., a BCMA polypeptide having the amino acid sequence SEQ ID NO: 71, or fragments thereof), which is designated as ET140-207 scFv (also referred to as “ET140-57 scFv”).

In certain embodiment, the extracellular antigen-binding domain is a human scFv, which comprises a heavy chain variable region comprising amino acids having the sequence set forth in SEQ ID NO: 37 and a light chain variable region comprising amino acids having the sequence set forth in SEQ ID NO: 38, optionally with (iii) a linker sequence, for example a linker peptide, between the heavy chain variable region and the light chain variable region. In certain embodiments, the linker comprises amino acids having the sequence set forth in SEQ ID NO: 98. In certain embodiments, the extracellular antigen-binding domain is a human scFv-Fc fusion protein or full length human IgG with V_{H} and V_{L} regions or CDRs selected from Table 10. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 37, as shown in Table 10. In certain embodiments, the extracellular antigen-binding domain comprises a V_{H} comprising amino acids having the sequence set forth in SEQ ID NO: 37, as shown in Table 10. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 38, as shown in Table 10. In certain embodiments, the extracellular antigen-binding domain comprises a V_{L} comprising amino acids having the sequence set forth in SEQ ID NO: 38, as shown in Table 10. In certain embodiments, the extracellular antigen-binding domain comprises a V_{H} comprising amino acids having the sequence set forth in SEQ ID NO: 37 and a V_{L} comprising amino acids having the sequence set forth in SEQ ID NO: 38, as shown in Table 10. In certain embodiments, the extracellular antigen-binding domain comprises a V_{H} CDR1 comprising amino acids having the sequence set forth in SEQ ID NO: 143 or conservative modifications thereof, a V_{H} CDR2 comprising amino acids having the sequence set forth in SEQ ID NO: 144 or conservative modifications thereof, and a V_{H} CDR3 comprising amino acids having the sequence set forth in SEQ ID NO: 145 or conservative modifications thereof, as shown in Table 10. In certain embodiments, the extracellular antigen-binding domain comprises a V_{L} CDR1 comprising amino acids having the sequence set forth in SEQ ID NO: 146 or conservative modifications thereof, a V_{L} CDR2 comprising amino acids having the sequence set forth in SEQ ID NO: 147 or conservative modifications thereof, and a V_{L} CDR3 comprising amino acids having the sequence set forth in SEQ ID NO: 148 or conservative modifications thereof, as shown in Table 10. In certain embodiments, the extracellular antigen-binding domain comprises a V_{H} CDR1 comprising amino acids having the sequence set forth in SEQ ID NO: 143 or conservative modifications thereof, a V_{H} CDR2 comprising amino acids having the sequence set forth in SEQ ID NO: 144 or conservative modifications thereof, a V_{H} CDR3 comprising amino acids having the sequence set forth in SEQ ID NO: 145 or conservative modifications thereof, a V_{L} CDR1 comprising amino acids having the sequence set forth in SEQ ID NO: 146 or conservative modifications thereof, a V_{L} CDR2 comprising amino acids having the sequence set forth in SEQ ID NO: 147 or conservative modifications thereof, and a V_{L} CDR3 comprising amino acids having the sequence set forth in SEQ ID NO: 148 or conservative modifications thereof, as shown in Table 10. In certain embodiments, the extracellular antigen-binding domain comprises a V_{H} CDR1 comprising amino acids having the sequence set forth in SEQ ID NO: 143, a V_{H} CDR2 comprising amino acids having the sequence set forth in SEQ ID NO: 144, a V_{H} CDR3 comprising amino acids having the sequence set forth in SEQ ID NO: 145, a V_{L} CDR1 comprising amino acids having the sequence set forth in SEQ ID NO: 146, a V_{L} CDR2 comprising amino acids having the sequence set forth in SEQ ID NO: 147, and a V_{L} CDR3 comprising amino acids having the sequence set forth in SEQ ID NO: 148.

TABLE 10

| | A BCMA polypeptide having the amino acid sequence of SEQ ID NO: 71 | | |
|---|---|---|---|
| Antigen | | | |
| CDRs | 1 | 2 | 3 |
| V_{H} | GGTFSSYA [SEQ | IIPIFSTA [SEQ | ARQPWTWYSPYDQ |
| | ID NO: 143] | ID NO: 144] | [SEQ ID NO: 145] |
| V_{L} | SGYSNYK [SEQ | VDTGGIVG [SEQ | GADHGSGSNFVWV |
| | ID NO: 146] | ID NO: 147] | [SEQ ID NO: 148] |

| | |
|---|---|
| Full V_{H} | QVQLVQSGAEVKKPGSSVKVSCKASGGTFSSYAISWVRQAPGQG |
| | LEWMGGIIPIFSTANYAQKFQGRVTMTTDTSTSTAYMELRSLRSD |
| | DTAVYYCARQPWTWYSPYDQWGQGTLVTVSS [SEQ ID NO: 37] |
| DNA | Caggtgcagctggtgcagtctggggctgaggtgaagaagcctgggtcctcggtgaaggtctcctgcaa |
| | ggcttctggaggcaccttcagcagctatgctatcagctgggtgcgacaggcccctggacaagggcttga |
| | gtggatgggagggatcatccctatctttagtacagcaaactacgcacagaagttccagggcagagtcacc |
| | atgaccacagacacatccacgagcacagcctacatggagctgaggagcctgagatctgacgacacggc |
| | cgtgtattactgtgcgcgccagccgtggacttggtactctccgtacgatcagtggggtcaaggtactct |
| | ggtgaccgtctcctca [SEQ ID NO: 39] |
| Full V_{L} | QPVLTQPPSASASLGASVTLTCTLSSGYSNYKVDWYQQRPGKGPR |
| | FLMRVDTGGIVGSKGDGIPDRFSVSGSGLNRYLTIKNIQEEDESDY |
| | HCGADHGSGSNFVWVFGGGTKLTVLG [SEQ ID NO: 38] |
| DNA | Cagcctgtgctgactcagccaccttctgcatcagcctccctgggagcctcggtcacactcacctgcaccc |
| | tgagcagcggctacagtaattataaagtggactggtatcaacagagaccagggaagggcccccggtttct |
| | gatgcgagtagacaccggtgggattgtgggatccaagggggatggcatccctgatcgcttctcagtctcg |
| | ggctcaggtctgaatcggtacctgaccatcaagaacattcaggaagaggatgagagtgactaccactgtg |
| | gggcagaccatggcagtgggagcaacttcgtgtgggtgttcggcggagggaccaagctgaccgtccta |
| | ggt [SEQ ID NO: 40] |
| scFv | QPVLTQPPSASASLGASVTLTCTLSSGYSNYKVDWYQQRPGKGPR |
| | FLMRVDTGGIVGSKGDGIPDRFSVSGSGLNRYLTIKNIQEEDESDY |
| | HCGADHGSGSNFVWVFGGGTKLTVLGSRGGGGSGGGGSGGGGS |
| | LEMAQVQLVQSGAEVKKPGSSVKVSCKASGGTFSSYAISWVRQA |
| | PGQGLEWMGGIIPIFSTANYAQKFQGRVTMTTDTSTSTAYMELRS |
| | LRSDDTAVYYCARQPWTWYSPYDQWGQGTLVTVSS [SEQ ID NO: 81] |

In certain embodiments, the extracellular antigen-binding domain is a human scFv, which comprises the amino acid sequence of SEQ ID NO: 82 and specifically binds to a BCMA polypeptide (e.g., a BCMA polypeptide having the amino acid sequence SEQ ID NO: 71, or fragments thereof), which is designated as ET140-165 scFv (also referred to as “ET140-15 scFv”).

In certain embodiments, the extracellular antigen-binding domain is a human scFv, which comprises a heavy chain variable region comprising amino acids having the sequence set forth in SEQ ID NO: 41 and a light chain variable region comprising amino acids having the sequence set forth in SEQ ID NO: 42, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 11. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 41, as shown in Table 11. In certain embodiments, the extracellular antigen-binding domain comprises a V_{H} comprising amino acids having the sequence set forth in SEQ ID NO: 41, as shown in Table 11. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 42, as shown in Table 11. In certain embodiments, the extracellular antigen-binding domain comprises a V_{L} comprising amino acids having the sequence set forth in SEQ ID NO: 42, as shown in Table 11. In certain embodiments, the extracellular antigen-binding domain comprises a V_{H} comprising amino acids having the sequence set forth in SEQ ID NO: 41 and a V_{L} comprising amino acids having the sequence set forth in SEQ ID NO: 42, as shown in Table 11. In certain embodiments, the extracellular antigen-binding domain comprises a V_{H} CDR1 comprising amino acids having the sequence set forth in SEQ ID NO: 149 or conservative modifications thereof, a V_{H} CDR2 comprising amino acids having the sequence set forth in SEQ ID NO: 150 or conservative modifications thereof, and a V_{H} CDR3 comprising amino acids having the sequence set forth in SEQ ID NO: 151 or conservative modifications thereof, as shown in Table 11. In certain embodiments, the extracellular antigen-binding domain comprises a V_{L} CDR1 comprising amino acids having the sequence set forth in SEQ ID NO: 152 or conservative modifications thereof, a V_{L} CDR2 comprising amino acids having the sequence set forth in SEQ ID NO: 153 or conservative modifications thereof, and a V_{L} CDR3 comprising amino acids having the sequence set forth in SEQ ID NO: 154 or conservative modifications thereof, as shown in Table 11. In certain embodiments, the extracellular antigen-binding domain comprises a V_{H} CDR1 comprising amino acids having the sequence set forth in SEQ ID NO: 147 or conservative modifications thereof, a V_{H} CDR2 comprising amino acids having the sequence set forth in SEQ ID NO: 150 or conservative modifications thereof, a V_{H} CDR3 comprising amino acids having the sequence set forth in SEQ ID NO: 151 or conservative modifications thereof, a V_{L} CDR1 comprising amino acids having the sequence set forth in SEQ ID NO: 152 or conservative modifications thereof, a V_{L} CDR2 comprising amino acids having the sequence set forth in SEQ ID NO: 153 or conservative modifications thereof, and a V_{L} CDR3 comprising amino acids having the sequence set forth in SEQ ID NO: 154 or conservative modifications thereof, as shown in Table 11. In certain embodiments, the extracellular antigen-binding domain comprises a V_{H} CDR1 comprising amino acids having the sequence set forth in SEQ ID NO: 149, a V_{H} CDR2 comprising amino acids having the sequence set forth in SEQ ID NO: 150, a V_{H} CDR3 comprising amino acids having the sequence set forth in SEQ ID NO: 151, a V_{L} CDR1 comprising amino acids having the sequence set forth in SEQ ID NO: 152, a V_{L} CDR2 comprising amino acids having the sequence set forth in SEQ ID NO: 153, and a V_{L} CDR3 comprising amino acids having the sequence set forth in SEQ ID NO: 154.

TABLE 11

| | A BCMA polypeptide having the amino acid sequence of SEQ ID NO: 71 | | |
|---|---|---|---|
| Antigen | | | |
| CDRs | 1 | 2 | 3 |
| V_{H} | GFTFSTYA [SEQ | ITPGGDRT [SEQ | ARYYGYMIDM [SEQ |
| | ID NO: 149] | ID NO: 150] | ID NO: 151] |
| V_{L} | QSLLHSNGYNY [SEQ | LGS [SEQ ID | MQALQTPLT [SEQ |
| | ID NO: 152] | NO: 153] | ID NO: 154] |

| | |
|---|---|
| Full V_{H} | EVQLVETGGGLVQPGGSLRLSCAASGFTFSTYAMTWVRQAPGKGL |
| | EWVSAITPGGDRTYYADSVKGRFTISRDNSRNTLYLQMNSLRAEDT |
| | AVYYCARYYGYMIDMWGQGTLVTVSS [SEQ ID NO: 41] |
| DNA | Gaggtgcagctggtggagactgggggaggcctggtacagcctggggggtccctgagactctcctgtgct |
| | gcctctggattcacctttagcacctatgccatgacctgggtccgccaggctccagggaaggggctggagt |
| | gggtctcagctattactcctggtggtgatcgcacatactacgcagactccgtgaagggccgtttcactatctc |
| | cagagacaattccaggaacacgctgtatctgcaaatgaacagcctgagagccgaggacacggccgtatat |
| | tactgtgcgcgctactacggttacatgatcgatatgtggggtcaaggtactctggtgaccgtctcctca |
| | [SEQ ID NO: 43] |
| Full V_{L} | DVVMTQSPLSLPVTPGEPASISCRSSQSLLHSNGYNYLDWYLQKPG |
| | QSPQLLIYLGSNRASGVPDRFSGSGSGTDFTLKISRVEAEDVGVYYC |
| | MQALQTPLTFGGGTKVEIKR [SEQ ID NO: 42] |
| DNA | Gatgttgtgatgactcagtctccactctccctgcccgtcacccctggagagccggcctccatctcctgcag |
| | gtctagtcagagcctcctgcatagtaatggatacaactatttggattggtacctgcagaagccagggcagtc |
| | tccacagctcctgatctatttgggttctaatcgggcctccggggtccctgacaggttcagtggcagtggatca |
| | ggcacagattttacactgaaaatcagcagagtggaggctgaggatgttggggtttattactgcatgcaagct |
| | ctacaaactcctctcactttcggcggagggaccaaggtggaaatcaaacgt [SEQ ID NO: 44] |
| scFv | DVVMTQSPLSLPVTPGEPASISCRSSQSLLHSNGYNYLDWYLQKPG |
| | QSPQLLIYLGSNRASGVPDRFSGSGSGTDFTLKISRVEAEDVGVYYC |
| | MQALQTPLTFGGGTKVEIKRSRGGGGSGGGGSGGGGSLEMAEVQL |
| | VETGGGLVQPGGSLRLSCAASGFTFSTYAMTWVRQAPGKGLEWV |
| | SAITPGGDRTYYADSVKGRFTISRDNSRNTLYLQMNSLRAEDTAVY |
| | YCARYYGYMIDMWGQGTLVTVSS [SEQ ID NO: 82] |

In certain embodiments, the extracellular antigen-binding domain is a human scFv, which comprises the amino acid sequence of SEQ ID NO: 83 and specifically binds to a BCMA polypeptide (e.g., a BCMA polypeptide having the amino acid sequence SEQ ID NO: 71, or fragments thereof), which is designated as ET140-188 scFv (also referred to as “ET140-38 scFv”).

In certain embodiments, the extracellular antigen-binding domain is a human scFv, which comprises a heavy chain variable region comprising amino acids having the sequence set forth in SEQ ID NO: 45 and a light chain variable region comprising amino acids having the sequence set forth in SEQ ID NO: 46, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 12. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 45, as shown in Table 12. In certain embodiments, the extracellular antigen-binding domain comprises a V_{H} comprising amino acids having the sequence set forth in SEQ ID NO: 45, as shown in Table 12. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 46, as shown in Table 12. In certain embodiments, the extracellular antigen-binding domain comprises a V_{L} comprising amino acids having the sequence set forth in SEQ ID NO: 46, as shown in Table 12. In certain embodiments, the extracellular antigen-binding domain comprises a V_{H} comprising amino acids having the sequence set forth in SEQ ID NO: 45 and a V_{L} comprising amino acids having the sequence set forth in SEQ ID NO: 46, as shown in Table 12. In certain embodiments, the extracellular antigen-binding domain comprises a V_{H} CDR1 comprising amino acids having the sequence set forth in SEQ ID NO: 155 or conservative modifications thereof, a V_{H} CDR2 comprising amino acids having the sequence set forth in SEQ ID NO: 156 or conservative modifications thereof, and a V_{H} CDR3 comprising amino acids having the sequence set forth in SEQ ID NO: 157 or conservative modifications thereof, as shown in Table 12. In certain embodiments, the extracellular antigen-binding domain comprises a V_{L} CDR1 comprising amino acids having the sequence set forth in SEQ ID NO: 158 or conservative modifications thereof, a V_{L} CDR2 comprising amino acids having the sequence set forth in SEQ ID NO: 159 or conservative modifications thereof, and a V_{L} CDR3 comprising amino acids having the sequence set forth in SEQ ID NO: 160 or conservative modifications thereof, as shown in Table 12. In certain embodiments, the extracellular antigen-binding domain comprises a V_{H} CDR1 comprising amino acids having the sequence set forth in SEQ ID NO: 155 or conservative modifications thereof, a V_{H} CDR2 comprising amino acids having the sequence set forth in SEQ ID NO: 156 or conservative modifications thereof, a V_{H} CDR3 comprising amino acids having the sequence set forth in SEQ ID NO: 157 or conservative modifications thereof, a V_{L} CDR1 comprising amino acids having the sequence set forth in SEQ ID NO: 158 or conservative modifications thereof, a V_{L} CDR2 comprising amino acids having the sequence set forth in SEQ ID NO: 159 or conservative modifications thereof, and a V_{L} CDR3 comprising amino acids having the sequence set forth in SEQ ID NO: 160 or conservative modifications thereof, as shown in Table 12. In certain embodiments, the extracellular antigen-binding domain comprises a V_{H} CDR1 comprising amino acids having the sequence set forth in SEQ ID NO: 155, a V_{H} CDR2 comprising amino acids having the sequence set forth in SEQ ID NO: 156, a V_{H} CDR3 comprising amino acids having the sequence set forth in SEQ ID NO: 157, a V_{L} CDR1 comprising amino acids having the sequence set forth in SEQ ID NO: 158, a V_{L} CDR2 comprising amino acids having the sequence set forth in SEQ ID NO: 159, and a V_{L} CDR3 comprising amino acids having the sequence set forth in SEQ ID NO: 160.

TABLE 12

| | A BCMA polypeptide having the amino acid sequence of SEQ ID NO: 71 | | |
|---|---|---|---|
| Antigen | | | |
| CDRs | 1 | 2 | 3 |
| V_{H} | GYTFTGYY [SEQ | INPNSGGT [SEQ | ARSQWGGTYDY |
| | ID NO: 155] | ID NO: 156] | [SEQ ID NO: 157] |
| V_{L} | SSNIGSNT [SEQ | SNN [SEQ ID | AAWDDSLNGWV |
| | ID NO: 158] | NO: 159] | [SEQ ID NO: 160] |

| | |
|---|---|
| Full V_{H} | QMQLVQSGAEVKKPGASVKVSCKASGYTFTGYYVHWLRQAPGQ |
| | GLEWMGWINPNSGGTNNAQEFQGRITMTRDTSINTAYMELSRLRS |
| | DDTAVYYCARSQWGGTYDYWGQGTLVTVSS [SEQ ID NO: 45] |
| DNA | Cagatgcagctggtgcagtctggggctgaggtgaagaagcctggggcctcagtgaaggtctcctgcaag |
| | gcttctggatacaccttcaccggctattatgtacactggttgcgacaggcccctggacaagggcttgagtgg |
| | atgggttggatcaaccctaacagtggcggcacaaacaatgcacaggagtttcaaggcaggatcaccatga |
| | ccagggacacgtccatcaacacagcctacatggagctgagcaggctgagatctgacgacacggccgtgt |
| | attactgtgcgcgctctcagtggggtggtacttacgattactggggtcaaggtactctggtgaccgtctcct |
| | ca [SEQ ID NO: 47] |
| Full V_{L} | SYVLTQPPSASGTPGQRVTISCSGSSSNIGSNTVNWYQQVPGTAPKL |
| | LIYSNNQRPSGVPDRFSGSKSGASASLAISWLQSEDEADYYCAAWD |
| | DSLNGWVFGGGTKLTVLG [SEQ ID NO: 46] |
| DNA | Tcctatgtgctgactcagccaccctcagcgtctgggacccccgggcagagggtcaccatctcttgttctgg |
| | aagcagctccaacatcggaagtaatactgtaaactggtaccagcaggtcccaggaacggcccccaaactc |
| | ctcatctatagtaataatcagcggccctcaggggtccctgaccgattctctggctccaagtctggcgcctca |
| | gcctccctggccatcagttggctccagtctgaggatgaggctgattattactgtgcagcatgggatgacagc |
| | ctgaatggttgggtgttcggcggagggaccaagctgaccgtcctaggt [SEQ ID NO: 48] |
| scFv | SYVLTQPPSASGTPGQRVTISCSGSSSNIGSNTVNWYQQVPGTAPKL |
| | LIYSNNQRPSGVPDRFSGSKSGASASLAISWLQSEDEADYYCAAWD |
| | DSLNGWVFGGGTKLTVLGSRGGGGSGGGGSGGGGSLEMAQMQL |
| | VQSGAEVKKPGASVKVSCKASGYTFTGYYVHWLRQAPGQGLEW |
| | MGWINPNSGGTNNAQEFQGRITMTRDTSINTAYMELSRLRSDDTA |
| | VYYCARSQWGGTYDYWGQGTLVTVSS [SEQ ID NO: 83] |

In certain embodiments, the extracellular antigen-binding domain is a human scFv, which comprises the amino acid sequence of SEQ ID NO: 84 and specifically binds to a BCMA polypeptide (e.g., a BCMA polypeptide having the amino acid sequence SEQ ID NO: 71, or fragments thereof), which is designated as ET140-196 scFv (also referred to as “ET140-46 scFv”). In certain embodiments, the extracellular antigen-binding domain is a human scFv, which comprises a heavy chain variable region comprising amino acids having the sequence set forth in SEQ ID NO: 49 and a light chain variable region comprising amino acids having the sequence set forth in SEQ ID NO: 50, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 13. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 49, as shown in Table 13. In certain embodiments, the extracellular antigen-binding domain comprises a V_{H} comprising amino acids having the sequence set forth in SEQ ID NO: 49, as shown in Table 13. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 50, as shown in Table 13. In certain embodiments, the extracellular antigen-binding domain comprises a V_{L} comprising amino acids having the sequence set forth in SEQ ID NO: 50, as shown in Table 13. In certain embodiments, the extracellular antigen-binding domain comprises a V_{H} comprising amino acids having the sequence set forth in SEQ ID NO: 49 and a V_{L} comprising amino acids having the sequence set forth in SEQ ID NO: 50, as shown in Table 13. In certain embodiments, the extracellular antigen-binding domain comprises a V_{H} CDR1 comprising amino acids having the sequence set forth in SEQ ID NO: 161 or conservative modifications thereof, a V_{H} CDR2 comprising amino acids having the sequence set forth in SEQ ID NO: 162 or conservative modifications thereof, and a V_{H} CDR3 comprising amino acids having the sequence set forth in SEQ ID NO: 163 or conservative modifications thereof, as shown in Table 13. In certain embodiments, the extracellular antigen-binding domain comprises a V_{L} CDR1 comprising amino acids having the sequence set forth in SEQ ID NO: 164 or conservative modifications thereof, a V_{L} CDR2 comprising amino acids having the sequence set forth in SEQ ID NO: 165 or conservative modifications thereof, and a V_{L} CDR3 comprising amino acids having the sequence set forth in SEQ ID NO: 166 or conservative modifications thereof, as shown in Table 13. In certain embodiments, the extracellular antigen-binding domain comprises a V_{H} CDR1 comprising amino acids having the sequence set forth in SEQ ID NO: 161 or conservative modifications thereof, a V_{H} CDR2 comprising amino acids having the sequence set forth in SEQ ID NO: 162 or conservative modifications thereof, a V_{H} CDR3 comprising amino acids having the sequence set forth in SEQ ID NO: 163 or conservative modifications thereof, a V_{L} CDR1 comprising amino acids having the sequence set forth in SEQ ID NO: 164 or conservative modifications thereof, a V_{L} CDR2 comprising amino acids having the sequence set forth in SEQ ID NO: 165 or conservative modifications thereof, and a V_{L} CDR3 comprising amino acids having the sequence set forth in SEQ ID NO: 166 or conservative modifications thereof, as shown in Table 13. In certain embodiments, the extracellular antigen-binding domain comprises a V_{H} CDR1 comprising amino acids having the sequence set forth in SEQ ID NO: 161, a V_{H} CDR2 comprising amino acids having the sequence set forth in SEQ ID NO: 162, a V_{H} CDR3 comprising amino acids having the sequence set forth in SEQ ID NO: 163, a V_{L} CDR1 comprising amino acids having the sequence set forth in SEQ ID NO: 164, a V_{L} CDR2 comprising amino acids having the sequence set forth in SEQ ID NO: 165, and a V_{L} CDR3 comprising amino acids having the sequence set forth in SEQ ID NO: 166.

TABLE 13

| | A BCMA polypeptide having the amino acid sequence of SEQ ID NO: 71 | | |
|---|---|---|---|
| Antigen | | | |
| CDRs | 1 | 2 | 3 |
| V_{H} | GYDFTTYW [SEQ | IYPGDSDT [SEQ ID | ARMWTFSQDG [SEQ |
| | ID NO: 161] | NO: 162] | ID NO: 163] |
| V_{L} | SSNIGSYT [SEQ | SNN [SEQ ID | AAWDDSLNGYV |
| | ID NO: 164] | NO: 165] | [SEQ ID NO: 166] |

| | |
|---|---|
| Full V_{H} | EVQLVQSGAEVKKPGESLKISCKGSGYDFTTYWIGWVRQMPGKG |
| | LEWMGIIYPGDSDTRYSPSVRGRVTISADKSINTAYLQWSSLEASD |
| | TAMYYCARMWTFSQDGWGQGTLVTVSS [SEQ ID NO: 49] |
| DNA | gaggtgcagctggtgcagtctggagcagaggtgaaaaagccgggggagtctctgaagatctcctgtaa |
| | gggttctggatatgactttaccacctactggatcgggtgggtgcgccagatgcccgggaagggcctgga |
| | gtggatggggatcatctatcctggtgactctgataccagatacagcccgtccgtccgaggccgggtcacc |
| | atctcagccgacaagtccatcaacaccgcctatttgcagtggagtagcctggaggcctccgacaccgcc |
| | atgtattactgtgcgcgcatgtggactttctctcaggatggttggggtcaaggtactctggtgaccgtct |
| | cctca [SEQ ID NO: 51] |
| Full V_{L} | QAVLTQPPSASGTPGQRVTISCSGSSSNIGSYTVSWYQQLPGTAPK |
| | FLIYSNNQRPSGVPDRFSGSKSGTSASLAISGLQSEDEADYYCAAW |
| | DDSLNGYVFGTGTKVTVLG [SEQ ID NO: 50] |
| DNA | Caggctgtgctgactcagccaccctcagcgtctgggacccccgggcagagggtcaccatctcttgttct |
| | ggaagcagctccaacatcggaagttatactgtaagctggtaccagcaactcccaggaacggcccccaaa |
| | ttcctcatctattctaataatcagcggccctcaggggtccctgaccgattctctggctccaagtctggcacct |
| | cagcctccctggccatcagtgggctccagtctgaggatgaggctgattattactgtgctgcatgggatgac |
| | agcctgaatggttatgtcttcggaactgggaccaaggtcaccgtcctaggt [SEQ ID NO: 52] |
| scFv | QAVLTQPPSASGTPGQRVTISCSGSSSNIGSYTVSWYQQLPGTAPK |
| | FLIYSNNQRPSGVPDRFSGSKSGTSASLAISGLQSEDEADYYCAAW |
| | DDSLNGYVFGTGTKVTVLGSRGGGGSGGGGSGGGGSLEMAEVQL |
| | VQSGAEVKKPGESLKISCKGSGYDFTTYWIGWVRQMPGKGLEW |
| | MGIIYPGDSDTRYSPSVRGRVTISADKSINTAYLQWSSLEASDTAM |
| | YYCARMWTFSQDGWGQGTLVTVSS [SEQ ID NO: 84] |

In certain embodiments, the extracellular antigen-binding domain is a human scFv, which comprises the amino acid sequence of SEQ ID NO: 85 and specifically binds to a BCMA polypeptide (e.g., a BCMA polypeptide having the amino acid sequence SEQ ID NO: 71, or fragments thereof), which is designated as ET140-204 scFv (also referred to as “ET140-54 scFv”).

In certain embodiments, the extracellular antigen-binding domain is a human scFv, which comprises a heavy chain variable region comprising amino acids having the sequence set forth in SEQ ID NO: 53 and a light chain variable region comprising amino acids having the sequence set forth in SEQ ID NO: 54, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 14. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 53, as shown in Table 14. In certain embodiments, the extracellular antigen-binding domain comprises a V_{H} comprising amino acids having the sequence set forth in SEQ ID NO: 53, as shown in Table 14. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 54, as shown in Table 14. In certain embodiments, the extracellular antigen-binding domain comprises a V_{L} comprising amino acids having the sequence set forth in SEQ ID NO: 54, as shown in Table 14. In certain embodiments, the extracellular antigen-binding domain comprises a V_{H} comprising amino acids having the sequence set forth in SEQ ID NO: 53 and a V_{L} comprising amino acids having the sequence set forth in SEQ ID NO: 54, as shown in Table 14. In certain embodiments, the extracellular antigen-binding domain comprises a V_{H} CDR1 comprising amino acids having the sequence set forth in SEQ ID NO: 167 or conservative modifications thereof, a V_{H} CDR2 comprising amino acids having the sequence set forth in SEQ ID NO: 168 or conservative modifications thereof, and a V_{H} CDR3 comprising amino acids having the sequence set forth in SEQ ID NO: 169 or conservative modifications thereof, as shown in Table 14. In certain embodiments, the extracellular antigen-binding domain comprises a V_{L} CDR1 comprising amino acids having the sequence set forth in SEQ ID NO: 170 or conservative modifications thereof, a V_{L} CDR2 comprising amino acids having the sequence set forth in SEQ ID NO: 171 or conservative modifications thereof, and a V_{L} CDR3 comprising amino acids having the sequence set forth in SEQ ID NO: 172 or conservative modifications thereof, as shown in Table 14. In certain embodiments, the extracellular antigen-binding domain comprises a V_{H} CDR1 comprising amino acids having the sequence set forth in SEQ ID NO: 167 or conservative modifications thereof, a V_{H} CDR2 comprising amino acids having the sequence set forth in SEQ ID NO: 168 or conservative modifications thereof, a V_{H} CDR3 comprising amino acids having the sequence set forth in SEQ ID NO: 169 or conservative modifications thereof, a V_{L} CDR1 comprising amino acids having the sequence set forth in SEQ ID NO: 170 or conservative modifications thereof, a V_{L} CDR2 comprising amino acids having the sequence set forth in SEQ ID NO: 171 or conservative modifications thereof, and a V_{L} CDR3 comprising amino acids having the sequence set forth in SEQ ID NO: 172 or conservative modifications thereof, as shown in Table 14. In certain embodiments, the extracellular antigen-binding domain comprises a V_{H} CDR1 comprising amino acids having the sequence set forth in SEQ ID NO: 167, a V_{H} CDR2 comprising amino acids having the sequence set forth in SEQ ID NO: 168, a V_{H} CDR3 comprising amino acids having the sequence set forth in SEQ ID NO: 169, a V_{L} CDR1 comprising amino acids having the sequence set forth in SEQ ID NO: 170, a V_{L} CDR2 comprising amino acids having the sequence set forth in SEQ ID NO: 171, and a V_{L} CDR3 comprising amino acids having the sequence set forth in SEQ ID NO: 172.

TABLE 14

| | A BCMA polypeptide having the amino acid sequence of SEQ ID NO: 71 | | |
|---|---|---|---|
| Antigen | | | |
| CDRs | 1 | 2 | 3 |
| V_{H} | GYTFIDYY [SEQ | INPNSGGT [SEQ ID | ARSQRDGYMDY |
| | ID NO: 167] | NO: 168] | [SEQ ID NO: 169] |
| V_{L} | ISCTGTSSD [SEQ | EDS [SEQ ID NO: | SSNTRSSTLV [SEQ |
| | ID NO: 170] | 171] | ID NO: 172] |

| | |
|---|---|
| Full V_{H} | EVQLVQSGAEMKKPGASLKLSCKASGYTFIDYYVYWMRQAPGQ |
| | GLESMGWINPNSGGTNYAQKFQGRVTMTRDTSISTAYMELSRLRS |
| | DDTAMYYCARSQRDGYMDYWGQGTLVTVSS [SEQ ID NO: 53] |
| DNA | Gaagtgcagctggtgcagtctggggctgagatgaagaagcctggggcctcactgaagctctcctgcaa |
| | ggcttctggatacaccttcatcgactactatgtatactggatgcgacaggcccctggacaagggcttgagt |
| | ccatgggatggatcaaccctaacagtggtggcacaaactatgcacagaagtttcagggcagggtcacca |
| | tgaccagggacacgtccatcagcacagcctacatggagctgagcaggctgagatctgacgacaccgcc |
| | atgtattactgtgcgcgctcccagcgtgacggttacatggattactggggtcaaggtactctggtgaccgt |
| | ctcctca [SEQ ID NO: 55] |
| Full V_{L} | QSALTQPASVSASPGQSIAISCTGTSSDVGWYQQHPGKAPKLMIYE |
| | DSKRPSGVSNRFSGSKSGNTASLTISGLQAEDEADYYCSSNTRSST |
| | LVFGGGTKLTVLG [SEQ ID NO: 54] |
| DNA | Caatctgccctgactcagcctgcctccgtgtctgcgtctcctggacagtcgatcgccatctcctgcactgg |
| | aaccagcagtgacgttggttggtatcaacagcacccaggcaaagcccccaaactcatgatttatgagga |
| | cagtaagcggccctcaggggtttctaatcgcttctctggctccaagtctggcaacacggcctccctgacc |
| | atctctgggctccaggctgaggacgaggctgattattactgcagctcaaatacaagaagcagcactttggt |
| | gttcggcggagggaccaagctgaccgtcctaggt [SEQ ID NO: 56] |
| scFv | QSALTQPASVSASPGQSIAISCTGTSSDVGWYQQHPGKAPKLMIYE |
| | DSKRPSGVSNRFSGSKSGNTASLTISGLQAEDEADYYCSSNTRSST |
| | LVFGGGTKLTVLGSRGGGGSGGGGSGGGGSLEMAEVQLVQSGAE |
| | MKKPGASLKLSCKASGYTFIDYYVYWMRQAPGQGLESMGWINP |
| | NSGGTNYAQKFQGRVTMTRDTSISTAYMELSRLRSDDTAMYYCA |
| | RSQRDGYMDYWGQGTLVTVSS [SEQ ID NO: 85] |

In certain embodiments, the extracellular antigen-binding domain is a human scFv, which comprises the amino acid sequence of SEQ ID NO: 86 and specifically binds to a BCMA polypeptide (e.g., a BCMA polypeptide having the amino acid sequence SEQ ID NO: 71, or fragments thereof), which is designated as ET140-190 scFv (also referred to as “ET140-40 scFv”).

In certain embodiments, the extracellular antigen-binding domain is a human scFv, which comprises a heavy chain variable region comprising amino acids having the sequence set forth in SEQ ID NO: 57 and a light chain variable region comprising amino acids having the sequence set forth in SEQ ID NO: 58, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 15. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 57, as shown in Table 15. In certain embodiments, the extracellular antigen-binding domain comprises a V_{H} comprising amino acids having the sequence set forth in SEQ ID NO: 57, as shown in Table 15. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 58, as shown in Table 15. In certain embodiments, the extracellular antigen-binding domain comprises a V_{L} comprising amino acids having the sequence set forth in SEQ ID NO: 58, as shown in Table 15. In certain embodiments, the extracellular antigen-binding domain comprises a V_{H} comprising amino acids having the sequence set forth in SEQ ID NO: 57 and a V_{L} comprising amino acids having the sequence set forth in SEQ ID NO: 58, as shown in Table 15. In certain embodiments, the extracellular antigen-binding domain comprises a V_{H} CDR1 comprising amino acids having the sequence set forth in SEQ ID NO: 173 or conservative modifications thereof, a V_{H} CDR2 comprising amino acids having the sequence set forth in SEQ ID NO: 174 or conservative modifications thereof, and a V_{H} CDR3 comprising amino acids having the sequence set forth in SEQ ID NO: 175 or conservative modifications thereof, as shown in Table 15. In certain embodiments, the extracellular antigen-binding domain comprises a V_{L} CDR1 comprising amino acids having the sequence set forth in SEQ ID NO: 176 or conservative modifications thereof, a V_{L} CDR2 comprising amino acids having the sequence set forth in SEQ ID NO: 177 or conservative modifications thereof, and a V_{L} CDR3 comprising amino acids having the sequence set forth in SEQ ID NO: 178 or conservative modifications thereof, as shown in Table 15. In certain embodiments, the extracellular antigen-binding domain comprises a V_{H} CDR1 comprising amino acids having the sequence set forth in SEQ ID NO: 173 or conservative modifications thereof, a V_{H} CDR2 comprising amino acids having the sequence set forth in SEQ ID NO: 174 or conservative modifications thereof, a V_{H} CDR3 comprising amino acids having the sequence set forth in SEQ ID NO: 175 or conservative modifications thereof, a V_{L} CDR1 comprising amino acids having the sequence set forth in SEQ ID NO: 176 or conservative modifications thereof, a V_{L} CDR2 comprising amino acids having the sequence set forth in SEQ ID NO: 177 or conservative modifications thereof, and a V_{L} CDR3 comprising amino acids having the sequence set forth in SEQ ID NO: 178 or conservative modifications thereof, as shown in Table 15. In certain embodiments, the extracellular antigen-binding domain comprises a V_{H} CDR1 comprising amino acids having the sequence set forth in SEQ ID NO: 173, a V_{H} CDR2 comprising amino acids having the sequence set forth in SEQ ID NO: 174, a V_{H} CDR3 comprising amino acids having the sequence set forth in SEQ ID NO: 175, a V_{L} CDR1 comprising amino acids having the sequence set forth in SEQ ID NO: 176, a V_{L} CDR2 comprising amino acids having the sequence set forth in SEQ ID NO: 177, and a V_{L} CDR3 comprising amino acids having the sequence set forth in SEQ ID NO: 178.

TABLE 15

| | A BCMA polypeptide having the amino acid sequence of SEQ ID NO: 71 | | |
|---|---|---|---|
| Antigen | | | |
| CDRs | 1 | 2 | 3 |
| V_{H} | GYTFTDYY [SEQ | INPNSGGT [SEQ | ARSPYSGVLDK [SEQ |
| | ID NO: 173] | ID NO: 174] | ID NO: 175] |
| V_{L} | SSNIGAGFD [SEQ | GNS [SEQ ID NO: | QSYDSSLSGYV [SEQ |
| | ID NO: 176] | 177] | ID NO: 178] |

| | |
|---|---|
| Full V_{H} | QVQLVQSGAEVKKPGASVKVSCKASGYTFTDYYMHWVRQAPGQ |
| | RLEWMGWINPNSGGTNYAQKFQDRITVTRDTSSNTGYMELTRLRS |
| | DDTAVYYCARSPYSGVLDKWGQGTLVTVSS [SEQ ID NO: 57] |
| DNA | Caggtccagctggtacagtctggggctgaggtgaagaagcctggggcctcagtgaaggtctcctgcaag |
| | gcttctggatacaccttcaccgactactatatgcactgggtgcgacaggcccctggacaacggcttgagtg |
| | gatgggatggatcaaccctaacagtggtggcacaaactatgcacagaagtttcaggacaggatcaccgtg |
| | accagggacacctccagcaacacaggctacatggagctgaccaggctgagatctgacgacacggccgt |
| | gtattactgtgcgcgctctccgtactctggtgttctggataaatggggtcaaggtactctggtgaccgtct |
| | cctca [SEQ ID NO: 59] |
| Full V_{L} | QSVLTQPPSVSGAPGQRVTISCTGSSSNIGAGFDVHWYQQLPGTAP |
| | KLLIYGNSNRPSGVPDRFSGSKSGTSASLAITGLQAEDEADYYCQS |
| | YDSSLSGYVFGTGTKVTVLG [SEQ ID NO: 58] |
| DNA | Cagtctgtgctgacgcagccgccctcagtgtctggggccccagggcagagggtcaccatctcctgcact |
| | gggagcagctccaacatcggggcaggttttgatgtacactggtaccagcagcttccaggaacagccccc |
| | aaactcctcatctatggtaacagcaatcggccctcaggggtccctgaccgattctctggctccaagtctggc |
| | acctcagcctccctggccatcactgggctccaggctgaggatgaggctgattattactgccagtcctatgac |
| | agcagcctgagtggttatgtcttcggaactgggaccaaggtcaccgtcctaggt [SEQ ID NO: 60] |
| scFv | QSVLTQPPSVSGAPGQRVTISCTGSSSNIGAGFDVHWYQQLPGTAP |
| | KLLIYGNSNRPSGVPDRFSGSKSGTSASLAITGLQAEDEADYYCQS |
| | YDSSLSGYVFGTGTKVTVLGSRGGGGSGGGGSGGGGSLEMAQVQ |
| | LVQSGAEVKKPGASVKVSCKASGYTFTDYYMEIWVRQAPGQRLE |
| | WMGWINPNSGGTNYAQKFQDRITVTRDTSSNTGYMELTRLRSDD |
| | TAVYYCARSPYSGVLDKWGQGTLVTVSS [SEQ ID NO: 86] |

In certain embodiments, the extracellular antigen-binding domain is a human scFv, which comprises the amino acid sequence of SEQ ID NO: 87 and specifically binds to a BCMA polypeptide (e.g., a BCMA polypeptide having the amino acid sequence SEQ ID NO: 71, or fragments thereof), which is designated as ET140-187 scFv (also referred to as “ET140-37 scFv”).

In certain embodiments, the extracellular antigen-binding domain is a human scFv, which comprises a heavy chain variable region comprising amino acids having the sequence set forth in SEQ ID NO: 61 and a light chain variable region comprising amino acids having the sequence set forth in SEQ ID NO: 62, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 16. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 61, as shown in Table 16. In certain embodiments, the extracellular antigen-binding domain comprises a V_{H} comprising amino acids having the sequence set forth in SEQ ID NO: 61, as shown in Table 16. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 62, as shown in Table 16. In certain embodiments, the extracellular antigen-binding domain comprises a V_{L} comprising amino acids having the sequence set forth in SEQ ID NO: 62, as shown in Table 16. In certain embodiments, the extracellular antigen-binding domain comprises a V_{H} comprising amino acids having the sequence set forth in SEQ ID NO: 61 and a V_{L} comprising amino acids having the sequence set forth in SEQ ID NO: 62, as shown in Table 16. In certain embodiments, the extracellular antigen-binding domain comprises a V_{H} CDR1 comprising amino acids having the sequence set forth in SEQ ID NO: 179 or conservative modifications thereof, a V_{H} CDR2 comprising amino acids having the sequence set forth in SEQ ID NO: 180 or conservative modifications thereof, and a V_{H} CDR3 comprising amino acids having the sequence set forth in SEQ ID NO: 181 or conservative modifications thereof, as shown in Table 16. In certain embodiments, the extracellular antigen-binding domain comprises a V_{L} CDR1 comprising amino acids having the sequence set forth in SEQ ID NO: 182 or conservative modifications thereof, a V_{L} CDR2 comprising amino acids having the sequence set forth in SEQ ID NO: 183 or conservative modifications thereof, and a V_{L} CDR3 comprising amino acids having the sequence set forth in SEQ ID NO: 184 or conservative modifications thereof, as shown in Table 16. In certain embodiments, the extracellular antigen-binding domain comprises a V_{H} CDR1 comprising amino acids having the sequence set forth in SEQ ID NO: 179 or conservative modifications thereof, a V_{H} CDR2 comprising amino acids having the sequence set forth in SEQ ID NO: 180 or conservative modifications thereof, a V_{H} CDR3 comprising amino acids having the sequence set forth in SEQ ID NO: 181 or conservative modifications thereof, a V_{L} CDR1 comprising amino acids having the sequence set forth in SEQ ID NO: 182 or conservative modifications thereof, a V_{L} CDR2 comprising amino acids having the sequence set forth in SEQ ID NO: 183 or conservative modifications thereof, and a V_{L} CDR3 comprising amino acids having the sequence set forth in SEQ ID NO: 184 or conservative modifications thereof, as shown in Table 16. In certain embodiments, the extracellular antigen-binding domain comprises a V_{H} CDR1 comprising amino acids having the sequence set forth in SEQ ID NO: 179, a V_{H} CDR2 comprising amino acids having the sequence set forth in SEQ ID NO: 180, a V_{H} CDR3 comprising amino acids having the sequence set forth in SEQ ID NO: 181, a V_{L} CDR1 comprising amino acids having the sequence set forth in SEQ ID NO: 182, a V_{L} CDR2 comprising amino acids having the sequence set forth in SEQ ID NO: 183 and a V_{L} CDR3 comprising amino acids having the sequence set forth in SEQ ID NO: 184.

TABLE 16

| | A BCMA polypeptide having the amino acid sequence of SEQ ID NO: 71 | | |
|---|---|---|---|
| Antigen | | | |
| CDRs | 1 | 2 | 3 |
| V_{H} | GGTFSSYA [SEQ | IIPILGTA [SEQ ID | ARSGYGSYRWEDS |
| | ID NO: 179] | NO: 180] | [SEQ ID NO: 181] |
| V_{L} | SSNIGSNY [SEQ | SNN [SEQ ID NO: | AAWDDSLSASYV |
| | ID NO: 182] | 183] | [SEQ ID NO: 184] |

| | |
|---|---|
| Full V_{H} | QVQLVQSGAEVKKPGSSVKVSCKASGGTFSSYAISWVRQAPGQG |
| | LEWMGRIIPILGTANYAQKFQGRVTITADESTSTAYMELSSLRSED |
| | TAVYYCARSGYGSYRWEDSWGQGTLVTVSS [SEQ ID NO: 61] |
| DNA | Caggtgcagctggtgcagtctggggctgaggtgaagaagcctgggtcctcggtgaaggtctcctgcaa |
| | ggcttctggaggcaccttcagcagctatgctatcagctgggtgcgacaggcccctggacaagggcttga |
| | gtggatgggaaggatcatccctatccttggtacagcaaactacgcacagaagttccagggcagagtcac |
| | gattaccgcggacgaatccacgagcacagcctacatggagctgagcagcctgagatctgaggacacgg |
| | ccgtgtattactgtgcgcgctctggttacggttcttaccgttgggaagattcttggggtcaaggtactc |
| | tggtgaccgtctcctca [SEQ ID NO: 63] |
| Full V_{L} | QAVLTQPPSASGTPGQRVTISCSGSSSNIGSNYVFWYQQLPGTAPK |
| | LLIYSNNQRPSGVPDRFSGSKSGTSASLAISGLRSEDEADYYCAAW |
| | DDSLSASYVFGTGTKVTVLG [SEQ ID NO: 62] |
| DNA | Caggctgtgctgactcagccaccctcagcgtctgggacccccgggcagagggtcaccatctcttgttct |
| | ggaagcagctccaacatcggaagtaattacgtattctggtaccagcagctcccaggaacggcccccaaa |
| | ctcctcatctatagtaataatcagcggccctcaggggtccctgaccgattctctggctccaagtctggcacc |
| | tcagcctccctggccatcagtgggctccggtccgaggatgaggctgattattactgtgcagcatgggatg |
| | acagcctgagtgcctcttatgttttcggaactgggaccaaggtcaccgtcctaggt [SEQ ID |
| | NO: 64] |
| scFv | QAVLTQPPSASGTPGQRVTISCSGSSSNIGSNYVFWYQQLPGTAPK |
| | LLIYSNNQRPSGVPDRFSGSKSGTSASLAISGLRSEDEADYYCAAW |
| | DDSLSASYVFGTGTKVTVLGSRGGGGSGGGGSGGGGSLEMAQVQ |
| | LVQSGAEVKKPGSSVKVSCKASGGTFSSYAISWVRQAPGQGLEW |
| | MGRIIPILGTANYAQKFQGRVTITADESTSTAYMELSSLRSEDTAV |
| | YYCARSGYGSYRWEDSWGQGTLVTVSS [SEQ ID NO: 87] |

In certain embodiments, the extracellular antigen-binding domain is a human scFv, which comprises the amino acid sequence of SEQ ID NO: 88 and specifically binds to a BCMA polypeptide (e.g., a BCMA polypeptide having the amino acid sequence SEQ ID NO: 71, or fragments thereof), which is designated as ET140-174 scFv (also referred to as “ET140-24 scFv”).

In certain embodiments, the extracellular antigen-binding domain is a human scFv, which comprises a heavy chain variable region comprising amino acids having the sequence set forth in SEQ ID NO: 65 and a light chain variable region comprising amino acids having the sequence set forth in SEQ ID NO: 66, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain is a human scFv-Fc fusion protein or full length human IgG with V_{H} and V_{L} regions or CDRs selected from Table 17. In certain embodiments, the extracellular antigen-binding domain comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 65, as shown in Table 17. In certain embodiments, the extracellular antigen-binding domain comprises a V_{H} comprising amino acids having the sequence set forth in SEQ ID NO: 65, as shown in Table 17. In certain embodiments, the extracellular antigen-binding domain comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 66, as shown in Table 17. In certain embodiments, the extracellular antigen-binding domain comprises a V_{L} comprising amino acids having the sequence set forth in SEQ ID NO: 66, as shown in Table 17. In certain embodiments, the extracellular antigen-binding domain comprises a V_{H} comprising amino acids having the sequence set forth in SEQ ID NO: 65 and a V_{L} comprising amino acids having the sequence set forth in SEQ ID NO: 66, as shown in Table 17. In certain embodiments, the extracellular antigen-binding domain comprises a V_{H} CDR1 comprising amino acids having the sequence set forth in SEQ ID NO: 185 or conservative modifications thereof, a V_{H} CDR2 comprising amino acids having the sequence set forth in SEQ ID NO: 186 or conservative modifications thereof, and a V_{H} CDR3 comprising amino acids having the sequence set forth in SEQ ID NO: 187 or conservative modifications thereof, as shown in Table 17. In certain embodiments, the extracellular antigen-binding domain comprises a V_{L} CDR1 comprising amino acids having the sequence set forth in SEQ ID NO: 188 or conservative modifications thereof, a V_{L} CDR2 comprising amino acids having the sequence set forth in SEQ ID NO: 189 or conservative modifications thereof, and a V_{L} CDR3 comprising amino acids having the sequence set forth in SEQ ID NO: 190 or conservative modifications thereof, as shown in Table 17. In certain embodiments, the extracellular antigen-binding domain comprises a V_{H} CDR1 comprising amino acids having the sequence set forth in SEQ ID NO: 185 or conservative modifications thereof, a V_{H} CDR2 comprising amino acids having the sequence set forth in SEQ ID NO: 186 or conservative modifications thereof, a V_{H} CDR3 comprising amino acids having the sequence set forth in SEQ ID NO: 187 or conservative modifications thereof, a V_{L} CDR1 comprising amino acids having the sequence set forth in SEQ ID NO: 188 or conservative modifications thereof, a V_{L} CDR2 comprising amino acids having the sequence set forth in SEQ ID NO: 189 or conservative modifications thereof, and a V_{L} CDR3 comprising amino acids having the sequence set forth in SEQ ID NO: 190 or conservative modifications thereof, as shown in Table 17. In certain embodiments, the extracellular antigen-binding domain comprises a V_{H} CDR1 comprising amino acids having the sequence set forth in SEQ ID NO: 185, a V_{H} CDR2 comprising amino acids having the sequence set forth in SEQ ID NO: 186, a V_{H} CDR3 comprising amino acids having the sequence set forth in SEQ ID NO: 187, a V_{L} CDR1 comprising amino acids having the sequence set forth in SEQ ID NO: 188, a V_{L} CDR2 comprising amino acids having the sequence set forth in SEQ ID NO: 189, and a V_{L} CDR3 comprising amino acids having the sequence set forth in SEQ ID NO: 190.

TABLE 17

| | A BCMA polypeptide having the amino acid sequence of SEQ ID NO: 71 | | |
|---|---|---|---|
| Antigen | | | |
| CDRs | 1 | 2 | 3 |
| V_{H} | GYSFTSYW [SEQ | IYPGDSDT [SEQ ID | ARYSGSFDN [SEQ |
| | ID NO: 185] | NO: 186] | ID NO: 187] |
| V_{L} | SSNIGSHS [SEQ | TNN [SEQ ID NO: | AAWDGSLNGLV |
| | ID NO: 188] | 189] | [SEQ ID NO: 190] |

| | |
|---|---|
| Full V_{H} | EVQLVQSGAEVKKPGESLKISCKGSGYSFTSYWIGWVRQMPGKG |
| | LEWMGIIYPGDSDTRYSPSFQGHVTISADKSISTAYLQWSSLKASD |
| | TAMYYCARYSGSFDNWGQGTLVTVSS [SEQ ID NO: 65] |
| DNA | Gaggtgcagctggtgcagtctggagcagaggtgaaaaagcccggggagtctctgaagatctcctgta |
| | agggttctggatacagctttaccagctactggatcggctgggtgcgccagatgcccgggaaaggcctg |
| | gagtggatggggatcatctatcctggtgactctgataccagatacagcccgtccttccaaggccacgtca |
| | ccatctcagctgacaagtccatcagcactgcctacctgcagtggagcagcctgaaggcctcggacacc |
| | gccatgtattactgtgcgcgctactctggttctttcgataactggggtcaaggtactctggtgaccgtct |
| | cctca [SEQ ID NO: 67] |
| Full V_{L} | SYELTQPPSASGTPGQRVTMSCSGTSSNIGSHSVNWYQQLPGTAP |
| | KLLIYTNNQRPSGVPDRFSGSKSGTSASLAISGLQSEDEADYYCA |
| | AWDGSLNGLVFGGGTKLTVLG [SEQ ID NO: 66] |
| DNA | Tcctatgagctgactcagccaccctcagcgtctgggacccccgggcagagggtcaccatgtcttgttct |
| | ggaaccagctccaacatcggaagtcactctgtaaactggtaccagcagctcccaggaacggcccccaa |
| | actcctcatctatactaataatcagcggccctcaggggtccctgaccgattctctggctccaagtctggca |
| | cctcagcctccctggccatcagtggcctccagtctgaggatgaggctgattattactgtgcagcatggga |
| | tggcagcctgaatggtctggtattcggcggagggaccaagctgaccgtcctaggt [SEQ ID |
| | NO: 68] |
| scFv | SYELTQPPSASGTPGQRVTMSCSGTSSNIGSHSVNWYQQLPGTAP |
| | KLLIYTNNQRPSGVPDRFSGSKSGTSASLAISGLQSEDEADYYCA |
| | AWDGSLNGLVFGGGTKLTVLGSRGGGGSGGGGSGGGGSLEMAE |
| | VQLVQSGAEVKKPGESLKISCKGSGYSFTSYWIGWVRQMPGKGL |
| | EWMGIIYPGDSDTRYSPSFQGHVTISADKSISTAYLQWSSLKASDT |
| | AMYYCARYSGSFDNWGQGTLVTVSS [SEQ ID NO: 88] |

An extracellular antigen-binding domain (e.g., scFv) comprising V_{H} and/or V_{L} regions having high (i.e., 80% or greater) homology to the V_{H} and V_{L} regions of the sequences set forth above, can be obtained by mutagenesis (e.g., site-directed or PCR-mediated mutagenesis), followed by testing of the encoded altered scFv for retained function (i.e., the binding affinity) using the binding assays described herein. In certain embodiments, a V_{H} sequence having at least 90%, 91%, 92%, 93%, 94%, 95%, 96%, 97%, 98% or 99% identity contains substitutions (e.g., conservative substitutions to generate conservative modifications of a sequence), insertions or deletions relative to the reference sequence, but an extracellular antigen-binding domain (e.g., scFv) comprising that sequence retains the ability to bind to a BCMA polypeptide. In certain embodiments, a V_{L} sequence having at least 90%, 91%, 92%, 93%, 94%, 95%, 96%, 97%, 98%, or 99% identity contains substitutions (e.g., conservative substitutions), insertions or deletions relative to the reference sequence, but an extracellular antigen-binding domain (e.g., scFv) comprising that sequence retains the ability to bind to a BCMA polypeptide. In certain embodiments, a total of about 1 to about 10 amino acids have been substituted, inserted and/or deleted in the disclosed sequences. For example, and not by way of limitation, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted. Non-limiting examples of conservative modifications are provided below, e.g., within Table 18.

As used herein, the term “conservative sequence modifications” refers to amino acid modifications that do not significantly affect or alter the binding characteristics of the presently disclosed CAR (e.g., the extracellular antigen-binding domain) comprising the amino acid sequence. Such conservative modifications include amino acid substitutions, additions and deletions. Modifications can be introduced into the human scFv of the presently disclosed subject matter by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered. Exemplary conservative amino acid substitutions are shown in Table 18.

TABLE 18

| | Exemplary |
|---|---|
| | conservative |
| Original | amino acid |
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

In certain non-limiting embodiments, an extracellular antigen-binding domain of the CAR can comprise a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). Non-limiting examples of peptide linkers are disclosed in Shen et al., Anal. Chem. 80(6):1910-1917 (2008) and WO 2014/087010.

In one non-limiting example, the linker comprises amino acids having the sequence set forth in SEQ ID NO: 69. In certain embodiments, the nucleotide sequence encoding the amino acid sequence of SEQ ID NO: 69 is set forth in SEQ ID NO: 70. In one non-limiting example, the linker is a G4S linker that comprises amino acids having the sequence set forth in SEQ ID NO: 210. In certain embodiments, the nucleotide sequence encoding the amino acid sequence of SEQ ID NO: 98 is set forth in SEQ ID NO: 211.

In certain embodiments, the linker comprises amino acids having the sequence set forth in SEQ ID NO: 212 as provided below.

[SEQ ID NO: 212]
GGGGS.

In certain embodiments, the linker comprises amino acids having the sequence set forth in SEQ ID NO: 213 as provided below.

[SEQ ID NO: 213]
SGGSGGS.

In certain embodiments, the linker comprises amino acids having the sequence set forth in SEQ ID NO: 214 as provided below.

[SEQ ID NO: 214]
GGGGSGGGS.

In certain embodiments, the linker comprises amino acids having the sequence set forth in SEQ ID NO: 215 as provided below.

[SEQ ID NO: 215]
GGGGSGGGGS.

In certain embodiments, the linker comprises amino acids having the sequence set forth in SEQ ID NO: 216 as provided below.

[SEQ ID NO: 216]
GGGGSGGGGSGGGGGGGS.

In certain embodiments, the linker comprises amino acids having the sequence set forth in SEQ ID NO: 217 as provided below.

[SEQ ID NO: 217]
GGGGSGGGGSGGGGSGGGGS.

In certain embodiments, the linker comprises amino acids having the sequence set forth in SEQ ID NO: 218 as provided below.

[SEQ ID NO: 218]
GGGGSGGGGSGGGGSGGGGSGGGGS.

In certain embodiments, the linker comprises amino acids having the sequence set forth in SEQ ID NO: 219 as provided below.

[SEQ ID NO: 219]
GGGGSGGGGSGGGGSGGGGSGGGGSGGGGS.

In certain embodiments, the linker comprises amino acids having the sequence set forth in SEQ ID NO: 220 as provided below.

[SEQ ID NO: 220]
GGGGSGGGGSGGGGSGGGGSGGGGSGGGGSGGGGS.

In certain embodiments, the linker comprises amino acids having the sequence set forth in SEQ ID NO: 220 as provided below.

[SEQ ID NO: 220]
EPKSCDKTHTCPPCP.

In certain embodiments, the linker comprises amino acids having the sequence set forth in SEQ ID NO: 221 as provided below.

[SEQ ID NO: 222]
GGGGSGGGSEPKSCDKTHTCPPCP.

In certain embodiments, the linker comprises amino acids having the sequence set forth in SEQ ID NO: 223 as provided below.

[SEQ ID NO: 223]
ELKTPLGDTTHTCPRCPEPKSCDTPPPCPRCPEPKSCDTPPPCPRCPEPK
SCDTPPPCPRCP.

In certain embodiments, the linker comprises amino acids having the sequence set forth in SEQ ID NO: 224 as provided below.

[SEQ ID NO: 224]
GSGSGS.

In certain embodiments, the linker comprises amino acids having the sequence set forth in SEQ ID NO: 225 as provided below.

[SEQ ID NO: 225]
AAA.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In non-limiting examples, the signal peptide is covalently joined to the 5′ terminus of the extracellular antigen-binding domain. In certain embodiments, the signal peptide comprises a CD8 polypeptide comprising amino acids having the sequence set forth in SEQ ID NO: 191 as provided below.

[SEQ ID NO: 191]
MALPVTALLLPLALLLHAAR

The nucleotide sequence encoding the amino acid sequence of SEQ ID NO: 191 is set forth in SEQ ID NO: 192, which is provided below:

[SEQ ID NO: 192]
ATGGCTCTCCCAGTGACTGCCCTACTGCTTCCCCTAGCGCTTCTCCTGCA
TGCAGCTCGT

In another embodiment, the signal peptide comprises amino acids having the sequence set forth in SEQ ID NO: 205 as provided below.

[SEQ ID NO: 205]
METDTLLLWVLLLWVPGSTG

The nucleotide sequence encoding the amino acid sequence of SEQ ID NO: 205 is set forth in SEQ ID NO: 206, which is provided below:

[SEQ ID NO: 206]
ATGGAAACCGACACCCTGCTGCTGTGGGTGCTGCTGCTGTGGGTGCCAGG
ATCCACAGGA

In certain embodiments, the human scFv comprises a heavy chain variable region, a light chain variable region, a linker peptide between the heavy chain variable region and the light chain variable region, and an His-tag and an HA-tag. In certain embodiments, the amino acid sequence of the His-tag and HA-tag comprises the amino acid sequence of SEQ ID NO: 275, which is provided below:

[SEQ ID NO: 275]
TSGQAGQHHHHHHGAYPYDVPDYAS

The nucleotide sequence encoding SEQ ID NO: 275 is SEQ ID NO: 276, which is provided below:

[SEQ ID NO: 276]
ACTAGTGGCCAGGCCGGCCAGCACCATCACCATCACCATGGCGCATACCC
GTACGACGTTCCGGACTACGCTTCT

In certain embodiments, the extracellular antigen-binding domain (e.g., a human scFv) binds to a human BCMA polypeptide comprising the amino acid sequence set forth in SEQ ID NO: 71. In certain embodiments, the extracellular antigen-binding domain (e.g., a human scFv) binds to one or more portion of the amino acid sequence set forth in SEQ ID NO: 71. In certain embodiments, the extracellular antigen-binding domain (e.g., a human scFv) binds to an epitope region comprising amino acids 14-22 of SEQ ID NO: 71. In certain embodiments, the extracellular antigen-binding domain (e.g., a human scFv) binds to one, two, three, four, five, six, or seven epitope region selected from the group consisting of amino acids 8-22, 9-23, 10-24, 11-25, 12-26, 13-27, 14-28 and 8-28 of SEQ ID NO: 71. In certain embodiments, the extracellular antigen-binding domain (e.g., a human scFv) that binds to an epitope region comprising amino acids 14-22 of SEQ ID NO: 71 comprises a heavy chain variable region comprising amino acids having the sequence set forth in SEQ ID NO: 9 and a light chain variable region comprising amino acids having the sequence set forth in SEQ ID NO: 10, optionally with (iii) a linker sequence, for example a linker peptide, between the heavy chain variable region and the light chain variable region. In certain embodiments, the linker comprises amino acids having the sequence set forth in SEQ ID NO: 69. In certain embodiments, the extracellular antigen-binding domain that binds to an epitope region comprising amino acids 14-22 of SEQ ID NO: 71 is a human scFv with V_{H} and V_{L} regions or CDRs selected from Table 6. In certain embodiments, the extracellular antigen-binding domain that binds to amino acids 14-22 of SEQ ID NO: 71 comprises is a human scFv-Fc fusion protein or full length human IgG with V_{H} and V_{L} regions or CDRs selected from Table 6. In certain embodiments, the extracellular antigen-binding domain (e.g., a human scFv) that binds to an epitope region comprising amino acids 14-22 of SEQ ID NO: 71 comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 21. In certain embodiments, the extracellular antigen-binding domain (e.g., a human scFv) that binds to an epitope region comprising amino acids 14-22 of SEQ ID NO: 71 comprises a V_{H} Comprising amino acids having the sequence set forth in SEQ ID NO: 21. In certain embodiments, the extracellular antigen-binding domain (e.g., a human scFv) that binds to an epitope region comprising amino acids 14-22 of SEQ ID NO: 71 comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO: 22. In certain embodiments, the extracellular antigen-binding domain (e.g., a human scFv) that binds to an epitope region comprising amino acids 14-22 of SEQ ID NO: 71 comprises a V_{L} comprising amino acids having the sequence set forth in SEQ ID NO: 22. In certain embodiments, the extracellular antigen-binding domain (e.g., a human scFv) that binds to an epitope region comprising amino acids 14-22 of SEQ ID NO: 71 comprises a V_{H} comprising amino acids having the sequence set forth in SEQ ID NO: 21 and a V_{L} Comprising amino acids having the sequence set forth in SEQ ID NO: 22. In certain embodiments, the extracellular antigen-binding domain (e.g., a human scFv) that binds to an epitope region comprising amino acids 14-22 of SEQ ID NO: 71 comprises a V_{H} CDR1 comprising amino acids having the sequence set forth in SEQ ID NO: 119 or conservative modifications thereof, a V_{H} CDR2 comprising amino acids having the sequence set forth in SEQ ID NO: 120 or conservative modifications thereof, and a V_{H} CDR3 comprising amino acids having the sequence set forth in SEQ ID NO: 121 or conservative modifications thereof. In certain embodiments, the extracellular antigen-binding domain (e.g., a human scFv) that binds to an epitope region comprising amino acids 14-22 of SEQ ID NO: 71 comprises a V_{L} CDR1 comprising amino acids having the sequence set forth in SEQ ID NO: 122 or conservative modifications thereof, a V_{L} CDR2 comprising amino acids having the sequence set forth in SEQ ID NO: 123 or conservative modifications thereof, and a V_{L} CDR3 comprising amino acids having the sequence set forth in SEQ ID NO: 124 or conservative modifications thereof. In certain embodiments, the extracellular antigen-binding domain (e.g., a human scFv) that binds to an epitope region comprising amino acids 14-22 of SEQ ID NO: 71 comprises a V_{H} CDR1 comprising amino acids having the sequence set forth in SEQ ID NO: 119 or conservative modifications thereof, a V_{H} CDR2 comprising amino acids having the sequence set forth in SEQ ID NO: 120 or conservative modifications thereof, a V_{H} CDR3 comprising amino acids having the sequence set forth in SEQ ID NO: 121 or conservative modifications thereof, a V_{L} CDR1 comprising amino acids having the sequence set forth in SEQ ID NO: 122 or conservative modifications thereof, a V_{L} CDR2 comprising amino acids having the sequence set forth in SEQ ID NO: 123 or conservative modifications thereof, and a V_{L} CDR3 comprising amino acids having the sequence set forth in SEQ ID NO: 124 or conservative modifications thereof. In certain embodiments, the extracellular antigen-binding domain (e.g., a human scFv) that binds to an epitope region comprising amino acids 14-22 of SEQ ID NO: 71 comprises a V_{H} CDR1 comprising amino acids having the sequence set forth in SEQ ID NO: 119, a V_{H} CDR2 comprising amino acids having the sequence set forth in SEQ ID NO: 120, a V_{H} CDR3 comprising amino acids having the sequence set forth in SEQ ID NO: 121, a V_{L} CDR1 comprising amino acids having the sequence set forth in SEQ ID NO: 122, a V_{L} CDR2 comprising amino acids having the sequence set forth in SEQ ID NO: 123, and a V_{L} CDR3 comprising amino acids having the sequence set forth in SEQ ID NO: 124. In certain embodiments, the extracellular antigen-binding domain is ET140-3 (or “ET140-153”) scFv.

### Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID No: 193), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 193 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 150 to 200, or 200 to 220 of SEQ ID NO: 193. In certain embodiments, the CAR of the presently disclosed comprises a transmembrane domain comprising a CD28 polypeptide, and an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 193.

SEQ ID NO: 193 is provided below:

| [SEQ ID NO: 193] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain and the intracellular domain (e.g., the co-stimulatory signaling region) of the presently disclosed CAR (amino acids 114 to 220 of SEQ ID NO: 193) comprises nucleic acids having the sequence set forth in SEQ ID NO: 194 as provided below.

[SEQ ID NO: 194]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCC

In certain embodiments, the transmembrane domain of a presently disclosed CAR comprises a CD8 polypeptide. The CD8 polypeptide can have an amino acid sequence that is at least about 85%, about 90%, about 95%, about 96%, about 97%, about 98%, about 99% or 100% homologous to the sequence having a NCBI Reference No: AAH25715 (SEQ ID No: 226), or fragments thereof, and/or may optionally comprise up to one or up to two or up to three conservative amino acid substitutions. In non-limiting embodiments, the CD8 polypeptide can have an amino acid sequence that is a consecutive portion of SEQ ID NO: 226 which is at least 20, or at least 30, or at least 40, or at least 50, or at least 70, or at least 100, or at least 150, or at least 200 and up to 235 amino acids in length. Alternatively or additionally, in non-limiting various embodiments, the CD28 polypeptide has an amino acid sequence of amino acids 1 to 235, 1 to 50, 50 to 100, 100 to 150, 150 to 200, 130 to 210, or 200 to 235 of SEQ ID NO: 226. In certain embodiments, the CD8 polypeptide comprised in the transmembrane domain has an amino acid sequence of amino acids 137 to 207 of SEQ ID NO: 226.

SEQ ID NO: 226 is provided below:

| [SEQ ID NO: 226] | |
|---|---|
| 1 | MALPVTALLL PLALLLHAAR PSQFRVSPLD RTWNLGETVE LKCQVLLSNP TSGCSWLFQP |
| 61 | RGAAASPTFL LYLSQNKPKA AEGLDTQRFS GKRLGDTFVL TLSDFRRENE GCYFCSALSN |
| 121 | SIMYFSHFVP VFLPAKPTTT PAPRPPTPAP TIASQPLSLR PEACRPAAGG AVHTRGLDFA |
| 181 | CDIYIWAPLA GTCGVLLLSL VITLYCNHRN RRRVCKCPRP VVKSGDKPSL SARYV. |

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (amino acids 137 to 207 of SEQ ID NO: 226) comprises nucleic acids having the sequence set forth in SEQ ID NO: 227 as provided below.

[SEQ ID NO: 227]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAAC

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3.

### Intracellular Domain of a CAR

In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises three ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence set forth in SEQ ID NO: 195, or fragments thereof, and/or may optionally comprise up to one or up to two or up to three conservative amino acid substitutions. In non-limiting embodiments, the CD3ζ polypeptide can have an amino acid sequence that is a consecutive portion of SEQ ID NO: 195 which is at least 20, or at least 30, or at least 40, or at least 50, and up to 163 amino acids in length. Alternatively or additionally, in non-limiting various embodiments, the CD3ζ polypeptide has an amino acid sequence of amino acids 1 to 163, 1 to 50, 50 to 100, 100 to 150, or 150 to 163 of SEQ ID NO: 195. In certain embodiments, the CD3ζ polypeptide comprised in the intracellular domain of a presently disclosed CAR has an amino acid sequence of amino acids 52 to 163 of SEQ ID NO: 195.

SEQ ID NO: 195 is provided below:

| [SEQ ID NO: 195] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP RRKNPQEGLY NELQKDKMAE |
| 121 | AYSEIGMKGE RRRGKGHDGL YQGLSTATKD TYDALHMQAL PPR |

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide comprised in the intracellular domain of a presently disclosed CARs (amino acid sequence of amino acids 52 to 163 of SEQ ID NO: 195) comprises nucleic acids having the sequence set forth in SEQ ID NO: 196 as provided below.

[SEQ ID NO: 196]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190 (e.g., the nucleotide sequence encoding 4-1BB is set forth in SEQ ID NO: 15, the nucleotide sequence encoding ICOS is set forth in SEQ ID NO: 16, and the nucleotide sequence encoding DAP-10 is set forth in SEQ ID NO: 17 in U.S. Pat. No. 7,446,190), which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO: 197) or fragments thereof, and/or may optionally comprise up to one or up to two or up to three conservative amino acid substitutions. In certain embodiments, the 4-1BB polypeptide comprised in the intracellular domain of a presently disclosed CAR has an amino acid sequence of amino acids 214-255 of SEQ ID NO: 197.

SEQ ID NO: 197 is provided below:

| [SEQ ID NO: 197] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide comprised in the intracellular domain of a presently disclosed CARs (amino acids 214-255 of SEQ ID NO: 197) comprises nucleic acids having the sequence set forth in SEQ ID NO: 228 as provided below.

[SEQ ID NO: 228]
aaacggggcagaaagaagctcctgtatatattcaaacaaccatttatgag
accagtacaaactactcaagaggaagatggctgtagctgccgatttccag
aagaagaagaaggaggatgtgaactg

An OX40 polypeptide can have an amino acid sequence that is at least about 85%, about 90%, about 95%, about 96%, about 97%, about 98%, about 99% or 100% homologous to the sequence having a NCBI Reference No: P43489 or NP_003318 (SEQ ID NO: 198), or fragments thereof, and/or may optionally comprise up to one or up to two or up to three conservative amino acid substitutions.

SEQ ID NO: 198 is provided below:

| [SEQ ID NO: 198] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 199) or fragments thereof, and/or may optionally comprise up to one or up to two or up to three conservative amino acid substitutions.

SEQ ID NO: 199 is provided below:

| [SEQ ID NO: 199] | |
|---|---|
| 1 | MKSGLWYFFL FCLRIKVLTG EINGSANYEM FIFHNGGVQI LCKYPDIVQQ FKMQLLKGGQ |
| 61 | ILCDLTKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 200) (homology herein may be determined using standard software such as BLAST or FASTA) or fragments thereof, and/or may optionally comprise up to one or up to two or up to three conservative amino acid substitutions.

SEQ ID NO: 200 is provided below:

| [SEQ ID NO: 200] | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system.

In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 201) or fragments thereof, and/or may optionally comprise up to one or up to two or up to three conservative amino acid substitutions.

SEQ ID NO: 201 is provided below:

| [SEQ ID NO: 201] | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 202) or fragments thereof, and/or may optionally comprise up to one or up to two or up to three conservative amino acid substitutions.

SEQ ID NO: 202 is provided below:

| [SEQ ID NO: 202] | |
|---|---|
| 1 | MWEAQFLGLL FLQPLWVAPV KPLQPGAEVP VVWAQEGAPA QLPCSPTIPL QDLSLLRRAG |
| 61 | VTWQHQPDSG PPAAAPGHPL APGPHPAAPS SWGPRPRRYT VLSVGPGGLR SGRLPLQPRV |
| 121 | QLDERGRQRG DFSLWLRPAR RADAGEYRAA VHLRDRALSC RLRLRLGQAS MTASPPGSLR |
| 181 | ASDWVILNCS FSRPDRPASV HWFRNRGQGR VPVRESPHHH LAESFLFLPQ VSPMDSGPWG |
| 241 | CILTYRDGFN VSIMYNLTVL GLEPPTPLTV YAGAGSRVGL PCRLPAGVGT RSFLTAKWTP |
| 301 | PGGGPDLLVT GDNGDFTLRL EDVSQAQAGT YTCHIHLQEQ QLNATVTLAI ITVTPKSFGS |
| 361 | PGSLGKLLCE VTPVSGQERF VWSSLDTPSQ RSFSGPWLEA QEAQLLSQPW QCQLYQGERL |
| 421 | LGAAVYFTEL SSPGAQRSGR APGALPAGHL LLFLILGVLS LLLLVTGAFG FHLWRRQWRP |
| 481 | RRFSALEQGI HPPQAQSKIE ELEQEPEPEP EPEPEPEPEP EPEQL |

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 203) or fragments thereof, and/or may optionally comprise up to one or up to two or up to three conservative amino acid substitutions.

SEQ ID NO: 203 is provided below:

| [SEQ ID NO: 203] | |
|---|---|
| 1 | MLGQVVTLIL LLLLKVYQGK GCQGSADHVV SISGVPLQLQ PNSIQTKVDS IAWKKLLPSQ |
| 61 | NGFHHILKWE NGSLPSNTSN DRFSFIVKNL SLLIKAAQQQ DSGLYCLEVT SISGKVQTAT |
| 121 | FQVFVFESLL PDKVEKPRLQ GQGKILDRGR CQVALSCLVS RDGNVSYAWY RGSKLIQTAG |
| 181 | NLTYLDEEVD INGTHTYTCN VSNPVSWESH TLNLTQDCQN AHQEFRFWPF LVIIVILSAL |
| 241 | FLGTLACFCV WRRKRKEKQS ETSPKEFLTI YEDVKDLKTR RNHEQEQTFP GGGSTIYSMI |
| 301 | QSQSSAPTSQ EPAYTLYSLI QPSRKSGSRK RNHSPSFNST IYEVIGKSQP KAQNPARLSR |
| 361 | KELENFDVYS |

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO: 204) or fragments thereof, and/or may optionally comprise up to one or up to two or up to three conservative amino acid substitutions.

SEQ ID NO: 204 is provided below:

| [SEQ ID NO: 204] | |
|---|---|
| 1 | MKTLPAMLGT GKLFWVFFLI PYLDIWNIHG KESCDVQLYI KRQSEHSILA GDPFELECPV |
| 61 | KYCANRPHVT WCKLNGTTCV KLEDRQTSWK EEKNISFFIL HFEPVLPNDN GSYRCSANFQ |
| 121 | SNLIESHSTT LYVTDVKSAS ERPSKDEMAS RPWLLYRLLP LGGLPLLITT CFCLFCCLRR |
| 181 | HQGKQNELSD TAGREINLVD AHLKSEQTEA STRQNSQVLL SETGIYDNDP DLCFRMQEGS |
| 241 | EVYSNPCLEE NKPGIVYASL NHSVIGPNSR LARNVKEAPT EYASICVRS |

In accordance with the presently disclosed subject matter, a “BTLA nucleic acid molecule” refers to a polynucleotide encoding a BTLA polypeptide.

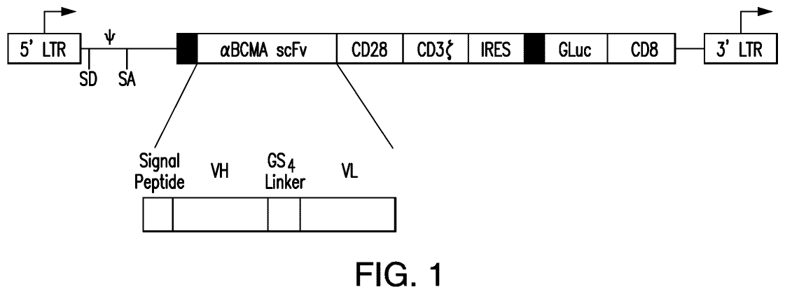

In certain embodiments, the CAR comprises an extracellular antigen-binding region that comprises a human scFv that specifically binds to human BCMA, a transmembrane domain comprising a CD28 polypeptide, and an intracellular domain comprising a CD3ζ polypeptide and a co-stimulatory signaling region that comprises a CD28 polypeptide, as shown in FIG. 1. As shown in FIG. 1, the CAR also comprises a signal peptide or a leader covalently joined to the 5′ terminus of the extracellular antigen-binding domain. In certain embodiments, the signal peptide comprises amino acids having the sequence set forth in SEQ ID NO: 205.

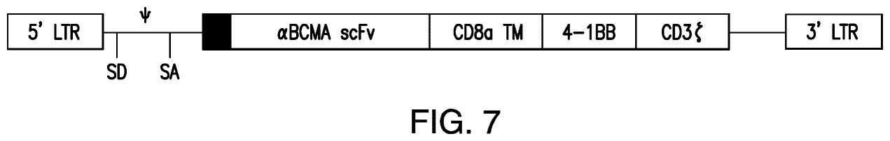

In certain embodiments, the CAR comprises an extracellular antigen-binding region that comprises a human scFv that specifically binds to human BCMA, a transmembrane domain comprising a CD8 polypeptide, and an intracellular domain comprising a CD3ζ polypeptide and a co-stimulatory signaling region that comprises a 4-1BB polypeptide, as shown in FIG. 7. As shown in FIG. 7, the CAR also comprises a signal peptide or a leader covalently joined to the 5′ terminus of the extracellular antigen-binding domain. In certain embodiments, the signal peptide comprises amino acids having the sequence set forth in SEQ ID NO: 205.

In certain embodiments, the CAR of the presently disclosed subject matter can further comprise an inducible promoter, for expressing nucleic acid sequences in human cells. Promoters for use in expressing CAR genes can be a constitutive promoter, such as ubiquitin C (UbiC) promoter.

The presently disclosed subject matter also provides isolated nucleic acid molecule encoding the BCMA-targeted CAR described herein or a functional portion thereof. In certain embodiments, the isolated nucleic acid molecule encodes a presently disclosed BCMA-targeted CAR comprising a human scFv that specifically binds to human BCMA, a transmembrane domain comprising a CD28 polypeptide, and an intracellular domain comprising a CD3ζ polypeptide and a co-stimulatory signaling region comprising a CD28 polypeptide. In one specific non-limiting example, the isolated nucleic acid molecule comprises nucleic acids having the sequence set forth in SEQ ID NO: 207 provided below:

[SEQ ID NO: 207]
caatctgccctgactcagcctgcctccgtgtctgcgtctcctggacagtcg
atcgccatctcctgcactggaaccagcagtgacgttggttggtatcaacag
cacccaggcaaagcccccaaactcatgatttatgaggacagtaagcggccc
tcaggggtttctaatcgcttctctggctccaagtctggcaacacggcctcc
ctgaccatctctgggctccaggctgaggacgaggctgattattactgcagc
tcaaatacaagaagcagcactttggtgttcggcggagggaccaagctgacc
gtcctaggttctagaggtggtggtggtagcggcggcggcggctctggtggt
ggtggatccctcgagatggccgaagtgcagctggtgcagtctggggctgag
atgaagaagcctggggcctcactgaagctctcctgcaaggcttctggatac
accttcatcgactactatgtatactggatgcgacaggcccctggacaaggg
cttgagtccatgggatggatcaaccctaacagtggtggcacaaactatgca
cagaagtttcagggcagggtcaccatgaccagggacacgtccatcagcaca
gcctacatggagctgagcaggctgagatctgacgacaccgccatgtattac
tgtgcgcgctcccagcgtgacggttacatggattactggggtcaaggtact
ctggtgaccgtctcctcagcggccgcaattgaagttatgtatcctcctcct
tacctagacaatgagaagagcaatggaaccattatccatgtgaaagggaaa
cacctttgtccaagtcccctatttcccggaccttctaagccdtttgggtgc
tggtggtggttggtggagtcctggcttgctatagcttgctagtaacagtgg
cctttattattttctgggtgaggagtaagaggagcaggctcctgcacagtg
actacatgaacatgactccccgccgccccgggcccacccgcaagcattacc
agccctatgccccaccacgcgacttcgcagcctatcgctccagagtgaagt
tcagcaggagcgcagacgcccccgcgtaccagcagggccagaaccagctct
ataacgagctcaatctaggacgaagagaggagtacgatgttttggacaaga
gacgtggccgggaccctgagatggggggaaagccgagaaggaagaaccctc
aggaaggcctgtacaatgaactgcagaaagataagatggcggaggcctaca
gtgagattgggatgaaaggcgagcgccggaggggcaaggggcacgatggcc
tttaccagggtctcagtacagccaccaaggacacctacgacgcccttcaca
tgcaggccctgccccctcgc

In one specific non-limiting example, the isolated nucleic acid molecule comprises nucleic acids having the sequence set forth in SEQ ID NO: 208 provided below:

[SEQ ID NO: 208]
cagtctgtgctgacgcagccgccctcagtgtctggggccccagggcagagg
gtcaccatctcctgcactgggagcagctccaacatcggggcaggttttgat
gtacactggtaccagcagcttccaggaacagcccccaaactcctcatctat
ggtaacagcaatcggccctcaggggtccctgaccgattctctggctccaag
tctggcacctcagcctccctggccatcactgggctccaggctgaggatgag
gctgattattactgccagtcctatgacagcagcctgagtggttatgtcttc
ggaactgggaccaaggtcaccgtcctaggttctagaggtggtggtggtagc
ggcggcggcggctctggtggtggtggatccctcgagatggcccaggtccag
ctggtacagtctggggctgaggtgaagaagcctggggcctcagtgaaggtc
tcctgcaaggcttctggatacaccttcaccgactactatatgcactgggtg
cgacaggcccctggacaacggcttgagtggatgggatggatcaaccctaac
agtggtggcacaaactatgcacagaagtttcaggacaggatcaccgtgacc
agggacacctccagcaacacaggctacatggagctgaccaggctgagatct
gacgacacggccgtgtattactgtgcgcgctctccgtactctggtgttctg
gataaatggggtcaaggtactctggtgaccgtctcctcagcggccgcaatt
gaagttatgtatcctcctccttacctagacaatgagaagagcaatggaacc
attatccatgtgaaagggaaacacctttgtccaagtcccctatttcccgga
ccttctaagcccttttgggtgctggtggtggttggtggagtcctggcttgc
tatagcttgctagtaacagtggcctttattattttctgggtgaggagtaag
aggagcaggctcctgcacagtgactacatgaacatgactccccgccgcccc
gggcccacccgcaagcattaccagccctatgccccaccacgcgacttcgca
gcctatcgctccagagtgaagttcagcaggagcgcagacgcccccgcgtac
cagcagggccagaaccagctctataacgagctcaatctaggacgaagagag
gagtacgatgttttggacaagagacgtggccgggaccctgagatgggggga
aagccgagaaggaagaaccctcaggaaggcctgtacaatgaactgcagaaa
gataagatggcggaggcctacagtgagattgggatgaaaggcgagcgccgg
aggggcaaggggcacgatggcctttaccagggtctcagtacagccaccaag
gacacctacgacgcccttcacatgcaggccctgccccctcgc

In one specific non-limiting example, the isolated nucleic acid molecule comprises nucleic acids having the sequence set forth in SEQ ID NO: 209 provided below:

[SEQ ID NO: 209]
tcctatgagctgactcagccaccctcagcgtctgggacccccgggcagagg
gtcaccatgtcttgttctggaaccagctccaacatcggaagtcactctgta
aactggtaccagcagctcccaggaacggcccccaaactcctcatctatact
aataatcagcggccctcaggggtccctgaccgattctctggctccaagtct
ggcacctcagcctccctggccatcagtggcctccagtctgaggatgaggct
gattattactgtgcagcatgggatggcagcctgaatggtctggtattcggc
ggagggaccaagctgaccgtcctaggttctagaggtggtggtggtagcggc
ggcggcggctctggtggtggtggatccctcgagatggccgaggtgcagctg
gtgcagtctggagcagaggtgaaaaagcccggggagtctctgaagatctcc
tgtaagggttctggatacagctttaccagctactggatcggctgggtgcgc
cagatgcccgggaaaggcctggagtggatggggatcatctatcctggtgac
tctgataccagatacagcccgtccttccaaggccacgtcaccatctcagct
gacaagtccatcagcactgcctacctgcagtggagcagcctgaaggcctcg
gacaccgccatgtattactgtgcgcgctactctggttctttcgataactgg
ggtcaaggtactctggtgaccgtctcctcagcggccgcaattgaagttatg
tatcctcctccttacctagacaatgagaagagcaatggaaccattatccat
gtgaaagggaaacacctttgtccaagtcccctatttcccggaccttctaag
ccctttgggtgctggtggtggttggtggagtcctggcttgctatagcttgc
tagtaacagtggcctttattattttctgggtgaggagtaagaggagcaggc
tcctgcacagtgactacatgaacatgactccccgccgccccgggcccaccc
gcaagcattaccagccctatgccccaccacgcgacttcgcagcctatcgct
ccagagtgaagttcagcaggagcgcagacgcccccgcgtaccagcagggcc
agaaccagctctataacgagctcaatctaggacgaagagaggagtacgatg
ttttggacaagagacgtggccgggaccctgagatggggggaaagccgagaa
ggaagaaccctcaggaaggcctgtacaatgaactgcagaaagataagatgg
cggaggcctacagtgagattgggatgaaaggcgagcgccggaggggcaagg
ggcacgatggcctttaccagggtctcagtacagccaccaaggacacctacg
acgcccttcacatgcaggccctgccccctcgc

In one specific non-limiting example, the isolated nucleic acid molecule comprises nucleic acids having the sequence set forth in SEQ ID NO: 229 provided below:

[SEQ ID NO: 229]
CCTTCTCTAGGCGCCCCCATATGGCCATATGAGATCTTATATGGGGCACCCCCGCCCCTTGTAAACTTCCCTGACCCTGA
CATGACAAGAGTTACTAACAGCCCCTCTCTCCAAGCTCACTTACAGGCTCTCTACTTAGTCCAGCACGAAGTCTGGAGAC
CTCTGGCGGCAGCCTACCAAGAACAACTGGACCGACCGGTGCCGCCACCATGGAAACCGACACCCTGCTGCTGTGGGTGC
TGCTGCTGTGGGTGCCAGGATCCACAGGACTGCCTGTGCTGACTCAGCCACCCTCAGCGTCTGGGACCCCCGGGCAGAGG
GTCACCATCTCTTGTTCTGGACGCAGTTCCAACATCGGGAGTAATTCTGTTAACTGGTATCGACAACTCCCAGGAGCGGC
CCCCAAACTCCTCATCTATAGTAATAATCAGCGGCCCCCAGGGGTCCCTGTGCGATTCTCTGGCTCCAAGTCTGGCACCT
CAGCCTCCCTGGCCATCAGTGGGCTCCAGTCTGAAGATGAGGCCACTTATTACTGTGCAACATGGGATGACAATCTGAAT
GTTCACTATGTCTTCGGAACTGGGACCAAGGTCACCGTCCTAGGTTCTAGAGGTGGTGGTGGTAGCGGCGGCGGCGGCTC
TGGTGGTGGTGGATCCCTCGAGATGGCCCAGGTGCAGCTGGTGCAGTCTGGGGCTGAGGTGAAGAAGCCTGGGTCCTCGG
TGAAGGTCTCCTGCAAGGCTTCTGGAGGCACCTTCAGCAGCTATGCTATCAGCTGGGTGCGACAGGCCCCTGGACAAGGG
CTTGAGTGGATGGGAAGGATCATCCCTATCCTTGGTATAGCAAACTACGCACAGAAGTTCCAGGGCAGAGTCACGATTAC
CGCGGACAAATCCACGAGCACAGCCTACATGGAGCTGAGCAGCCTGAGATCTGAGGACACGGCCGTGTATTACTGTGCGC
GCGGTGGTTACTACTCTCATGACATGTGGTCTGAAGATTGGGGTCAAGGTACTCTGGTGACCGTCTCCTCAGCggccgca
cccaccacgacgccagcgccgcgaccaccaaccccggcgcccacgatcgcgtcgcagcccctgtccctgcgcccagaggc
gtgccggccagcggcggggggcgcagtgcacacgagggggctggacttcgcctgtgatatctacatctgggcgcccctgg
ccgggacttgtggggtccttctcctgtcactggttatcaccctttactgcaacaaacggggcagaaagaagctcctgtat
atattcaaacaaccatttatgagaccagtacaaactactcaagaggaagatggctgtagctgccgatttccagaagaaga
agaaggaggatgtgaactgagagtgaagttgagcaggagcgcagagccccccgcgtaccagcagggccagaaccagctct
ataacgagctcaatctaggacgaagagaggagtacgatgttttggacaagagacgtggccgggaccctgagatgggggga
aagccgagaaggaagaaccctcaggaaggcctgtacaatgaactgcagaaagataagatggcggaggcctacagtgagat
tgggatgaaaggcgagcgccggaggggcaaggggcacgatggcctttaccagggtctcagtacagccaccaaggacacct
acgacgcccttcacatgcaggccctgccccctcgctaacagccactcgaggatccggattagtccaatttgttaaagaca
ggatatcagtggtccaggctctagttttgactcaacaatatcaccagctgaagcctatagagtacgagccatagataaaa
taaaagattttatttagtctccagaaaaaggggggaatgaaagaccccacctgtaggtttggcaagctagcttaagtaac
gccattttgcaaggcatggaaaaatacataactgagaatagagaagttcagatcaaggtcaggaacagatggaacagctg
aatatgggccaaacaggatatctgtggtaagcagttcctgccccggctcagggccaagaacagatggaacagctgaatat
gggccaaacaggatatctgtggtaagcagttcctgccccggctcagggccaagaacagatggtccccagatgcggtccag
ccctgagcagtttctagagaaccatcagatgtttccagggtgccccaaggacctgaaatgaccctgtgccttatttgaac
taaccaatcagttcgcttctcgcttctgttcgcgcgcttctgctccccgagctcaataaaagagcccacaacccctcact
cggggcgccagtcctccgattgactgagtcgcccgggtacccgtgtatccaataaaccctcttgcagttgcatccgactt
gtggtctcgctgttccttgggagggtctcctctgagtgattgactacccgtcagcgggggtctttcacacatgcagcatg
tatcaaaattaatttggttttttttcttaagtatttacattaaatggccatagtacttaaagttacattggcttccttga
aataaacatggagtattcagaatgtgtcataaatatttctaattttaagatagtatctccattggctttctactttttct
tttatttttttttgtcctctgtcttccatttgttgttgttgttgtttgtttgtttgtttgttggttggttggttaatttt
tttttaaagatcctacactatagttcaagctagactattagctactctgtaacccagggtgaccttgaagtcatgggtag
cctgctgttttagccttcccacatctaagattacaggtatgagctatcatttttggtatattgattgattgattgattga
tgtgtgtgtgtgtgattgtgtttgtgtgtgtgactgtgaaaatgtgtgtatgggtgtgtgtgaatgtgtgtatgtatgtg
tgtgtgtgagtgtgtgtgtgtgtgtgtgcatgtgtgtgtgtgtgactgtgtctatgtgtatgactgtgtgtgtgtgtgtg
tgtgtgtgtgtgtgtgtgtgtgtgtgtgtgttgtgaaaaaatattctatggtagtgagagccaacgctccggctcaggtg
tcaggttggtttttgagacagagtctttcacttagcttggAATTCACTGGCCGTCGTTTTACAACGTCGTGACTGGGAAA
ACCCTGGCGTTACCCAACTTAATCGCCTTGCAGCACATCCCCCTTTCGCCAGCTGGCGTAATAGCGAAGAGGCCCGCACC
GATCGCCCTTCCCAACAGTTGCGCAGCCTGAATGGCGAATGGCGCCTGATGCGGTATTTTCTCCTTACGCATCTGTGCGG
TATTTCACACCGCATATGGTGCACTCTCAGTACAATCTGCTCTGATGCCGCATAGTTAAGCCAGCCCCGACACCCGCCAA
CACCCGCTGACGCGCCCTGACGGGCTTGTCTGCTCCCGGCATCCGCTTACAGACAAGCTGTGACCGTCTCCGGGAGCTGC
ATGTGTCAGAGGTTTTCACCGTCATCACCGAAACGCGCGATGACGAAAGGGCCTCGTGATACGCCTATTTTTATAGGTTA
ATGTCATGATAATAATGGTTTCTTAGACGTCAGGTGGCACTTTTCGGGGAAATGTGCGCGGAACCCCTATTTGTTTATTT
TTCTAAATACATTCAAATATGTATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAAGTTCTGCTATGTGGCGCGGTATTATC
CCGTATTGACGCCGGGCAAGAGCAACTCGGTCGCCGCATACACTATTCTCAGAATGACTTGGTTGAGTACTCACCAGTCA
CAGAAAAGCATCTTACGGATGGCATGACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCACGATGCCTGTAGCAATGGCAACAA
CGTTGCGCAAACTATTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCATTGAGAAAGCGCCAC
GCTTCCCGAAGGGAGAAAGGCGGACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCCAATACGCAAACCGCCTCTCCCCGCGCGTTGG
CCGATTCATTAATGCAGCTGGCACGACAGGTTTCCCGACTGGAAAGCGGGCAGTGAGCGCAACGCAATTAATGTGAGTTA
GCTCACTCATTAGGCACCCCAGGCTTTACACTTTATGCTTCCGGCTCGTATGTTGTGTGGAATTGTGAGCGGATAACAAT
TTCACACAGGAAACAGCTATGACCATGATTACGCCAAGCTTTGCTCTTAGGAGTTTCCTAATACATCCCAAACTCAAATA
TATAAAGCATTTGACTTGTTCTATGCCCTAGGGGGCGGGGGGAAGCTAAGCCAGCTTTTTTTAACATTTAAAATGTTAAT
TCCATTTTAAATGCACAGATGTTTTTATTTCATAAGGGTTTCAATGTGCATGAATGCTGCAATATTCCTGTTACCAAAGC
TAGTATAAATAAAAATAGATAAACGTGGAAATTACTTAGAGTTTCTGTCATTAACGTTTCCTTCCTCAGTTGACAACATA
AATGCGCTGCTGAGCAAGCCAGTTTGCATCTGTCAGGATCAATTTCCCATTATGCCAGTCATATTAATTACTAGTCAATT
AGTTGATTTTTATTTTTGACATATACATGTGAATGAAAGACCCCACCTGTAGGTTTGGCAAGCTAGCTTAAGTAACGCCA
TTTTGCAAGGCATGGAAAAATACATAACTGAGAATAGAAAAGTTCAGATCAAGGTCAGGAACAGATGGAACAGCTGAATA
TGGGCCAAACAGGATATCTGTGGTAAGCAGTTCCTGCCCCGGCTCAGGGCCAAGAACAGATGGAACAGCTGAATATGGGC
CAAACAGGATATCTGTGGTAAGCAGTTCCTGCCCCGGCTCAGGGCCAAGAACAGATGGTCCCCAGATGCGGTCCAGCCCT
CAGCAGTTTCTAGAGAACCATCAGATGTTTCCAGGGTGCCCCAAGGACCTGAAATGACCCTGTGCCTTATTTGAACTAAC
CAATCAGTTCGCTTCTCGCTTCTGTTCGCGCGCTTATGCTCCCCGAGCTCAATAAAAGAGCCCACAACCCCTCACTCGGG
GCGCCAGTCCTCCGATTGACTGAGTCGCCCGGGTACCCGTGTATCCAATAAACCCTCTTGCAGTTGCATCCGACTTGTGG
TCTCGCTGTTCCTTGGGAGGGTCTCCTCTGAGTGATTGACTACCCGTCAGCGGGGGTCTTTCATTTGGGGGCTCGTCCGG
GATCGGGAGACCCCTGCCCAGGGACCACCGACCCACCACCGGGAGGTAAGCTGGCCAGCAACTTATCTGTGTCTGTCCGA
TTGTCTAGTGTCTATGACTGATTTTATGCGCCTGCGTCGGTACTAGTTAGCTAACTAGCTCTGTATCTGGCGGACCCGTG
GTGGAACTGACGAGTTCGGAACACCCGGCCGCAACCCTGGGAGACGTCCCAGGGACTTCGGGGGCCGTTTTTGTGGCCCG
ACCTGAGTCCTAAAATCCCGATCGTTTAGGACTCTTTGGTGCACCCCCCTTAGAGGAGGGATATGTGGTTCTGGTAGGAG
ACGAGAACCTAAAACAGTTCCCGCCTCCGTCTGAATTTTTGCTTTCGGTTTGGGACCGAAGCCGCGCCGCGCGTCTTGTC
TGCTGCAGCATCGTTCTGTGTTGTCTCTGTCTGACTGTGTTTCTGTATTTGTCTGAAAATATGGGCCCGGGCTAGACTGT
TACCACTCCCTTAAGTTTGACCTTAGGTCACTGGAAAGATGTCGAGCGGATCGCTCACAACCAGTCGGTAGATGTCAAGA
AGAGACGTTGGGTTACCTTCTGCTCTGCAGAATGGCCAACCTTTAACGTCGGATGGCCGCGAGACGGCACCTTTAACCGA
GACCTCATCACCCAGGTTAAGATCAAGGTCTTTTCACCTGGCCCGCATGGACACCCAGACCAGGTCCCCTACATCGTGAC
CTGGGAAGCCTTGGCTTTTGACCCCCCTCCCTGGGTCAAGCCCTTTGTACACCCTAAGCCTCCGCCTCCTCTTCCTCCAT
CCGCCCCGTCTCTCCCCCTTGAACCTCCTCGTTCGACCCCGCCTCGATCCTCCCTTTATCCAGCCCTCACT

In one specific non-limiting example, the isolated nucleic acid molecule comprises nucleic acids having the sequence set forth in SEQ ID NO: 230 provided below:

[SEQ ID NO: 230]
GGCCCTCTAGGCGCCCCCATATGGCCATATGAGATCTTATATGGGGCACCCCCGCCCCTTGTAAACTTCCCTGACCCTGA
CATGACAAGAGTTACTAACAGCCCCTCTCTCCAAGCTCACTTACAGGCTCTCTACTTAGTCCAGCACGAAGTCTGGAGAC
CTCTGGCGGCAGCCTACCAAGAACAACTGGACCGACCGGTGCCGCCACCATGGAAACCGACACCCTGCTGCTGTGGGTGC
TGCTGCTGTGGGTGCCAGGATCCACAGGAtcctatgagctgactcagccaccctcagcgtctgggacccccgggcagagg
gtcaccatgtcttgttctggaaccagctccaacatcggaagtcactctgtaaactggtaccagcagctcccaggaacggc
ccccaaactcctcatctatactaataatcagcggccctcaggggtccctgaccgattctctggctccaagtctggcacct
cagcctccctggccatcagtggcctccagtctgaggatgaggctgattattactgtgcagcatgggatggcagcctgaat
ggtctggtattcggcggagggaccaagctgaccgtcctaggttctagaggtggtggtggtagcggcggcggcggctctgg
tggtggtggatccctcgagatggccgaggtgcagctggtgcagtctggagcagaggtgaaaaagcccggggagtctctga
agatctcctgtaagggttctggatacagctttaccagctactggatcggctgggtgcgccagatgcccgggaaaggcctg
gagtggatggggatcatctatcctggtgactctgataccagatacagcccgtccttccaaggccacgtcaccatctcagc
tgacaagtccatcagcactgcctacctgcagtggagcagcctgaaggcctcggacaccgccatgtattactgtgcgcgct
actctggttctttcgataactggggtcaaggtactctggtgaccgtctcctcagcggccgcacccaccacgacgccagcg
ccgcgaccaccaaccccggcgcccacgatcgcgtcgcagcccctgtccctgcgcccagaggcgtgccggccagcggcggg
gggcgcagtgcacacgagggggctggacttcgcctgtgatatctacatctgggcgcccctggccgggacttgtggggtcc
ttctcctgtcactggttatcaccctttactgcaacaaacggggcagaaagaagctcctgtatatattcaaacaaccattt
atgagaccagtacaaactactcaagaggaagatggctgtagctgccgatttccagaagaagaagaaggaggatgtgaact
gagagtgaagttcaggaggagcgcagagccccccgcgtaccaggagggccagaaccagctctataacgagctcaatctag
gacgaagagaggagtacgatgttttggacaagagacgtggccgggaccctgagatggggggaaagccgagaaggaagaac
cctcaggaaggcctgtacaatgaactgcagaaagataagatggcggaggcctacagtgagattgggatgaaaggcgagcg
ccggaggggcaaggggcacgatggcctttaccagggtctcagtacagccaccaaggacacctacgacgcccttcacatgc
aggccctgccccctcgctaacagccactcgaggatccggattagtccaatttgttaaagacaggatatcagtggtccagg
ctctagttttgactcaacaatatcaccagctgaagcctatagagtacgagccatagataaaataaaagattttatttagt
ctccagaaaaaggggggaatgaaagaccccacctgtaggtttggcaagctagcttaagtaacgccattttgcaaggcatg
gaaaaatacataactgagaatagagaagttcagatcaaggtcaggaacagatggaacagctgaatatgggccaaacagga
tatctgtggtaagcagttcctgccccggctcagggccaagaacagatggaacagctgaatatgggccaaacaggatatct
gtggtaaggagttcctgccccggctcagggccaagaacagatggtccccagatgcggtccagccctcaggagtttctaga
gaaccatcagatgtttccagggtgccccaaggacctgaaatgaccctgtgccttatttgaactaaccaatcagttcgctt
ctcgcttctgttcgcgcgcttctgctccccgagctcaataaaagagcccacaacccctcactcggggcgccagtcctccg
attgactgagtcgcccgggtacccgtgtatccaataaaccctcttgcagttgcatccgacttgtggtctcgctgttcctt
gggagggtctcctctgagtgattgactacccgtcagcgggggtctttcacacatgcagcatgtatcaaaattaatttggt
tttttttcttaagtatttacattaaatggccatagtacttaaagttacattggcttccttgaaataaacatggagtattc
agaatgtgtcataaatatttctaattttaagatagtatctccattggctttctactttttcttttatttttttttgtcct
ctgtcttccatttgttgttgttgttgtttgtttgtttgtttgttggttggttggttaatttttttttaaagatcctacac
tatagttcaagctagactattagctactctgtaacccagggtgaccttgaagtcatgggtagcctgctgttttagccttc
ccacatctaagattacaggtatgagctatcatttttggtatattgattgattgattgattgatgtgtgtgtgtgtgattg
tgtttgtgtgtgtgactgtgaaaatgtgtgtatgggtgtgtgtgaatgtgtgtatgtatgtgtgtgtgtgagtgtgtgtg
tgtgtgtgtgcatgtgtgtgtgtgtgactgtgtctatgtgtatgactgtgtgtgtgtgtgtgtgtgtgtgtgtgtgtgtg
tgtgtgtgtgtgttgtgaaaaaatattctatggtagtgagagccaacgctccggctcaggtgtcaggttggtttttgaga
cagagtctttcacttagcttggAATTCACTGGCCGTCGTTTTACAACGTCGTGACTGGGAAAACCCTGGCGTTACCCAAC
TTAATCGCCTTGCAGCACATCCCCCTTTCGCCAGCTGGCGTAATAGCGAAGAGGCCCGCACCGATCGCCCTTCCCAACAG
TTGCGCAGCCTGAATGGCGAATGGCGCCTGATGCGGTATTTTCTCCTTACGCATCTGTGCGGTATTTCACACCGCATATG
GTGCACTCTCAGTACAATCTGCTCTGATGCCGCATAGTTAAGCCAGCCCCGACACCCGCCAACACCCGCTGACGCGCCCT
GACGGGCTTGTCTGCTCCCGGCATCCGCTTACAGACAAGCTGTGACCGTCTCCGGGAGCTGCATGTGTCAGAGGTTTTCA
CCGTCATCACCGAAACGCGCGATGACGAAAGGGCCTCGTGATACGCCTATTTTTATAGGTTAATGTCATGATAATAATGG
TTTCTTAGACGTCAGGTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGGTATTATCCCGTATTGACGCCGGGCA
AGAGCAACTCGGTCGCCGCATACACTATTCTCAGAATGACTTGGTTGAGTACTCACCAGTCACAGAAAAGCATCTTACGG
ATGGCATGACAGTAAGAGAATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCTTGATCGTTGGGAACCGGA
GCTGAATGAAGCCATACCAAACGACGAGCGTGACACCACGATGCCTGTAGCAATGGCAACAACGTTGCGCAAACTATTAA
CTGGCGAACTACTTACTCTAGCTTCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTGGGTCTCGCGGTATCATTGCAGC
ACTGGGGCCAGATGGTAAGCCCTCCCGTATCGTAGTTATCTACACGACGGGGAGTCAGGCAACTATGGATGAACGAAATA
GACAGATCGCTGAGATAGGTGCCTCACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCTACCAACTCTTTTTCCG
AAGGTAACTGGCTTCAGCAGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTTCGTGCACACAGCCCAGC
TTGGAGCGAACGACCTACACCGAACTGAGATACCTACAGCGTGAGCATTGAGAAAGCGCCACGCTTCCCGAAGGGAGAAA
GGCGGACAGGTATCCGGTAAGCGGCAGGGTCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCGCAGCCGAACGACCGAGCGCAGCG
AGTCAGTGAGCGAGGAAGCGGAAGAGCGCCCAATACGCAAACCGCCTCTCCCCGCGCGTTGGCCGATTCATTAATGCAGC
TGGCACGACAGGTTTCCCGACTGGAAAGCGGGCAGTGAGCGCAACGCAATTAATGTGAGTTAGCTCACTCATTAGGCACC
CCAGGCTTTACACTTTATGCTTCCGGCTCGTATGTTGTGTGGAATTGTGAGCGGATAACAATTTCACACAGGAAACAGCT
ATGACCATGATTACGCCAAGCTTTGCTCTTAGGAGTTTCCTAATACATCCCAAACTCAAATATATAAAGCATTTGACTTG
TTCTATGCCCTAGGGGGCGGGGGGAAGCTAAGCCAGCTTTTTTTAACATTTAAAATGTTAATTCCATTTTAAATGCACAG
ATGTTTTTATTTCATAAGGGTTTCAATGTGCATGAATGCTGCAATATTCCTGTTACCAAAGCTAGTATAAATAAAAATAG
ATAAACGTGGAAATTACTTAGAGTTTCTGTCATTAACGTTTCCTTCCTCAGTTGACAACATAAATGCGCTGCTGAGCAAG
CCAGTTTGCATCTGTCAGGATCAATTTCCCATTATGCCAGTCATATTAATTACTAGTCAATTAGTTGATTTTTATTTTTG
ACATATACATGTGAATGAAAGACCCCACCTGTAGGTTTGGCAAGCTAGCTTAAGTAACGCCATTTTGCAAGGCATGGAAA
AATACATAACTGAGAATAGAAAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGGATATCTGTGG
TAAGCAGTTCCTGCCCCGGCTCAGGGCCAAGAACAGATGGTCCCCAGATGCGGTCCAGCCCTCAGCAGTTTCTAGAGAAC
CATCAGATGTTTCCAGGGTGCCCCAAGGACCTGAAATGACCCTGTGCCTTATTTGAACTAACCAATCAGTTCGCTTCTCG
CTTCTGTTCGCGCGCTTATGCTCCCCGAGCTCAATAAAAGAGCCCACAACCCCTCACTCGGGGCGCCAGTCCTCCGATTG
ACTGAGTCGCCCGGGTACCCGTGTATCCAATAAACCCTCTTGCAGTTGCATCCGACTTGTGGTCTCGCTGTTCCTTGGGA
GGGTCTCCTCTGAGTGATTGACTACCCGTCAGCGGGGGTCTTTCATTTGGGGGCTCGTCCGGGATCGGGAGACCCCTGCC
CAGGGACCACCGACCCACCACCGGGAGGTAAGCTGGCCAGCAACTTATCTGTGTCTGTCCGATTGTCTAGTGTCTATGAC
TGATTTTATGCGCCTGCGTCGGTACTAGTTAGCTAACTAGCTCTGTATCTGGCGGACCCGTGGTGGAACTGACGAGTTCG
GAACACCCGGCCGCAACCCTGGGAGACGTCCCAGGGACTTCGGGGGCCGTTTTTGTGGCCCGACCTGAGTCCTAAAATCC
CGATCGTTTAGGACTCTTTGGTGCACCCCCCTTAGAGGAGGGATATGTGGTTCTGGTAGGAGACGAGAACCTAAAACAGT
TCCCGCCTCCGTCTGAATTTTTGCTTTCGGTTTGGGACCGAAGCCGCGCCGCGCGTCTTGTCTGCTGCAGCATCGTTCTG
TGTTGTCTCTGTCTGACTGTGTTTCTGTATTTGTCTGAAAATATGGGCCCGGGCTAGACTGTTACCACTCCCTTAAGTTT
GACCTTAGGTCACTGGAAAGATGTCGAGCGGATCGCTCACAACCAGTCGGTAGATGTCAAGAAGAGACGTTGGGTTACCT
TCTGCTCTGCAGAATGGCCAACCTTTAACGTCGGATGGCCGCGAGACGGCACCTTTAACCGAGACCTCATCACCCAGGTT
AAGATCAAGGTCTTTTCACCTGGCCCGCATGGACACCCAGACCAGGTCCCCTACATCGTGACCTGGGAAGCCTTGGCTTT
TGACCCCCCTCCCTGGGTCAAGCCCTTTGTACACCCTAAGCCTCCGCCTCCTCTTCCTCCATCCGCCCCGTCTCTCCCCC
TTGAACCTCCTCGTTCGACCCCGCCTCGATCCTCCCTTTATCCAGCCCTCACT

In one specific non-limiting example, the isolated nucleic acid molecule comprises nucleic acids having the sequence set forth in SEQ ID NO: 231 provided below:

[SEQ ID NO: 231]
CCTTCTCTAGGCGCCCCCATATGGCCATATGAGATCTTATATGGGGCACCCCCGCCCCTTGTAAACTTCCCTGACCCTGA
CATGACAAGAGTTACTAACAGCCCCTCTCTCCAAGCTCACTTACAGGCTCTCTACTTAGTCCAGCACGAAGTCTGGAGAC
CTCTGGCGGCAGCCTACCAAGAACAACTGGACCGACCGGTGCCGCCACCATGGAAACCGACACCCTGCTGCTGTGGGTGC
TGCTGCTGTGGGTGCCAGGATCCACAGGACAGGCTGTGCTGACTCAGCCACCCTCAGCGTCTGGGACCCCCGGGCAGAGG
GTCACCATCTCTTGTTCTGGAAGCAGCTCCAACATCGGAAGTAATTACGTATTCTGGTACCAGCAGCTCCCAGGAACGGC
CCCCAAACTCCTCATCTATAGTAATAATCAGCGGCCCTCAGGGGTCCCTGACCGATTCTCTGGCTCCAAGTCTGGCACCT
CAGCCTCCCTGGCCATCAGTGGGCTCCGGTCCGAGGATGAGGCTGATTATTACTGTGCAGCATGGGATGACAGCCTGAGT
GCCTCTTATGTTTTCGGAACTGGGACCAAGGTCACCGTCCTAGGTTCTAGAGGTGGTGGTGGTAGCGGCGGCGGCGGCTC
TGGTGGTGGTGGATCCCTCGAGATGGCCCAGGTGCAGCTGGTGCAGTCTGGGGCTGAGGTGAAGAAGCCTGGGTCCTCGG
TGAAGGTCTCCTGCAAGGCTTCTGGAGGCACCTTCAGCAGCTATGCTATCAGCTGGGTGCGACAGGCCCCTGGACAAGGG
CTTGAGTGGATGGGAAGGATCATCCCTATCCTTGGTACAGCAAACTACGCACAGAAGTTCCAGGGCAGAGTCACGATTAC
CGCGGACGAATCCACGAGCACAGCCTACATGGAGCTGAGCAGCCTGAGATCTGAGGACACGGCCGTGTATTACTGTGCGC
GCTCTGGTTACGGTTCTTACCGTTGGGAAGATTCTTGGGGTCAAGGTACTCTGGTGACCGTCTCCTCAGCggccgcaccc
accacgacgccagcgccgcgaccaccaaccccggcgcccacgatcgcgtcgcagcccctgtccctgcgcccagaggcgtg
ccggccagcggcggggggcgcagtgcacacgagggggctggacttcgcctgtgatatctacatctgggcgcccctggccg
ggacttgtggggtccttctcctgtcactggttatcaccctttactgcaacaaacggggcagaaagaagctcctgtatata
ttcaaacaaccatttatgagaccagtacaaactactcaagaggaagatggctgtagctgccgatttccagaagaagaaga
aggaggatgtgaactgagagtgaagttcagcaggagcgcagagccccccgcgtaccagcagggccagaaccagctctata
acgagctcaatctaggacgaagagaggagtacgatgttttggacaagagacgtggccgggaccctgagatggggggaaag
ccgagaaggaagaaccctcaggaaggcctgtacaatgaactgcagaaagataagatggcggaggcctacagtgagattgg
gatgaaaggcgagcgccggaggggcaaggggcacgatggcctttaccagggtctcagtacagccaccaaggacacctacg
acgcccttcacatgcaggccctgccccctcgctaacagccactcgaggatccggattagtccaatttgttaaagacagga
tatcagtggtccaggctctagttttgactcaacaatatcaccagctgaagcctatagagtacgagccatagataaaataa
aagattttatttagtctccagaaaaaggggggaatgaaagaccccacctgtaggtttggcaagctagcttaagtaacgcc
attttgcaaggcatggaaaaatacataactgagaatagagaagttcagatcaaggtcaggaacagatggaacagctgaat
atgggccaaacaggatatctgtggtaagcagttcctgccccggctcagggccaagaacagatggaacagctgaatatggg
ccaaacaggatatctgtggtaagcagttcctgccccggctcagggccaagaacagatggtccccagatgcggtccagccc
tgagcagtttctagagaaccatcagatgtttccagggtgccccaaggacctgaaatgaccctgtgccttatttgaactaa
ccaatcagttcgcttctcgcttctgttcgcgcgcttctgctccccgagctcaataaaagagcccacaacccctcactcgg
ggcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcacacatgcagcatgtat
caaaattaatttggttttttttcttaagtatttacattaaatggccatagtacttaaagttacattggcttccttgaaat
aaacatggagtattcagaatgtgtcataaatatttctaattttaagatagtatctccattggctttctactttttctttt
atttttttttgtcctctgtcttccatttgttgttgttgttgtttgtttgtttgtttgttggttggttggttaattttttt
ttaaagatcctacactatagttcaagctagactattagctactctgtaacccagggtgaccttgaagtcatgggtagcct
gctgttttagccttcccacatctaagattacaggtatgagctatcatttttggtatattgattgattgattgattgatgt
gtgtgtgtgtgattgtgtttgtgtgtgtgactgtgaaaatgtgtgtatgggtgtgtgtgaatgtgtgtatgtatgtgtgt
gtgtgagtgtgtgtgtgtgtgtgtgcatgtgtgtgtgtgtgactgtgtctatgtgtatgactgtgtgtgtgtgtgtgtgt
gtgtgtgtgtgtgtgtgtgtgtgtgtgttgtgaaaaaatattctatggtagtgagagccaacgctccggctcaggtgtca
ggttggtttttgagacagagtctttcacttagcttggAATTCACTGGCCGTCGTTTTACAACGTCGTGACTGGGAAAACC
CTGGCGTTACCCAACTTAATCGCCTTGCAGCACATCCCCCTTTCGCCAGCTGGCGTAATAGCGAAGAGGCCCGCACCGAT
CGCCCTTCCCAACAGTTGCGCAGCCTGAATGGCGAATGGCGCCTGATGCGGTATTTTCTCCTTACGCATCTGTGCGGTAT
TTCACACCGCATATGGTGCACTCTCAGTACAATCTGCTCTGATGCCGCATAGTTAAGCCAGCCCCGACACCCGCCAACAC
CCGCTGACGCGCCCTGACGGGCTTGTCTGCTCCCGGCATCCGCTTACAGACAAGCTGTGACCGTCTCCGGGAGCTGCATG
TGTCAGAGGTTTTCACCGTCATCACCGAAACGCGCGATGACGAAAGGGCCTCGTGATACGCCTATTTTTATAGGTTAATG
TCATGATAATAATGGTTTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATTGAAAAAGGAAGAGTAT
GAGTATTCAACATTTCCGTGTCGCCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCGAACTGGATCTCAACAGCGGTAAGATC
CTTGAGAGTTTTCGCCCCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACCATGAGTGATAACACTGCGGCCAAC
TTACTTCTGACAACGATCGGAGGACCGAAGGAGCTAACCGCTTTTTTGCACAACATGGGGGATCATGTAACTCGCCTTGA
TCGTTGGGAACCGGAGCTGAATGAAGCCATACCAAACGACGAGCGTGACACCACGATGCCTGTAGCAATGGCAACAACGT
TGCGCAAACTATTAACTGGCGAACTACTTACTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTCCTTCTAGTGTAGCCGTAGTTAGG
CCACCACTTCAAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCATTGAGAAAGCGCCACGCT
TCCCGAAGGGAGAAAGGCGGACAGGTATCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCTTTTGCTGGCCTTTTGCTCACATGTT
CTTTCCTGCGTTATCCCCTGATTCTGTGGATAACCGTATTACCGCCTTTGAGTGAGCTGATACCGCTCGCCGCAGCCGAA
CGACCGAGCGCAGCGAGTCAGTGAGCGAGGAAGCGGAAGAGCGCCCAATACGCAAACCGCCTCTCCCCGCGCGTTGGCCG
ATTCATTAATGCAGCTGGCACGACAGGTTTCCCGACTGGAAAGCGGGCAGTGAGCGCAACGCAATTAATGTGAGTTAGCT
CACTCATTAGGCACCCCAGGCTTTACACTTTATGCTTCCGGCTCGTATGTTGTGTGGAATTGTGAGCGGATAACAATTTC
ACACAGGAAACAGCTATGACCATGATTACGCCAAGCTTTGCTCTTAGGAGTTTCCTAATACATCCCAAACTCAAATATAT
AAAGCATTTGACTTGTTCTATGCCCTAGGGGGCGGGGGGAAGCTAAGCCAGCTTTTTTTAACATTTAAAATGTTAATTCC
ATTTTAAATGCACAGATGTTTTTATTTCATAAGGGTTTCAATGTGCATGAATGCTGCAATATTCCTGTTACCAAAGCTAG
TATAAATAAAAATAGATAAACGTGGAAATTACTTAGAGTTTCTGTCATTAACGTTTCCTTCCTCAGTTGACAACATAAAT
GCGCTGCTGAGCAAGCCAGTTTGCATCTGTCAGGATCAATTTCCCATTATGCCAGTCATATTAATTACTAGTCAATTAGT
TGATTTTTATTTTTGACATATACATGTGAATGAAAGACCCCACCTGTAGGTTTGGCAAGCTAGCTTAAGTAACGCCATTT
TGCAAGGCATGGAAAAATACATAACTGAGAATAGAAAAGTTCAGATCAAGGTCAGGAACAGATGGAACAGCTGAATATGG
GCCAAACAGGATATCTGTGGTAAGCAGTTCCTGCCCCGGCTCAGGGCCAAGAACAGATGGAACAGCTGAATATGGGCCAA
ACAGGATATCTGTGGTAAGCAGTTCCTGCCCCGGCTCAGGGCCAAGAACAGATGGTCCCCAGATGCGGTCCAGCCCTCAG
CAGTTTCTAGAGAACCATCAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCACAACCCCTCACTCGGGGCG
CCAGTCCTCCGATTGACTGAGTCGCCCGGGTACCCGTGTATCCAATAAACCCTCTTGCAGTTGCATCCGACTTGTGGTCT
CGCTGTTCCTTGGGAGGGTCTCCTCTGAGTGATTGACTACCCGTCAGCGGGGGTCTTTCATTTGGGGGCTCGTCCGGGAT
CGGGAGACCCCTGCCCAGGGACCACCGACCCACCACCGGGAGGTAAGCTGGCCAGCAACTTATCTGTGTCTGTCCGATTG
TCTAGTGTCTATGACTGATTTTATGCGCCTGCGTCGGTACTAGTTAGCTAACTAGCTCTGTATCTGGCGGACCCGTGGTG
GAACTGACGAGTTCGGAACACCCGGCCGCAACCCTGGGAGACGTCCCAGGGACTTCGGGGGCCGTTTTTGTGGCCCGACC
TGAGTCCTAAAATCCCGATCGTTTAGGACTCTTTGGTGCACCCCCCTTAGAGGAGGGATATGTGGTTCTGGTAGGAGACG
AGAACCTAAAACAGTTCCCGCCTCCGTCTGAATTTTTGCTTTCGGTTTGGGACCGAAGCCGCGCCGCGCGTCTTGTCTGC
TGCAGCATCGTTCTGTGTTGTCTCTGTCTGACTGTGTTTCTGTATTTGTCTGAAAATATGGGCCCGGGCTAGACTGTTAC
CACTCCCTTAAGTTTGACCTTAGGTCACTGGAAAGATGTCGAGCGGATCGCTCACAACCAGTCGGTAGATGTCAAGAAGA
GACGTTGGGTTACCTTCTGCTCTGCAGAATGGCCAACCTTTAACGTCGGATGGCCGCGAGACGGCACCTTTAACCGAGAC
CTCATCACCCAGGTTAAGATCAAGGTCTTTTCACCTGGCCCGCATGGACACCCAGACCAGGTCCCCTACATCGTGACCTG
GGAAGCCTTGGCTTTTGACCCCCCTCCCTGGGTCAAGCCCTTTGTACACCCTAAGCCTCCGCCTCCTCTTCCTCCATCCG
CCCCGTCTCTCCCCCTTGAACCTCCTCGTTCGACCCCGCCTCGATCCTCCCTTTATCCAGCCCTCACT

In one specific non-limiting example, the isolated nucleic acid molecule comprises nucleic acids having the sequence set forth in SEQ ID NO: 232 provided below:

[SEQ ID NO: 232]
CCTTCTCTAGGCGCCCCCATATGGCCATATGAGATCTTATATGGGGCACCCCCGCCCCTTGTAAACTTCCCTGACCCTGA
CATGACAAGAGTTACTAACAGCCCCTCTCTCCAAGCTCACTTACAGGCTCTCTACTTAGTCCAGCACGAAGTCTGGAGAC
CTCTGGCGGCAGCCTACCAAGAACAACTGGACCGACCGGTGCCGCCACCATGGAAACCGACACCCTGCTGCTGTGGGTGC
TGCTGCTGTGGGTGCCAGGATCCACAGGAcagtctgtgctgacgcagccgccctcagtgtctggggccccagggcagagg
gtcaccatctcctgcactgggaggagctccaacatcggggcaggttttgatgtacactggtaccaggagcttccaggaac
agcccccaaactcctcatctatggtaacagcaatcggccctcaggggtccctgaccgattctctggctccaagtctggca
cctcagcctccctggccatcactgggctccaggctgaggatgaggctgattattactgccagtcctatgacagcagcctg
agtggttatgtcttcggaactgggaccaaggtcaccgtcctaggttctagaggtggtggtggtagcggcggcggcggctc
tggtggtggtggatccctcgagatggcccaggtccagctggtacagtctggggctgaggtgaagaagcctggggcctcag
tgaaggtctcctgcaaggcttctggatacaccttcaccgactactatatgcactgggtgcgacaggcccctggacaacgg
cttgagtggatgggatggatcaaccctaacagtggtggcacaaactatgcacagaagtttcaggacaggatcaccgtgac
cagggacacctccagcaacacaggctacatggagctgaccaggctgagatctgacgacacggccgtgtattactgtgcgc
gctctccgtactctggtgttctggataaatggggtcaaggtactctggtgaccgtctcctcagcggccgcacccaccacg
acgccagcgccgcgaccaccaaccccggcgcccacgatcgcgtcgcagcccctgtccctgcgcccagaggcgtgccggcc
agcggcggggggcgcagtgcacacgagggggctggacttcgcctgtgatatctacatctgggcgcccctggccgggactt
gtggggtccttctcctgtcactggttatcaccctttactgcaacaaacggggcagaaagaagctcctgtatatattcaaa
caaccatttatgagaccagtacaaactactcaagaggaagatggctgtagctgccgatttccagaagaagaagaaggagg
atgtgaactgagagtgaagttcaggaggagcgcagagccccccgcgtaccaggagggccagaaccagctctataacgagc
tcaatctaggacgaagagaggagtacgatgttttggacaagagacgtggccgggaccctgagatggggggaaagccgaga
aggaagaaccctcaggaaggcctgtacaatgaactgcagaaagataagatggcggaggcctacagtgagattgggatgaa
aggcgagcgccggaggggcaaggggcacgatggcctttaccagggtctcagtacagccaccaaggacacctacgacgccc
ttcacatgcaggccctgccccctcgctaacagccactcgaggatccggattagtccaatttgttaaagacaggatatcag
tggtccaggctctagttttgactcaacaatatcaccagctgaagcctatagagtacgagccatagataaaataaaagatt
ttatttagtctccagaaaaaggggggaatgaaagaccccacctgtaggtttggcaagctagcttaagtaacgccattttg
caaggcatggaaaaatacataactgagaatagagaagttcagatcaaggtcaggaacagatggaacagctgaatatgggc
caaacaggatatctgtggtaagcagttcctgccccggctcagggccaagaacagatggaacagctgaatatgggccaaac
aggatatctgtggtaagcagttcctgccccggctcagggccaagaacagatggtccccagatgcggtccagccctcagca
gtttctagagaaccatcagatgtttccagggtgccccaaggacctgaaatgaccctgtgccttatttgaactaaccaatc
agttcgcttctcgcttctgttcgcgcgcttctgctccccgagctcaataaaagagcccacaacccctcactcggggcgcc
agtcctccgattgactgagtcgcccgggtacccgtgtatccaataaaccctcttgcagttgcatccgacttgtggtctcg
ctgttccttgggagggtctcctctgagtgattgactacccgtcagcgggggtctttcacacatgcagcatgtatcaaaat
taatttggttttttttcttaagtatttacattaaatggccatagtacttaaagttacattggcttccttgaaataaacat
ggagtattcagaatgtgtcataaatatttctaattttaagatagtatctccattggctttctactttttcttttattttt
ttttgtcctctgtcttccatttgttgttgttgttgtttgtttgtttgtttgttggttggttggttaatttttttttaaag
atcctacactatagttcaagctagactattagctactctgtaacccagggtgaccttgaagtcatgggtagcctgctgtt
ttagccttcccacatctaagattacaggtatgagctatcatttttggtatattgattgattgattgattgatgtgtgtgt
gtgtgattgtgtttgtgtgtgtgactgtgaaaatgtgtgtatgggtgtgtgtgaatgtgtgtatgtatgtgtgtgtgtga
gtgtgtgtgtgtgtgtgtgcatgtgtgtgtgtgtgactgtgtctatgtgtatgactgtgtgtgtgtgtgtgtgtgtgtgt
gtgtgtgtgtgtgtgtgtgtgttgtgaaaaaatattctatggtagtgagagccaacgctccggctcaggtgtcaggttgg
tttttgagacagagtctttcacttagcttggAATTCACTGGCCGTCGTTTTACAACGTCGTGACTGGGAAAACCCTGGCG
TTACCCAACTTAATCGCCTTGCAGCACATCCCCCTTTCGCCAGCTGGCGTAATAGCGAAGAGGCCCGCACCGATCGCCCT
TCCCAACAGTTGCGCAGCCTGAATGGCGAATGGCGCCTGATGCGGTATTTTCTCCTTACGCATCTGTGCGGTATTTCACA
CCGCATATGGTGCACTCTCAGTACAATCTGCTCTGATGCCGCATAGTTAAGCCAGCCCCGACACCCGCCAACACCCGCTG
ACGCGCCCTGACGGGCTTGTCTGCTCCCGGCATCCGCTTACAGACAAGCTGTGACCGTCTCCGGGAGCTGCATGTGTCAG
AGGTTTTCACCGTCATCACCGAAACGCGCGATGACGAAAGGGCCTCGTGATACGCCTATTTTTATAGGTTAATGTCATGA
TAATAATGGTTTCTTAGACGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAAAAGGAAGAGTATGAGTAT
TCAACATTTCCGTGTCGCCCTTATTCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGATCATGTAACTCGCCTTGATCGTTG
GGAACCGGAGCTGAATGAAGCCATACCAAACGACGAGCGTGACACCACGATGCCTGTAGCAATGGCAACAACGTTGCGCA
AACTATTAACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACACGACGGGGAGTCAGGCAACTATGGATG
AACGAAATAGACAGATCGCTGAGATAGGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTCCTTCTAGTGTAGCCGTAGTTAGGCCACCA
CTTCAAGAACTCTGTAGCACCGCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCATTGAGAAAGCGCCACGCTTCCCGA
AGGGAGAAAGGCGGACAGGTATCCGGTAAGCGGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATACCGCTCGCCGCAGCCGAACGACCG
AGCGCAGCGAGTCAGTGAGCGAGGAAGCGGAAGAGCGCCCAATACGCAAACCGCCTCTCCCCGCGCGTTGGCCGATTCAT
TAATGCAGCTGGCACGACAGGTTTCCCGACTGGAAAGCGGGCAGTGAGCGCAACGCAATTAATGTGAGTTAGCTCACTCA
TTAGGCACCCCAGGCTTTACACTTTATGCTTCCGGCTCGTATGTTGTGTGGAATTGTGAGCGGATAACAATTTCACACAG
GAAACAGCTATGACCATGATTACGCCAAGCTTTGCTCTTAGGAGTTTCCTAATACATCCCAAACTCAAATATATAAAGCA
TTTGACTTGTTCTATGCCCTAGGGGGCGGGGGGAAGCTAAGCCAGCTTTTTTTAACATTTAAAATGTTAATTCCATTTTA
AATGCACAGATGTTTTTATTTCATAAGGGTTTCAATGTGCATGAATGCTGCAATATTCCTGTTACCAAAGCTAGTATAAA
TAAAAATAGATAAACGTGGAAATTACTTAGAGTTTCTGTCATTAACGTTTCCTTCCTCAGTTGACAACATAAATGCGCTG
CTGAGCAAGCCAGTTTGCATCTGTCAGGATCAATTTCCCATTATGCCAGTCATATTAATTACTAGTCAATTAGTTGATTT
TTATTTTTGACATATACATGTGAATGAAAGACCCCACCTGTAGGTTTGGCAAGCTAGCTTAAGTAACGCCATTTTGCAAG
GCATGGAAAAATACATAACTGAGAATAGAAAAGTTCAGATCAAGGTCAGGAACAGATGGAACAGCTGAATATGGGCCAAA
CAGGATATCTGTGGTAAGCAGTTCCTGCCCCGGCTCAGGGCCAAGAACAGATGGAACAGCTGAATATGGGCCAAACAGGA
TATCTGTGGTAAGCAGTTCCTGCCCCGGCTCAGGGCCAAGAACAGATGGTCCCCAGATGCGGTCCAGCCCTCAGCAGTTT
CTAGAGAACCATCAGATGTTTCCAGGGTGCCCCAAGGACCTGAAATGACCCTGTGCCTTATTTGAACTAACCAATCAGTT
CGCTTCTCGCTTCTGTTCGCGCGCTTATGCTCCCCGAGCTCAATAAAAGAGCCCACAACCCCTCACTCGGGGCGCCAGTC
CTCCGATTGACTGAGTCGCCCGGGTACCCGTGTATCCAATAAACCCTCTTGCAGTTGCATCCGACTTGTGGTCTCGCTGT
TCCTTGGGAGGGTCTCCTCTGAGTGATTGACTACCCGTCAGCGGGGGTCTTTCATTTGGGGGCTCGTCCGGGATCGGGAG
ACCCCTGCCCAGGGACCACCGACCCACCACCGGGAGGTAAGCTGGCCAGCAACTTATCTGTGTCTGTCCGATTGTCTAGT
GTCTATGACTGATTTTATGCGCCTGCGTCGGTACTAGTTAGCTAACTAGCTCTGTATCTGGCGGACCCGTGGTGGAACTG
ACGAGTTCGGAACACCCGGCCGCAACCCTGGGAGACGTCCCAGGGACTTCGGGGGCCGTTTTTGTGGCCCGACCTGAGTC
CTAAAATCCCGATCGTTTAGGACTCTTTGGTGCACCCCCCTTAGAGGAGGGATATGTGGTTCTGGTAGGAGACGAGAACC
TAAAACAGTTCCCGCCTCCGTCTGAATTTTTGCTTTCGGTTTGGGACCGAAGCCGCGCCGCGCGTCTTGTCTGCTGCAGC
ATCGTTCTGTGTTGTCTCTGTCTGACTGTGTTTCTGTATTTGTCTGAAAATATGGGCCCGGGCTAGACTGTTACCACTCC
CTTAAGTTTGACCTTAGGTCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACCTTTAACCGAGACCTCATC
ACCCAGGTTAAGATCAAGGTCTTTTCACCTGGCCCGCATGGACACCCAGACCAGGTCCCCTACATCGTGACCTGGGAAGC
CTTGGCTTTTGACCCCCCTCCCTGGGTCAAGCCCTTTGTACACCCTAAGCCTCCGCCTCCTCTTCCTCCATCCGCCCCGT
CTCTCCCCCTTGAACCTCCTCGTTCGACCCCGCCTCGATCCTCCCTTTATCCAGCCCTCACT

In one specific non-limiting example, the isolated nucleic acid molecule comprises nucleic acids having the sequence set forth in SEQ ID NO: 233 provided below:

[SEQ ID NO: 233]
CCTTCTCTAGGCGCCCCCATATGGCCATATGAGATCTTATATGGGGCACCCCCGCCCCTTGTAAACTTCCCTGACCCTGA
CATGACAAGAGTTACTAACAGCCCCTCTCTCCAAGCTCACTTACAGGCTCTCTACTTAGTCCAGCACGAAGTCTGGAGAC
CTCTGGCGGCAGCCTACCAAGAACAACTGGACCGACCGGTGCCGCCACCATGGAAACCGACACCCTGCTGCTGTGGGTGC
TGCTGCTGTGGGTGCCAGGATCCACAGGAcaatctgccctgactcagcctgcctccgtgtctgcgtctcctggacagtcg
atcgccatctcctgcactggaaccagcagtgacgttggttggtatcaacagcacccaggcaaagcccccaaactcatgat
ttatgaggacagtaagcggccctcaggggtttctaatcgcttctctggctccaagtctggcaacacggcctccctgacca
tctctgggctccaggctgaggacgaggctgattattactgcagctcaaatacaagaagcagcactttggtgttcggcgga
gggaccaagctgaccgtcctaggttctagaggtggtggtggtagcggcggcggcggctctggtggtggtggatccctcga
gatggccgaagtgcagctggtgcagtctggggctgagatgaagaagcctggggcctcactgaagctctcctgcaaggctt
ctggatacaccttcatcgactactatgtatactggatgcgacaggcccctggacaagggcttgagtccatgggatggatc
aaccctaacagtggtggcacaaactatgcacagaagtttcagggcagggtcaccatgaccagggacacgtccatcagcac
agcctacatggagctgagcaggctgagatctgacgacaccgccatgtattactgtgcgcgctcccagcgtgacggttaca
tggattactggggtcaaggtactctggtgaccgtctcctcagcggccgcacccaccacgacgccagcgccgcgaccacca
accccggcgcccacgatcgcgtcgcagcccctgtccctgcgcccagaggcgtgccggccagcggcggggggcgcagtgca
cacgagggggctggacttcgcctgtgatatctacatctgggcgcccctggccgggacttgtggggtccttctcctgtcac
tggttatcaccctttactgcaacaaacggggcagaaagaagctcctgtatatattcaaacaaccatttatgagaccagta
caaactactcaagaggaagatggctgtagctgccgatttccagaagaagaagaaggaggatgtgaactgagagtgaagtt
caggaggagcgcagagccccccgcgtaccaggagggccagaaccagctctataacgagctcaatctaggacgaagagagg
agtacgatgttttggacaagagacgtggccgggaccctgagatggggggaaagccgagaaggaagaaccctcaggaaggc
ctgtacaatgaactgcagaaagataagatggcggaggcctacagtgagattgggatgaaaggcgagcgccggaggggcaa
ggggcacgatggcctttaccagggtctcagtacagccaccaaggacacctacgacgcccttcacatgcaggccctgcccc
ctcgctaacagccactcgaggatccggattagtccaatttgttaaagacaggatatcagtggtccaggctctagttttga
ctcaacaatatcaccagctgaagcctatagagtacgagccatagataaaataaaagattttatttagtctccagaaaaag
gggggaatgaaagaccccacctgtaggtttggcaagctagcttaagtaacgccattttgcaaggcatggaaaaatacata
actgagaatagagaagttcagatcaaggtcaggaacagatggaacagctgaatatgggccaaacaggatatctgtggtaa
gcagttcctgccccggctcagggccaagaacagatggaacagctgaatatgggccaaacaggatatctgtggtaagcagt
tcctgccccggctcagggccaagaacagatggtccccagatgcggtccagccctcagcagtttctagagaaccatcagat
gtttccagggtgccccaaggacctgaaatgaccctgtgccttatttgaactaaccaatcagttcgcttctcgcttctgtt
cgcgcgcttctgctccccgagctcaataaaagagcccacaacccctcactcggggcgccagtcctccgattgactgagtc
gcccgggtacccgtgtatccaataaaccctcttgcagttgcatccgacttgtggtctcgctgttccttgggagggtctcc
tctgagtgattgactacccgtcagcgggggtctttcacacatgcagcatgtatcaaaattaatttggttttttttcttaa
gtatttacattaaatggccatagtacttaaagttacattggcttccttgaaataaacatggagtattcagaatgtgtcat
aaatatttctaattttaagatagtatctccattggctttctactttttcttttatttttttttgtcctctgtcttccatt
tgttgttgttgttgtttgtttgtttgtttgttggttggttggttaatttttttttaaagatcctacactatagttcaagc
tagactattagctactctgtaacccagggtgaccttgaagtcatgggtagcctgctgttttagccttcccacatctaaga
ttacaggtatgagctatcatttttggtatattgattgattgattgattgatgtgtgtgtgtgtgattgtgtttgtgtgtg
tgactgtgaaaatgtgtgtatgggtgtgtgtgaatgtgtgtatgtatgtgtgtgtgtgagtgtgtgtgtgtgtgtgtgca
tgtgtgtgtgtgtgactgtgtctatgtgtatgactgtgtgtgtgtgtgtgtgtgtgtgtgtgtgtgtgtgtgtgtgtgtg
ttgtgaaaaaatattctatggtagtgagagccaacgctccggctcaggtgtcaggttggtttttgagacagagtctttca
cttagcttggAATTCACTGGCCGTCGTTTTACAACGTCGTGACTGGGAAAACCCTGGCGTTACCCAACTTAATCGCCTTG
CAGCACATCCCCCTTTCGCCAGCTGGCGTAATAGCGAAGAGGCCCGCACCGATCGCCCTTCCCAACAGTTGCGCAGCCTG
AATGGCGAATGGCGCCTGATGCGGTATTTTCTCCTTACGCATCTGTGCGGTATTTCACACCGCATATGGTGCACTCTCAG
TACAATCTGCTCTGATGCCGCATAGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCGTCATCACCG
AAACGCGCGATGACGAAAGGGCCTCGTGATACGCCTATTTTTATAGGTTAATGTCATGATAATAATGGTTTCTTAGACGT
CAGGTGGCACTTTTCGGGGAAATGTGCGCGGAACCCCTATTTGTTTATTTTTCTAAATACATTCAAATATGTATCCGCTC
ATGAGACAATAACCCTGATAAATGCTTCAATAATATTGAAAAAGGAAGAGTATGAGTATTCAACATTTCCGTGTCGCCCT
TATTCCCTTTTTTGCGGCATTTTGCCTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCCTTGAGAGTTTTCGCCCCGAAGAACGT
TTTCCAATGATGAGCACTTTTAAAGTTCTGCTATGTGGCGCGGTATTATCCCGTATTGACGCCGGGCAAGAGCAACTCGG
TCGCCGCATACACTATTCTCAGAATGACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGAACCGGAGCTGAATGAAGC
CATACCAAACGACGAGCGTGACACCACGATGCCTGTAGCAATGGCAACAACGTTGCGCAAACTATTAACTGGCGAACTAC
TTACTCTAGCTTCCCGGCAACAATTAATAGACTGGATGGAGGCGGATAAAGTTGCAGGACCACTTCTGCGCTCGGCCCTT
CCGGCTGGCTGGTTTATTGCTGATAAATCTGGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATACTTTAGATTGATTTAAAACTT
CATTTTTAATTTAAAAGGATCTAGGTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACACAGCCCAGCTTGGAGCGAACG
ACCTACACCGAACTGAGATACCTACAGCGTGAGCATTGAGAAAGCGCCACGCTTCCCGAAGGGAGAAAGGCGGACAGGTA
TCCGGTAAGCGGCAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATGTTCTTTCCTGCGTTATCCCCTGATTCTGT
GGATAACCGTATTACCGCCTTTGAGTGAGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCGATTCATTAATGCAGCTGGCACGACAGG
TTTCCCGACTGGAAAGCGGGCAGTGAGCGCAACGCAATTAATGTGAGTTAGCTCACTCATTAGGCACCCCAGGCTTTACA
CTTTATGCTTCCGGCTCGTATGTTGTGTGGAATTGTGAGCGGATAACAATTTCACACAGGAAACAGCTATGACCATGATT
ACGCCAAGCTTTGCTCTTAGGAGTTTCCTAATACATCCCAAACTCAAATATATAAAGCATTTGACTTGTTCTATGCCCTA
GGGGGCGGGGGGAAGCTAAGCCAGCTTTTTTTAACATTTAAAATGTTAATTCCATTTTAAATGCACAGATGTTTTTATTT
CATAAGGGTTTCAATGTGCATGAATGCTGCAATATTCCTGTTACCAAAGCTAGTATAAATAAAAATAGATAAACGTGGAA
ATTACTTAGAGTTTCTGTCATTAACGTTTCCTTCCTCAGTTGACAACATAAATGCGCTGCTGAGCAAGCCAGTTTGCATC
TGTCAGGATCAATTTCCCATTATGCCAGTCATATTAATTACTAGTCAATTAGTTGATTTTTATTTTTGACATATACATGT
GAATGAAAGACCCCACCTGTAGGTTTGGCAAGCTAGCTTAAGTAACGCCATTTTGCAAGGCATGGAAAAATACATAACTG
AGAATAGAAAAGTTCAGATCAAGGTCAGGAACAGATGGAACAGCTGAATATGGGCCAAACAGGATATCTGTGGTAAGCAG
TTCCTGCCCCGGCTCAGGGCCAAGAACAGATGGAACAGCTGAATATGGGCCAAACAGGATATCTGTGGTAAGCAGTTCCT
GCCCCGGCTCAGGGCCAAGAACAGATGGTCCCCAGATGCGGTCCAGCCCTCAGCAGTTTCTAGAGAACCATCAGATGTTT
CCAGGGTGCCCCAAGGACCTGAAATGACCCTGTGCCTTATTTGAACTAACCAATCAGTTCGCTTCTCGCTTCTGTTCGCG
CGCTTATGCTCCCCGAGCTCAATAAAAGAGCCCACAACCCCTCACTCGGGGCGCCAGTCCTCCGATTGACTGAGTCGCCC
GGGTACCCGTGTATCCAATAAACCCTCTTGCAGTTGCATCCGACTTGTGGTCTCGCTGTTCCTTGGGAGGGTCTCCTCTG
AGTGATTGACTACCCGTCAGCGGGGGTCTTTCATTTGGGGGCTCGTCCGGGATCGGGAGACCCCTGCCCAGGGACCACCG
ACCCACCACCGGGAGGTAAGCTGGCCAGCAACTTATCTGTGTCTGTCCGATTGTCTAGTGTCTATGACTGATTTTATGCG
CCTGCGTCGGTACTAGTTAGCTAACTAGCTCTGTATCTGGCGGACCCGTGGTGGAACTGACGAGTTCGGAACACCCGGCC
GCAACCCTGGGAGACGTCCCAGGGACTTCGGGGGCCGTTTTTGTGGCCCGACCTGAGTCCTAAAATCCCGATCGTTTAGG
ACTCTTTGGTGCACCCCCCTTAGAGGAGGGATATGTGGTTCTGGTAGGAGACGAGAACCTAAAACAGTTCCCGCCTCCGT
CTGAATTTTTGCTTTCGGTTTGGGACCGAAGCCGCGCCGCGCGTCTTGTCTGCTGCAGCATCGTTCTGTGTTGTCTCTGT
CTGACTGTGTTTCTGTATTTGTCTGAAAATATGGGCCCGGGCTAGACTGTTACCACTCCCTTAAGTTTGACCTTAGGTCA
CTGGAAAGATGTCGAGCGGATCGCTCACAACCAGTCGGTAGATGTCAAGAAGAGACGTTGGGTTACCTTCTGCTCTGCAG
AATGGCCAACCTTTAACGTCGGATGGCCGCGAGACGGCACCTTTAACCGAGACCTCATCACCCAGGTTAAGATCAAGGTC
TTTTCACCTGGCCCGCATGGACACCCAGACCAGGTCCCCTACATCGTGACCTGGGAAGCCTTGGCTTTTGACCCCCCTCC
CTGGGTCAAGCCCTTTGTACACCCTAAGCCTCCGCCTCCTCTTCCTCCATCCGCCCCGTCTCTCCCCCTTGAACCTCCTC
GTTCGACCCCGCCTCGATCCTCCCTTTATCCAGCCCTCACT

In one specific non-limiting example, the isolated nucleic acid molecule comprises nucleic acids having the sequence set forth in SEQ ID NO: 234 provided below:

[SEQ ID NO: 234]
atggaaaccgacaccctgctgctgtgggtgctgctgctgtgggtgccagga
tccacaggactgcctgtgctgactcagccaccctcagcgtctgggaccccc
gggcagagggtcaccatctcttgttctggacgcagttccaacatcgggagt
aattctgttaactggtatcgacaactcccaggagcggcccccaaactcctc
atctatagtaataatcagcggcccccaggggtccctgtgcgattctctggc
tccaagtctggcacctcagcctccctggccatcagtgggctccagtctgaa
gatgaggccacttattactgtgcaacatgggatgacaatctgaatgttcac
tatgtcttcggaactgggaccaaggtcaccgtcctaggttctagaggtggt
ggtggtagcggcggcggcggctctggtggtggtggatccctcgagatggcc
caggtgcagctggtgcagtctggggctgaggtgaagaagcctgggtcctcg
gtgaaggtctcctgcaaggcttctggaggcaccttcagcagctatgctatc
agctgggtgcgacaggcccctggacaagggcttgagtggatgggaaggatc
atccctatccttggtatagcaaactacgcacagaagttccagggcagagtc
acgattaccgcggacaaatccacgagcacagcctacatggagctgagcagc
ctgagatctgaggacacggccgtgtattactgtgcgcgcggtggttactac
tctcatgacatgtggtctgaagattggggtcaaggtactctggtgaccgtc
tcctcagcggccgcaattgaagttatgtatcctcctccttacctagacaat
gagaagagcaatggaaccattatccatgtgaaagggaaacacctttgtcca
agtcccctatttcccggaccttctaagcccttttgggtgctggtggtggtt
ggtggagtcctggcttgctatagcttgctagtaacagtggcctttattatt
ttctgggtgaggagtaagaggagcaggctcctgcacagtgactacatgaac
atgactccccgccgccccgggcccacccgcaagcattaccagccctatgcc
ccaccacgcgacttcgcagcctatcgctccagagtgaagttcagcaggagc
gcagacgcccccgcgtaccagcagggccagaaccagctctataacgagctc
aatctaggacgaagagaggagtacgatgttttggacaagagacgtggccgg
gaccctgagatggggggaaagccgagaaggaagaaccctcaggaaggcctg
tacaatgaactgcagaaagataagatggcggaggcctacagtgagattggg
atgaaaggcgagcgccggaggggcaaggggcacgatggcctttaccagggt
ctcagtacagccaccaaggacacctacgacgcccttcacatgcaggccctg
ccccctcgctaa

In one specific non-limiting example, the isolated nucleic acid molecule comprises nucleic acids having the sequence set forth in SEQ ID NO: 235 provided below:

[SEQ ID NO: 235]
atggaaaccgacaccctgctgctgtgggtgctgctgctgtgggtgccagga
tccacaggacaggctgtgctgactcagccaccctcagcgtctgggaccccc
gggcagagggtcaccatctcttgttctggaagcagctccaacatcggaagt
aattacgtattctggtaccagcagctcccaggaacggcccccaaactcctc
atctatagtaataatcagcggccctcaggggtccctgaccgattctctggc
tccaagtctggcacctcagcctccctggccatcagtgggctccggtccgag
gatgaggctgattattactgtgcagcatgggatgacagcctgagtgcctct
tatgttttcggaactgggaccaaggtcaccgtcctaggttctagaggtggt
ggtggtagcggcggcggcggctctggtggtggtggatccctcgagatggcc
caggtgcagctggtgcagtctggggctgaggtgaagaagcctgggtcctcg
gtgaaggtctcctgcaaggcttctggaggcaccttcagcagctatgctatc
agctgggtgcgacaggcccctggacaagggcttgagtggatgggaaggatc
atccctatccttggtacagcaaactacgcacagaagttccagggcagagtc
acgattaccgcggacgaatccacgagcacagcctacatggagctgagcagc
ctgagatctgaggacacggccgtgtattactgtgcgcgctctggttacggt
tcttaccgttgggaagattcttggggtcaaggtactctggtgaccgtctcc
tcagcggccgcaattgaagttatgtatcctcctccttacctagacaatgag
aagagcaatggaaccattatccatgtgaaagggaaacacctttgtccaagt
cccctatttcccggaccttctaagcccttttgggtgctggtggtggttggt
ggagtcctggcttgctatagcttgctagtaacagtggcctttattattttc
tgggtgaggagtaagaggagcaggctcctgcacagtgactacatgaacatg
actccccgccgccccgggcccacccgcaagcattaccagccctatgcccca
ccacgcgacttcgcagcctatcgctccagagtgaagttcagcaggagcgca
gacgcccccgcgtaccagcagggccagaaccagctctataacgagctcaat
ctaggacgaagagaggagtacgatgttttggacaagagacgtggccgggac
cctgagatggggggaaagccgagaaggaagaaccctcaggaaggcctgtac
aatgaactgcagaaagataagatggcggaggcctacagtgagattgggatg
aaaggcgagcgccggaggggcaaggggcacgatggcctttaccagggtctc
agtacagccaccaaggacacctacgacgcccttcacatgcaggccctgccc
cctcgctaa

The isolated nucleic acid molecule having the nucleotide sequence of SEQ ID NO: 207 encodes a BCMA-targeted CAR (designated as BCMA-targeted 28z CAR54) comprising a human scFv that comprises a heavy chain variable region comprising amino acids having the sequence set forth in SEQ ID NO: 53, a light chain variable region comprising amino acids having the sequence set forth in SEQ ID NO: 54, and a linker having an amino acid sequence of SEQ ID NO: 69 positioned between the heavy chain variable region and the light chain variable region, a transmembrane domain comprising a CD28 polypeptide, and an intracellular domain comprising a CD3ζ polypeptide comprising amino acid sequence of amino acids 52 to 163 of SEQ ID NO: 195, and a co-stimulatory signaling region comprising a CD28 polypeptide, wherein the CD28 region comprising the transmembrance domain and the co-stimulatory signaling region comprises amino acids 114 to 220 of SEQ ID NO: 193.

The isolated nucleic acid molecule having the nucleotide sequence of SEQ ID NO: 208 encodes a BCMA-targeted CAR (designated as BCMA-targeted 28z CAR40) comprising a human scFv that comprises a heavy chain variable region comprising amino acids having the sequence set forth in SEQ ID NO: 57, a light chain variable region comprising amino acids having the sequence set forth in SEQ ID NO: 58, and a linker having an amino acid sequence of SEQ ID NO: 69 positioned between the heavy chain variable region and the light chain variable region, a transmembrane domain comprising a CD28 polypeptide, and an intracellular domain comprising a CD3ζ polypeptide comprising amino acid sequence of amino acids 52 to 163 of SEQ ID NO: 195, and a co-stimulatory signaling region comprising a CD28 polypeptide, wherein the CD28 region comprising the transmembrance domain and the co-stimulatory signaling region comprises amino acids 114 to 220 of SEQ ID NO: 193.

The isolated nucleic acid molecule having the nucleotide sequence of SEQ ID NO: 209 encodes a BCMA-targeted CAR (designated as BCMA-targeted 28z CAR24) comprising a human scFv that comprises a heavy chain variable region comprising amino acids having the sequence set forth in SEQ ID NO: 65, a light chain variable region comprising amino acids having the sequence set forth in SEQ ID NO: 66, and a linker having an amino acid sequence of SEQ ID NO: 69 positioned between the heavy chain variable region and the light chain variable region, a transmembrane domain comprising a CD28 polypeptide, and an intracellular domain comprising a CD3ζ polypeptide comprising amino acid sequence of amino acids 52 to 163 of SEQ ID NO: 195, and a co-stimulatory signaling region comprising a CD28 polypeptide, wherein the CD28 region comprising the transmembrance domain and the co-stimulatory signaling region comprises amino acids 114 to 220 of SEQ ID NO: 193.

The isolated nucleic acid molecule having the nucleotide sequence of SEQ ID NO: 234 encodes a BCMA-targeted CAR (designated as BCMA-targeted 28z CAR3) comprising a human scFv that comprises a heavy chain variable region comprising amino acids having the sequence set forth in SEQ ID NO: 21, a light chain variable region comprising amino acids having the sequence set forth in SEQ ID NO: 22, and a linker having an amino acid sequence of SEQ ID NO: 69 positioned between the heavy chain variable region and the light chain variable region, a transmembrane domain comprising a CD28 polypeptide, and an intracellular domain comprising a CD3ζ polypeptide comprising amino acid sequence of amino acids 52 to 163 of SEQ ID NO: 195, and a co-stimulatory signaling region comprising a CD28 polypeptide, wherein the CD28 region comprising the transmembrance domain and the co-stimulatory signaling region comprises amino acids 114 to 220 of SEQ ID NO: 193.

The isolated nucleic acid molecule having the nucleotide sequence of SEQ ID NO: 235 encodes a BCMA-targeted CAR (designated as BCMA-targeted 28z CAR37) comprising a human scFv that comprises a heavy chain variable region comprising amino acids having the sequence set forth in SEQ ID NO: 61, a light chain variable region comprising amino acids having the sequence set forth in SEQ ID NO: 62, and a linker having an amino acid sequence of SEQ ID NO: 69 positioned between the heavy chain variable region and the light chain variable region, a transmembrane domain comprising a CD28 polypeptide, and an intracellular domain comprising a CD3ζ polypeptide comprising amino acid sequence of amino acids 52 to 163 of SEQ ID NO: 195, and a co-stimulatory signaling region comprising a CD28 polypeptide, wherein the CD28 region comprising the transmembrance domain and the co-stimulatory signaling region comprises amino acids 114 to 220 of SEQ ID NO: 193.

The isolated nucleic acid molecule having the nucleotide sequence of SEQ ID NO: 229 encodes a BCMA-targeted CAR (designated as BCMA-targeted BBz CAR3) comprising a human scFv that comprises a heavy chain variable region comprising amino acids having the sequence set forth in SEQ ID NO: 21, a light chain variable region comprising amino acids having the sequence set forth in SEQ ID NO: 22, and a linker having an amino acid sequence of SEQ ID NO: 69 positioned between the heavy chain variable region and the light chain variable region, a transmembrane domain comprising a CD8 polypeptide having amino acids 137 to 207 of SEQ ID NO: 226, and an intracellular domain comprising a CD3ζ polypeptide comprising amino acid sequence of amino acids 52 to 163 of SEQ ID NO: 195, and a co-stimulatory signaling region comprising a 4-1BB polypeptide having the amino acids 214-255 of SEQ ID NO: 197. Nucleotide sequences 270-1031 of SEQ ID NO: 229 encodes the human scFv. Nucleotide sequences 1041-1253 of SEQ ID NO: 229 encodes the CD8 polypeptide comprised in the transmembrane domain. Nucleotide sequences 1254-1379 of SEQ ID NO: 229 encodes the 4-1BB polypeptide comprised in the intracellular domain. Nucleotide sequences 1380-1718 of SEQ ID NO: 229 encodes the CD3zeta polypeptide comprised in the intracellular domain. Other portions of SEQ ID NO: 229 are shown in Table 19.

TABLE 19

| | nucleotide Sequence positions | number of |
|---|---|---|
| Portions | of SEQ ID NO: 229 | nucleotides |
| Kappa sp | 210 . . . 269 | 60 |
| LTR | 1998 . . . 2467 | 470 |
| M13 fwd | 3166 . . . 3182 | 17 |
| AmpR promoter | 3657 . . . 3761 | 105 |
| AmpR | 3762 . . . 4622 | 861 |
| ori | 4793 . . . 5381 | 589 |
| CAP binding site | 5669 . . . 5690 | 22 |
| lac promoter | 5705 . . . 5735 | 31 |
| lac operator | 5743 . . . 5759 | 17 |
| M13 rev | 5767 . . . 5783 | 17 |
| LTR | 6192 . . . 6785 | 594 |
| MMLV Psi | 6848 . . . 7205 | 358 |
| gag (truncated) | 7270 . . . 15 | 417 |

The isolated nucleic acid molecule having the nucleotide sequence of SEQ ID NO: 230 encodes a BCMA-targeted CAR (designated as BCMA-targeted BBz CAR24) comprising a human scFv that comprises a heavy chain variable region comprising amino acids having the sequence set forth in SEQ ID NO: 65, a light chain variable region comprising amino acids having the sequence set forth in SEQ ID NO: 66, and a linker having an amino acid sequence of SEQ ID NO: 69 positioned between the heavy chain variable region and the light chain variable region, a transmembrane domain comprising a CD8 polypeptide having amino acids 137 to 207 of SEQ ID NO: 226, and an intracellular domain comprising a CD3ζ polypeptide comprising amino acid sequence of amino acids 52 to 163 of SEQ ID NO: 195, and a co-stimulatory signaling region comprising a 4-1BB polypeptide having the amino acids 214-255 of SEQ ID NO: 197. Nucleotide sequences 270-1015 of SEQ ID NO: 230 encodes the human scFv. Nucleotide sequences 1023-1235 of SEQ ID NO: 230 encodes the CD8 polypeptide comprised in the transmembrane domain. Nucleotide sequences 1236-1361 of SEQ ID NO: 230 encodes the 4-1BB polypeptide comprised in the intracellular domain. Nucleotide sequences 1362-1700 of SEQ ID NO: 230 encodes the CD3zeta polypeptide comprised in the intracellular domain. Other portions of SEQ ID NO: 230 are shown in Table 20.

TABLE 20

| | nucleotide Sequence positions | number of |
|---|---|---|
| Portions | of SEQ ID NO: 230 | nucleotides |
| Kappa sp | 210 . . . 269 | 60 |
| LTR | 1980 . . . 2449 | 470 |
| M13 fwd | 3148 . . . 3164 | 17 |
| AmpR promoter | 3639 . . . 3743 | 105 |
| AmpR | 3744 . . . 4604 | 861 |
| ori | 4775 . . . 5363 | 589 |
| CAP binding site | 5651 . . . 5672 | 22 |
| lac promoter | 5687 . . . 5717 | 31 |
| lac operator | 5725 . . . 5741 | 17 |
| M13 rev | 5749 . . . 5765 | 17 |
| LTR | 6174 . . . 6767 | 594 |
| MMLV Psi | 6830 . . . 7187 | 358 |
| gag (truncated) | 7252 . . . 15 | 417 |

The isolated nucleic acid molecule having the nucleotide sequence of SEQ ID NO: 231 encodes a BCMA-targeted CAR (designated as BCMA-targeted BBz CAR37) comprising a human scFv that comprises a heavy chain variable region comprising amino acids having the sequence set forth in SEQ ID NO: 61, a light chain variable region comprising amino acids having the sequence set forth in SEQ ID NO: 62, and a linker having an amino acid sequence of SEQ ID NO: 69 positioned between the heavy chain variable region and the light chain variable region, a transmembrane domain comprising a CD8 polypeptide having amino acids 137 to 207 of SEQ ID NO: 226, and an intracellular domain comprising a CD3ζ polypeptide comprising amino acid sequence of amino acids 52 to 163 of SEQ ID NO: 195, and a co-stimulatory signaling region comprising a 4-1BB polypeptide having the amino acids 214-255 of SEQ ID NO: 197. Nucleotide sequences 270-1028 of SEQ ID NO: 231 encodes the human scFv. Nucleotide sequences 1038-1250 of SEQ ID NO: 231 encodes the CD8 polypeptide comprised in the transmembrane domain. Nucleotide sequences 1251-1376 of SEQ ID NO: 231 encodes the 4-1BB polypeptide comprised in the intracellular domain. Nucleotide sequences 1377-1715 of SEQ ID NO: 231 encodes the CD3zeta polypeptide comprised in the intracellular domain. Other portions of SEQ ID NO: 231 are shown in Table 21.

TABLE 21

| | nucleotide Sequence positions | number of |
|---|---|---|
| Portions | of SEQ ID NO: 231 | nucleotides |
| Kappa sp | 210 . . . 269 | 60 |
| LTR | 1995 . . . 2464 | 470 |
| M13 fwd | 3163 . . . 3179 | 17 |
| AmpR promoter | 3654 . . . 3758 | 105 |
| AmpR | 3759 . . . 4619 | 861 |
| ori | 4790 . . . 5378 | 589 |
| CAP binding site | 5666 . . . 5687 | 22 |
| lac promoter | 5702 . . . 5732 | 31 |
| lac operator | 5740 . . . 5756 | 17 |
| M13 rev | 5764 . . . 5780 | 17 |
| LTR | 6189 . . . 6782 | 594 |
| MMLV Psi | 6845 . . . 7202 | 358 |
| gag (truncated) | 7267 . . . 15 | 417 |

The isolated nucleic acid molecule having the nucleotide sequence of SEQ ID NO: 232 encodes a BCMA-targeted CAR (designated as BCMA-targeted BBz CAR40) comprising a human scFv that comprises a heavy chain variable region comprising amino acids having the sequence set forth in SEQ ID NO: 57, a light chain variable region comprising amino acids having the sequence set forth in SEQ ID NO: 58, and a linker having an amino acid sequence of SEQ ID NO: 69 positioned between the heavy chain variable region and the light chain variable region, a transmembrane domain comprising a CD8 polypeptide having amino acids 137 to 207 of SEQ ID NO: 226, and an intracellular domain comprising a CD3ζ polypeptide comprising amino acid sequence of amino acids 52 to 163 of SEQ ID NO: 195, and a co-stimulatory signaling region comprising a 4-1BB polypeptide having the amino acids 214-255 of SEQ ID NO: 197. Nucleotide sequences 270-1024 of SEQ ID NO: 232 encodes the human scFv. Nucleotide sequences 1032-1244 of SEQ ID NO: 232 encodes the CD8 polypeptide comprised in the transmembrane domain. Nucleotide sequences 1245-1370 of SEQ ID NO: 232 encodes the 4-1BB polypeptide comprised in the intracellular domain. Nucleotide sequences 1371-1709 of SEQ ID NO: 232 encodes the CD3zeta polypeptide comprised in the intracellular domain. Other portions of SEQ ID NO: 232 are shown in Table 22.

TABLE 22

| | nucleotide Sequence positions | number of |
|---|---|---|
| Portions | of SEQ ID NO: 232 | nucleotides |
| Kappa sp | 210 . . . 269 | 60 |
| LTR | 1989 . . . 2458 | 470 |
| M13 fwd | 3157 . . . 3173 | 17 |
| AmpR promoter | 3648 . . . 3752 | 105 |
| AmpR | 3753 . . . 4613 | 861 |
| ori | 4784 . . . 5372 | 589 |
| CAP binding site | 5660 . . . 5681 | 22 |
| lac promoter | 5696 . . . 5726 | 31 |
| lac operator | 5734 . . . 5750 | 17 |
| M13 rev | 5758 . . . 5774 | 17 |
| LTR | 6183 . . . 6776 | 594 |
| MMLV Psi | 6839 . . . 7196 | 358 |
| gag (truncated) | 7261 . . . 15 | 417 |

The isolated nucleic acid molecule having the nucleotide sequence of SEQ ID NO: 233 encodes a BCMA-targeted CAR (designated as BCMA-targeted BBz CAR54) comprising a human scFv that comprises a heavy chain variable region comprising amino acids having the sequence set forth in SEQ ID NO: 53, a light chain variable region comprising amino acids having the sequence set forth in SEQ ID NO: 54, and a linker having an amino acid sequence of SEQ ID NO: 69 positioned between the heavy chain variable region and the light chain variable region, a transmembrane domain comprising a CD8 polypeptide having amino acids 137 to 207 of SEQ ID NO: 226, and an intracellular domain comprising a CD3ζ polypeptide comprising amino acid sequence of amino acids 52 to 163 of SEQ ID NO: 195, and a co-stimulatory signaling region comprising a 4-1BB polypeptide having the amino acids 214-255 of SEQ ID NO: 197. Nucleotide sequences 270-1003 of SEQ ID NO: 233 encodes the human scFv. Nucleotide sequences 1011-1223 of SEQ ID NO: 233 encodes the CD8 polypeptide comprised in the transmembrane domain. Nucleotide sequences 1224-1349 of SEQ ID NO: 233 encodes the 4-1BB polypeptide comprised in the intracellular domain. Nucleotide sequences 1350-1688 of SEQ ID NO: 233 encodes the CD3zeta polypeptide comprised in the intracellular domain. Other portions of SEQ ID NO: 233 are shown in Table 23.

TABLE 23

| | nucleotide Sequence positions | number of |
|---|---|---|
| Portions | of SEQ ID NO: 233 | nucleotides |
| Kappa sp | 210 . . . 269 | 60 |
| LTR | 1968 . . . 2437 | 470 |
| M13 fwd | 3136 . . . 3152 | 17 |
| AmpR promoter | 3627 . . . 3731 | 105 |
| AmpR | 3732 . . . 4592 | 861 |
| ori | 4763 . . . 5351 | 589 |
| CAP binding site | 5639 . . . 5660 | 22 |
| lac promoter | 5675 . . . 5705 | 31 |
| lac operator | 5713 . . . 5729 | 17 |
| M13 rev | 5737 . . . 5753 | 17 |
| LTR | 6162 . . . 6755 | 594 |
| MMLV Psi | 6818 . . . 7175 | 358 |
| gag (truncated) | 7240 . . . 15 | 417 |

In certain embodiments, the isolated nucleic acid molecule encodes a functional portion of a presently disclosed BCMA-targeted CAR. As used herein, the term “functional portion” refers to any portion, part or fragment of a presently disclosed BCMA-targeted CAR, which portion, part or fragment retains the biological activity of the BCMA-targeted CAR (the parent CAR). For example, functional portions encompass the portions, parts or fragments of a presently disclosed BCMA-targeted CAR that retains the ability to recognize a target cell, to treat a disease, e.g., multiple myeloma, to a similar, same, or even a higher extent as the parent CAR. In certain embodiments, an isolated nucleic acid molecule encoding a functional portion of a presently disclosed BCMA-targeted CAR can encode a protein comprising, e.g., about 10%, 20%, 25%, 30%, 35%, 40%, 45%, 50%, 55%, 60%, 65%, 70%, 75%, 80%, 85%, 90%, and 95%, or more of the parent CAR.

One Phase I clinical trial (NCT02215967) operated by National Cancer Institute (NCI) used anti-BCMA CAR-transduced T cells for treating multiple myeloma.^{33,34} The anti-BCMA CAR applied in the NCI's clinical trial comprises a murine scFv binding to human BCMA. Using a mouse antibody or a mouse scFv for treating humans can lead to anti-mouse antibody (HAMA) response, which may be life-threatening. Unlike NCI clinical trial, in certain embodiments, the presently disclosed BCMA-targeted CAR comprises a human scFv, and thus, affords a much decreased risk of immunogenicity, compared with CARs comprising murine antibodies (see Maus et al., Cancer Immunol Res (2003); 1(1):26-31), which reports that the potential immunogenicity of CARs derived from murine antibodies may be a safety issue for mRNA CARs).

### III. Immunoresponsive Cells

The presently disclosed subject matter provides immunoresponsive cells expressing a CAR that comprises an extracellular antigen-binding domain, a transmembrane domain and an intracellular domain, where the extracellular antigen-binding domain specifically binds to BCMA (e.g., human BCMA) as described above. The immunoresponsive cells can be transduced with a presently disclosed CAR such that the cells express the CAR. The presently disclosed subject matter also provides methods of using such cells for the treatment of a tumor, e.g., multiple myeloma (MM). The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, cytotoxic T lymphocytes (CTLs), regulatory T cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM}cells and T_{EMRA} cells), Regulatory T cells (also known as suppressor T cells), Natural killer T cells, Mucosal associated invariant T cells, and γδ T cells. In certain embodiments, the CAR-expressing T cells express Foxp3 to achieve and maintain a T regulatory phenotype. Natural killer (NK) cells can be lymphocytes that are part of cell-mediated immunity and act during the innate immune response. NK cells do not require prior activation in order to perform their cytotoxic effect on target cells. Cytotoxic T cells (CTL or killer T cells) are a subset of T lymphocytes capable of inducing the death of infected somatic or tumor cells.

The immunoresponsive cells of the presently disclosed subject matter can express an extracellular antigen-binding domain (e.g., a human scFV, a Fab that is optionally crosslinked, or a F(ab)₂) that specifically binds to BCMA (e.g., human BCMA), for the treatment of multiple myeloma. Such immunoresponsive cells can be administered to a subject (e.g., a human subject) in need thereof for the treatment of multiple myeloma. In certain embodiments, the immunoresponsive cell is a T cell. The T cell can be a CD4⁺ T cell or a CD8⁺ T cell. In certain embodiments, the T cell is a CD4⁺ T cell. In another embodiment, the T cell is a CD8⁺ T cell.

A presently disclosed immunoresponsive cell can be further transduced with at least one co-stimulatory ligand, such that the immunoresponsive cell co-expresses or is induced to co-express the BCMA-targeted CAR and the at least one co-stimulatory ligand. The interaction between the BCMA-targeted CAR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell is transduced with one co-stimulatory ligand that is 4-1BBL. In certain embodiments, the immunoresponsive cell is transduced with two co-stimulatory ligands that are 4-1BBL and CD80. CARs transduced with at least one co-stimulatory ligand are described in U.S. Pat. No. 8,389,282, which is incorporated by reference in its entirety.

Furthermore, a presently disclosed immunoresponsive cell can be further transduced with at least one cytokine, such that the immunoresponsive cell secretes the at least one cytokine as well as expresses the BCMA-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The BCMA-specific or BCMA-targeted human lymphocytes that can be used in peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, a presently disclosed immunoresponsive cell (e.g., T cell) expresses from about 1 to about 4, from about 2 to about 4, from about 3 to about 4, from about 1 to about 2, from about 1 to about 3, or from about 2 to about 3 vector copy numbers/cell of a presently disclosed BCMA-targeted CAR.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

### IV. Vectors

Genetic modification of immunoresponsive cells (e.g., T cells, CTL cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the BCMA-targeted CAR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide BCMA-targeted CAR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a co-stimulatory ligand (e.g., 4-1BBL and IL-12) in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263 267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

In certain non-limiting embodiments, the vector expressing a presently disclosed BCMA-targeted CAR is a retroviral vector, e.g., a 293galv9 retroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1 α enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### V. Polypeptides and Analogs and Polynucleotides

Also included in the presently disclosed subject matter are extracellular antigen-binding domains that specifically binds to a BCMA (e.g., human BCMA) (e.g., an scFv (e.g., a human scFv), a Fab, or a (Fab)₂), CD3ζ, CD8, CD28, etc. polypeptides or fragments thereof, and polynucleotides encoding thereof that are modified in ways that enhance their anti-tumor activity when expressed in an immunoresponsive cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity with all or part of a naturally-occurring amino, acid sequence of the presently disclosed subject matter. The length of sequence comparison is at least 5, 10, 15, 20, 25, 50, 75, 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least 5, 10, 13, or 15 amino acids. In certain embodiments, a fragment is at least 20 contiguous amino acids, at least 30 contiguous amino acids, or at least 50 contiguous amino acids. In certain embodiments, a fragment is at least 60 to 80, 100, 200, 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to BCMA (e.g., human BCMA) (e.g., an scFv (e.g., a human scFv), a Fab, or a (Fab)₂), CD3ζ, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotids of the presently disclosed subject matter, including, but not limited to, OptimumGene™, Encor optimization, and Blue Heron.

### VI. Administration

BCMA-targeted CARs and immunoresponsive cells expressing thereof of the presently disclosed subject matter can be provided systemically or directly to a subject for treating or preventing a neoplasia. In certain embodiments, the BCMA-targeted CARs and immunoresponsive cells expressing thereof are directly injected into an organ of interest (e.g., an organ affected by a neoplasia). Alternatively or additionally, the BCMA-targeted CARs and immunoresponsive cells expressing thereof are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

BCMA-targeted CARs and immunoresponsive cells expressing thereof of the presently disclosed subject matter can be administered in any physiologically acceptable vehicle, normally intravascularly, although they may also be introduced into bone or other convenient site where the cells may find an appropriate site for regeneration and differentiation (e.g., thymus). Usually, at least 1×10⁵ cells can be administered, eventually reaching 1×10¹⁰ or more. A cell population comprising immunoresponsive cells expressing a BCMA-targeted CAR can comprise a purified population of cells. Those skilled in the art can readily determine the percentage of immunoresponsive cells in a cell population using various well-known methods, such as fluorescence activated cell sorting (FACS). The ranges of purity in cell populations comprising genetically modified immunoresponsive cells expressing a BCMA-specific CAR can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The immunoresponsive cells can be introduced by injection, catheter, or the like. If desired, factors can also be included, including, but not limited to, interleukins, e.g. IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

Compositions of the presently disclosed subject matter comprise pharmaceutical compositions comprising immunoresponsive cells expressing a BCMA-targeted CAR and a pharmaceutically acceptable carrier. Administration can be autologous or non-autologous. For example, immunoresponsive cells expressing a BCMA-targeted CAR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells expressing a BCMA-targeted CAR), it can be formulated in a unit dosage injectable form (solution, suspension, emulsion).

### VII. Formulations

Immunoresponsive cells expressing a generally BCMA-targeted CAR and compositions comprising thereof of the presently disclosed subject matter can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions comprising immunoresponsive cells expressing a generally BCMA-targeted CAR of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the present invention, however, any vehicle, diluent, or additive used would have to be compatible with the immunoresponsive cells expressing a generally BCMA-targeted CAR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as describe in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the immunoresponsive cells of the presently disclosed subject matter is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In certain embodiments, at least about 1×10⁸, about 2×10⁸, about 3×10⁸, about 4×10⁸, and about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### VIII. Methods of Treatment

Tumor Microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

Challenges in tumor immunology. Effective tumor immunity requires recognition of tumor antigens and unopposed tumor elimination by immune effector cells. Tumor antigens must contain peptide epitopes that are presented by the tumor and can be recognized by specific cytotoxic T lymphocytes (CTLs). The primed CTLs must expand to a sufficient number and migrate to tumor sites, wherein they mature into effectors to perform their functions, which are enhanced by helper T cells and dampened by Tregs and inhibitory macrophages.

Targeted T cell therapy with engineered T lymphocytes. T cell engineering is a groundbreaking strategy to potentially resolve many previously observed shortcomings of earlier immunotherapeutic approaches. Within the past year, researchers have reported dramatic complete remissions in relapsed^{16,17}, chemorefractory leukemia and metastatic melanoma¹⁸⁻²⁰, obtained with autologous peripheral blood T cells targeted to a defined antigen (CD19 and NY-ESO-1, respectively).

Rationale for a genetic approach: Cell engineering can be used to redirect T cells toward tumor antigens and to enhance T cell function. One impetus for genetic T cell modification is the potential to enhance T cell survival and expansion and to offset T cell death, anergy, and immune suppression. The genetic targeting of T cells can also be refined to prevent undesired destruction of normal tissues.

Chimeric antigen receptors (CARs): Tumor-specific T cells can be generated by the transfer of genes that encode CARs²¹⁻²⁶. Second-generation CARs comprise a tumor antigen-binding domain fused to an intracellular signaling domain capable of activating T cells and a co-stimulatory domain designed to augment T cell potency and persistence²⁷. CAR design can therefore reconcile antigen recognition with signal transduction, two functions that are physiologically borne by two separate complexes, the TCR heterodimer and the CD3 complex. The CAR's extracellular antigen-binding domain is usually derived from a murine monoclonal antibody (mAb) or from receptors or their ligands. Antigen recognition is therefore not MHC-restricted^{28,29} and is therefore applicable to any patient expressing the target antigen, using the same CAR. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation. Because MHC restriction of antigen recognition is bypassed, the function of CAR-targeted T cells is not affected by HLA downregulation or defects in the antigen-processing machinery.

T cell requirements for expansion and survival: Proliferation of tumor-specific T cells is needed ex vivo and is arguably desirable in vivo. T cell proliferation must be accompanied by T cell survival to permit absolute T cell expansion and persistence. To proliferate in response to antigen, T cells must receive two signals. One is provided by TCR recognition of antigenic peptide/MHC complexes displayed on the surface of antigen-presenting cells (APCs)²⁵. The other is provided by a T cell co-stimulatory receptor, such as the CD28 or 4-1BB receptors. Whereas the cytolytic activity of T cells does not require concomitant co-stimulation, there is a critical need for the provision of co-stimulatory signals to sustain the antitumor functions of adoptively transferred T cells, as previously demonstrated^{23,27,30-3}2.

Immune monitoring: Lymphocytes are multifunctional “drugs” that exhibit dynamically evolving effects after infusion. Upon antigen encounter, tumor-specific T cells activate and/or release a variety of proteins that can trigger tumor killing, T cell proliferation, and recruitment or immunomodulation of other immune cells. Thus, measuring which proteins are secreted from which cells, in what quantity, and at what time point yields profound insights into why a particular patient is or is not responding and provides critical feedback for designing more-effective trials. These assay systems will permit direct and meaningful comparisons of clinical approaches and thus help design rational, next-generation therapeutic strategies.

For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered. For adoptive immunotherapy using antigen-specific T cells, cell doses in the range of about 10⁶ to about 10¹⁰ (e.g., about 10⁹) are typically infused. Upon administration of the immunoresponsive cells into the subject and subsequent differentiation, the immunoresponsive cells are induced that are specifically directed against one specific antigen (e.g., BCMA). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are intravenously administered to the subject in need.

The presently disclosed subject matter provides various methods of using the immunoresponsive cells (e.g., T cells) expressing a BCMA-targeted CAR. For example, the presently disclosed subject matter provides methods of reducing tumor burden in a subject. In one non-limiting example, the method of reducing tumor burden comprises administering an effective amount of the presently disclosed immunoresponsive cell to the subject, thereby inducing tumor cell death in the subject. The presently disclosed immunoresponsive cell can reduce the number of tumor cells, reduce tumor size, and/or eradicate the tumor in the subject. Non-limiting examples of suitable tumor include multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the tumor is multiple myeloma.

The presently disclosed subject matter also provides methods of increasing or lengthening survival of a subject having a neoplasia. In one non-limiting example, the method of increasing or lengthening survival of a subject having neoplasia comprises administering an effective amount of the presently disclosed immunoresponsive cell to the subject, thereby increasing or lengthening survival of the subject. The method can reduce or eradicate tumor burden in the subject. The presently disclosed subject matter further provides methods for treating or preventing a neoplasia in a subject, comprising administering the presently disclosed immunoresponsive cell to the subject.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

Cancers whose growth may be inhibited using the immunoresponsive cells of the presently disclosed subject matter comprise cancers typically responsive to immunotherapy. Non-limiting examples of cancers for treatment include multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the cancer is multiple myeloma.

Additionally, the presently disclosed subject matter provides methods of increasing immune-activating cytokine production in response to a cancer cell in a subject. In one non-limiting example, the method comprises administering the presently disclosed immunoresponsive cell to the subject. The immune-activating cytokine can be granulocyte macrophage colony stimulating factor (GM-CSF), IFN-α, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the immunoresponsive cells including a BCMA-specific CAR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor (e.g., multiple myeloma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., multiple myeloma).

A second group of suitable subjects is known in the art as the “adjuvant group.” These are individuals who have had a history of neoplasia (e.g., multiple myeloma), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor (e.g., multiple myeloma) has invaded neighboring tissues, or who show involvement of lymph nodes. Another group has a genetic predisposition to neoplasia (e.g., multiple myeloma) but has not yet evidenced clinical signs of neoplasia (e.g., multiple myeloma). For instance, women testing positive for a genetic mutation associated with breast cancer, but still of childbearing age, can wish to receive one or more of the antigen-binding fragments described herein in treatment prophylactically to prevent the occurrence of neoplasia until it is suitable to perform preventive surgery.

The subjects can have an advanced form of disease (e.g., multiple myeloma), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the BCMA-targeted CAR-expressing immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the 3′ terminus of the intracellular domain of the BCMA-targeted CAR. The suicide gene can be included within the vector comprising nucleic acids encoding the presently disclosed BCMA-targeted CARs. In this way, administration of a prodrug designed to activate the suicide gene (e.g., a prodrug (e.g., AP1903 that can activates iCasp-9) during malignant T-cell transformation (e.g., GVHD) triggers apoptosis in the suicide gene-activated CAR-expressing T cells.

### IX. Kits

The presently disclosed subject matter provides kits for the treatment or prevention of a neoplasia (e.g., multiple myeloma). In certain embodiments, the kit comprises a therapeutic or prophylactic composition containing an effective amount of an immunoresponsive cell comprising a BCMA-targeted CAR in unit dosage form. In particular embodiments, the cells further expresses at least one co-stimulatory ligand. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the immunoresponsive cell is provided together with instructions for administering the cell to a subject having or at risk of developing a neoplasia (e.g., multiple myeloma). The instructions will generally include information about the use of the composition for the treatment or prevention of a neoplasia (e.g., multiple myeloma). In other embodiments, the instructions include at least one of the following: description of the therapeutic agent; dosage schedule and administration for treatment or prevention of a neoplasia (e.g., multiple myeloma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1—BCMA Expression in Various Tissues

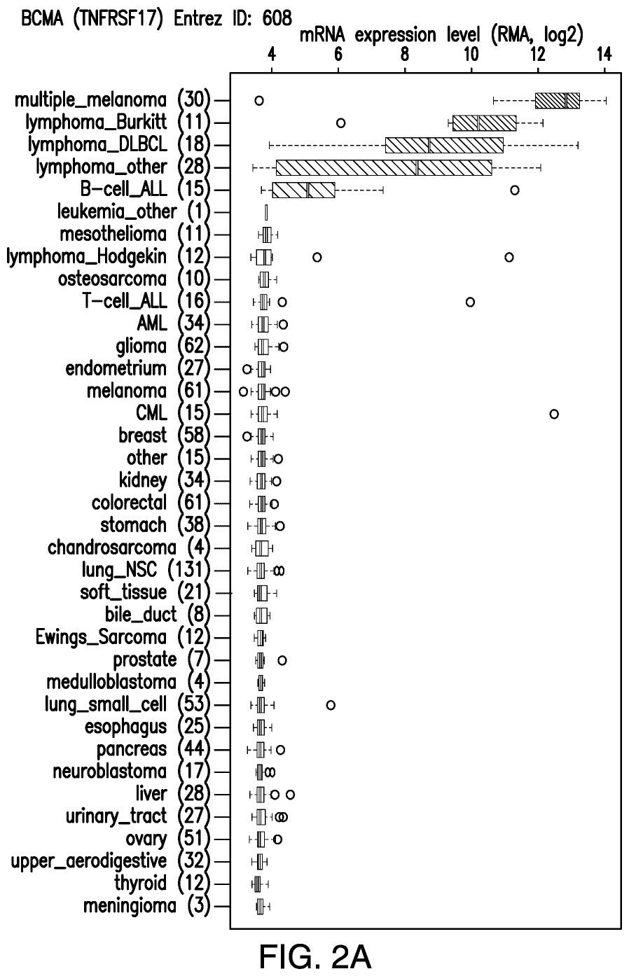

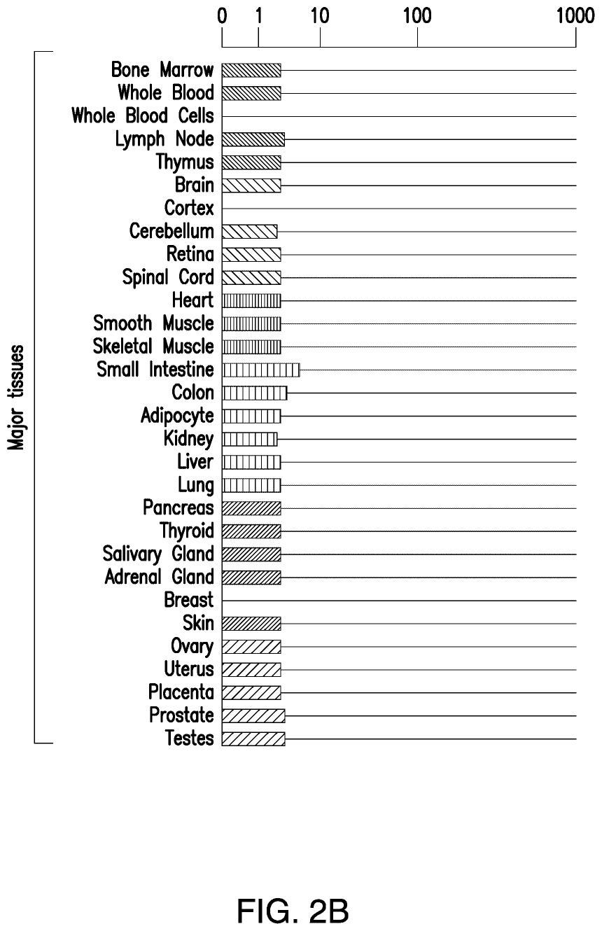

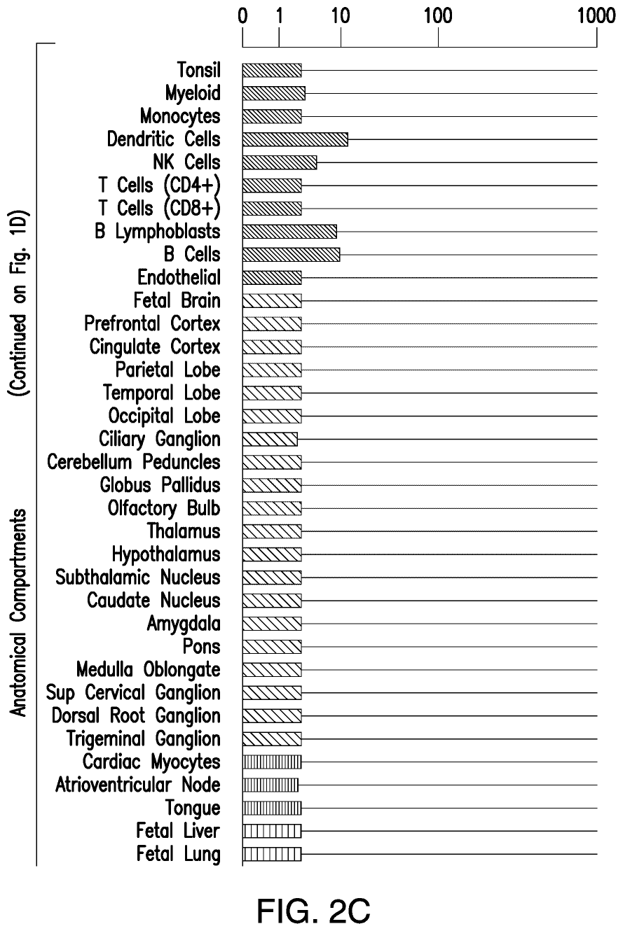

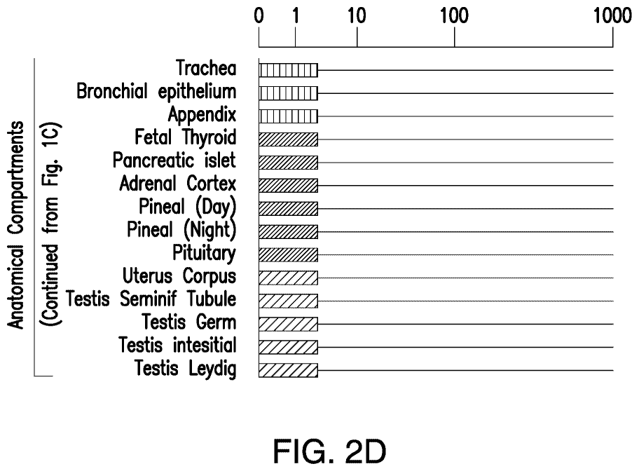

The Expression of human BCMA was evaluated in various malignant and normal tissues by investigating gene expression profiles in databases such as the cancer cell line encyclopedia and BioGPS. As shown in FIGS. 2A-2D, human BCMA was highly expressed in lymphoma and multiple myeloma, but not in other malignant tissues. Normal expression appeared limited to B-cells and plasma cells. Potential BCMA targeted CAR T cell eradication of these normal cell types may not have significant adverse effects based on inventors' patient experience with CD19 targeted CAR T cells. Any lack of physiologic antibody production can be addressed with intravenous immunoglobulin treatment.

### Example 2—Construct of BCMA-specific28z CARs

Multiple unique fully human scFv's to BCMA were generated, and CARs based on these scFv's were generated. Multiple scFv's were identified by screening a fully human scFv phage library (>6×10¹⁰ scFv's) with BCMA-Fc fusion protein and then 3T3 cells expressing human BCMA. After sequencing, 57 unique and BCMA-Fc positive clones were found out of 79 sequenced positive clones; the unique clone rate was 72%. FACS analysis of phage antibody clones against BCMA-3T3 and parental 3T3 cell lines resulted in confirming 25 unique positive clones.

ET140-153 scFv (or “ET140-3 scFv”), ET140-174 scFv (or “ET140-24 scFv”), ET140-187 scFv (or “ET140-37 scFv”), ET140-190 scFv (or “ET140-40 scFv”), and ET140-204 scFv (or “ET140-54 scFv”) were used to generate BCMA-targeted 28z CARs 3, 24, 37, 40, and 54, respectively. These BCMA-targeted 28z CARs have similar structure, e.g., each has a transmembrane domain comprising a CD28 polypeptide, and an intracellular domain comprising a CD3ζ polypeptide and a co-stimulatory signaling region that comprises a CD28 polypeptide, as shown in FIG. 1. Each of these BCMA-targeted CARs were cloned into a retroviral vector. These viral vectors were then transduced into HEK 293galv9 viral packaging cells in order to generate a stable packaging line for generation of CAR⁺ T cells.

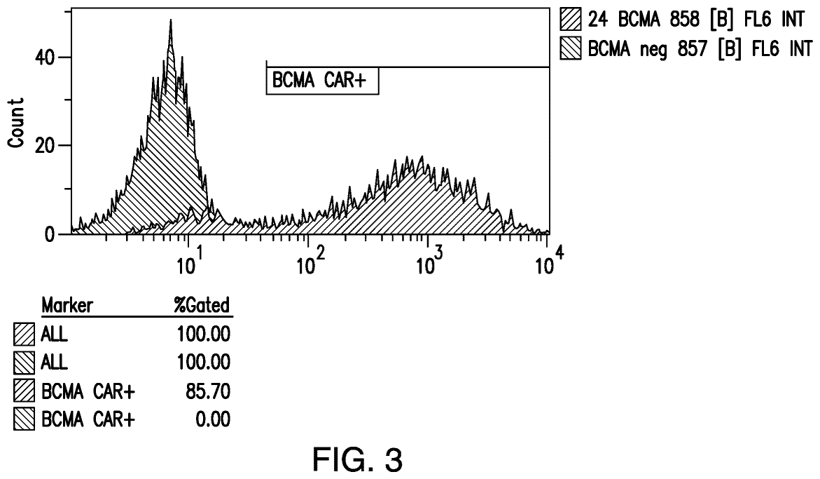

Human T cells (unselected (CD4 and CD8) human T cells from a healthy donor) were transduced with retrovirus in order to express each of these BCMA-targeted CARs such that the T cells expressed these BCMA-targeted 28z CARs. The cell surface expression of BCMA-targeted CARs on human T cells was determined via binding A647 conjugated BCMA-Fc fusion protein. The cell surface expression of BCMA-targeted 28z CAR24 was assessed, and cell surface detection was valided by flow cytometry, as shown in FIG. 3.

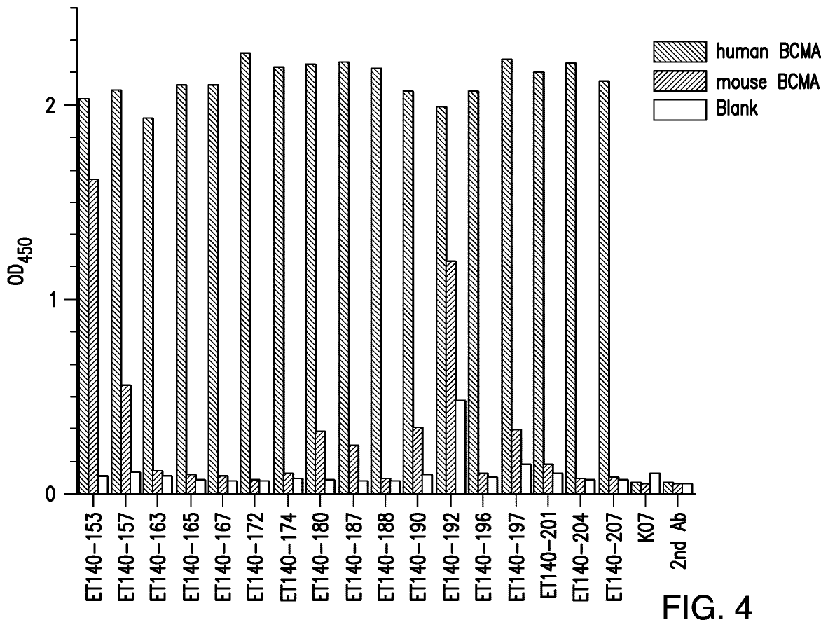

The cross-reacting activity of seventeen human scFv's between human BCMA and mouse BCMA was assessed. As shown in FIG. 4, certain scFv's, e.g., ET140-153 scFv (or “ET140-3 scFv”) and ET140-192 scFv (or “ET140-42 scFv”) cross-reacted with mouse BCMA, thus, this scFv can be used for syngeneic mouse studies.

### Example 3—Activity of BCMA-specific CARs

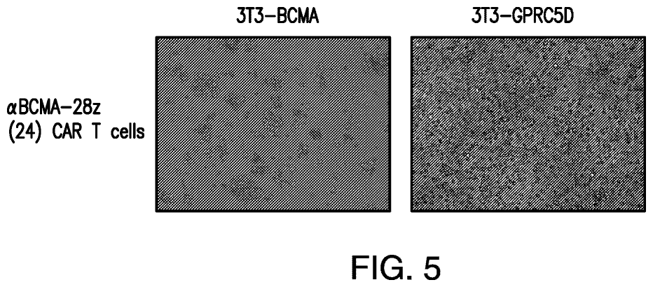

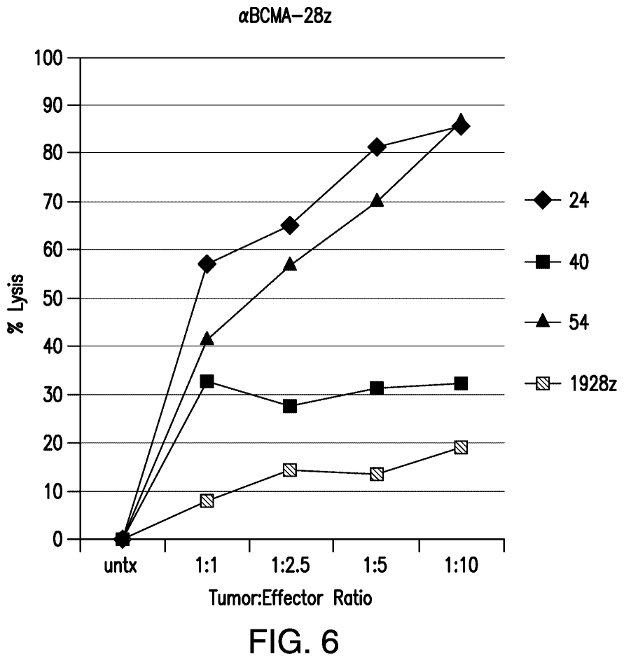

The anti-tumor activity of the presently disclosed BCMA-specific 28z CARs was evaluated. The in vitro data showed that the BCMA-specific CARs specifically killed BCMA presenting cells, including MM cell lines. For example, as shown in FIG. 5, the T cells expressing the BCMA-specific 28z CAR24 killed 3T3 cells overexpressing BCMA (but not control 3T3s overexpressing an irrelevant antigen). As shown in FIG. 6, the T cells expressing the BCMA-specific 28z CARs 24, 40, and 54 killed human MM cell lines.

### Example 4—Screening Data for Anti-BCMA Antibodies

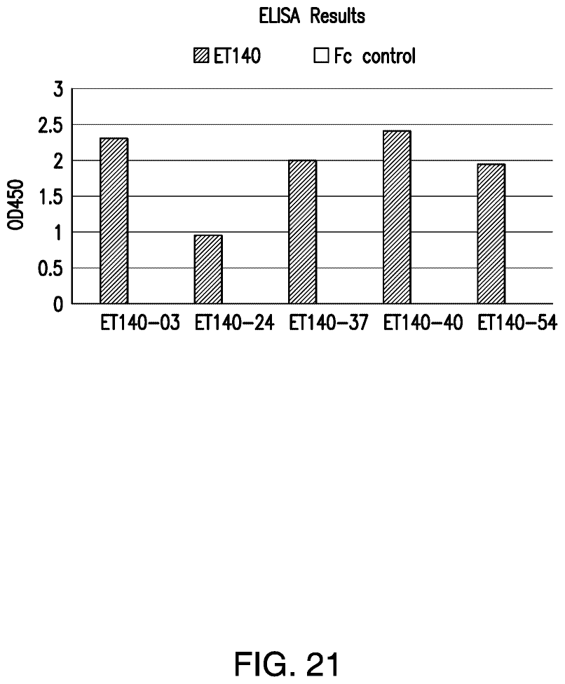

ELISA Screening: FIG. 21 shows the representative results of protein ELISA screening against BCMA antigen using specific scFv phage antibody clones (ET140-3, ET140-24, ET140-37, ET140-40 and ET140-54). ELISA plates were coated with human BCMA ECD-Fc fusion protein, control-Fc fusion protein, or PBS alone as blank control, respectively. Individual phage clones from enriched phage display panning pools against BCMA ECD-Fc fusion protein were incubated in the coated plates. Binding of the phage clones was detected by HRP-conjugated anti-M13 antibodies and developed using TMB substrate. The absorbance was read at 450 nm.

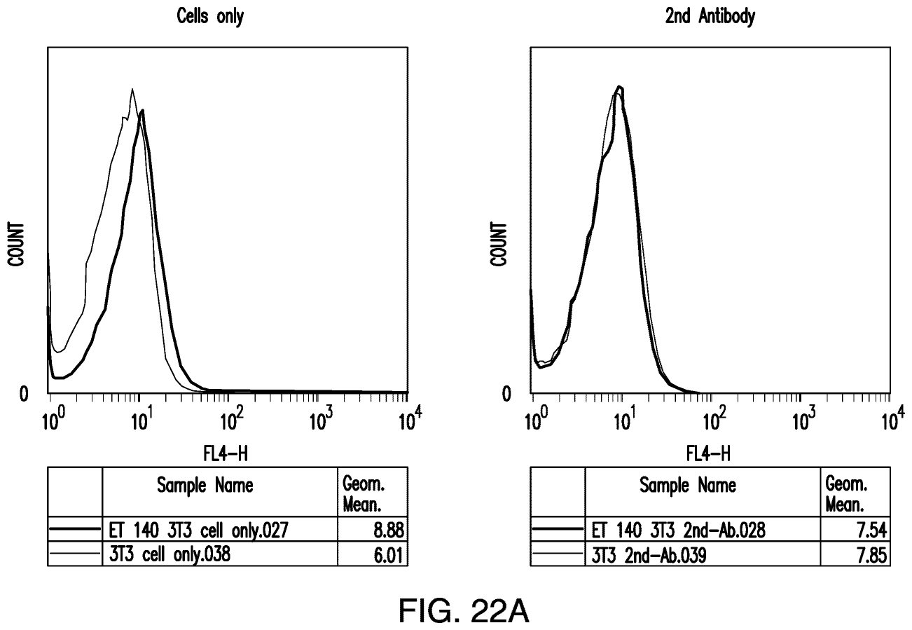

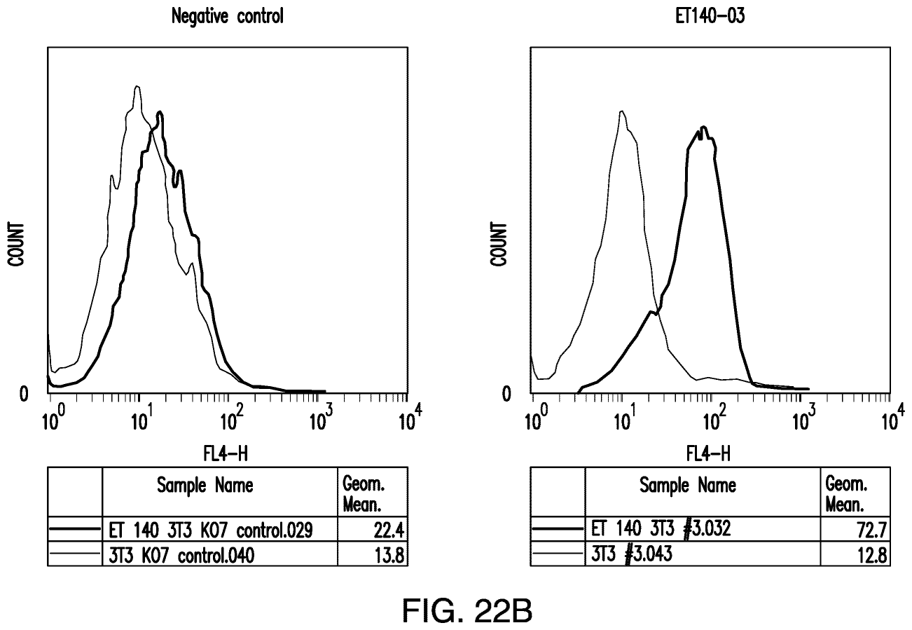

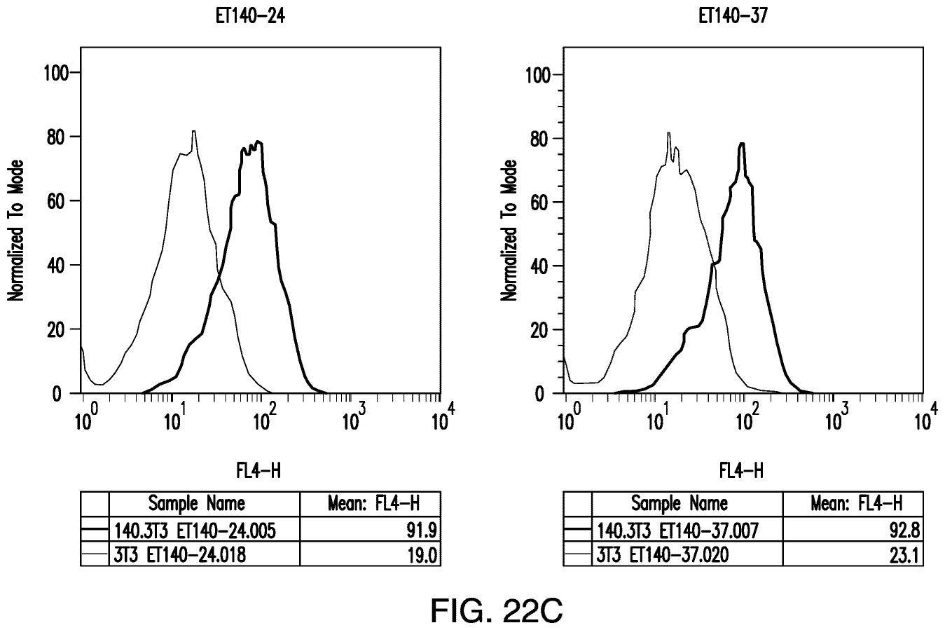

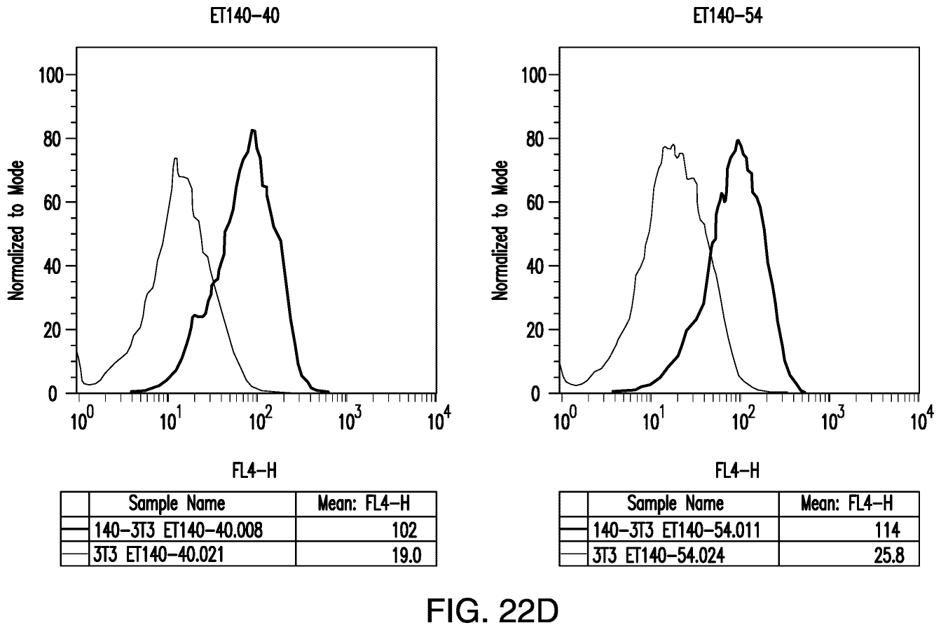

FACS Screening: FIGS. 22A-22D show a representative figure of a FACS analysis of the BCMA-specific phage antibody clones ET140-3, ET140-24, ET140-37, ET140-40 and ET140-54. Phage clones were incubated with 3T3-BCMA cell line, then with anti-M13 mouse antibody. Finally APC-labeled anti-mouse IgG 2nd antibody was added to the reaction after washing again. The binding was measured by FACS and expressed as mean fluorescence intensity (MFI). Cells incubated with 2nd antibody alone, M13 K07 helper phage and cells only were used as negative controls.

### Example 5—Construct of BCMA-specific BBz CARs

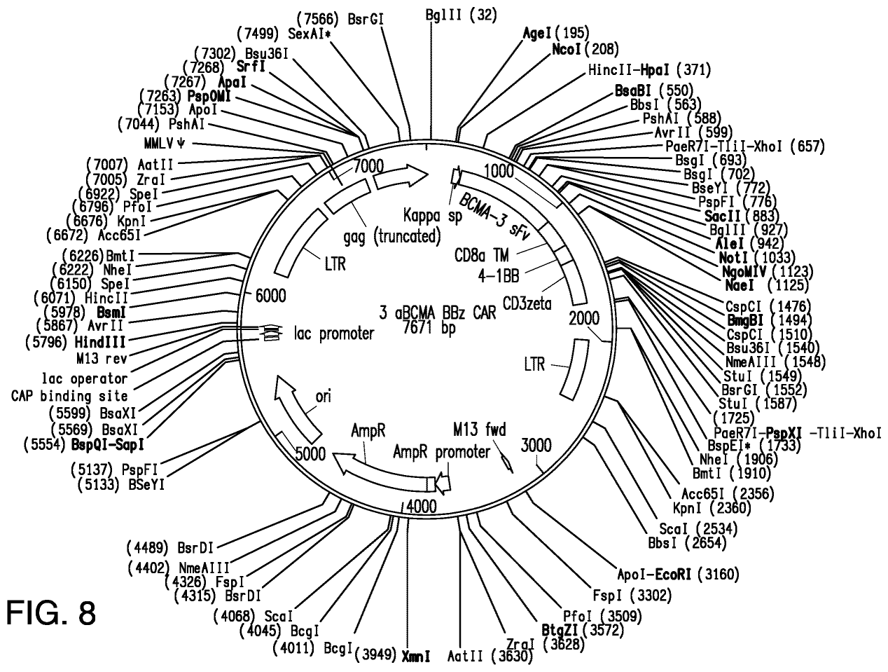

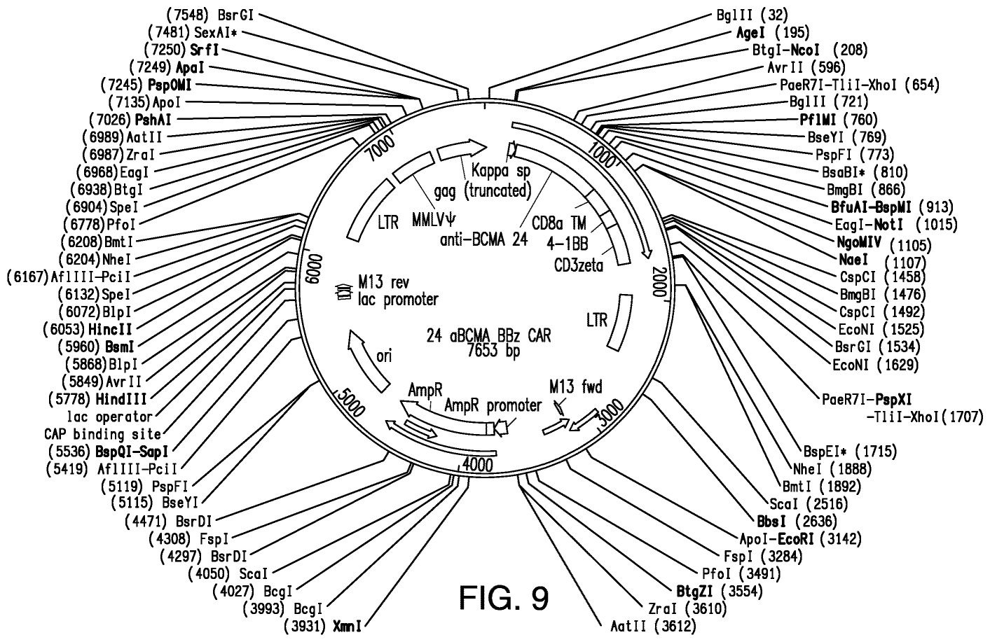

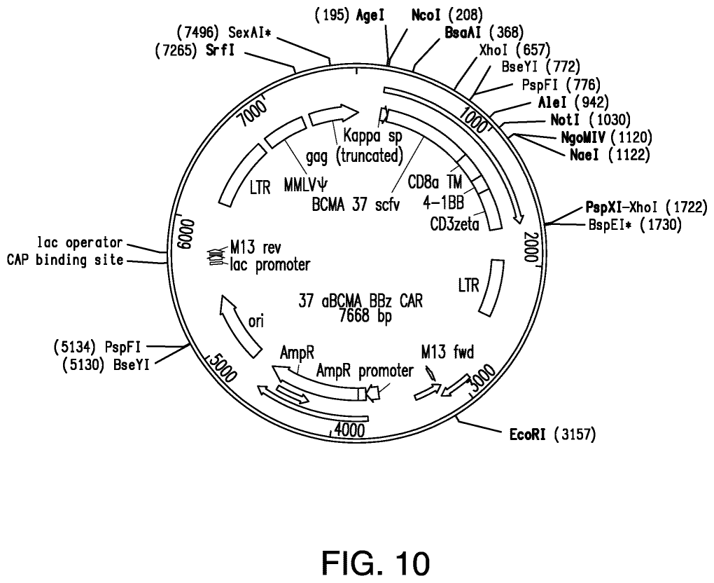

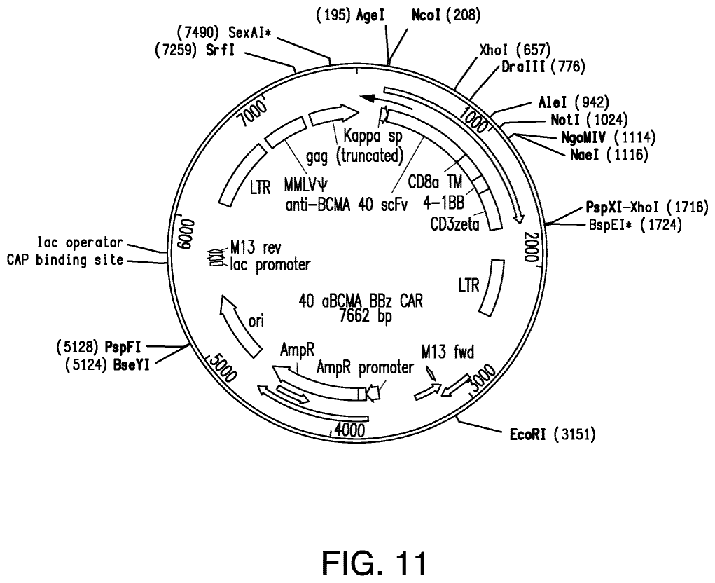

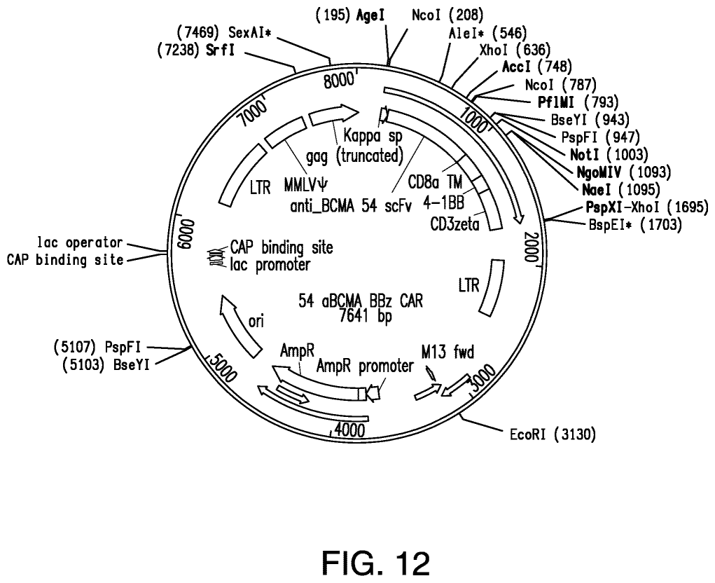

Multiple unique fully human scFv's to BCMA were generated as described in Example 2. ET140-153 scFv (or “ET140-3 scFv”), ET140-174 scFv (or “ET140-24 scFv”), ET140-187 scFv (or “ET140-37 scFv”), ET140-190 scFv (or “ET140-40 scFv”), and ET140-204 scFv (or “ET140-54 scFv”) were used to generate BCMA-targeted BBz CARs 3, 24, 37, 40, and 54, respectively. Each of these BCMA-targeted BBz CARs has a transmembrane domain comprising a CD8 polypeptide, and an intracellular domain comprising a CD3ζ polypeptide and a co-stimulatory signaling region that comprises a 4-1BB polypeptide, as shown in FIG. 7. Each of these BCMA-targeted CARs were cloned into an SFG retroviral vector, as an example the 4-1BB containing CAR vectors are shown in FIGS. 8-12.

### Example 6—Activity of BCMA-targeted CAR T Cells

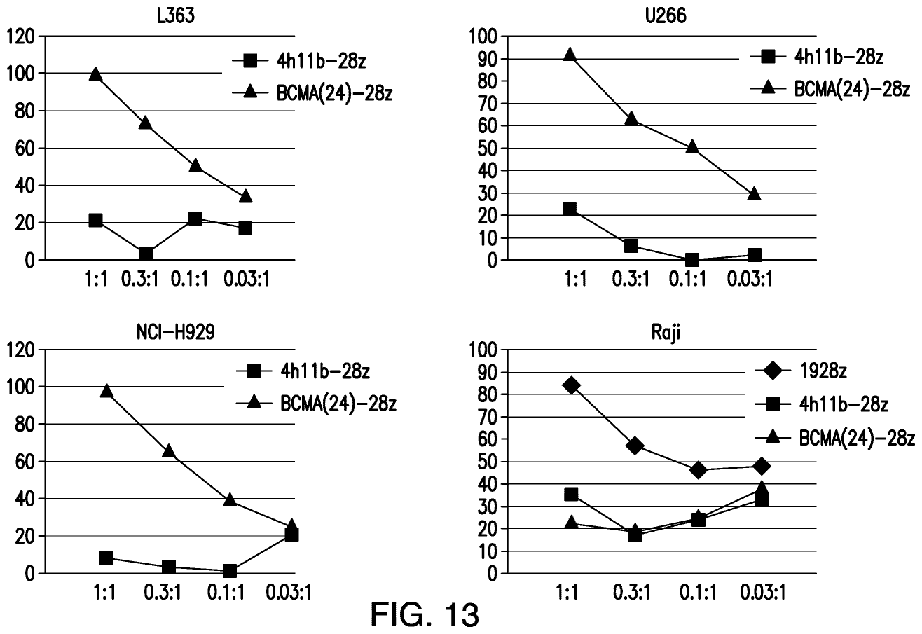

As shown in FIG. 13, BCMA-specific 28z CAR24 lysed human MM cell lines L363, NCL-H929, and U266, compared to irrelevantly targeted 4h11-28z MUC16 targeted CAR T cells. The cytotoxicity exhibited by observed BCMA-specific 28z CAR24 was specific to BCMA, as it did not lyse BCMA negative CD19 positive Raji Burkett lymphoma cell line, as shown in FIG. 13.

### Example 7—Induction of Cytokine Secretion by BCMA-targeted CAR T Cells

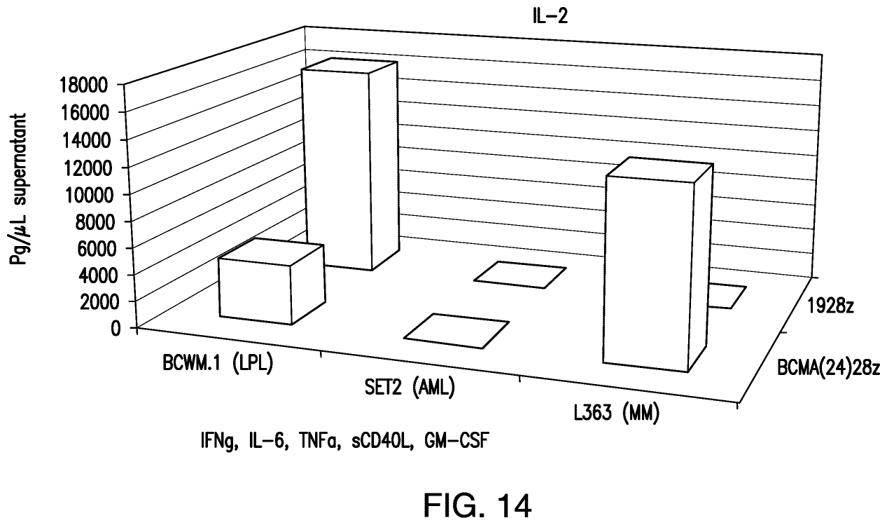

Co-culture of BCMA targeted 28z CAR24 T cells specifically with MM cell line induced cytokine secretion profile consistent with T cell activation. FIG. 14 shows the IL-2 secretion after 24 h co-culture of CAR T cells with human tumor cell lines (E:T ratio 1:1). The lymphoplasmacytic lymphoma (CD19⁺) with CD19 targeted CAR T cells (positive control) and the MM cell line with the BCMA targeted 28z CAR24 T cells displayed increased cytokine production. IFNg, IL-6, TNFa, sCD40L, GM-CSF all had similar secretion profiles (data not shown).

### Example 8—Anti-tumor Activity of BCMA-targeted CAR T cells

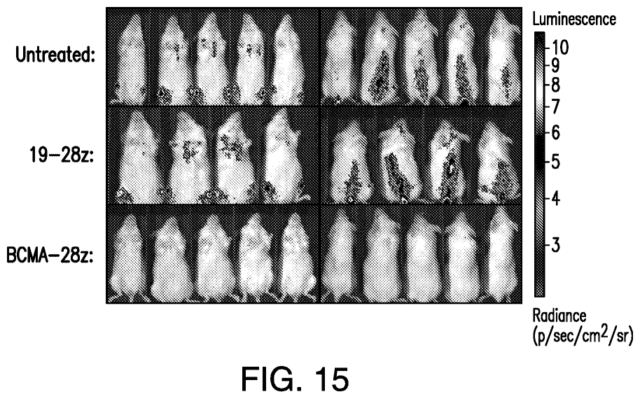

BCMA targeted 28z CAR54 T cells mediated an anti-myeloma immune response. 1×10⁷ U266 human myeloma cell line cells were injected IV into NSG mice on day 0. On day 4 1×10⁶ BCMA targeted or CD19 targeted second generation CAR T cells were injected IV. Imaging on day 11 (day 7 s/p CAR T cell injection) shows that, unlike irrelevant (CD19) targeted CAR T cells; BCMA targeted 28z CAR54 T cells can mediate an anti-tumor response. See FIG. 15.

### Example 9—Activity of BCMA-targeted CAR T Cells

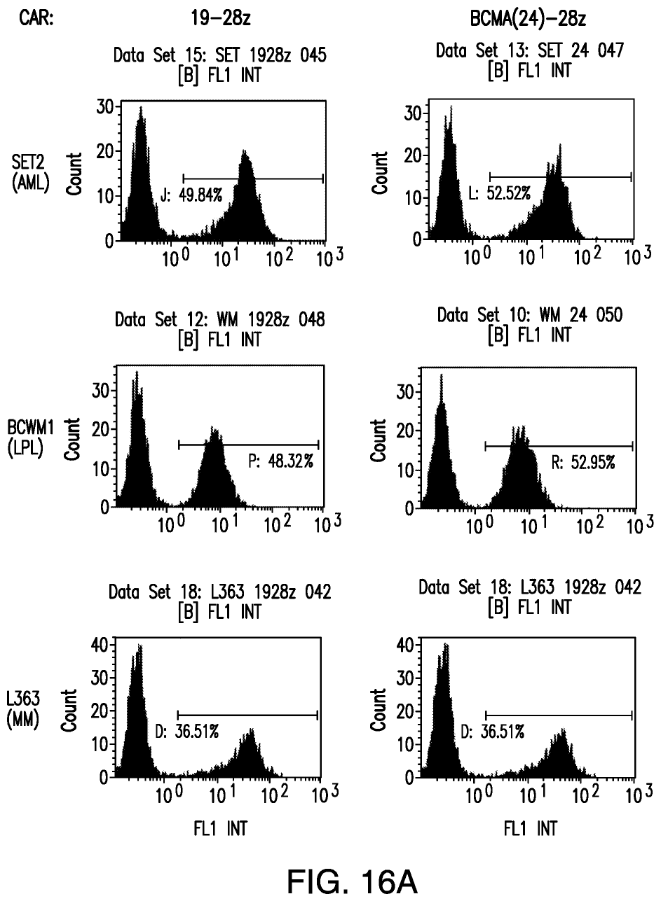

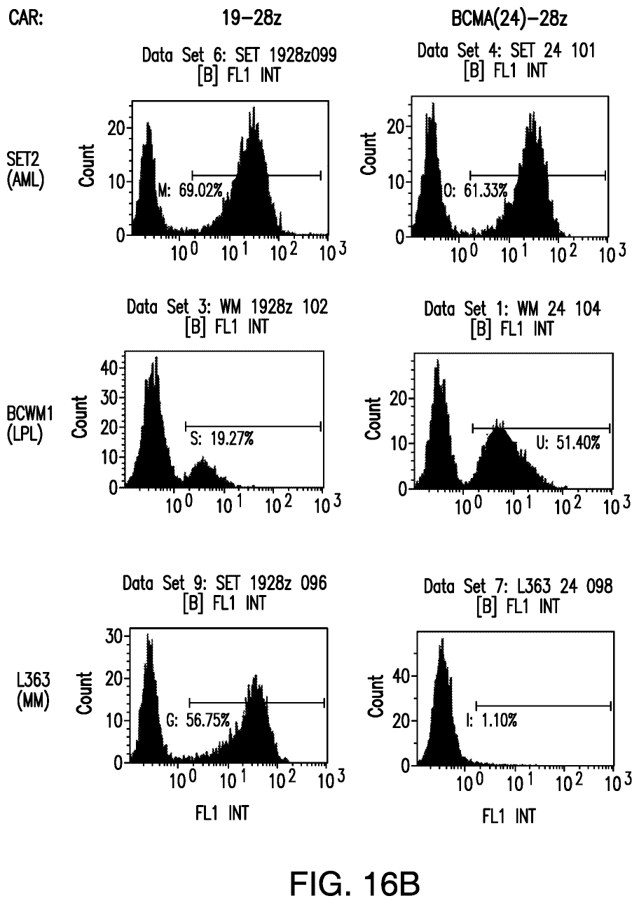

The ability of BCMA targeted CAR T cells to specifically lyse human myeloma cell line (HMCL) was tested. CD19 targeted CAR T cells or BCMA targeted 28z CAR24 T cells were incubated with GFP expressing tumor cell lines SET2 (Acute myeloid leukemia (AML), CD19⁻ BCMA⁻); BCWM1 (Lymphoplasmacytic Lymphoma (LPL), CD19-BCMA⁻); L363 (Multiple Myeloma (MM), CD19-BCMA⁺). At time 0, the percent of GFP⁺ tumor line is shown in FIG. 16A. At 36 h the positive control CD19 targeted CAR T cells have specifically killed the GFP⁺ LPL line, and similarly the BCMA targeted 28z CAR24 T cells have specifically killed the GFP⁺ MM line. See FIG. 16B.

### Example 10—Epitope Mapping of Anti-BCMA Antibodies

BCMA peptides were ordered based on the LCD sequence with N-terminal Biotin+SGSG linker+15 amino acids with 1 amino acid space. The peptide library is shown in Table 24.

TABLE 24

| | |
|---|---|
| ET140-pl | SGSGLQMAGQCSQNEYFDS [SEQ ID NO: 236] |
| ET140-p2 | SGSGQMAGQCSQNEYFDSL [SEQ ID NO: 237] |
| ET140-p3 | SGSGMAGQCSQNEYFDSLL [SEQ ID NO: 238] |
| ET140-p4 | SGSGAGQCSQNEYFDSLLH [SEQ ID NO: 239] |
| ET140-p5 | SGSGGQCSQNEYFDSLLHA [SEQ ID NO: 240] |
| ET140-p6 | SGSGQCSQNEYFDSLLHAC [SEQ ID NO: 241] |
| ET140-p7 | SGSGCSQNEYFDSLLHACI [SEQ ID NO: 242] |
| ET140-p8 | SGSGSQNEYFDSLLHACIP [SEQ ID NO: 243] |
| ET140-p9 | SGSGQNEYFDSLLHACIPC [SEQ ID NO: 244] |
| ET140-p10 | SGSGNEYFDSLLHACIPCQ [SEQ ID NO: 245] |
| ET140-pll | SGSGEYFDSLLHACIPCQL [SEQ ID NO: 246] |
| ET140-p12 | SGSGYFDSLLHACIPCQLR [SEQ ID NO: 247] |
| ET140-p13 | SGSGFDSLLHACIPCQLRC [SEQ ID NO: 248] |
| ET140-p14 | SGSGDSLLHACIPCQLRCS [SEQ ID NO: 249] |
| ET140-p15 | SGSGSLLHACIPCQLRCSS [SEQ ID NO. 250] |
| ET140-p16 | SGSGLLHACIPCQLRCSSN [SEQ ID NO: 251] |
| ET140-p17 | SGSGLHACIPCQLRCSSNT [SEQ ID NO: 252] |
| ET140-p18 | SGSGHACIPCQLRCSSNTP [SEQ ID NO: 253] |
| ET140-p19 | SGSGACIPCQLRCSSNTPP [SEQ ID NO: 254] |
| ET140-p20 | SGSGCIPCQLRCSSNTPPL [SEQ ID NO: 255] |
| ET140-p21 | SGSGIPCQLRCSSNTPPLT [SEQ ID NO: 256] |
| ET140-p22 | SGSGPCQLRCSSNTPPLTC [SEQ ID NO: 257] |
| ET140-p23 | SGSGCQLRCSSNTPPLTCQ [SEQ ID NO: 258] |
| ET140-p24 | SGSGQLRCSSNTPPLTCQR [SEQ ID NO: 259] |
| ET140-p25 | SGSGLRCSSNTPPLTCQRY [SEQ ID NO: 260] |
| ET140-p26 | SGSGRCSSNTPPLTCQRYC [SEQ ID NO: 261] |
| ET140-p27 | SGSGCSSNTPPLTCQRYCN [SEQ ID NO: 262] |
| ET140-p28 | SGSGSSNTPPLTCQRYCNA [SEQ ID NO: 263] |
| ET140-p29 | SGSGSNTPPLTCQRYCNAS [SEQ ID NO: 264] |
| ET140-p30 | SGSGNTPPLTCQRYCNASV [SEQ ID NO: 265] |
| ET140-p31 | SGSGTPPLTCQRYCNASVT [SEQ ID NO: 266] |
| ET140-p32 | SGSGPPLTCQRYCNASVTN [SEQ ID NO: 267] |
| ET140-p33 | SGSGPLTCQRYCNASVTNS [SEQ ID NO: 268] |
| ET140-p34 | SGSGLTCQRYCNASVTNSV [SEQ ID NO: 269] |
| ET140-p35 | SGSGTCQRYCNASVTNSVK [SEQ ID NO: 270] |
| ET140-p36 | SGSGCQRYCNASVTNSVKG [SEQ ID NO: 271] |
| ET140-p37 | SGSGQRYCNASVTNSVKGT [SEQ ID NO: 272] |
| ET140-p38 | SGSGRYCNASVTNSVKGTN [SEQ ID NO: 273] |
| ET140-p39 | SGSGYCNASVTNSVKGTNA [SEQ ID NO: 274] |

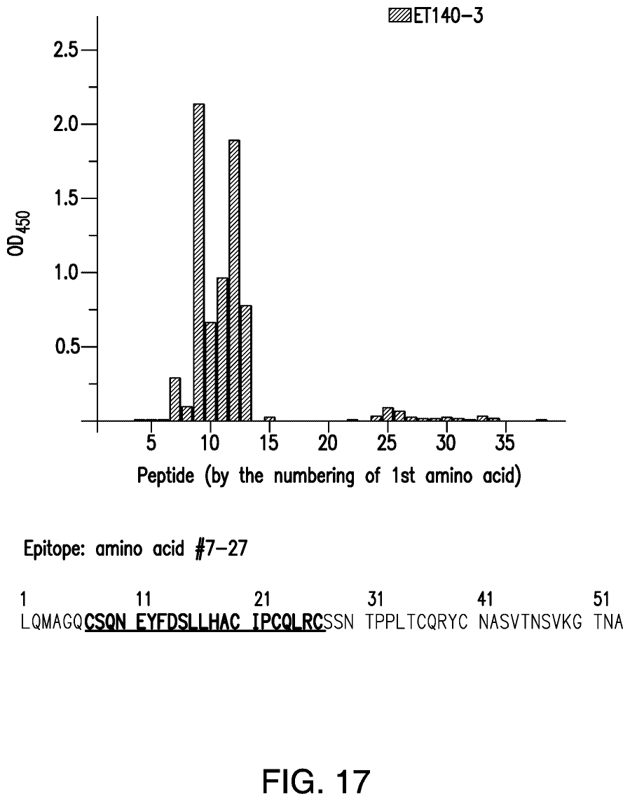

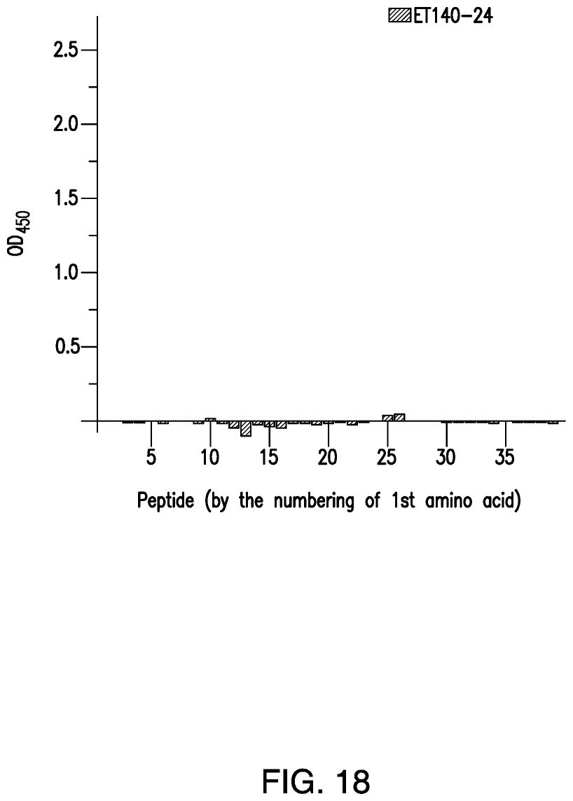

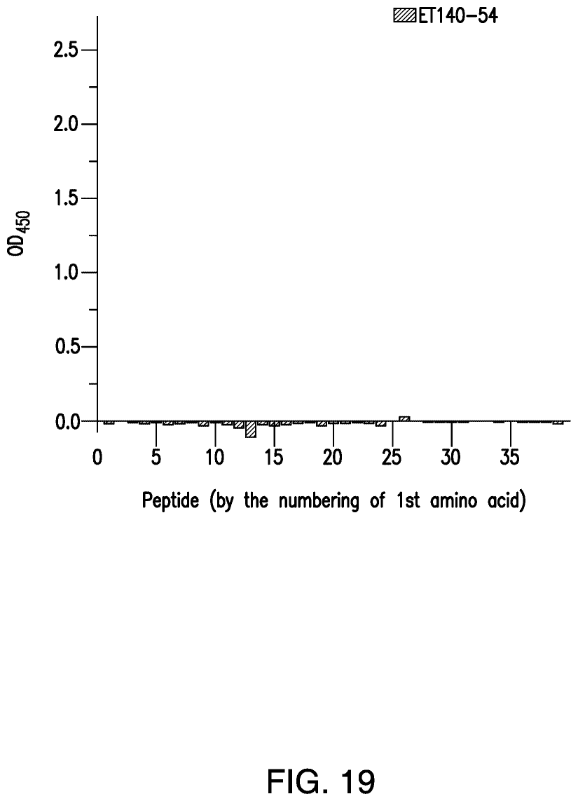

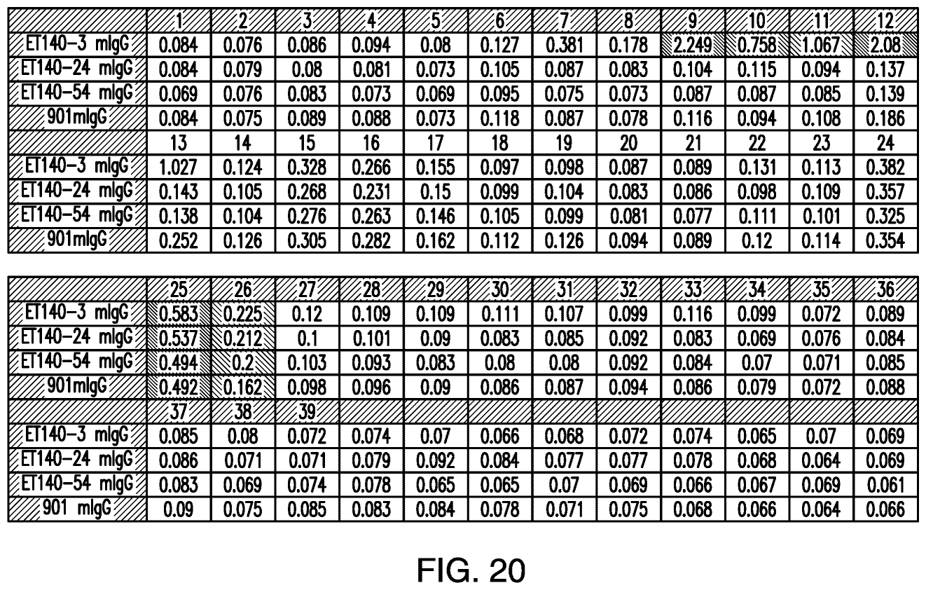

The peptides were coated onto Streptavidin plates at 2 ug/mL in PBST (PBS+0.05% Tween-20). After washing and blocking with 3% BSA. After washing, 1 ug/mL ET140-3, ET140-24, ET140-54 or ET9O1 mIgG1 was added to the wells, respectively. “mIgG1” used in all Examples represents that the variable region is fully human and the Fc part is mouse IgG1. Then HRP anti-mouse IgG detection antibody was added to each well. Finally, the color was developed using TMB substrate. A₄₅₀ was recorded for data analysis. The results are shown in FIGS. 17-20. As shown in FIGS. 17 and 20, ET140-3 bound to peptides 7-13 (i.e., amino acids 8-22, 9-23, 10-24, 11-25, 12-26, 13-27, and 14-28) of SEQ ID NO: 71. As shown in FIGS. 18 and 19, no linear epitopes found for ET140-24 or ET140-54.

Summary: 3 ET140 antibodies (mIgG1) were tested together with isotype control ET901 mIgG1 for their binding epitope towards BCMA-ECD. A peptide library consisting of 39 peptides (N-terminal biotin+SGSG linker+15 amino acids, with 1 amino acid offset) was used for epitope mapping ELISA. This allows to search for the linear binding epitope of BCMA-ECD. ET901 mIgG1 was used as background reference for each peptide. Only ET140-3 can be identified for its epitope region: a region comprising amino acids 14-22 of SEQ ID NO: 71, e.g., amino acids 8-28 of SEQ ID NO: 71.

ET140-24 and ET140-54 did not show any significant binding towards peptide library. This indicated that these two antibodies may recognize conformational epitope rather than linear epitope of BCMA.

### Example 11—Anti-BCMA Antibodies Recombinant Antigen by Surface Plasmon Resonance

Kinetics of interaction between ET140-153 mIgG1 (or “ET140-3 mIgG1”), ET140-174 mIgG1 (or “ET140-24 mIgG1”), ET140-204 mIgG1 (or “ET140-54 mIgG1”) and BCMA recombinant antigen was measured using a BIAcore X100 instrument. In brief, 50 μg/mL of modified streptavidin was immobilized onto a Sensor Chip CAP by flowing the Biotin CAPture Reagent through the flow cells at 2 μL/min for 5 minutes. 10 ug/mL biotinylated BCMA-Fc protein was loaded onto the flow cell at a rate of 30 μL/min for 3 minutes. Following the standard protocol for kinetics, a series of injection of ESK1 was performed between 0.6 and 15 μg/mL, each step consisting of a 3 minute injection at 30 μL/min and 3 minute disassociation. Afterwards, the surface was regenerated for 2 minutes with a solution consisting of 75% v/v of 8M guanidine-HCl and 25% v/v 1M NaOH. Kinetic constants were derived by the global fitting (1:1 Langmuir binding model) using BIAcore X100 Evaluation Software (Version 2.0.1). The binding affinity data are shown in Table 25.

TABLE 25

| Protein | KD |
|---|---|
| ET140-24 mIgGl | KD: 4.8 nM (BiaCore) |
| ET140-54 mIgGl | KD: 8.1 nM (BiaCore) |
| ET140-3 mIgGl | KD: 1.2 nM (BiaCore) |

Although the foregoing presently disclosed subject matter has been described in some detail by way of illustration and example for purposes of clarity of understanding, the descriptions and examples should not be construed as limiting the scope of the presently disclosed subject matter. The disclosures of all patent and scientific literature cited herein are expressly incorporated in their entirety by reference.

### REFERENCES

1. Frigyesi, I., et al. Robust isolation of malignant plasma cells in multiple myeloma. Blood 123, 1336-1340 (2014).
2. Tai, Y. T., et al. Novel afucosylated anti-B cell maturation antigen-monomethyl auristatin F antibody-drug conjugate (GSK2857916) induces potent and selective anti-multiple myeloma activity. Blood (2014).
3. Carpenter, R. O., et al. B-cell maturation antigen is a promising target for adoptive T-cell therapy of multiple myeloma. Clinical cancer research: an official journal of the American Association for Cancer Research 19, 2048-2060 (2013).
4. Brentjens, R. J., et al. Safety and persistence of adoptively transferred autologous CD19-targeted T cells in patients with relapsed or chemotherapy refractory B-cell leukemias. Blood 118, 4817-4828 (2011).
5. Brentjens, R. J., et al. Eradication of systemic B-cell tumors by genetically targeted human T lymphocytes co-stimulated by CD80 and interleukin-15. Nature medicine 9, 279-286 (2003).
6. Brentjens, R. J., et al. CD19-Targeted T Cells Rapidly Induce Molecular Remissions in Adults with Chemotherapy-Refractory Acute Lymphoblastic Leukemia. Science translational medicine 5, 177ra138 (2013).
7. Davila, M. L., et al. Efficacy and Toxicity Management of 19-28z CAR T Cell Therapy in B Cell Acute Lymphoblastic Leukemia. Science translational medicine 6, 224ra225 (2014).
8. Siegel, R., Naishadham, D. & Jemal, A. Cancer statistics, 2013. CA: a cancer journal for clinicians 63, 11-30 (2013).
9. Boyd, K. D., et al. The clinical impact and molecular biology of del(17p) in multiple myeloma treated with conventional or thalidomide-based therapy. Genes, chromosomes & cancer 50, 765-774 (2011).
10. Shaughnessy, J. D., Jr., et al. A validated gene expression model of high-risk multiple myeloma is defined by deregulated expression of genes mapping to chromosome 1. Blood 109, 2276-2284 (2007).
11. Gahrton, G., et al. Allogeneic bone marrow transplantation in multiple myeloma. European Group for Bone Marrow Transplantation. The New England journal of medicine 325, 1267-1273 (1991).
12. Pegram, H. J., et al. Tumor-targeted T cells modified to secrete IL-12 eradicate systemic tumors without need for prior conditioning. Blood 119, 4133-4141 (2012).
13. Sabrina Bertilaccio, M. T., et al. Low-Dose Lenalidomide Improves CAR-Based Immunotherapy In CLL By Reverting T-Cell Defects In Vivo. Blood 122, 4171 (2013).
14. Bataille, R., et al. The phenotype of normal, reactive and malignant plasma cells. Identification of “many and multiple myelomas” and of new targets for myeloma therapy. Haematologica 91, 1234-1240 (2006).
15. Morgan, R. A., et al. Case report of a serious adverse event following the administration of T cells transduced with a chimeric antigen receptor recognizing ERBB2. Molecular therapy: the journal of the American Society of Gene Therapy 18, 843-851 (2010).
16. Brentjens, R. J., et al. Safety and persistence of adoptively transferred autologous CD19-targeted T cells in patients with relapsed or chemotherapy refractory B-cell leukemias. Blood 118, 4817-4828 (2011).
17. Brentjens, R. J., et al. CD19-targeted T cells rapidly induce molecular remissions in adults with chemotherapy-refractory acute lymphoblastic leukemia. Science translational medicine 5, 177ra138 (2013).
18. Hunder, N. N., et al. Treatment of metastatic melanoma with autologous CD4⁺ T cells against NY-ESO-1. N.Engl.J.Med. 358, 2698-2703 (2008).
19. Rosenberg, S. A., Restifo, N. P., Yang, J. C., Morgan, R. A. & Dudley, M. E. Adoptive cell transfer: a clinical path to effective cancer immunotherapy. Nat.Rev.Cancer 8, 299-308 (2008).
20. Dudley, M. E., et al. Adoptive cell therapy for patients with metastatic melanoma: evaluation of intensive myeloablative chemoradiation preparative regimens. J Clin Oncol 26, 5233-5239 (2008).
21. Brentjens, R. J., et al. Genetically targeted T cells eradicate systemic acute lymphoblastic leukemia xenografts. Clin.Cancer Res. 13, 5426-5435 (2007).
22. Gade, T. P., et al. Targeted elimination of prostate cancer by genetically directed human T lymphocytes. Cancer Res. 65, 9080-9088 (2005).
23. Maher, J., Brentjens, R. J., Gunset, G., Riviere, I. & Sadelain, M. Human T-lymphocyte cytotoxicity and proliferation directed by a single chimeric TCRzeta/CD28 receptor. Nat.Biotechnol. 20, 70-75 (2002).
24. Kershaw, M. H., et al. Gene-engineered T cells as a superior adjuvant therapy for metastatic cancer. J Immunol 173, 2143-2150 (2004).
25. Sadelain, M., Brentjens, R. & Riviere, I. The promise and potential pitfalls of chimeric antigen receptors. Curr Opin Immunol (2009).
26. Hollyman, D., et al. Manufacturing validation of biologically functional T cells targeted to CD19 antigen for autologous adoptive cell therapy. J Immunother 32, 169-180 (2009).
27. Sadelain, M., Brentjens, R. & Riviere, I. The basic principles of chimeric antigen receptor design. Cancer discovery 3, 388-398 (2013).
28. Riviere, I., Sadelain, M. & Brentjens, R. J. Novel strategies for cancer therapy: the potential of genetically modified T lymphocytes. Curr Hematol Rep 3, 290-297 (2004).
29. Stephan, M. T., et al. T cell-encoded CD80 and 4-1BBL induce auto- and transco-stimulation, resulting in potent tumor rejection. Nat.Med. 13, 1440-1449 (2007).
30. Krause, A., et al. Antigen-dependent CD28 signaling selectively enhances survival and proliferation in genetically modified activated human primary T lymphocytes. J Exp Med 188, 619-626 (1998).
31. Gong, M. C., et al. Cancer patient T cells genetically targeted to prostate-specific membrane antigen specifically lyse prostate cancer cells and release cytokines in response to prostate-specific membrane antigen. Neoplasia. 1, 123-127 (1999).
32. Lyddane, C., et al. Cutting Edge: CD28 controls dominant regulatory T cell activity during active immunization. J.Immunol. 176, 3306-3310 (2006).
33. Carpenter et al., B-cell maturation antigen is a promising target for adoptive T-cell therapy of multiple myeloma. Clin Cancer Res. 2013 Apr. 15; 19(8):2048-60.
34. WO2013/154760
35. Maus et al., Cancer Immunol Res (2003); 1(1):26-31

From the foregoing description, it will be apparent that variations and modifications may be made to the invention described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

What is claimed:

1. A chimeric antigen receptor (CAR), comprising an extracellular antigen-binding domain that specifically binds to B cell maturation antigen (BCMA), a transmembrane domain and an intracellular domain, wherein the extracellular antigen-binding domain comprises a heavy chain variable region and a light chain variable region, wherein:
(a) the heavy chain variable region comprises an amino acid sequence that is at least about 90%, about 91%, about 92%, about 93%, about 94%, about 95%, about 96%, about 97%, about 98% or about 99% homologous to an amino acid sequence selected from the group consisting of: SEQ ID NOS: 1, 5, 9, 13, 17, 21, 25, 29, 33, 37, 41, 45, 49, 53, 57, 61, and 65; and/or the light chain variable region comprises an amino acid sequence that is at least about 90%, about 91%, about 92%, about 93%, about 94%, about 95%, about 96%, about 97%, about 98% or about 99% homologous to an amino acid sequence selected from the group consisting of SEQ ID NOS: 2, 6, 10, 14, 18, 22, 26, 30, 34, 38, 42, 46, 50, 54, 58, 62, and 66; and/or
(b) the heavy chain variable region comprises a CDR1, a CDR2, and a CDR3 of a heavy chain variable region comprising the amino acid sequence set forth in SEQ ID NO: 1, 5, 9, 13, 17, 21, 25, 29, 33, 37, 41, 45, 49, 53, 57, 61, or 65, and/or the light chain variable region comprises a CDR1, a CDR2, and a CDR3 of a light chain variable region comprising the amino acid sequence set forth in SEQ ID NO: 2, 6, 10, 14, 18, 22, 26, 30, 34, 38, 42, 46, 50, 54, 58, 62, or 66; and/or
(c) the heavy chain variable region comprises an amino acid sequence selected from the group consisting of SEQ ID NOs: 91, 97, 103, 109, 115, 121, 127, 133, 139, 145, 151, 157, 163, 169, 175, 181, and 187, and conservative modifications thereof; and/or the light chain variable region comprises an amino acid sequence selected from the group consisting of SEQ ID NOs: 94, 100, 106, 112, 118, 124, 130, 136, 142, 148, 154, 160, 166, 172, 178, 184, and 190, and conservative modifications thereof.

2. The CAR of claim 1, wherein the extracellular antigen-binding domain of the CAR binds to B cell maturation antigen (BCMA) with a binding affinity (K_{D}) of from about 1×10⁻⁹ M to about 3 ×10⁻⁶ M.

3. The CAR of claim 1, wherein the extracellular antigen-binding domain of the CAR binds to B cell maturation antigen (BCMA) with a binding affinity (K_{D}) of from about 1×10⁻⁹ M to about 1 ×10⁻⁸ M.

4. The CAR of claim 1, wherein
(a) the heavy chain variable region comprises an amino acid sequence that is at least about 90%, about 91%, about 92%, about 93%, about 94%, about 95%, about 96%, about 97%, about 98% or about 99% homologous to an amino acid sequence selected from the group consisting of: SEQ ID NOS: 21, 53, 57, 61, and 65; and/or
(b) the light chain variable region comprises an amino acid sequence that is at least about 90%, about 91%, about 92%, about 93%, about 94%, about 95%, about 96%, about 97%, about 98% or about 99% homologous to an amino acid sequence selected from the group consisting of SEQ ID NOS: 22, 54, 58, 62, and 66.

5. The CAR of claim 1, wherein
(a) the heavy chain variable region comprises the CDR1, CDR2, and CDR3 of a heavy chain variable region comprising the amino acid sequence set forth in SEQ ID NO: 21, 53, 57, 61, or 65; and
(b) the light chain variable region comprises the CDR1, CDR2, and CDR3 of a light chain variable region comprising the amino acid sequence set forth in SEQ ID NO: 22, 54, 58, 62, or 66.

6. The CAR of claim 1, wherein
(a) the heavy chain variable region comprises an amino acid sequence selected from the group consisting of: SEQ ID NOS: 1, 5, 9, 13, 17, 21, 25, 29, 33, 37, 41, 45, 49, 53, 57, 61, and 65 and conservative modifications thereof; and/or
(b) the light chain variable region comprises an amino acid sequence selected from the group consisting of: SEQ ID NOS: 2, 6, 10, 14, 18, 22, 26, 30, 34, 38, 42, 46, 50, 54, 58, 62, and 66 and conservative modifications thereof.

7. The CAR of claim 6, wherein
(a) the heavy chain variable region comprises the amino acid sequence set forth in SEQ ID NO: 21, SEQ ID NO: 53, SEQ ID NO: 57, SEQ ID NO: 61, or SEQ ID NO: 65; and/or
(b) the light chain variable region comprises the amino acid sequence set forth in SEQ ID NO: 22, SEQ ID NO: 54, SEQ ID NO: 58, SEQ ID NO: 62 or SEQ ID NO: 6.

8. The CAR of claim 6, wherein the extracellular antigen-binding domain comprises:
(a) a heavy chain variable region comprising the amino acid sequence set forth in SEQ ID NO: 1, and a light chain variable region comprising the amino acid sequence set forth in SEQ ID NO: 2;
(b) a heavy chain variable region comprising the amino acid sequence set forth in SEQ ID NO: 5, and a light chain variable region comprising the amino acid sequence set forth in SEQ ID NO: 6;
(c) a heavy chain variable region comprising the amino acid sequence set forth in SEQ ID NO: 9, and a light chain variable region comprising the amino acid sequence set forth in SEQ ID NO: 10;
(d) a heavy chain variable region comprising the amino acid sequence set forth in SEQ ID NO: 13, and a light chain variable region comprising the amino acid sequence set forth in SEQ ID NO: 14;
(e) a heavy chain variable region comprising the amino acid sequence set forth in SEQ ID NO: 17, and a light chain variable region comprising the amino acid sequence set forth in SEQ ID NO: 18;
(f) a heavy chain variable region comprising the amino acid sequence set forth in SEQ ID NO: 21, and a light chain variable region comprising the amino acid sequence set forth in SEQ ID NO: 22;
(g) a heavy chain variable region comprising the amino acid sequence set forth in SEQ ID NO: 25, and a light chain variable region comprising the amino acid sequence set forth in SEQ ID NO: 26;
(h) a heavy chain variable region comprising the amino acid sequence set forth in SEQ ID NO: 29, and a light chain variable region comprising the amino acid sequence set forth in SEQ ID NO: 30;
(i) a heavy chain variable region comprising the amino acid sequence set forth in SEQ ID NO: 33, and a light chain variable region comprising the amino acid sequence set forth in SEQ ID NO: 34;
(j) a heavy chain variable region comprising the amino acid sequence set forth in SEQ ID NO: 37, and a light chain variable region comprising the amino acid sequence set forth in SEQ ID NO: 38;
(k) a heavy chain variable region comprising the amino acid sequence set forth in SEQ ID NO: 41, and a light chain variable region comprising the amino acid sequence set forth in SEQ ID NO: 42;
(1) a heavy chain variable region comprising the amino acid sequence set forth in SEQ ID NO: 45, and a light chain variable region comprising the amino acid sequence set forth in SEQ ID NO: 46;
(m) a heavy chain variable region comprising the amino acid sequence set forth in SEQ ID NO: 49, and a light chain variable region comprising the amino acid sequence set forth in SEQ ID NO: 50;
(n) a heavy chain variable region comprising the amino acid sequence set forth in SEQ ID NO: 53, and a light chain variable region comprising the amino acid sequence set forth in SEQ ID NO: 54;
(o) a heavy chain variable region comprising the amino acid sequence set forth in SEQ ID NO: 57, and a light chain variable region comprising the amino acid sequence set forth in SEQ ID NO: 58;
(p) a heavy chain variable region comprising the amino acid sequence set forth in SEQ ID NO: 61, and a light chain variable region comprising the amino acid sequence set forth in SEQ ID NO: 62; or
(q) a heavy chain variable region comprising the amino acid sequence set forth in SEQ ID NO: 65, and a light chain variable region comprising the amino acid sequence set forth in SEQ ID NO: 66.

9. The CAR of claim 8, wherein the extracellular antigen-binding domain comprises:
(a) a heavy chain variable region comprising the amino acid sequence set forth in SEQ ID NO: 21, and a light chain variable region comprising the amino acid sequence set forth in SEQ ID NO: 22;
(b) a heavy chain variable region comprising the amino acid sequence set forth in SEQ ID NO: 53; and a light chain variable region comprising the amino acid sequence set forth in SEQ ID NO: 54;
(c) a heavy chain variable region comprising the amino acid sequence set forth in SEQ ID NO: 57; and a light chain variable region comprising the amino acid sequence set forth in SEQ ID NO: 58;
(d) a heavy chain variable region comprising the amino acid sequence set forth in SEQ ID NO: 61, and a light chain variable region comprising the amino acid sequence set forth in SEQ ID NO: 62; or
(e) a heavy chain variable region comprising the amino acid sequence set forth in SEQ ID NO: 65; and a light chain variable region comprising the amino acid sequence set forth in SEQ ID NO: 66.

10. The CAR of claim 1, wherein the extracellular antigen-binding domain comprises a linker between the heavy chain variable region and the light chain variable region.

11. The CAR of claim 1, wherein (a) the heavy chain variable region comprises an amino acid sequence selected from the group consisting of SEQ ID NOS: 91, 97, 103, 109, 115, 121, 127, 133, 139, 145, 151, 157, 163, 169, 175, 181, and 187, and conservative modifications thereof; and (b) the light chain variable region comprises an amino acid sequence selected from the group consisting of SEQ ID NOS: 94, 100, 106, 112, 118, 124, 130, 136, 142, 148, 154, 160, 166, 172, 178, 184, and 190, and conservative modifications thereof.

12. The CAR of claim 11, wherein (a) the heavy chain variable region comprises an amino acid sequence selected from the group consisting of SEQ ID NOs: 90, 96, 102, 108, 114, 120, 126, 132, 138, 144, 150, 156, 162, 168, 174, 180, and 186, and conservative modifications thereof; and (b) the light chain variable region comprises an amino acid sequence selected from the group consisting of SEQ ID NOS: 93, 99, 105, 111, 117, 123, 129, 135, 141, 147, 153, 159, 165, 171, 177, 183, and 189, and conservative modifications thereof.

13. The CAR of claim 11, wherein (a) the heavy chain variable region comprises an amino acid sequence selected from the group consisting of SEQ ID NOs: 89, 95, 101, 107, 113, 119, 125, 131, 137, 143, 149, 155, 161, 167, 173, 179, and 185, and conservative modifications thereof; and (b) the light chain variable region comprises an amino acid sequence selected from the group consisting of SEQ ID NOS: 92, 98, 104, 110, 116, 122, 128, 134, 140, 146, 152, 158, 164, 170, 176, 182, and 188, and conservative modifications thereof.

14. The CAR of claim 1, wherein the heavy chain variable region comprises:
(a) the amino acid sequence set forth in SEQ ID NO: 89 or a conservative modification thereof, the amino acid sequence set forth in SEQ ID NO: 90 or a conservative modification thereof, and the amino acid sequence set forth in SEQ ID NO: 91 or a conservative modification thereof;
(b) the amino acid sequence set forth in SEQ ID NO: 95 or a conservative modification thereof, the amino acid sequence set forth in SEQ ID NO: 96 or a conservative modification thereof, and the amino acid sequence set forth in SEQ ID NO: 97 or a conservative modification thereof;
(c) the amino acid sequence set forth in SEQ ID NO: 101 or a conservative modification thereof, the amino acid sequence set forth in SEQ ID NO: 102 or a conservative modification thereof, and the amino acid sequence set forth in SEQ ID NO: 103 or a conservative modification thereof;
(d) the amino acid sequence set forth in SEQ ID NO: 107 or a conservative modification thereof, the amino acid sequence set forth in SEQ ID NO: 108 or a conservative modification thereof, and the amino acid sequence set forth in SEQ ID NO: 109 or a conservative modification thereof;
(e) the amino acid sequence set forth in SEQ ID NO: 113 or a conservative modification thereof, the amino acid sequence set forth in SEQ ID NO: 114 or a conservative modification thereof, and the amino acid sequence set forth in SEQ ID NO: 115 or a conservative modification thereof;
(f) the amino acid sequence set forth in SEQ ID NO: 119 or a conservative modification thereof, the amino acid sequence set forth in SEQ ID NO: 120 or a conservative modification thereof, and the amino acid sequence set forth in SEQ ID NO: 121 or a conservative modification thereof;
(g) the amino acid sequence set forth in SEQ ID NO: 125 or a conservative modification thereof, the amino acid sequence set forth in SEQ ID NO: 126 or a conservative modification thereof, and the amino acid sequence set forth in SEQ ID NO: 127 or a conservative modification thereof;
(h) the amino acid sequence set forth in SEQ ID NO: 131 or a conservative modification thereof, the amino acid sequence set forth in SEQ ID NO: 132 or a conservative modification thereof, and the amino acid sequence set forth in SEQ ID NO: 133 or a conservative modification thereof;
(i) the amino acid sequence set forth in SEQ ID NO: 137 or a conservative modification thereof, the amino acid sequence set forth in SEQ ID NO: 138 or a conservative modification thereof, and the amino acid sequence set forth in SEQ ID NO: 139 or a conservative modification thereof;
(j) the amino acid sequence set forth in SEQ ID NO: 143 or a conservative modification thereof, the amino acid sequence set forth in SEQ ID NO: 144 or a conservative modification thereof, and the amino acid sequence set forth in SEQ ID NO: 145 or a conservative modification thereof;
(k) the amino acid sequence set forth in SEQ ID NO: 149 or a conservative modification thereof, the amino acid sequence set forth in SEQ ID NO: 150 or a conservative modification thereof, and the amino acid sequence set forth in SEQ ID NO: 151 or a conservative modification thereof;
(1) the amino acid sequence set forth in SEQ ID NO: 155 or a conservative modification thereof, the amino acid sequence set forth in SEQ ID NO: 156 or a conservative modification thereof, and the amino acid sequence set forth in SEQ ID NO: 157 or a conservative modification thereof;
(m) the amino acid sequence set forth in SEQ ID NO: 161 or a conservative modification thereof, the amino acid sequence set forth in SEQ ID NO: 162 or a conservative modification thereof, and the amino acid sequence set forth in SEQ ID NO: 163 or a conservative modification thereof;
(n) the amino acid sequence set forth in SEQ ID NO: 167 or a conservative modification thereof, the amino acid sequence set forth in SEQ ID NO: 168 or a conservative modification thereof, and the amino acid sequence set forth in SEQ ID NO: 169 or a conservative modification thereof;
(o) the amino acid sequence set forth in SEQ ID NO: 173 or a conservative modification thereof, the amino acid sequence set forth in SEQ ID NO: 174 or a conservative modification thereof, and the amino acid sequence set forth in SEQ ID NO: 175 or a conservative modification thereof;
(p) the amino acid sequence set forth in SEQ ID NO: 179 or a conservative modification thereof, the amino acid sequence set forth in SEQ ID NO: 180 or a conservative modification thereof, and the amino acid sequence set forth in SEQ ID NO: 181 or a conservative modification thereof; or
(q) the amino acid sequence set forth in SEQ ID NO: 185 or a conservative modification thereof, the amino acid sequence set forth in SEQ ID NO: 186 or a conservative modification thereof, and the amino acid sequence set forth in SEQ ID NO: 187 or a conservative modification thereof.

15. The CAR of claim 14, wherein the heavy chain variable region comprises:
(a) the amino acid sequence set forth in SEQ ID NO: 167 or a conservative modification thereof, the amino acid sequence set forth in SEQ ID NO: 168 or a conservative modification thereof, and the amino acid sequence set forth in SEQ ID NO: 169 or a conservative modification thereof;
(b) the amino acid sequence set forth in SEQ ID NO: 173 or a conservative modification thereof, the amino acid sequence set forth in SEQ ID NO: 174 or a conservative modification thereof, and the amino acid sequence set forth in SEQ ID NO: 175 or a conservative modification thereof;
(c) the amino acid sequence set forth in SEQ ID NO: 185 or a conservative modification thereof, the amino acid sequence set forth in SEQ ID NO: 186 or a conservative modification thereof, and the amino acid sequence set forth in SEQ ID NO: 187 or a conservative modification thereof;
(d) the amino acid sequence set forth in SEQ ID NO: 119 or a conservative modification thereof, the amino acid sequence set forth in SEQ ID NO: 120 or a conservative modification thereof, and the amino acid sequence set forth in SEQ ID NO: 121 or a conservative modification thereof; or
(e) the amino acid sequence set forth in SEQ ID NO: 179 or a conservative modification thereof, the amino acid sequence set forth in SEQ ID NO: 180 or a conservative modification thereof, and the amino acid sequence set forth in SEQ ID NO: 181 or a conservative modification thereof.

16. The CAR of claim 1, wherein the light chain variable region comprises:
(a) the amino acid sequence set forth in SEQ ID NOs: 92 or a conservative modification thereof, the amino acid sequence set forth in SEQ ID NO: 93 or a conservative modification thereof, and the amino acid sequence set forth in SEQ ID NO: 94 or a conservative modification thereof;
(b) the amino acid sequence set forth in SEQ ID NO: 98 or a conservative modification thereof, the amino acid sequence set forth in SEQ ID NO: 99 or a conservative modification thereof, and the amino acid sequence set forth in SEQ ID NO: 100 or a conservative modification thereof;
(c) the amino acid sequence set forth in SEQ ID NO: 104 or a conservative modification thereof, the amino acid sequence set forth in SEQ ID NO: 105 or a conservative modification thereof, and the amino acid sequence set forth in SEQ ID NO: 106 or a conservative modification thereof;
(d) the amino acid sequence set forth in SEQ ID NO: 110 or a conservative modification thereof, the amino acid sequence set forth in SEQ ID NO: 111 or a conservative modification thereof, and the amino acid sequence set forth in SEQ ID NO: 112 or a conservative modification thereof;
(e) the amino acid sequence set forth in SEQ ID NO: 116 or a conservative modification thereof, the amino acid sequence set forth in SEQ ID NO: 117 or a conservative modification thereof, and the amino acid sequence set forth in SEQ ID NO: 118 or a conservative modification thereof;
(f) the amino acid sequence set forth in SEQ ID NO: 122 or a conservative modification thereof, the amino acid sequence set forth in SEQ ID NO: 123 or a conservative modification thereof, and the amino acid sequence set forth in SEQ ID NO: 124 or a conservative modification thereof;
(g) the amino acid sequence set forth in SEQ ID NO: 128 or a conservative modification thereof, the amino acid sequence set forth in SEQ ID NO: 129 or a conservative modification thereof, and the amino acid sequence set forth in SEQ ID NO: 130 or a conservative modification thereof;
(h) the amino acid sequence set forth in SEQ ID NO: 134 or a conservative modification thereof, the amino acid sequence set forth in SEQ ID NO: 135 or a conservative modification thereof, and the amino acid sequence set forth in SEQ ID NO: 136 or a conservative modification thereof;
(i) the amino acid sequence set forth in SEQ ID NO: 140 or a conservative modification thereof, the amino acid sequence set forth in SEQ ID NO: 141 or a conservative modification thereof, and the amino acid sequence set forth in SEQ ID NO: 142 or a conservative modification thereof;
(j) the amino acid sequence set forth in SEQ ID NO: 146 or a conservative modification thereof, the amino acid sequence set forth in SEQ ID NO: 147 or a conservative modification thereof, and the amino acid sequence set forth in SEQ ID NO: 148 or a conservative modification thereof;
(k) the amino acid sequence set forth in SEQ ID NO: 152 or a conservative modification thereof, the amino acid sequence set forth in SEQ ID NO: 153 or a conservative modification thereof, and the amino acid sequence set forth in SEQ ID NO: 154 or a conservative modification thereof;
(1) the amino acid sequence set forth in SEQ ID NO: 158 or a conservative modification thereof, the amino acid sequence set forth in SEQ ID NO: 159 or a conservative modification thereof, and the amino acid sequence set forth in SEQ ID NO: 160 or a conservative modification thereof;
(m) the amino acid sequence set forth in SEQ ID NO: 164 or a conservative modification thereof, the amino acid sequence set forth in SEQ ID NO: 165 or a conservative modification thereof, and the amino acid sequence set forth in SEQ ID NO: 166 or a conservative modification thereof;
(n) the amino acid sequence set forth in SEQ ID NO: 170 or a conservative modification thereof, the amino acid sequence set forth in SEQ ID NO: 171 or a conservative modification thereof, and the amino acid sequence set forth in SEQ ID NO: 172 or a conservative modification thereof;
(o) the amino acid sequence set forth in SEQ ID NO: 176 or a conservative modification thereof, the amino acid sequence set forth in SEQ ID NO: 177 or a conservative modification thereof, and the amino acid sequence set forth in SEQ ID NO: 178 or a conservative modification thereof;
(p) the amino acid sequence set forth in SEQ ID NO: 182 or a conservative modification thereof, the amino acid sequence set forth in SEQ ID NO: 183 or a conservative modification thereof, and the amino acid sequence set forth in SEQ ID NO: 184 or a conservative modification thereof; or
(q) the amino acid sequence set forth in SEQ ID NO: 188 or a conservative modification thereof, the amino acid sequence set forth in SEQ ID NO: 189 or a conservative modification thereof, and the amino acid sequence set forth in SEQ ID NO: 190 or a conservative modification thereof.

17. The CAR of claim 16, wherein the light chain variable region comprises:
(a) the amino acid sequence set forth in SEQ ID NO: 170 or a conservative modification thereof, the amino acid sequence set forth in SEQ ID NO: 171 or a conservative modification thereof, and the amino acid sequence set forth in SEQ ID NO: 172 or a conservative modification thereof;
(b) the amino acid sequence set forth in SEQ ID NO: 176 or a conservative modification thereof, the amino acid sequence set forth in SEQ ID NO: 177 or a conservative modification thereof, and the amino acid sequence set forth in SEQ ID NO: 178 or a conservative modification thereof;
(c) the amino acid sequence set forth in SEQ ID NO: 188 or a conservative modification thereof, the amino acid sequence set forth in SEQ ID NO: 189 or a conservative modification thereof, and the amino acid sequence set forth in SEQ ID NO: 190 or a conservative modification thereof;
(d) the amino acid sequence set forth in SEQ ID NO: 122 or a conservative modification thereof, the amino acid sequence set forth in SEQ ID NO: 123 or a conservative modification thereof, and the amino acid sequence set forth in SEQ ID NO: 124 or a conservative modification thereof; or
(e) the amino acid sequence set forth in SEQ ID NO: 182 or a conservative modification thereof, the amino acid sequence set forth in SEQ ID NO: 183 or a conservative modification thereof, and the amino acid sequence set forth in SEQ ID NO: 184 or a conservative modification thereof.

18. The CAR of claim 1, wherein the extracellular antigen-binding domain comprises:
(a) a heavy chain variable region comprising the amino acid sequences set forth in SEQ ID NOs: 89, 90, and 91; and a light chain variable region comprising the amino acid sequences set forth in SEQ ID NOs: 92, 93, and 94;
(b) a heavy chain variable region comprising the amino acid sequences set forth in SEQ ID NOs: 95, 96, and 97; and a light chain variable region comprising the amino acid sequences set forth in SEQ ID NOs: 98, 99, and 100;
(c) a heavy chain variable region comprising the amino acid sequences set forth in SEQ ID NOs: 101, 102, and 103; and a light chain variable region comprising the amino acid sequences set forth in SEQ ID NOs: 104, 105, and 106;
(d) a heavy chain variable region comprising the amino acid sequences set forth in SEQ ID NOs: 107, 108, and 109; and a light chain variable region comprising the amino acid sequences set forth in SEQ ID NOs: 110, 111, and 112;
(e) a heavy chain variable region comprising the amino acid sequences set forth in SEQ ID NOs: 113, 114, and 115; and a light chain variable region comprising the amino acid sequences set forth in SEQ ID NOs: 116, 117, and 118;
(f) a heavy chain variable region comprising the amino acid sequences set forth in SEQ ID NOs: 119, 120, and 121; and a light chain variable region comprising the amino acid sequences set forth in SEQ ID NOs: 122, 123, and 124;
(g) a heavy chain variable region comprising the amino acid sequences set forth in SEQ ID NOs: 125, 126, and 127; and a light chain variable region comprising the amino acid sequences set forth in SEQ ID NOs: 128, 129, and 130;
(h) a heavy chain variable region comprising the amino acid sequences set forth in SEQ ID NOs: 131, 132, and 133; and a light chain variable region comprising the amino acid sequences set forth in SEQ ID NOs: 134, 135, and 136;
(i) a heavy chain variable region comprising the amino acid sequences set forth in SEQ ID NOs: 137, 138, and 139; and a light chain variable region comprising the amino acid sequences set forth in SEQ ID NOs: 140, 141, and 142;
(j) a heavy chain variable region comprising the amino acid sequences set forth in SEQ ID NOs: 143, 144, and 145; and a light chain variable region comprising the amino acid sequences set forth in SEQ ID NOs: 146, 147, and 148;
(k) a heavy chain variable region comprising the amino acid sequences set forth in SEQ ID NOs: 149, 150, and 151; and a light chain variable region comprising the amino acid sequences set forth in SEQ ID NOs: 152, 153, and 154;
(1) a heavy chain variable region comprising the amino acid sequences set forth in SEQ ID NOs: 155, 156, and 157; and a light chain variable region comprising the amino acid sequences set forth in SEQ ID NOs: 158, 159, and 160;
(m) a heavy chain variable region comprising the amino acid sequences set forth in SEQ ID NOs: 161, 162, and 163; and a light chain variable region comprising the amino acid sequences set forth in SEQ ID NOs: 164, 165, and 166;
(n) a heavy chain variable region comprising the amino acid sequences set forth in SEQ ID NOs: 167, 168, and 169; and a light chain variable region comprising the amino acid sequences set forth in SEQ ID NOs: 170, 171, and 172;
(o) a heavy chain variable region comprising the amino acid sequences set forth in SEQ ID NOs: 173, 174, and 175; and a light chain variable region comprising the amino acid sequences set forth in SEQ ID NOs: 176, 177, and 178;
(p) a heavy chain variable region comprising the amino acid sequences set forth in SEQ ID NOs: 179, 180, and 181; and a light chain variable region comprising the amino acid sequences set forth in SEQ ID NOs: 182, 183, and 184; or
(q) a heavy chain variable region comprising the amino acid sequences set forth in SEQ ID NOs: 185, 186, and 187; and a light chain variable region comprising the amino acid sequences set forth in SEQ ID NOs: 188, 189, and 190.

19. The CAR of claim 18, wherein the extracellular antigen-binding domain comprises:
(a) a heavy chain variable region comprising the amino acid sequences set forth in SEQ ID NOs: 167, 168, and 169; and a light chain variable region comprising the amino acid sequences set forth in SEQ ID NOs: 170, 171, and 172;
(b) a heavy chain variable region comprising the amino acid sequences set forth in SEQ ID NOs: 173, 174, and 175; and a light chain variable region comprising the amino acid sequences set forth in SEQ ID NOs: 176, 177, and 178;
(c) a heavy chain variable region comprising the amino acid sequences set forth in SEQ ID NOs: 185, 186, and 187; and a light chain variable region comprising the amino acid sequences set forth in SEQ ID NOs: 188, 189, and 190;
(d) a heavy chain variable region comprising the amino acid sequences set forth in SEQ ID NOs: 119, 120, and 121; and a light chain variable region comprising the amino acid sequences set forth in SEQ ID NOs: 122, 123, and 124; or
(e) a heavy chain variable region comprising the amino acid sequences set forth in SEQ ID NOs: 179, 180, and 181; and a light chain variable region comprising the amino acid sequences set forth in SEQ ID NOs: 182, 183, and 184.

20. The CAR of claim 1, wherein
a) the heavy chain variable region comprises an amino acid sequence that is at least about 95% homologous to, or a CDR1, a CDR2, and a CDR3 of, a heavy chain variable region sequence set forth in SEQ ID NO: 21 and the light chain variable region comprises an amino acid sequence that is at least about 95% homologous to, or a CDR1, a CDR2, and a CDR3 of, a light chain variable region sequence set forth in SEQ ID NO: 22;
b) the heavy chain variable region comprises an amino acid sequence that is at least about 95% homologous to, or a CDR1, a CDR2, and a CDR3 of, a heavy chain variable region sequence set forth in SEQ ID NO: 53 and the light chain variable region comprises an amino acid sequence that is at least about 95% homologous to, or a CDR1, a CDR2, and a CDR3 of, a light chain variable region sequence set forth in SEQ ID NO: 54;
c) the heavy chain variable region comprises an amino acid sequence that is at least about 95% homologous to, or a CDR1, a CDR2, and a CDR3 of, a heavy chain variable region sequence set forth in SEQ ID NO: 57 and the light chain variable region comprises an amino acid sequence that is at least about 95% homologous to, or a CDR1, a CDR2, and a CDR3 of, a light chain variable region sequence set forth in SEQ ID NO: 58;
d) the heavy chain variable region comprises an amino acid sequence that is at least about 95% homologous to, or a CDR1, a CDR2, and a CDR3 of, a heavy chain variable region sequence set forth in SEQ ID NO: 61 and the light chain variable region comprises an amino acid sequence that is at least about 95% homologous to, or a CDR1, a CDR2, and a CDR3 of, a light chain variable region sequence set forth in SEQ ID NO: 62; or
e) the heavy chain variable region comprises an amino acid sequence that is at least about 95% homologous to, or a CDR1, a CDR2, and a CDR3 of, a heavy chain variable region sequence set forth in SEQ ID NO: 65 and the light chain variable region comprises an amino acid sequence that is at least about 95% homologous to, or a CDR1, a CDR2, and a CDR3 of, a light chain variable region sequence set forth in SEQ ID NO: 66.

21. The CAR of claim 1, wherein the BCMA comprises the amino acid sequence set forth in SEQ ID NO: 71.

22. The CAR of claim 1, wherein the extracellular antigen-binding domain binds to an epitope region comprising residues within amino acids 14-22 of SEQ ID NO: 71.

23. The CAR of claim 1, wherein the extracellular antigen-binding domain comprises a signal peptide that is covalently joined to the N-terminus of the extracellular antigen-binding domain.

24. The CAR of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof.

25. The CAR of claim 24, wherein the transmembrane domain comprises a CD8 polypeptide or a CD28 polypeptide.

26. The CAR of claim 1, wherein the intracellular domain comprises a CD3ζ polypeptide.

27. The CAR of claim 26, wherein the intracellular domain comprises at least one signaling region.

28. The CAR of claim 27, wherein the at least one signaling region comprises a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

29. The CAR of claim 27, wherein the at least one signaling region comprises a CD3ζ polypeptide and a co-stimulatory domain, which optionally comprises a 4-1BB polypeptide or a CD28 polypeptide.

30. The CAR of claim 27, wherein the at least one signaling region comprises a co-stimulatory signaling region.

31. The CAR of claim 30, wherein the co-stimulatory signaling region comprises a CD28 polypeptide, a 4-1BB polypeptide, an OX40 polypeptide, an ICOS polypeptide, a DAP-10 polypeptide, or a combination thereof.

32. The CAR of claim 31, wherein the co-stimulatory signaling region comprises a CD28 polypeptide or a 4-1BB polypeptide.

33. The CAR of claim 32, wherein
(a) the transmembrane domain comprises a CD28 polypeptide, the intracellular domain comprises a CD3ζ polypeptide, and the co-stimulatory signaling domain comprises a CD28 polypeptide; or
(b) the transmembrane domain comprises a CD8 polypeptide, the intracellular domain comprises a CD3ζ polypeptide, and the co-stimulatory signaling domain comprises a 4-1BB polypeptide.

34. The CAR of claim 1, wherein the CAR is recombinantly expressed or expressed from a vector.

35. An immunoresponsive cell comprising the CAR of claim 1.

36. The immunoresponsive cell of claim 35, wherein the CAR is constitutively expressed on the surface of the immunoresponsive cell.

37. The immunoresponsive cell of claim 35, wherein the isolated immunoresponsive cell is further transduced with at least one co-stimulatory ligand such that the immunoresponsive cell expresses the at least one co-stimulatory ligand.

38. The immunoresponsive cell of claim 35, wherein the immunoresponsive cell is selected from the group consisting of a T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated.

39. A nucleic acid molecule encoding the chimeric antigen receptor (CAR) of claim 1.

40. The nucleic acid molecule of claim 39, comprising the nucleotide sequence set forth in SEQ ID NO: 207, SEQ ID NO: 208, SEQ ID NO: 209, SEQ ID NO: 230, SEQ ID NO: 231, SEQ ID NO: 232, SEQ ID NO: 233, SEQ ID NO: 234, or SEQ ID NO: 235, or a sequence that is at least about 90%, at least about 95%, at least about 96%, at least about 97%, at least about 98%, or at least about 99% homologous thereto.

41. A vector comprising the nucleic acid molecule of claim 39.

42. A host cell comprising the nucleic acid molecule of claim 39.

43. A method of reducing tumor burden in a subject, or of reducing or lengthening survival of a subject having a neoplasm, the method comprising administering an effective amount of the immunoresponsive cell of claim 35 to the subject.

44. The method of claim 43, wherein the method reduces the number of tumor cells, reduces tumor size, or eradicates the tumor in the subject.

45. The method of claim 43, wherein the tumor or neoplasm is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

46. A method for producing an immunoresponsive cell that binds to B cell maturation antigen (BCMA), comprising introducing into the immunoresponsive cell a nucleic acid sequence that encodes the chimeric antigen receptor (CAR) of claim 1.

47. A pharmaceutical composition comprising an effective amount of the immunoresponsive cell of claim 35 and a pharmaceutically acceptable excipient.

48. A kit for treating a neoplasm, the kit comprising the immunoresponsive cell of claim 35 and written instructions for using the immunoresponsive cell for treating a subject having a neoplasm.

49. The kit of claim 48, wherein the neoplasm is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

50. The CAR of claim 1, wherein the extracellular antigen-binding domain is a single-chain variable fragment (scFv), a Fab that is optionally crosslinked, or a F(ab)₂.

51. The CAR of claim 1, wherein the extracellular antigen-binding domain is a human scFv, and/or the heavy chain variable region is a human heavy chain variable region, and/or the light chain variable region is a human light chain variable region.

52. The CAR of claim 1, wherein the extracellular antigen-binding domain binds to a non-linear epitope of BCMA.

53. A CAR comprising an extracellular antigen-binding domain that specifically binds to B cell maturation antigen (BCMA), a transmembrane domain and an intracellular domain, wherein the extracellular antigen-binding domain binds to BCMA with a binding affinity (K_{D}) of from about 1×10⁻⁹ M to about 3×10⁻⁶ M and comprises a human heavy chain variable region and a human light chain variable region.

54. The CAR of claim 53, wherein the extracellular antigen-binding domain of the CAR binds to BCMA with a binding affinity (K_{D}) of from about 1×10⁻⁹ M to about 1×10⁻⁸ M.

55. The CAR of claim 53, wherein the extracellular antigen-binding domain binds to a non-linear epitope of BCMA.

56. The CAR of claim 53, wherein the heavy chain variable region comprises an amino acid sequence that is at least about 95% homologous to, or a CDR1, a CDR2, and a CDR3 of, a heavy chain variable region sequence set forth in SEQ ID NO: 21, 53, 57, 61, or 65; and the light chain variable region comprises an amino acid sequence that is at least about 95% homologous to, or a CDR1, a CDR2, and a CDR3 of, a light chain variable region sequence set forth in SEQ ID NO: 22, 54, 58, 62, or 66.

57. The CAR of claim 53, wherein:
a) the heavy chain variable region comprises an amino acid sequence that is at least about 95% homologous to, or a CDR1, a CDR2, and a CDR3 of, a heavy chain variable region sequence set forth in SEQ ID NO: 21, and the light chain variable region comprises an amino acid sequence that is at least about 95% homologous to, or a CDR1, a CDR2, and a CDR3 of, a light chain variable region sequence set forth in SEQ ID NO: 22;
b) the heavy chain variable region comprises an amino acid sequence that is at least about 95% homologous to, or a CDR1, a CDR2, and a CDR3 of, a heavy chain variable region sequence set forth in SEQ ID NO: 53 and the light chain variable region comprises an amino acid sequence that is at least about 95% homologous to, or a CDR1, a CDR2, and a CDR3 of, a light chain variable region sequence set forth in SEQ ID NO: 54;
c) the heavy chain variable region comprises an amino acid sequence that is at least about 95% homologous to, or a CDR1, a CDR2, and a CDR3 of, a heavy chain variable region sequence set forth in SEQ ID NO: 57 and the light chain variable region comprises an amino acid sequence that is at least about 95% homologous to, or a CDR1, a CDR2, and a CDR3 of, a light chain variable region sequence set forth in SEQ ID NO: 58;
d) the heavy chain variable region comprises an amino acid sequence that is at least about 95% homologous to, or a CDR1, a CDR2, and a CDR3 of, a heavy chain variable region sequence set forth in SEQ ID NO: 61 and the light chain variable region comprises an amino acid sequence that is at least about 95% homologous to, or a CDR1, a CDR2, and a CDR3 of, a light chain variable region sequence set forth in SEQ ID NO: 62; or
e) the heavy chain variable region comprises an amino acid sequence that is at least about 95% homologous to, or a CDR1, a CDR2, and a CDR3 of, a heavy chain variable region sequence set forth in SEQ ID NO: 65 and the light chain variable region comprises an amino acid sequence that is at least about 95% homologous to, or a CDR1, a CDR2, and a CDR3 of, a light chain variable region sequence set forth in SEQ ID NO: 66.

58. The CAR of claim 53, wherein:
a) the heavy chain variable region comprises the amino acid sequence set forth in SEQ ID NO: 21 and the light chain variable region comprises the amino acid sequence set forth in SEQ ID NO: 22;
b) the heavy chain variable region comprises the amino acid sequence set forth in SEQ ID NO: 53 and the light chain variable region comprises the amino acid sequence set forth in SEQ ID NO: 54;
c) the heavy chain variable region comprises the amino acid sequence set forth in SEQ ID NO: 57 and the light chain variable region comprises the amino acid sequence set forth in SEQ ID NO: 58;
d) the heavy chain variable region comprises the amino acid sequence set forth in SEQ ID NO: 61 and the light chain variable region comprises the amino acid sequence set forth in SEQ ID NO: 62; or
e) the heavy chain variable region comprises the amino acid sequence set forth in SEQ ID NO: 65 and the light chain variable region comprises the amino acid sequence set forth in SEQ ID NO: 66.


SEQUENCE LISTING (280 sequences)

SEQ ID NO: 1 - protein, 123 aa, synthetic construct
  REGION 1..123: note = Description of Artificial Sequence: Synthetic polypeptide
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARQGYSYYGYSDVWGQGTLVTVSS

SEQ ID NO: 2 - protein, 111 aa, synthetic construct
  REGION 1..111: note = Description of Artificial Sequence: Synthetic polypeptide
QSVLTQPPSVSVAPRQRVTISCSGSSSNIGHNDVSWYQHLPGKAPRLLIYFDDLLPSGVSDRFSASKSGTSASLAISGLQSEDEADYYCAAWDGSLNAFVFGTGTKVTVLG

SEQ ID NO: 3 - DNA, 369 bp, synthetic construct
  misc_feature 1..369: note = Description of Artificial Sequence: Synthetic polynucleotide
  source 1..369: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcgcgccagggttactcttactacggttactctgatgtttggggtcaaggtactctggtgaccgtctcctca

SEQ ID NO: 4 - DNA, 333 bp, synthetic construct
  misc_feature 1..333: note = Description of Artificial Sequence: Synthetic polynucleotide
  source 1..333: mol_type = other DNA
cagtctgtgctgactcagccaccctcggtgtctgtagcccccaggcagagggtcaccatctcgtgttctggaagcagctccaacatcggacataatgatgtaagctggtaccagcatctcccagggaaggctcccagactcctcatctattttgatgacctgctgccgtcaggggtctctgaccgattctctgcctccaagtctggcacctcagcctccctggccatcagtgggctccagtctgaggatgaggctgattattactgtgcagcatgggatggcagcctgaatgcctttgtcttcggaactgggaccaaggtcaccgtcctaggt

SEQ ID NO: 5 - protein, 123 aa, synthetic construct
  REGION 1..123: note = Description of Artificial Sequence: Synthetic polypeptide
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARYGFSGSRFYDTWGQGTLVTVSS

SEQ ID NO: 6 - protein, 111 aa, synthetic construct
  REGION 1..111: note = Description of Artificial Sequence: Synthetic polypeptide
QPVLTQPPSVSEAPRQRVTISCSGSSSNIGNNAVNWYQQLPGKAPKLLIYFDDLLSSGVSDRFSGSKSGTSASLAISGLQSEDEADYYCAAWDDSLNGYVFGTGTKVTVLG

SEQ ID NO: 7 - DNA, 369 bp, synthetic construct
  misc_feature 1..369: note = Description of Artificial Sequence: Synthetic polynucleotide
  source 1..369: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcgcgctacggtttctctggttctcgtttctacgatacttggggtcaaggtactctggtgaccgtctcctca

SEQ ID NO: 8 - DNA, 333 bp, synthetic construct
  misc_feature 1..333: note = Description of Artificial Sequence: Synthetic polynucleotide
  source 1..333: mol_type = other DNA
cagcctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctattttgatgatctgctgtcctcaggggtctctgaccgattctctggctccaagtctggcacctcagcctccctggccatcagtgggctccagtctgaagatgaggctgattattactgtgcagcatgggatgacagcctgaatggttatgtcttcggaactgggaccaaggtcaccgtcctaggt

SEQ ID NO: 9 - protein, 122 aa, synthetic construct
  REGION 1..122: note = Description of Artificial Sequence: Synthetic polypeptide
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 10 - protein, 111 aa, synthetic construct
  REGION 1..111: note = Description of Artificial Sequence: Synthetic polypeptide
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 11 - DNA, 366 bp, synthetic construct
  misc_feature 1..366: note = Description of Artificial Sequence: Synthetic polynucleotide
  source 1..366: mol_type = other DNA
gaggtccagctggtgcagtctggagctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcaccatgaccgaggacacatctacagacacagcctacatggagctgagcagcctgagatctgaggacacggccgtgtattactgtgcgcgctctggttactctaaatctatcgtttcttacatggattactggggtcaaggtactctggtgaccgtctcctca

SEQ ID NO: 12 - DNA, 333 bp, synthetic construct
  misc_feature 1..333: note = Description of Artificial Sequence: Synthetic polynucleotide
  source 1..333: mol_type = other DNA
ctgcctgtgctgactcagcccccctccacgtctgggacccccgggcagagggtcaccgtctcttgttctggaagcagctccaacatcggaagtaatgttgtattctggtaccagcagctcccaggcacggcccccaaacttgtcatctataggaataatcaacggccctcaggggtccctgaccgattctctgtctccaagtctggcacctcagcctccctggccatcagtgggctccggtccgaggacgaggctgattattattgtgcagcttgggatgacagcctgagtggttatgtcttcggaactgggaccaaggtcaccgtcctaggt

SEQ ID NO: 13 - protein, 118 aa, synthetic construct
  REGION 1..118: note = Description of Artificial Sequence: Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSQWGGVLDYWGQGTLVTVSS

SEQ ID NO: 14 - protein, 112 aa, synthetic construct
  REGION 1..112: note = Description of Artificial Sequence: Synthetic polypeptide
QSVVTQPPSVSGAPGQRVTISCSGSSSNIGARYDVQWYQQLPGTAPKLLIFGNNNRPSGVPDRFSGSKSGTSASLAITGLQAEDEADYYCQSYDSSLSASVFGGGTKLTVLG

SEQ ID NO: 15 - DNA, 354 bp, synthetic construct
  misc_feature 1..354: note = Description of Artificial Sequence: Synthetic polynucleotide
  source 1..354: mol_type = other DNA
gaggtccagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgcgctctcagtggggtggtgttctggattactggggtcaaggtactctggtgaccgtctcctca

SEQ ID NO: 16 - DNA, 336 bp, synthetic construct
  misc_feature 1..336: note = Description of Artificial Sequence: Synthetic polynucleotide
  source 1..336: mol_type = other DNA
cagtctgtcgtgacgcagccgccctcagtgtctggggccccagggcagagggtcaccatctcctgcagtgggagcagctccaacatcggggcacgttatgatgttcagtggtaccagcagcttccaggaacagcccccaaactcctcatctttggtaacaacaatcggccctcaggggtccctgaccgattctctggctccaagtctggcacgtcagcctccctggccatcactgggctccaggctgaggatgaggctgattattactgccagtcctatgacagcagcctgagtgcttcggtgttcggcggagggaccaagctgaccgtcctaggt

SEQ ID NO: 17 - protein, 123 aa, synthetic construct
  REGION 1..123: note = Description of Artificial Sequence: Synthetic polypeptide
QVQLVESGAEVKKPGSSVKVSCKASGGTFSSYAISWVRQAPGQGLEWMGRIIPILGIANYAQKFQGRVTITADESTSTAYMELSSLRSEDTAVYYCARTGYESWGSYEVIDRWGQGTLVTVSS

SEQ ID NO: 18 - protein, 111 aa, synthetic construct
  REGION 1..111: note = Description of Artificial Sequence: Synthetic polypeptide
QAVLTQPPSASGTPGQRVTISCSGSSSNIGSNTVNWYRQLPGTAPKLLIYSNNQRPSGVPDRFSGSKSGTSASLAISGLQSEDEADYYCAAWDDSLNGVVFGGGTKLTVLG

SEQ ID NO: 19 - DNA, 369 bp, synthetic construct
  misc_feature 1..369: note = Description of Artificial Sequence: Synthetic polynucleotide
  source 1..369: mol_type = other DNA
caggtgcagctggtggagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacgaatccacgagcacagcctacatggagctgagcagcctgagatctgaggacacggccgtatattactgtgcgcgcactggttacgaatcttggggttcttacgaagttatcgatcgttggggtcaaggtactctggtgaccgtctcctca

SEQ ID NO: 20 - DNA, 333 bp, synthetic construct
  misc_feature 1..333: note = Description of Artificial Sequence: Synthetic polynucleotide
  source 1..333: mol_type = other DNA
caggctgtgctgactcagccaccctcagcgtctgggacccccgggcagagggtcaccatctcttgttctggaagcagctccaacatcggaagtaatactgtaaactggtaccggcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctgaccgtcctaggt

SEQ ID NO: 21 - protein, 121 aa, synthetic construct
  REGION 1..121: note = Description of Artificial Sequence: Synthetic polypeptide
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 22 - protein, 112 aa, synthetic construct
  REGION 1..112: note = Description of Artificial Sequence: Synthetic polypeptide
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 23 - DNA, 363 bp, synthetic construct
  misc_feature 1..363: note = Description of Artificial Sequence: Synthetic polynucleotide
  source 1..363: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgcgcggtggttactactctcatgacatgtggtctgaagattggggtcaaggtactctggtgaccgtctcctca

SEQ ID NO: 24 - DNA, 336 bp, synthetic construct
  misc_feature 1..336: note = Description of Artificial Sequence: Synthetic polynucleotide
  source 1..336: mol_type = other DNA
ctgcctgtgctgactcagccaccctcagcgtctgggacccccgggcagagggtcaccatctcttgttctggacgcagttccaacatcgggagtaattctgttaactggtatcgacaactcccaggagcggcccccaaactcctcatctatagtaataatcagcggcccccaggggtccctgtgcgattctctggctccaagtctggcacctcagcctccctggccatcagtgggctccagtctgaagatgaggccacttattactgtgcaacatgggatgacaatctgaatgttcactatgtcttcggaactgggaccaaggtcaccgtcctaggt

SEQ ID NO: 25 - protein, 125 aa, synthetic construct
  REGION 1..125: note = Description of Artificial Sequence: Synthetic polypeptide
QVQLQESGPGLVKPSGTLSLTCGVSGGSISNSNWWSWVRQPPGKGLEWIGEIYHSGSTKYNPSLRSRVTISVDKSKNQFSLKLSSVTAADTAVYYCARRDNWKTPTTKIDGFDIWGQGTMVTVSS

SEQ ID NO: 26 - protein, 117 aa, synthetic construct
  REGION 1..117: note = Description of Artificial Sequence: Synthetic polypeptide
QPVLTQPPSASASLGASVTLTCTLSSGYSNYKVDWYQQRPGKGPRFVMRVGTGGIVGSKGDGIPDRFSVLGSGLNRYLTIKNIQEEDEGDYHCGADHGSGSNFVYVFGTGTKVTVLG

SEQ ID NO: 27 - DNA, 375 bp, synthetic construct
  misc_feature 1..375: note = Description of Artificial Sequence: Synthetic polynucleotide
  source 1..375: mol_type = other DNA
caggtgcagctgcaggagtcgggcccaggactggtgaagccttcggggaccctgtccctcacctgcggtgtctctggtggctccatcagcaatagtaactggtggagttgggtccgccagccccccgggaaggggctggagtggattggggaaatctatcatagtgggagcaccaagtacaacccgtccctcaggagtcgagtcaccatatcagtagacaagtccaagaaccagttctccctaaaattgagctctgtgaccgccgcggacacggccgtatattactgtgcgagacgagataactggaagacccccactaccaaaattgatggttttgatatctggggccaagggacaatggtcaccgtctcttca

SEQ ID NO: 28 - DNA, 351 bp, synthetic construct
  misc_feature 1..351: note = Description of Artificial Sequence: Synthetic polynucleotide
  source 1..351: mol_type = other DNA
cagcctgtgctgactcagccaccttctgcatcagcctccctgggagcctcggtcacactcacctgcaccctgagcagcggctacagtaattataaagtggactggtaccagcagagaccagggaagggcccccggtttgtgatgcgagtgggcactggtgggattgtgggatccaagggggatggcatccctgatcgcttctcagtcttgggctcaggcctgaatcggtacctgaccatcaagaacatccaggaagaagatgagggtgactatcactgtggggcagaccatggcagtgggagcaacttcgtgtatgtcttcggaactgggaccaaggtcaccgtcctaggt

SEQ ID NO: 29 - protein, 118 aa, synthetic construct
  REGION 1..118: note = Description of Artificial Sequence: Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSQWGSSWDYWGQGTLVTVSS

SEQ ID NO: 30 - protein, 108 aa, synthetic construct
  REGION 1..108: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKR

SEQ ID NO: 31 - DNA, 354 bp, synthetic construct
  misc_feature 1..354: note = Description of Artificial Sequence: Synthetic polynucleotide
  source 1..354: mol_type = other DNA
caggtccagctggtacagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgcgctctcagtggggttcttcttgggattactggggtcaaggtactctggtgaccgtctcctca

SEQ ID NO: 32 - DNA, 324 bp, synthetic construct
  misc_feature 1..324: note = Description of Artificial Sequence: Synthetic polynucleotide
  source 1..324: mol_type = other DNA
gacatccagttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgacgttcggccaagggaccaaggtggagatcaaacgt

SEQ ID NO: 33 - protein, 119 aa, synthetic construct
  REGION 1..119: note = Description of Artificial Sequence: Synthetic polypeptide
EVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARSSYHLYGYDSWGQGTLVTVSS

SEQ ID NO: 34 - protein, 117 aa, synthetic construct
  REGION 1..117: note = Description of Artificial Sequence: Synthetic polypeptide
QPVLTQPPSASASLGASVTLTCTLSNDYTNYKVDWYQQRPGKGPRFVMRVGPGGIVGSKGDGIPDRFSVLGSGLNRYLTIKNIQEEDESDYHCGADHGTGSNFVYVFGGGTKLTVLG

SEQ ID NO: 35 - DNA, 357 bp, synthetic construct
  misc_feature 1..357: note = Description of Artificial Sequence: Synthetic polynucleotide
  source 1..357: mol_type = other DNA
gaggtgcagctggtggagtccggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtatattactgtgcgcgctcttcttaccatctgtacggttacgattcttggggtcaaggtactctggtgaccgtctcctca

SEQ ID NO: 36 - DNA, 351 bp, synthetic construct
  misc_feature 1..351: note = Description of Artificial Sequence: Synthetic polynucleotide
  source 1..351: mol_type = other DNA
cagcctgtgctgactcagccaccttctgcatcagcctccctgggagcctcggtcactctcacctgcaccctgagcaacgactacactaattataaagtggactggtaccagcagagaccagggaagggcccccggtttgtgatgcgagtgggccctggtgggattgtgggatccaagggggatggcatccctgatcgcttctcagtcttgggctcaggcctgaatcgatacctgaccatcaagaacatccaggaggaggatgagagtgactaccactgtggggcggaccatggcaccgggagcaacttcgtgtacgtgttcggcggagggaccaagctgaccgtcctaggt

SEQ ID NO: 37 - protein, 120 aa, synthetic construct
  REGION 1..120: note = Description of Artificial Sequence: Synthetic polypeptide
QVQLVQSGAEVKKPGSSVKVSCKASGGTFSSYAISWVRQAPGQGLEWMGGIIPIFSTANYAQKFQGRVTMTTDTSTSTAYMELRSLRSDDTAVYYCARQPWTWYSPYDQWGQGTLVTVSS

SEQ ID NO: 38 - protein, 117 aa, synthetic construct
  REGION 1..117: note = Description of Artificial Sequence: Synthetic polypeptide
QPVLTQPPSASASLGASVTLTCTLSSGYSNYKVDWYQQRPGKGPRFLMRVDTGGIVGSKGDGIPDRFSVSGSGLNRYLTIKNIQEEDESDYHCGADHGSGSNFVWVFGGGTKLTVLG

SEQ ID NO: 39 - DNA, 360 bp, synthetic construct
  misc_feature 1..360: note = Description of Artificial Sequence: Synthetic polynucleotide
  source 1..360: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttagtacagcaaactacgcacagaagttccagggcagagtcaccatgaccacagacacatccacgagcacagcctacatggagctgaggagcctgagatctgacgacacggccgtgtattactgtgcgcgccagccgtggacttggtactctccgtacgatcagtggggtcaaggtactctggtgaccgtctcctca

SEQ ID NO: 40 - DNA, 351 bp, synthetic construct
  misc_feature 1..351: note = Description of Artificial Sequence: Synthetic polynucleotide
  source 1..351: mol_type = other DNA
cagcctgtgctgactcagccaccttctgcatcagcctccctgggagcctcggtcacactcacctgcaccctgagcagcggctacagtaattataaagtggactggtatcaacagagaccagggaagggcccccggtttctgatgcgagtagacaccggtgggattgtgggatccaagggggatggcatccctgatcgcttctcagtctcgggctcaggtctgaatcggtacctgaccatcaagaacattcaggaagaggatgagagtgactaccactgtggggcagaccatggcagtgggagcaacttcgtgtgggtgttcggcggagggaccaagctgaccgtcctaggt

SEQ ID NO: 41 - protein, 117 aa, synthetic construct
  REGION 1..117: note = Description of Artificial Sequence: Synthetic polypeptide
EVQLVETGGGLVQPGGSLRLSCAASGFTFSTYAMTWVRQAPGKGLEWVSAITPGGDRTYYADSVKGRFTISRDNSRNTLYLQMNSLRAEDTAVYYCARYYGYMIDMWGQGTLVTVSS

SEQ ID NO: 42 - protein, 113 aa, synthetic construct
  REGION 1..113: note = Description of Artificial Sequence: Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKR

SEQ ID NO: 43 - DNA, 351 bp, synthetic construct
  misc_feature 1..351: note = Description of Artificial Sequence: Synthetic polynucleotide
  source 1..351: mol_type = other DNA
gaggtgcagctggtggagactgggggaggcctggtacagcctggggggtccctgagactctcctgtgctgcctctggattcacctttagcacctatgccatgacctgggtccgccaggctccagggaaggggctggagtgggtctcagctattactcctggtggtgatcgcacatactacgcagactccgtgaagggccgtttcactatctccagagacaattccaggaacacgctgtatctgcaaatgaacagcctgagagccgaggacacggccgtatattactgtgcgcgctactacggttacatgatcgatatgtggggtcaaggtactctggtgaccgtctcctca

SEQ ID NO: 44 - DNA, 339 bp, synthetic construct
  misc_feature 1..339: note = Description of Artificial Sequence: Synthetic polynucleotide
  source 1..339: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggaaatcaaacgt

SEQ ID NO: 45 - protein, 118 aa, synthetic construct
  REGION 1..118: note = Description of Artificial Sequence: Synthetic polypeptide
QMQLVQSGAEVKKPGASVKVSCKASGYTFTGYYVHWLRQAPGQGLEWMGWINPNSGGTNNAQEFQGRITMTRDTSINTAYMELSRLRSDDTAVYYCARSQWGGTYDYWGQGTLVTVSS

SEQ ID NO: 46 - protein, 111 aa, synthetic construct
  REGION 1..111: note = Description of Artificial Sequence: Synthetic polypeptide
SYVLTQPPSASGTPGQRVTISCSGSSSNIGSNTVNWYQQVPGTAPKLLIYSNNQRPSGVPDRFSGSKSGASASLAISWLQSEDEADYYCAAWDDSLNGWVFGGGTKLTVLG

SEQ ID NO: 47 - DNA, 354 bp, synthetic construct
  misc_feature 1..354: note = Description of Artificial Sequence: Synthetic polynucleotide
  source 1..354: mol_type = other DNA
cagatgcagctggtgcagtctggggctgaggtgaagaagcctggggcctcagtgaaggtctcctgcaaggcttctggatacaccttcaccggctattatgtacactggttgcgacaggcccctggacaagggcttgagtggatgggttggatcaaccctaacagtggcggcacaaacaatgcacaggagtttcaaggcaggatcaccatgaccagggacacgtccatcaacacagcctacatggagctgagcaggctgagatctgacgacacggccgtgtattactgtgcgcgctctcagtggggtggtacttacgattactggggtcaaggtactctggtgaccgtctcctca

SEQ ID NO: 48 - DNA, 333 bp, synthetic construct
  misc_feature 1..333: note = Description of Artificial Sequence: Synthetic polynucleotide
  source 1..333: mol_type = other DNA
tcctatgtgctgactcagccaccctcagcgtctgggacccccgggcagagggtcaccatctcttgttctggaagcagctccaacatcggaagtaatactgtaaactggtaccagcaggtcccaggaacggcccccaaactcctcatctatagtaataatcagcggccctcaggggtccctgaccgattctctggctccaagtctggcgcctcagcctccctggccatcagttggctccagtctgaggatgaggctgattattactgtgcagcatgggatgacagcctgaatggttgggtgttcggcggagggaccaagctgaccgtcctaggt

SEQ ID NO: 49 - protein, 117 aa, synthetic construct
  REGION 1..117: note = Description of Artificial Sequence: Synthetic polypeptide
EVQLVQSGAEVKKPGESLKISCKGSGYDFTTYWIGWVRQMPGKGLEWMGIIYPGDSDTRYSPSVRGRVTISADKSINTAYLQWSSLEASDTAMYYCARMWTFSQDGWGQGTLVTVSS

SEQ ID NO: 50 - protein, 111 aa, synthetic construct
  REGION 1..111: note = Description of Artificial Sequence: Synthetic polypeptide
QAVLTQPPSASGTPGQRVTISCSGSSSNIGSYTVSWYQQLPGTAPKFLIYSNNQRPSGVPDRFSGSKSGTSASLAISGLQSEDEADYYCAAWDDSLNGYVFGTGTKVTVLG

SEQ ID NO: 51 - DNA, 351 bp, synthetic construct
  misc_feature 1..351: note = Description of Artificial Sequence: Synthetic polynucleotide
  source 1..351: mol_type = other DNA
gaggtgcagctggtgcagtctggagcagaggtgaaaaagccgggggagtctctgaagatctcctgtaagggttctggatatgactttaccacctactggatcgggtgggtgcgccagatgcccgggaagggcctggagtggatggggatcatctatcctggtgactctgataccagatacagcccgtccgtccgaggccgggtcaccatctcagccgacaagtccatcaacaccgcctatttgcagtggagtagcctggaggcctccgacaccgccatgtattactgtgcgcgcatgtggactttctctcaggatggttggggtcaaggtactctggtgaccgtctcctca

SEQ ID NO: 52 - DNA, 333 bp, synthetic construct
  misc_feature 1..333: note = Description of Artificial Sequence: Synthetic polynucleotide
  source 1..333: mol_type = other DNA
caggctgtgctgactcagccaccctcagcgtctgggacccccgggcagagggtcaccatctcttgttctggaagcagctccaacatcggaagttatactgtaagctggtaccagcaactcccaggaacggcccccaaattcctcatctattctaataatcagcggccctcaggggtccctgaccgattctctggctccaagtctggcacctcagcctccctggccatcagtgggctccagtctgaggatgaggctgattattactgtgctgcatgggatgacagcctgaatggttatgtcttcggaactgggaccaaggtcaccgtcctaggt

SEQ ID NO: 53 - protein, 118 aa, synthetic construct
  REGION 1..118: note = Description of Artificial Sequence: Synthetic polypeptide
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 54 - protein, 105 aa, synthetic construct
  REGION 1..105: note = Description of Artificial Sequence: Synthetic polypeptide
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 55 - DNA, 354 bp, synthetic construct
  misc_feature 1..354: note = Description of Artificial Sequence: Synthetic polynucleotide
  source 1..354: mol_type = other DNA
gaagtgcagctggtgcagtctggggctgagatgaagaagcctggggcctcactgaagctctcctgcaaggcttctggatacaccttcatcgactactatgtatactggatgcgacaggcccctggacaagggcttgagtccatgggatggatcaaccctaacagtggtggcacaaactatgcacagaagtttcagggcagggtcaccatgaccagggacacgtccatcagcacagcctacatggagctgagcaggctgagatctgacgacaccgccatgtattactgtgcgcgctcccagcgtgacggttacatggattactggggtcaaggtactctggtgaccgtctcctca

SEQ ID NO: 56 - DNA, 315 bp, synthetic construct
  misc_feature 1..315: note = Description of Artificial Sequence: Synthetic polynucleotide
  source 1..315: mol_type = other DNA
caatctgccctgactcagcctgcctccgtgtctgcgtctcctggacagtcgatcgccatctcctgcactggaaccagcagtgacgttggttggtatcaacagcacccaggcaaagcccccaaactcatgatttatgaggacagtaagcggccctcaggggtttctaatcgcttctctggctccaagtctggcaacacggcctccctgaccatctctgggctccaggctgaggacgaggctgattattactgcagctcaaatacaagaagcagcactttggtgttcggcggagggaccaagctgaccgtcctaggt

SEQ ID NO: 57 - protein, 118 aa, synthetic construct
  REGION 1..118: note = Description of Artificial Sequence: Synthetic polypeptide
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 58 - protein, 112 aa, synthetic construct
  REGION 1..112: note = Description of Artificial Sequence: Synthetic polypeptide
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 59 - DNA, 354 bp, synthetic construct
  misc_feature 1..354: note = Description of Artificial Sequence: Synthetic polynucleotide
  source 1..354: mol_type = other DNA
caggtccagctggtacagtctggggctgaggtgaagaagcctggggcctcagtgaaggtctcctgcaaggcttctggatacaccttcaccgactactatatgcactgggtgcgacaggcccctggacaacggcttgagtggatgggatggatcaaccctaacagtggtggcacaaactatgcacagaagtttcaggacaggatcaccgtgaccagggacacctccagcaacacaggctacatggagctgaccaggctgagatctgacgacacggccgtgtattactgtgcgcgctctccgtactctggtgttctggataaatggggtcaaggtactctggtgaccgtctcctca

SEQ ID NO: 60 - DNA, 336 bp, synthetic construct
  misc_feature 1..336: note = Description of Artificial Sequence: Synthetic polynucleotide
  source 1..336: mol_type = other DNA
cagtctgtgctgacgcagccgccctcagtgtctggggccccagggcagagggtcaccatctcctgcactgggagcagctccaacatcggggcaggttttgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttatgtcttcggaactgggaccaaggtcaccgtcctaggt

SEQ ID NO: 61 - protein, 120 aa, synthetic construct
  REGION 1..120: note = Description of Artificial Sequence: Synthetic polypeptide
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 62 - protein, 112 aa, synthetic construct
  REGION 1..112: note = Description of Artificial Sequence: Synthetic polypeptide
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 63 - DNA, 360 bp, synthetic construct
  misc_feature 1..360: note = Description of Artificial Sequence: Synthetic polynucleotide
  source 1..360: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtacagcaaactacgcacagaagttccagggcagagtcacgattaccgcggacgaatccacgagcacagcctacatggagctgagcagcctgagatctgaggacacggccgtgtattactgtgcgcgctctggttacggttcttaccgttgggaagattcttggggtcaaggtactctggtgaccgtctcctca

SEQ ID NO: 64 - DNA, 336 bp, synthetic construct
  misc_feature 1..336: note = Description of Artificial Sequence: Synthetic polynucleotide
  source 1..336: mol_type = other DNA
caggctgtgctgactcagccaccctcagcgtctgggacccccgggcagagggtcaccatctcttgttctggaagcagctccaacatcggaagtaattacgtattctggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtgcctcttatgttttcggaactgggaccaaggtcaccgtcctaggt

SEQ ID NO: 65 - protein, 116 aa, synthetic construct
  REGION 1..116: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 66 - protein, 111 aa, synthetic construct
  REGION 1..111: note = Description of Artificial Sequence: Synthetic polypeptide
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 67 - DNA, 348 bp, synthetic construct
  misc_feature 1..348: note = Description of Artificial Sequence: Synthetic polynucleotide
  source 1..348: mol_type = other DNA
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccacgtcaccatctcagctgacaagtccatcagcactgcctacctgcagtggagcagcctgaaggcctcggacaccgccatgtattactgtgcgcgctactctggttctttcgataactggggtcaaggtactctggtgaccgtctcctca

SEQ ID NO: 68 - DNA, 333 bp, synthetic construct
  misc_feature 1..333: note = Description of Artificial Sequence: Synthetic polynucleotide
  source 1..333: mol_type = other DNA
tcctatgagctgactcagccaccctcagcgtctgggacccccgggcagagggtcaccatgtcttgttctggaaccagctccaacatcggaagtcactctgtaaactggtaccagcagctcccaggaacggcccccaaactcctcatctatactaataatcagcggccctcaggggtccctgaccgattctctggctccaagtctggcacctcagcctccctggccatcagtggcctccagtctgaggatgaggctgattattactgtgcagcatgggatggcagcctgaatggtctggtattcggcggagggaccaagctgaccgtcctaggt

SEQ ID NO: 69 - protein, 21 aa, synthetic construct
  REGION 1..21: note = Description of Artificial Sequence: Synthetic peptide
SRGGGGSGGGGSGGGGSLEMA

SEQ ID NO: 70 - DNA, 63 bp, synthetic construct
  misc_feature 1..63: note = Description of Artificial Sequence: Synthetic oligonucleotide
  source 1..63: mol_type = other DNA
tctagaggtggtggtggtagcggcggcggcggctctggtggtggtggatccctcgagatggcc

SEQ ID NO: 71 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 72 - protein, 255 aa, synthetic construct
  REGION 1..255: note = Description of Artificial Sequence: Synthetic polypeptide
QSVLTQPPSVSVAPRQRVTISCSGSSSNIGHNDVSWYQHLPGKAPRLLIYFDDLLPSGVSDRFSASKSGTSASLAISGLQSEDEADYYCAAWDGSLNAFVFGTGTKVTVLGSRGGGGSGGGGSGGGGSLEMAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARQGYSYYGYSDVWGQGTLVTVSS

SEQ ID NO: 73 - protein, 255 aa, synthetic construct
  REGION 1..255: note = Description of Artificial Sequence: Synthetic polypeptide
QPVLTQPPSVSEAPRQRVTISCSGSSSNIGNNAVNWYQQLPGKAPKLLIYFDDLLSSGVSDRFSGSKSGTSASLAISGLQSEDEADYYCAAWDDSLNGYVFGTGTKVTVLGSRGGGGSGGGGSGGGGSLEMAQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARYGFSGSRFYDTWGQGTLVTVSS

SEQ ID NO: 74 - protein, 254 aa, synthetic construct
  REGION 1..254: note = Description of Artificial Sequence: Synthetic polypeptide
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLGSRGGGGSGGGGSGGGGSLEMAEVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 75 - protein, 251 aa, synthetic construct
  REGION 1..251: note = Description of Artificial Sequence: Synthetic polypeptide
QSVVTQPPSVSGAPGQRVTISCSGSSSNIGARYDVQWYQQLPGTAPKLLIFGNNNRPSGVPDRFSGSKSGTSASLAITGLQAEDEADYYCQSYDSSLSASVFGGGTKLTVLGSRGGGGSGGGGSGGGGSLEMAEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSQWGGVLDYWGQGTLVTVSS

SEQ ID NO: 76 - protein, 255 aa, synthetic construct
  REGION 1..255: note = Description of Artificial Sequence: Synthetic polypeptide
QAVLTQPPSASGTPGQRVTISCSGSSSNIGSNTVNWYRQLPGTAPKLLIYSNNQRPSGVPDRFSGSKSGTSASLAISGLQSEDEADYYCAAWDDSLNGVVFGGGTKLTVLGSRGGGGSGGGGSGGGGSLEMAQVQLVESGAEVKKPGSSVKVSCKASGGTFSSYAISWVRQAPGQGLEWMGRIIPILGIANYAQKFQGRVTITADESTSTAYMELSSLRSEDTAVYYCARTGYESWGSYEVIDRWGQGTLVTVSS

SEQ ID NO: 77 - protein, 254 aa, synthetic construct
  REGION 1..254: note = Description of Artificial Sequence: Synthetic polypeptide
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 78 - protein, 263 aa, synthetic construct
  REGION 1..263: note = Description of Artificial Sequence: Synthetic polypeptide
QPVLTQPPSASASLGASVTLTCTLSSGYSNYKVDWYQQRPGKGPRFVMRVGTGGIVGSKGDGIPDRFSVLGSGLNRYLTIKNIQEEDEGDYHCGADHGSGSNFVYVFGTGTKVTVLGSRGGGGSGGGGSGGGGSLEMAQVQLQESGPGLVKPSGTLSLTCGVSGGSISNSNWWSWVRQPPGKGLEWIGEIYHSGSTKYNPSLRSRVTISVDKSKNQFSLKLSSVTAADTAVYYCARRDNWKTPTTKIDGFDIWGQGTMVTVSS

SEQ ID NO: 79 - protein, 247 aa, synthetic construct
  REGION 1..247: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRSRGGGGSGGGGSGGGGSLEMAQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSQWGSSWDYWGQGTLVTVSS

SEQ ID NO: 80 - protein, 257 aa, synthetic construct
  REGION 1..257: note = Description of Artificial Sequence: Synthetic polypeptide
QPVLTQPPSASASLGASVTLTCTLSNDYTNYKVDWYQQRPGKGPRFVMRVGPGGIVGSKGDGIPDRFSVLGSGLNRYLTIKNIQEEDESDYHCGADHGTGSNFVYVFGGGTKLTVLGSRGGGGSGGGGSGGGGSLEMAEVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARSSYHLYGYDSWGQGTLVTVSS

SEQ ID NO: 81 - protein, 258 aa, synthetic construct
  REGION 1..258: note = Description of Artificial Sequence: Synthetic polypeptide
QPVLTQPPSASASLGASVTLTCTLSSGYSNYKVDWYQQRPGKGPRFLMRVDTGGIVGSKGDGIPDRFSVSGSGLNRYLTIKNIQEEDESDYHCGADHGSGSNFVWVFGGGTKLTVLGSRGGGGSGGGGSGGGGSLEMAQVQLVQSGAEVKKPGSSVKVSCKASGGTFSSYAISWVRQAPGQGLEWMGGIIPIFSTANYAQKFQGRVTMTTDTSTSTAYMELRSLRSDDTAVYYCARQPWTWYSPYDQWGQGTLVTVSS

SEQ ID NO: 82 - protein, 251 aa, synthetic construct
  REGION 1..251: note = Description of Artificial Sequence: Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRSRGGGGSGGGGSGGGGSLEMAEVQLVETGGGLVQPGGSLRLSCAASGFTFSTYAMTWVRQAPGKGLEWVSAITPGGDRTYYADSVKGRFTISRDNSRNTLYLQMNSLRAEDTAVYYCARYYGYMIDMWGQGTLVTVSS

SEQ ID NO: 83 - protein, 250 aa, synthetic construct
  REGION 1..250: note = Description of Artificial Sequence: Synthetic polypeptide
SYVLTQPPSASGTPGQRVTISCSGSSSNIGSNTVNWYQQVPGTAPKLLIYSNNQRPSGVPDRFSGSKSGASASLAISWLQSEDEADYYCAAWDDSLNGWVFGGGTKLTVLGSRGGGGSGGGGSGGGGSLEMAQMQLVQSGAEVKKPGASVKVSCKASGYTFTGYYVHWLRQAPGQGLEWMGWINPNSGGTNNAQEFQGRITMTRDTSINTAYMELSRLRSDDTAVYYCARSQWGGTYDYWGQGTLVTVSS

SEQ ID NO: 84 - protein, 249 aa, synthetic construct
  REGION 1..249: note = Description of Artificial Sequence: Synthetic polypeptide
QAVLTQPPSASGTPGQRVTISCSGSSSNIGSYTVSWYQQLPGTAPKFLIYSNNQRPSGVPDRFSGSKSGTSASLAISGLQSEDEADYYCAAWDDSLNGYVFGTGTKVTVLGSRGGGGSGGGGSGGGGSLEMAEVQLVQSGAEVKKPGESLKISCKGSGYDFTTYWIGWVRQMPGKGLEWMGIIYPGDSDTRYSPSVRGRVTISADKSINTAYLQWSSLEASDTAMYYCARMWTFSQDGWGQGTLVTVSS

SEQ ID NO: 85 - protein, 244 aa, synthetic construct
  REGION 1..244: note = Description of Artificial Sequence: Synthetic polypeptide
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 86 - protein, 251 aa, synthetic construct
  REGION 1..251: note = Description of Artificial Sequence: Synthetic polypeptide
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 87 - protein, 253 aa, synthetic construct
  REGION 1..253: note = Description of Artificial Sequence: Synthetic polypeptide
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 88 - protein, 248 aa, synthetic construct
  REGION 1..248: note = Description of Artificial Sequence: Synthetic polypeptide
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 89 - protein, 9 aa, synthetic construct
  REGION 1..9: note = Description of Artificial Sequence: Synthetic peptide
VSSNSAAWN

SEQ ID NO: 90 - protein, 7 aa, synthetic construct
  REGION 1..7: note = Description of Artificial Sequence: Synthetic peptide
YRSKWYN

SEQ ID NO: 91 - protein, 13 aa, synthetic construct
  REGION 1..13: note = Description of Artificial Sequence: Synthetic peptide
ARQGYSYYGYSDV

SEQ ID NO: 92 - protein, 8 aa, synthetic construct
  REGION 1..8: note = Description of Artificial Sequence: Synthetic peptide
SSNIGHND

SEQ ID NO: 93 - skipped

SEQ ID NO: 94 - protein, 11 aa, synthetic construct
  REGION 1..11: note = Description of Artificial Sequence: Synthetic peptide
AAWDGSLNAFV

SEQ ID NO: 95 - protein, 9 aa, synthetic construct
  REGION 1..9: note = Description of Artificial Sequence: Synthetic peptide
VSSNSAAWN

SEQ ID NO: 96 - protein, 7 aa, synthetic construct
  REGION 1..7: note = Description of Artificial Sequence: Synthetic peptide
YRSKWYN

SEQ ID NO: 97 - protein, 13 aa, synthetic construct
  REGION 1..13: note = Description of Artificial Sequence: Synthetic peptide
ARYGFSGSRFYDT

SEQ ID NO: 98 - protein, 8 aa, synthetic construct
  REGION 1..8: note = Description of Artificial Sequence: Synthetic peptide
SSNIGNNA

SEQ ID NO: 99 - skipped

SEQ ID NO: 100 - protein, 11 aa, synthetic construct
  REGION 1..11: note = Description of Artificial Sequence: Synthetic peptide
AAWDDSLNGYV

SEQ ID NO: 101 - protein, 8 aa, synthetic construct
  REGION 1..8: note = Description of Artificial Sequence: Synthetic peptide
GGTFSSYA

SEQ ID NO: 102 - protein, 8 aa, synthetic construct
  REGION 1..8: note = Description of Artificial Sequence: Synthetic peptide
IIPILGIA

SEQ ID NO: 103 - protein, 15 aa, synthetic construct
  REGION 1..15: note = Description of Artificial Sequence: Synthetic peptide
ARSGYSKSIVSYMDY

SEQ ID NO: 104 - protein, 8 aa, synthetic construct
  REGION 1..8: note = Description of Artificial Sequence: Synthetic peptide
SSNIGSNV

SEQ ID NO: 105 - skipped

SEQ ID NO: 106 - protein, 11 aa, synthetic construct
  REGION 1..11: note = Description of Artificial Sequence: Synthetic peptide
AAWDDSLSGYV

SEQ ID NO: 107 - protein, 8 aa, synthetic construct
  REGION 1..8: note = Description of Artificial Sequence: Synthetic peptide
GYTFTSYY

SEQ ID NO: 108 - protein, 8 aa, synthetic construct
  REGION 1..8: note = Description of Artificial Sequence: Synthetic peptide
INPSGGST

SEQ ID NO: 109 - protein, 11 aa, synthetic construct
  REGION 1..11: note = Description of Artificial Sequence: Synthetic peptide
ARSQWGGVLDY

SEQ ID NO: 110 - protein, 9 aa, synthetic construct
  REGION 1..9: note = Description of Artificial Sequence: Synthetic peptide
SSNIGARYD

SEQ ID NO: 111 - skipped

SEQ ID NO: 112 - protein, 11 aa, synthetic construct
  REGION 1..11: note = Description of Artificial Sequence: Synthetic peptide
QSYDSSLSASV

SEQ ID NO: 113 - protein, 8 aa, synthetic construct
  REGION 1..8: note = Description of Artificial Sequence: Synthetic peptide
GGTFSSYA

SEQ ID NO: 114 - protein, 8 aa, synthetic construct
  REGION 1..8: note = Description of Artificial Sequence: Synthetic peptide
IIPILGIA

SEQ ID NO: 115 - protein, 16 aa, synthetic construct
  REGION 1..16: note = Description of Artificial Sequence: Synthetic peptide
ARTGYESWGSYEVIDR

SEQ ID NO: 116 - protein, 8 aa, synthetic construct
  REGION 1..8: note = Description of Artificial Sequence: Synthetic peptide
SSNIGSNT

SEQ ID NO: 117 - skipped

SEQ ID NO: 118 - protein, 11 aa, synthetic construct
  REGION 1..11: note = Description of Artificial Sequence: Synthetic peptide
AAWDDSLNGVV

SEQ ID NO: 119 - protein, 8 aa, synthetic construct
  REGION 1..8: note = Description of Artificial Sequence: Synthetic peptide
GGTFSSYA

SEQ ID NO: 120 - protein, 8 aa, synthetic construct
  REGION 1..8: note = Description of Artificial Sequence: Synthetic peptide
IIPILGIA

SEQ ID NO: 121 - protein, 14 aa, synthetic construct
  REGION 1..14: note = Description of Artificial Sequence: Synthetic peptide
ARGGYYSHDMWSED

SEQ ID NO: 122 - protein, 8 aa, synthetic construct
  REGION 1..8: note = Description of Artificial Sequence: Synthetic peptide
SSNIGSNS

SEQ ID NO: 123 - skipped

SEQ ID NO: 124 - protein, 12 aa, synthetic construct
  REGION 1..12: note = Description of Artificial Sequence: Synthetic peptide
ATWDDNLNVHYV

SEQ ID NO: 125 - protein, 9 aa, synthetic construct
  REGION 1..9: note = Description of Artificial Sequence: Synthetic peptide
GGSISNSNW

SEQ ID NO: 126 - protein, 7 aa, synthetic construct
  REGION 1..7: note = Description of Artificial Sequence: Synthetic peptide
IYHSGST

SEQ ID NO: 127 - protein, 18 aa, synthetic construct
  REGION 1..18: note = Description of Artificial Sequence: Synthetic peptide
ARRDNWKTPTTKIDGFDI

SEQ ID NO: 128 - protein, 7 aa, synthetic construct
  REGION 1..7: note = Description of Artificial Sequence: Synthetic peptide
SGYSNYK

SEQ ID NO: 129 - protein, 8 aa, synthetic construct
  REGION 1..8: note = Description of Artificial Sequence: Synthetic peptide
VGTGGIVG

SEQ ID NO: 130 - protein, 13 aa, synthetic construct
  REGION 1..13: note = Description of Artificial Sequence: Synthetic peptide
GADHGSGSNFVYV

SEQ ID NO: 131 - protein, 8 aa, synthetic construct
  REGION 1..8: note = Description of Artificial Sequence: Synthetic peptide
GYTFTGYY

SEQ ID NO: 132 - protein, 8 aa, synthetic construct
  REGION 1..8: note = Description of Artificial Sequence: Synthetic peptide
INPNSGGT

SEQ ID NO: 133 - protein, 11 aa, synthetic construct
  REGION 1..11: note = Description of Artificial Sequence: Synthetic peptide
ARSQWGSSWDY

SEQ ID NO: 134 - protein, 6 aa, synthetic construct
  REGION 1..6: note = Description of Artificial Sequence: Synthetic peptide
QSISSY

SEQ ID NO: 135 - skipped

SEQ ID NO: 136 - protein, 9 aa, synthetic construct
  REGION 1..9: note = Description of Artificial Sequence: Synthetic peptide
QQSYSTPPT

SEQ ID NO: 137 - protein, 8 aa, synthetic construct
  REGION 1..8: note = Description of Artificial Sequence: Synthetic peptide
GYTFTGYY

SEQ ID NO: 138 - protein, 8 aa, synthetic construct
  REGION 1..8: note = Description of Artificial Sequence: Synthetic peptide
INPNSGGT

SEQ ID NO: 139 - protein, 12 aa, synthetic construct
  REGION 1..12: note = Description of Artificial Sequence: Synthetic peptide
ARSSYHLYGYDS

SEQ ID NO: 140 - protein, 7 aa, synthetic construct
  REGION 1..7: note = Description of Artificial Sequence: Synthetic peptide
NDYTNYK

SEQ ID NO: 141 - protein, 8 aa, synthetic construct
  REGION 1..8: note = Description of Artificial Sequence: Synthetic peptide
VGPGGIVG

SEQ ID NO: 142 - protein, 13 aa, synthetic construct
  REGION 1..13: note = Description of Artificial Sequence: Synthetic peptide
GADHGTGSNFVYV

SEQ ID NO: 143 - protein, 8 aa, synthetic construct
  REGION 1..8: note = Description of Artificial Sequence: Synthetic peptide
GGTFSSYA

SEQ ID NO: 144 - protein, 8 aa, synthetic construct
  REGION 1..8: note = Description of Artificial Sequence: Synthetic peptide
IIPIFSTA

SEQ ID NO: 145 - protein, 13 aa, synthetic construct
  REGION 1..13: note = Description of Artificial Sequence: Synthetic peptide
ARQPWTWYSPYDQ

SEQ ID NO: 146 - protein, 7 aa, synthetic construct
  REGION 1..7: note = Description of Artificial Sequence: Synthetic peptide
SGYSNYK

SEQ ID NO: 147 - protein, 8 aa, synthetic construct
  REGION 1..8: note = Description of Artificial Sequence: Synthetic peptide
VDTGGIVG

SEQ ID NO: 148 - protein, 13 aa, synthetic construct
  REGION 1..13: note = Description of Artificial Sequence: Synthetic peptide
GADHGSGSNFVWV

SEQ ID NO: 149 - protein, 8 aa, synthetic construct
  REGION 1..8: note = Description of Artificial Sequence: Synthetic peptide
GFTFSTYA

SEQ ID NO: 150 - protein, 8 aa, synthetic construct
  REGION 1..8: note = Description of Artificial Sequence: Synthetic peptide
ITPGGDRT

SEQ ID NO: 151 - protein, 10 aa, synthetic construct
  REGION 1..10: note = Description of Artificial Sequence: Synthetic peptide
ARYYGYMIDM

SEQ ID NO: 152 - protein, 11 aa, synthetic construct
  REGION 1..11: note = Description of Artificial Sequence: Synthetic peptide
QSLLHSNGYNY

SEQ ID NO: 153 - skipped

SEQ ID NO: 154 - protein, 9 aa, synthetic construct
  REGION 1..9: note = Description of Artificial Sequence: Synthetic peptide
MQALQTPLT

SEQ ID NO: 155 - protein, 8 aa, synthetic construct
  REGION 1..8: note = Description of Artificial Sequence: Synthetic peptide
GYTFTGYY

SEQ ID NO: 156 - protein, 8 aa, synthetic construct
  REGION 1..8: note = Description of Artificial Sequence: Synthetic peptide
INPNSGGT

SEQ ID NO: 157 - protein, 11 aa, synthetic construct
  REGION 1..11: note = Description of Artificial Sequence: Synthetic peptide
ARSQWGGTYDY

SEQ ID NO: 158 - protein, 8 aa, synthetic construct
  REGION 1..8: note = Description of Artificial Sequence: Synthetic peptide
SSNIGSNT

SEQ ID NO: 159 - skipped

SEQ ID NO: 160 - protein, 11 aa, synthetic construct
  REGION 1..11: note = Description of Artificial Sequence: Synthetic peptide
AAWDDSLNGWV

SEQ ID NO: 161 - protein, 8 aa, synthetic construct
  REGION 1..8: note = Description of Artificial Sequence: Synthetic peptide
GYDFTTYW

SEQ ID NO: 162 - protein, 8 aa, synthetic construct
  REGION 1..8: note = Description of Artificial Sequence: Synthetic peptide
IYPGDSDT

SEQ ID NO: 163 - protein, 10 aa, synthetic construct
  REGION 1..10: note = Description of Artificial Sequence: Synthetic peptide
ARMWTFSQDG

SEQ ID NO: 164 - protein, 8 aa, synthetic construct
  REGION 1..8: note = Description of Artificial Sequence: Synthetic peptide
SSNIGSYT

SEQ ID NO: 165 - skipped

SEQ ID NO: 166 - protein, 11 aa, synthetic construct
  REGION 1..11: note = Description of Artificial Sequence: Synthetic peptide
AAWDDSLNGYV

SEQ ID NO: 167 - protein, 8 aa, synthetic construct
  REGION 1..8: note = Description of Artificial Sequence: Synthetic peptide
GYTFIDYY

SEQ ID NO: 168 - protein, 8 aa, synthetic construct
  REGION 1..8: note = Description of Artificial Sequence: Synthetic peptide
INPNSGGT

SEQ ID NO: 169 - protein, 11 aa, synthetic construct
  REGION 1..11: note = Description of Artificial Sequence: Synthetic peptide
ARSQRDGYMDY

SEQ ID NO: 170 - protein, 9 aa, synthetic construct
  REGION 1..9: note = Description of Artificial Sequence: Synthetic peptide
ISCTGTSSD

SEQ ID NO: 171 - skipped

SEQ ID NO: 172 - protein, 10 aa, synthetic construct
  REGION 1..10: note = Description of Artificial Sequence: Synthetic peptide
SSNTRSSTLV

SEQ ID NO: 173 - protein, 8 aa, synthetic construct
  REGION 1..8: note = Description of Artificial Sequence: Synthetic peptide
GYTFTDYY

SEQ ID NO: 174 - protein, 8 aa, synthetic construct
  REGION 1..8: note = Description of Artificial Sequence: Synthetic peptide
INPNSGGT

SEQ ID NO: 175 - protein, 11 aa, synthetic construct
  REGION 1..11: note = Description of Artificial Sequence: Synthetic peptide
ARSPYSGVLDK

SEQ ID NO: 176 - protein, 9 aa, synthetic construct
  REGION 1..9: note = Description of Artificial Sequence: Synthetic peptide
SSNIGAGFD

SEQ ID NO: 177 - skipped

SEQ ID NO: 178 - protein, 11 aa, synthetic construct
  REGION 1..11: note = Description of Artificial Sequence: Synthetic peptide
QSYDSSLSGYV

SEQ ID NO: 179 - protein, 8 aa, synthetic construct
  REGION 1..8: note = Description of Artificial Sequence: Synthetic peptide
GGTFSSYA

SEQ ID NO: 180 - protein, 8 aa, synthetic construct
  REGION 1..8: note = Description of Artificial Sequence: Synthetic peptide
IIPILGTA

SEQ ID NO: 181 - protein, 13 aa, synthetic construct
  REGION 1..13: note = Description of Artificial Sequence: Synthetic peptide
ARSGYGSYRWEDS

SEQ ID NO: 182 - protein, 8 aa, synthetic construct
  REGION 1..8: note = Description of Artificial Sequence: Synthetic peptide
SSNIGSNY

SEQ ID NO: 183 - skipped

SEQ ID NO: 184 - protein, 12 aa, synthetic construct
  REGION 1..12: note = Description of Artificial Sequence: Synthetic peptide
AAWDDSLSASYV

SEQ ID NO: 185 - protein, 8 aa, synthetic construct
  REGION 1..8: note = Description of Artificial Sequence: Synthetic peptide
GYSFTSYW

SEQ ID NO: 186 - protein, 8 aa, synthetic construct
  REGION 1..8: note = Description of Artificial Sequence: Synthetic peptide
IYPGDSDT

SEQ ID NO: 187 - protein, 9 aa, synthetic construct
  REGION 1..9: note = Description of Artificial Sequence: Synthetic peptide
ARYSGSFDN

SEQ ID NO: 188 - protein, 8 aa, synthetic construct
  REGION 1..8: note = Description of Artificial Sequence: Synthetic peptide
SSNIGSHS

SEQ ID NO: 189 - skipped

SEQ ID NO: 190 - protein, 11 aa, synthetic construct
  REGION 1..11: note = Description of Artificial Sequence: Synthetic peptide
AAWDGSLNGLV

SEQ ID NO: 191 - protein, 20 aa, synthetic construct
  REGION 1..20: note = Description of Artificial Sequence: Synthetic peptide
MALPVTALLLPLALLLHAAR

SEQ ID NO: 192 - DNA, 60 bp, synthetic construct
  misc_feature 1..60: note = Description of Artificial Sequence: Synthetic oligonucleotide
  source 1..60: mol_type = other DNA
atggctctcccagtgactgccctactgcttcccctagcgcttctcctgcatgcagctcgt

SEQ ID NO: 193 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 194 - DNA, 321 bp, Homo sapiens
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 195 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - DNA, 339 bp, Homo sapiens
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 197 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 198 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 199 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 200 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 201 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 202 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 203 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 204 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 205 - protein, 20 aa, synthetic construct
  REGION 1..20: note = Description of Artificial Sequence: Synthetic peptide
METDTLLLWVLLLWVPGSTG

SEQ ID NO: 206 - DNA, 60 bp, synthetic construct
  misc_feature 1..60: note = Description of Artificial Sequence: Synthetic oligonucleotide
  source 1..60: mol_type = other DNA
atggaaaccgacaccctgctgctgtgggtgctgctgctgtgggtgccaggatccacagga

SEQ ID NO: 207 - DNA, 1398 bp, synthetic construct
  misc_feature 1..1398: note = Description of Artificial Sequence: Synthetic polynucleotide
  source 1..1398: mol_type = other DNA
caatctgccctgactcagcctgcctccgtgtctgcgtctcctggacagtcgatcgccatctcctgcactggaaccagcagtgacgttggttggtatcaacagcacccaggcaaagcccccaaactcatgatttatgaggacagtaagcggccctcaggggtttctaatcgcttctctggctccaagtctggcaacacggcctccctgaccatctctgggctccaggctgaggacgaggctgattattactgcagctcaaatacaagaagcagcactttggtgttcggcggagggaccaagctgaccgtcctaggttctagaggtggtggtggtagcggcggcggcggctctggtggtggtggatccctcgagatggccgaagtgcagctggtgcagtctggggctgagatgaagaagcctggggcctcactgaagctctcctgcaaggcttctggatacaccttcatcgactactatgtatactggatgcgacaggcccctggacaagggcttgagtccatgggatggatcaaccctaacagtggtggcacaaactatgcacagaagtttcagggcagggtcaccatgaccagggacacgtccatcagcacagcctacatggagctgagcaggctgagatctgacgacaccgccatgtattactgtgcgcgctcccagcgtgacggttacatggattactggggtcaaggtactctggtgaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 208 - DNA, 1419 bp, synthetic construct
  misc_feature 1..1419: note = Description of Artificial Sequence: Synthetic polynucleotide
  source 1..1419: mol_type = other DNA
cagtctgtgctgacgcagccgccctcagtgtctggggccccagggcagagggtcaccatctcctgcactgggagcagctccaacatcggggcaggttttgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttatgtcttcggaactgggaccaaggtcaccgtcctaggttctagaggtggtggtggtagcggcggcggcggctctggtggtggtggatccctcgagatggcccaggtccagctggtacagtctggggctgaggtgaagaagcctggggcctcagtgaaggtctcctgcaaggcttctggatacaccttcaccgactactatatgcactgggtgcgacaggcccctggacaacggcttgagtggatgggatggatcaaccctaacagtggtggcacaaactatgcacagaagtttcaggacaggatcaccgtgaccagggacacctccagcaacacaggctacatggagctgaccaggctgagatctgacgacacggccgtgtattactgtgcgcgctctccgtactctggtgttctggataaatggggtcaaggtactctggtgaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 209 - DNA, 1410 bp, synthetic construct
  misc_feature 1..1410: note = Description of Artificial Sequence: Synthetic polynucleotide
  source 1..1410: mol_type = other DNA
tcctatgagctgactcagccaccctcagcgtctgggacccccgggcagagggtcaccatgtcttgttctggaaccagctccaacatcggaagtcactctgtaaactggtaccagcagctcccaggaacggcccccaaactcctcatctatactaataatcagcggccctcaggggtccctgaccgattctctggctccaagtctggcacctcagcctccctggccatcagtggcctccagtctgaggatgaggctgattattactgtgcagcatgggatggcagcctgaatggtctggtattcggcggagggaccaagctgaccgtcctaggttctagaggtggtggtggtagcggcggcggcggctctggtggtggtggatccctcgagatggccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccacgtcaccatctcagctgacaagtccatcagcactgcctacctgcagtggagcagcctgaaggcctcggacaccgccatgtattactgtgcgcgctactctggttctttcgataactggggtcaaggtactctggtgaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 210 - protein, 15 aa, synthetic construct
  REGION 1..15: note = Description of Artificial Sequence: Synthetic peptide
GGGGSGGGGSGGGGS

SEQ ID NO: 211 - DNA, 45 bp, synthetic construct
  misc_feature 1..45: note = Description of Artificial Sequence: Synthetic oligonucleotide
  source 1..45: mol_type = other DNA
ggtggaggtggatcaggtggaggtggatctggtggaggtggatct

SEQ ID NO: 212 - protein, 5 aa, synthetic construct
  REGION 1..5: note = Description of Artificial Sequence: Synthetic peptide
GGGGS

SEQ ID NO: 213 - protein, 7 aa, synthetic construct
  REGION 1..7: note = Description of Artificial Sequence: Synthetic peptide
SGGSGGS

SEQ ID NO: 214 - protein, 9 aa, synthetic construct
  REGION 1..9: note = Description of Artificial Sequence: Synthetic peptide
GGGGSGGGS

SEQ ID NO: 215 - protein, 10 aa, synthetic construct
  REGION 1..10: note = Description of Artificial Sequence: Synthetic peptide
GGGGSGGGGS

SEQ ID NO: 216 - protein, 18 aa, synthetic construct
  REGION 1..18: note = Description of Artificial Sequence: Synthetic peptide
GGGGSGGGGSGGGGGGGS

SEQ ID NO: 217 - protein, 20 aa, synthetic construct
  REGION 1..20: note = Description of Artificial Sequence: Synthetic peptide
GGGGSGGGGSGGGGSGGGGS

SEQ ID NO: 218 - protein, 25 aa, synthetic construct
  REGION 1..25: note = Description of Artificial Sequence: Synthetic peptide
GGGGSGGGGSGGGGSGGGGSGGGGS

SEQ ID NO: 219 - protein, 30 aa, synthetic construct
  REGION 1..30: note = Description of Artificial Sequence: Synthetic polypeptide
GGGGSGGGGSGGGGSGGGGSGGGGSGGGGS

SEQ ID NO: 220 - protein, 35 aa, synthetic construct
  REGION 1..35: note = Description of Artificial Sequence: Synthetic polypeptide
GGGGSGGGGSGGGGSGGGGSGGGGSGGGGSGGGGS

SEQ ID NO: 221 - protein, 15 aa, synthetic construct
  REGION 1..15: note = Description of Artificial Sequence: Synthetic peptide
EPKSCDKTHTCPPCP

SEQ ID NO: 222 - protein, 24 aa, synthetic construct
  REGION 1..24: note = Description of Artificial Sequence: Synthetic peptide
GGGGSGGGSEPKSCDKTHTCPPCP

SEQ ID NO: 223 - protein, 62 aa, synthetic construct
  REGION 1..62: note = Description of Artificial Sequence: Synthetic polypeptide
ELKTPLGDTTHTCPRCPEPKSCDTPPPCPRCPEPKSCDTPPPCPRCPEPKSCDTPPPCPRCP

SEQ ID NO: 224 - protein, 6 aa, synthetic construct
  REGION 1..6: note = Description of Artificial Sequence: Synthetic peptide
GSGSGS

SEQ ID NO: 225 - skipped

SEQ ID NO: 226 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 227 - DNA, 213 bp, Homo sapiens
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 228 - DNA, 126 bp, Homo sapiens
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 229 - DNA, 7671 bp, synthetic construct
  misc_feature 1..7671: note = Description of Artificial Sequence: Synthetic polynucleotide
  source 1..7671: mol_type = other DNA
ccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtgccgccaccatggaaaccgacaccctgctgctgtgggtgctgctgctgtgggtgccaggatccacaggactgcctgtgctgactcagccaccctcagcgtctgggacccccgggcagagggtcaccatctcttgttctggacgcagttccaacatcgggagtaattctgttaactggtatcgacaactcccaggagcggcccccaaactcctcatctatagtaataatcagcggcccccaggggtccctgtgcgattctctggctccaagtctggcacctcagcctccctggccatcagtgggctccagtctgaagatgaggccacttattactgtgcaacatgggatgacaatctgaatgttcactatgtcttcggaactgggaccaaggtcaccgtcctaggttctagaggtggtggtggtagcggcggcggcggctctggtggtggtggatccctcgagatggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgcgcggtggttactactctcatgacatgtggtctgaagattggggtcaaggtactctggtgaccgtctcctcagcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcact

SEQ ID NO: 230 - DNA, 7653 bp, synthetic construct
  misc_feature 1..7653: note = Description of Artificial Sequence: Synthetic polynucleotide
  source 1..7653: mol_type = other DNA
ggccctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtgccgccaccatggaaaccgacaccctgctgctgtgggtgctgctgctgtgggtgccaggatccacaggatcctatgagctgactcagccaccctcagcgtctgggacccccgggcagagggtcaccatgtcttgttctggaaccagctccaacatcggaagtcactctgtaaactggtaccagcagctcccaggaacggcccccaaactcctcatctatactaataatcagcggccctcaggggtccctgaccgattctctggctccaagtctggcacctcagcctccctggccatcagtggcctccagtctgaggatgaggctgattattactgtgcagcatgggatggcagcctgaatggtctggtattcggcggagggaccaagctgaccgtcctaggttctagaggtggtggtggtagcggcggcggcggctctggtggtggtggatccctcgagatggccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccacgtcaccatctcagctgacaagtccatcagcactgcctacctgcagtggagcagcctgaaggcctcggacaccgccatgtattactgtgcgcgctactctggttctttcgataactggggtcaaggtactctggtgaccgtctcctcagcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcact

SEQ ID NO: 231 - DNA, 7668 bp, synthetic construct
  misc_feature 1..7668: note = Description of Artificial Sequence: Synthetic polynucleotide
  source 1..7668: mol_type = other DNA
ccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtgccgccaccatggaaaccgacaccctgctgctgtgggtgctgctgctgtgggtgccaggatccacaggacaggctgtgctgactcagccaccctcagcgtctgggacccccgggcagagggtcaccatctcttgttctggaagcagctccaacatcggaagtaattacgtattctggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtgcctcttatgttttcggaactgggaccaaggtcaccgtcctaggttctagaggtggtggtggtagcggcggcggcggctctggtggtggtggatccctcgagatggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtacagcaaactacgcacagaagttccagggcagagtcacgattaccgcggacgaatccacgagcacagcctacatggagctgagcagcctgagatctgaggacacggccgtgtattactgtgcgcgctctggttacggttcttaccgttgggaagattcttggggtcaaggtactctggtgaccgtctcctcagcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcact

SEQ ID NO: 232 - DNA, 7662 bp, synthetic construct
  misc_feature 1..7662: note = Description of Artificial Sequence: Synthetic polynucleotide
  source 1..7662: mol_type = other DNA
ccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtgccgccaccatggaaaccgacaccctgctgctgtgggtgctgctgctgtgggtgccaggatccacaggacagtctgtgctgacgcagccgccctcagtgtctggggccccagggcagagggtcaccatctcctgcactgggagcagctccaacatcggggcaggttttgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttatgtcttcggaactgggaccaaggtcaccgtcctaggttctagaggtggtggtggtagcggcggcggcggctctggtggtggtggatccctcgagatggcccaggtccagctggtacagtctggggctgaggtgaagaagcctggggcctcagtgaaggtctcctgcaaggcttctggatacaccttcaccgactactatatgcactgggtgcgacaggcccctggacaacggcttgagtggatgggatggatcaaccctaacagtggtggcacaaactatgcacagaagtttcaggacaggatcaccgtgaccagggacacctccagcaacacaggctacatggagctgaccaggctgagatctgacgacacggccgtgtattactgtgcgcgctctccgtactctggtgttctggataaatggggtcaaggtactctggtgaccgtctcctcagcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcact

SEQ ID NO: 233 - DNA, 7641 bp, synthetic construct
  misc_feature 1..7641: note = Description of Artificial Sequence: Synthetic polynucleotide
  source 1..7641: mol_type = other DNA
ccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtgccgccaccatggaaaccgacaccctgctgctgtgggtgctgctgctgtgggtgccaggatccacaggacaatctgccctgactcagcctgcctccgtgtctgcgtctcctggacagtcgatcgccatctcctgcactggaaccagcagtgacgttggttggtatcaacagcacccaggcaaagcccccaaactcatgatttatgaggacagtaagcggccctcaggggtttctaatcgcttctctggctccaagtctggcaacacggcctccctgaccatctctgggctccaggctgaggacgaggctgattattactgcagctcaaatacaagaagcagcactttggtgttcggcggagggaccaagctgaccgtcctaggttctagaggtggtggtggtagcggcggcggcggctctggtggtggtggatccctcgagatggccgaagtgcagctggtgcagtctggggctgagatgaagaagcctggggcctcactgaagctctcctgcaaggcttctggatacaccttcatcgactactatgtatactggatgcgacaggcccctggacaagggcttgagtccatgggatggatcaaccctaacagtggtggcacaaactatgcacagaagtttcagggcagggtcaccatgaccagggacacgtccatcagcacagcctacatggagctgagcaggctgagatctgacgacaccgccatgtattactgtgcgcgctcccagcgtgacggttacatggattactggggtcaaggtactctggtgaccgtctcctcagcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcact

SEQ ID NO: 234 - DNA, 1491 bp, synthetic construct
  misc_feature 1..1491: note = Description of Artificial Sequence: Synthetic polynucleotide
  source 1..1491: mol_type = other DNA
atggaaaccgacaccctgctgctgtgggtgctgctgctgtgggtgccaggatccacaggactgcctgtgctgactcagccaccctcagcgtctgggacccccgggcagagggtcaccatctcttgttctggacgcagttccaacatcgggagtaattctgttaactggtatcgacaactcccaggagcggcccccaaactcctcatctatagtaataatcagcggcccccaggggtccctgtgcgattctctggctccaagtctggcacctcagcctccctggccatcagtgggctccagtctgaagatgaggccacttattactgtgcaacatgggatgacaatctgaatgttcactatgtcttcggaactgggaccaaggtcaccgtcctaggttctagaggtggtggtggtagcggcggcggcggctctggtggtggtggatccctcgagatggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgcgcggtggttactactctcatgacatgtggtctgaagattggggtcaaggtactctggtgaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 235 - DNA, 1488 bp, synthetic construct
  misc_feature 1..1488: note = Description of Artificial Sequence: Synthetic polynucleotide
  source 1..1488: mol_type = other DNA
atggaaaccgacaccctgctgctgtgggtgctgctgctgtgggtgccaggatccacaggacaggctgtgctgactcagccaccctcagcgtctgggacccccgggcagagggtcaccatctcttgttctggaagcagctccaacatcggaagtaattacgtattctggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtgcctcttatgttttcggaactgggaccaaggtcaccgtcctaggttctagaggtggtggtggtagcggcggcggcggctctggtggtggtggatccctcgagatggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtacagcaaactacgcacagaagttccagggcagagtcacgattaccgcggacgaatccacgagcacagcctacatggagctgagcagcctgagatctgaggacacggccgtgtattactgtgcgcgctctggttacggttcttaccgttgggaagattcttggggtcaaggtactctggtgaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 236 - protein, 19 aa, synthetic construct
  REGION 1..19: note = Description of Artificial Sequence: Synthetic peptide
SGSGLQMAGQCSQNEYFDS

SEQ ID NO: 237 - protein, 19 aa, synthetic construct
  REGION 1..19: note = Description of Artificial Sequence: Synthetic peptide
SGSGQMAGQCSQNEYFDSL

SEQ ID NO: 238 - protein, 19 aa, synthetic construct
  REGION 1..19: note = Description of Artificial Sequence: Synthetic peptide
SGSGMAGQCSQNEYFDSLL

SEQ ID NO: 239 - protein, 19 aa, synthetic construct
  REGION 1..19: note = Description of Artificial Sequence: Synthetic peptide
SGSGAGQCSQNEYFDSLLH

SEQ ID NO: 240 - protein, 19 aa, synthetic construct
  REGION 1..19: note = Description of Artificial Sequence: Synthetic peptide
SGSGGQCSQNEYFDSLLHA

SEQ ID NO: 241 - protein, 19 aa, synthetic construct
  REGION 1..19: note = Description of Artificial Sequence: Synthetic peptide
SGSGQCSQNEYFDSLLHAC

SEQ ID NO: 242 - protein, 19 aa, synthetic construct
  REGION 1..19: note = Description of Artificial Sequence: Synthetic peptide
SGSGCSQNEYFDSLLHACI

SEQ ID NO: 243 - protein, 19 aa, synthetic construct
  REGION 1..19: note = Description of Artificial Sequence: Synthetic peptide
SGSGSQNEYFDSLLHACIP

SEQ ID NO: 244 - protein, 19 aa, synthetic construct
  REGION 1..19: note = Description of Artificial Sequence: Synthetic peptide
SGSGQNEYFDSLLHACIPC

SEQ ID NO: 245 - protein, 19 aa, synthetic construct
  REGION 1..19: note = Description of Artificial Sequence: Synthetic peptide
SGSGNEYFDSLLHACIPCQ

SEQ ID NO: 246 - protein, 19 aa, synthetic construct
  REGION 1..19: note = Description of Artificial Sequence: Synthetic peptide
SGSGEYFDSLLHACIPCQL

SEQ ID NO: 247 - protein, 19 aa, synthetic construct
  REGION 1..19: note = Description of Artificial Sequence: Synthetic peptide
SGSGYFDSLLHACIPCQLR

SEQ ID NO: 248 - protein, 19 aa, synthetic construct
  REGION 1..19: note = Description of Artificial Sequence: Synthetic peptide
SGSGFDSLLHACIPCQLRC

SEQ ID NO: 249 - protein, 19 aa, synthetic construct
  REGION 1..19: note = Description of Artificial Sequence: Synthetic peptide
SGSGDSLLHACIPCQLRCS

SEQ ID NO: 250 - protein, 19 aa, synthetic construct
  REGION 1..19: note = Description of Artificial Sequence: Synthetic peptide
SGSGSLLHACIPCQLRCSS

SEQ ID NO: 251 - protein, 19 aa, synthetic construct
  REGION 1..19: note = Description of Artificial Sequence: Synthetic peptide
SGSGLLHACIPCQLRCSSN

SEQ ID NO: 252 - protein, 19 aa, synthetic construct
  REGION 1..19: note = Description of Artificial Sequence: Synthetic peptide
SGSGLHACIPCQLRCSSNT

SEQ ID NO: 253 - protein, 19 aa, synthetic construct
  REGION 1..19: note = Description of Artificial Sequence: Synthetic peptide
SGSGHACIPCQLRCSSNTP

SEQ ID NO: 254 - protein, 19 aa, synthetic construct
  REGION 1..19: note = Description of Artificial Sequence: Synthetic peptide
SGSGACIPCQLRCSSNTPP

SEQ ID NO: 255 - protein, 19 aa, synthetic construct
  REGION 1..19: note = Description of Artificial Sequence: Synthetic peptide
SGSGCIPCQLRCSSNTPPL

SEQ ID NO: 256 - protein, 19 aa, synthetic construct
  REGION 1..19: note = Description of Artificial Sequence: Synthetic peptide
SGSGIPCQLRCSSNTPPLT

SEQ ID NO: 257 - protein, 19 aa, synthetic construct
  REGION 1..19: note = Description of Artificial Sequence: Synthetic peptide
SGSGPCQLRCSSNTPPLTC

SEQ ID NO: 258 - protein, 19 aa, synthetic construct
  REGION 1..19: note = Description of Artificial Sequence: Synthetic peptide
SGSGCQLRCSSNTPPLTCQ

SEQ ID NO: 259 - protein, 19 aa, synthetic construct
  REGION 1..19: note = Description of Artificial Sequence: Synthetic peptide
SGSGQLRCSSNTPPLTCQR

SEQ ID NO: 260 - protein, 19 aa, synthetic construct
  REGION 1..19: note = Description of Artificial Sequence: Synthetic peptide
SGSGLRCSSNTPPLTCQRY

SEQ ID NO: 261 - protein, 19 aa, synthetic construct
  REGION 1..19: note = Description of Artificial Sequence: Synthetic peptide
SGSGRCSSNTPPLTCQRYC

SEQ ID NO: 262 - protein, 19 aa, synthetic construct
  REGION 1..19: note = Description of Artificial Sequence: Synthetic peptide
SGSGCSSNTPPLTCQRYCN

SEQ ID NO: 263 - protein, 19 aa, synthetic construct
  REGION 1..19: note = Description of Artificial Sequence: Synthetic peptide
SGSGSSNTPPLTCQRYCNA

SEQ ID NO: 264 - protein, 19 aa, synthetic construct
  REGION 1..19: note = Description of Artificial Sequence: Synthetic peptide
SGSGSNTPPLTCQRYCNAS

SEQ ID NO: 265 - protein, 19 aa, synthetic construct
  REGION 1..19: note = Description of Artificial Sequence: Synthetic peptide
SGSGNTPPLTCQRYCNASV

SEQ ID NO: 266 - protein, 19 aa, synthetic construct
  REGION 1..19: note = Description of Artificial Sequence: Synthetic peptide
SGSGTPPLTCQRYCNASVT

SEQ ID NO: 267 - protein, 19 aa, synthetic construct
  REGION 1..19: note = Description of Artificial Sequence: Synthetic peptide
SGSGPPLTCQRYCNASVTN

SEQ ID NO: 268 - protein, 19 aa, synthetic construct
  REGION 1..19: note = Description of Artificial Sequence: Synthetic peptide
SGSGPLTCQRYCNASVTNS

SEQ ID NO: 269 - protein, 19 aa, synthetic construct
  REGION 1..19: note = Description of Artificial Sequence: Synthetic peptide
SGSGLTCQRYCNASVTNSV

SEQ ID NO: 270 - protein, 19 aa, synthetic construct
  REGION 1..19: note = Description of Artificial Sequence: Synthetic peptide
SGSGTCQRYCNASVTNSVK

SEQ ID NO: 271 - protein, 19 aa, synthetic construct
  REGION 1..19: note = Description of Artificial Sequence: Synthetic peptide
SGSGCQRYCNASVTNSVKG

SEQ ID NO: 272 - protein, 19 aa, synthetic construct
  REGION 1..19: note = Description of Artificial Sequence: Synthetic peptide
SGSGQRYCNASVTNSVKGT

SEQ ID NO: 273 - protein, 19 aa, synthetic construct
  REGION 1..19: note = Description of Artificial Sequence: Synthetic peptide
SGSGRYCNASVTNSVKGTN

SEQ ID NO: 274 - protein, 19 aa, synthetic construct
  REGION 1..19: note = Description of Artificial Sequence: Synthetic peptide
SGSGYCNASVTNSVKGTNA

SEQ ID NO: 275 - protein, 25 aa, synthetic construct
  REGION 1..25: note = Description of Artificial Sequence: Synthetic peptide
TSGQAGQHHHHHHGAYPYDVPDYAS

SEQ ID NO: 276 - DNA, 75 bp, synthetic construct
  misc_feature 1..75: note = Description of Artificial Sequence: Synthetic oligonucleotide
  source 1..75: mol_type = other DNA
actagtggccaggccggccagcaccatcaccatcaccatggcgcatacccgtacgacgttccggactacgcttct

SEQ ID NO: 277 - protein, 4 aa, Homo sapiens
YVKM

SEQ ID NO: 278 - protein, 4 aa, synthetic construct
  REGION 1..4: note = Description of Artificial Sequence: Synthetic peptide
SGSG

SEQ ID NO: 279 - protein, 5 aa, synthetic construct
  REGION 1..5: note = Description of Artificial Sequence: Synthetic peptide
GSSSS

SEQ ID NO: 280 - protein, 53 aa, synthetic construct
  REGION 1..53: note = Description of Artificial Sequence: Synthetic polypeptide
LQMAGQCSQNEYFDSLLHACIPCQLRCSSNTPPLTCQRYCNASVTNSVKGTNA